SEQUENCE LISTING (150 sequences)

SEQ ID NO: 1 - protein, 480 aa, Lotus japonicus
MQGFKTAQGNSHLYCHPSSFLLRGDDTSRNHTRFSDLGELQHSSNIFQQDDAVDLSSSNLPCSMYSVKSCNVVGGGSNMQYGTLNTNLGSAEIVSTGAGCLETGQQLMYHKEISMATMPMGNGHVENWADSSGMADNSQQTDDTSTDVDTDDKIQSNRDKRGTHMVVHSRDQTKSKPEDQKILRRLAQNREAAKKSRLRKKAYVQQLENSRLKLVQLEQELQRARQQGVFMASPGDQSHLAVGGANGASAFDMEYAHWVDEHQRLLTDLRSALNSQMGDNELHLLVDGVMAHYDELFRLKSLGAKADVFHMHSGLWKTPAERCFMWLGGFRSTELLKIVKNHLEPLTEPQLMGIDNLLRCAQQGEDALTQGMEALQQALVDTLSSTSLGPAGAGNVADYMGQMAIAMGKLATLESFLHSADLVKQQTLQQLQRILTTRQMARALLVRNDYISRLRALSSLWLARPRESSVMSSLPPSNIE

SEQ ID NO: 2 - protein, 445 aa, Amborella trichopoda
MQSFAPPPGSYALGAESSRGHSRVSDAGELEQSTGFRVDDAVNLSRGIIFNPKPGSPVVTSSPLKFGALNKQIDSSDIPSSTNRGELGRTSSLKGLQTNTGSLSNNQFDSWGDSGMADTSPRTDTSTDVDTDEKNQQFLEGQRASSDSLDRSKDKTGDQKTLRRLAQNREAARKSRLRKKAYVQQLETSRLKLQQLEQELQRARQQGIFIAGAGDQNHSMGGNGALAFDMEYARWLDEHQRQINDLRSAMNSHVGENELRILVDAVMQHYDEIFRIKGTGTKSDVFHMLSGMWKTPAERCFMWLGGFRSSELLKILLSHLEPLTEQQLMGICNLQQSSQQAEDALSQGMEALQQSLAETLASGSLGPPGSSGNVANYMGQMAMAMGKLGTLENFLRQADNLRQQTLQQLHRILTTRQSARALLAISDYFSRLRALSSLWLARPRE

SEQ ID NO: 3 - protein, 353 aa, Aquilegia coerulea
MALPSSSGNGGVYEAWGESGVVNANLQTDTSTDFDTDDRNQLIQAWHGALVSENSIEKSNGKKEEQKSLRRLAQNREAARKSRLRKKTYVQQLESGRLKLNQLEEELQRARQQGIVIPRGCIGDQSHSTGGNGSLAFDMEYTQWLDEHQRQINDLRSAINSHVEDNELRLLVDSIMTHYEELFKSKGRGAKCDVFHMLSGIWRTPAERFFMWLGGFRSSELLKIVGNHLEPLTDQQLMGICNLQQSSQQAEDALSQGMEALQQSLADTLSSGSLGPSANGNVANYMGQMALAMGKLATLENFLHQADLLRQQTLQQLYRILTIRQAARALLVISDYISRLRTLSSLWLARPKD

SEQ ID NO: 4 - protein, 452 aa, Arabidopsis thaliana
MQSSFKTVPFTPDFYSQSSYFFRGDSCLEEFHQPVNGFHHEEAIDLSPNVTIASANLHYTTFDTVMDCGGGGGGGLRERLEGGEEECLDTGQLVYQKGTRLVGGGVGEVNSSWCDSVSAMADNSQHTDTSTDIDTDDKTQLNGGHQGMLLATNCSDQSNVKSSDQRTLRRLAQNREAARKSRLRKKAYVQQLENSRIRLAQLEEELKRARQQGSLVERGVSADHTHLAAGNGVFSFELEYTRWKEEHQRMINDLRSGVNSQLGDNDLRVLVDAVMSHYDEIFRLKGIGTKVDVFHMLSGMWKTPAERFFMWLGGFRSSELLKILGNHVDPLTDQQLIGICNLQQSSQQAEDALSQGMEALQQSLLETLSSASMGPNSSANVADYMGHMAMAMGKLGTLENFLRQADLLRQQTLQQLHRILTTRQAARAFLVIHDYISRLRALSSLWLARPRD

SEQ ID NO: 5 - protein, 442 aa, Brassica oleracea
MQSSFKTVPFNPDFYSQASFFFRGDSCLDEFHQPINGFHHDEAVGLSPNVTVAASNNLHYTTFDTVMDYGMRERLEGEDECLDTGELMYQRGTRLVGGGGGEVNSSLDKWCDSVSAMADNSQHTDTSTDIDTDDKSQLNGVNQGMLLATNCSDQSKTLRRLAQNREAARKSRLRKKAYVQQLENSRIRLAQLEEELKRARQQGCSSERGVSRENPHVAAGNGVFSFELEYARWMEEHQRLINDLRAGVHSQLGDNELRVLVDAVMSHYDEIFRLKGIGTKVDVFHMLSGMWQTPAERFFMWLGGFRSSELLKILGNHVDPLTDQQLIGICNLQQSSQQAEDALSQGMEALQQSLLETLSSASMGPNSSANVADYMGHMAMAMGKLGTLENFLRQADLLRQQTLQQLHRILTTRQAARAFLVIHDYICRLRALSSLWLARPRD

SEQ ID NO: 6 - protein, 456 aa, Glycine max
MQSFNTTESESTSPLYSHSPFYHRGNDPSRNPTRFSDLQHSFRQHDAVDLSSSSVFGAKSSNVAVVASNLQCGTFNTNLGCAEFGSIEQRGMFQRGTNSATTVSMGNRHVENWAEDSQHTEDTCTDIDTDDKNQCFSTVSWCNGVGDGALVVVDSQDQSKTKVKAEDQKTVRRLAQNREAARKSRLRKKAYVQQLETSRVRLAQLEQELQRARQQGAFIATGNQGDRSHSAVGNGALAFDMDYARWFDEHQRLINDIRSAINSQMDENELHLLVDGVMAHYDELFRLKSIGAKADVFHILSGMWKTPAERCFIWLGGFRSSELLKIVRNQLEPLTEQQLMGIYNLQQSSQQAEDALSQGMDALQQSLSETLSSSSLGPSGSGNVAEYMGQMAIALGKLATLENFLHQADLLRQQTLQQMRRILTTFQAARALLVINDYVSRLRALNSLWLACPREY

SEQ ID NO: 7 - protein, 455 aa, Glycine max
MQSFNTTTESESISPLYCHSPFFHRGNDTSRNPSRFSDLQHSFRQHDAVDLSSSSVFGGKSSNVAVVASNLQCGTFNTNLGCAEFGSTEQGPMFQRGTTVSMATGNQHVENWADDSHPTEDTCTDIDTDDKNQCFSTVSWCHGVGDGALVVVDSHDQSKTKVKAEDQKTVRRLAQNREAAKKSRLRKKAYVQQLENSRVRLAQLEQELQRARQQGAFIATGIPGDRGHSSVANGALAFDMDYARWVDEHQRLIIDIRSAINSQMGENELHLLVDGAMAHYDELFRLKSIGAKVDVFHILSGMWKTPAERCFIWLGGFRSSELLKIVRNQLEPLTEQQLMGIYNLQQSSQQAEDALSQGMEALQQSLSETLSSSSLGPSGSENVAEYMGQMAIALGKLATLENFLHQADLLRQQTLQQMRRILTTCQAARALLVINDYVTRLRALNSLWLACPREY

SEQ ID NO: 8 - protein, 467 aa, Glycine max
MKNMQGFKTAQQPSSQQQLYCHSSFLLRGNDPNRNTTRFSDLGELHHSSSVFHHEDAADLSSSSMFSVKSNNVVGGSNIQYGTLNTLSTNVGSAEIGTSVRGCMDTGQQLMYHKGVTTAALPLGNGQVENWDDSGLADNSQQTDHTSTDIDTDDIIQCNRVKNGTRMVVHSKDATKVKPGDQKTLRRLAQNREAARKSRLRKKAYVQQLESSRVKLVHLEQELQRARQQGIFIATPGDQGHLAVGNGALAFDIDYAHWVDEHQRLLNDLRTAINSQMSDSDLHILVDSVMAHYNELFRLKSIGAKADVLHIHNGMWKTPVERCFMWLGGLRSSELLKIIKNHLEPLTDQQLMGICNLQQSSQQAEDALNQGMEALQQSLVEILSSTSLGPNGSGNVADYMGQMALAMGKLAVLGSFLHKADLLKQETLEQLQRILTTRQTARALLVQNDYISRLRALSSLWLARPRE

SEQ ID NO: 9 - protein, 486 aa, Glycine max
MPFSSQRHSRIFLPSHTSLFLFSLMKNMQGFKTAQPSSQQLYCHSSFLLRGNDPNQNPARFSDLGEHHHFSAVFHQEDAADLSSSSMFSVKSSNVVGGSNMQYGTLNTNVGHAEIGSSGGGCMDTRQQLMYHKGVTMAALPLGNGQVENWADSGKADNSQQTDDTSTDIDTDDIIPCNRVKNGTRMVVHSKDETKVKPGDQKTLRRLAQNREAARKSRLRKKAYVQQLESSRVKLVQLEQELQRARQQGIFIATPGDQGHLAVGNGALAFDIDYAHWVDEHQRLLNDLRTAVNSQMSDSDLHILVDSVMAHYNELFRLKSLGTKADVLHIHNGMWKTPVERCFMWLGGFRSSELLKIIKNHLEPLTDQQLMGIYNLQQSSQQAEDALSQGLEALQQSLVETLSSISLGPTDSGNVVDYMGQMALAMGKLADLESFVRQADLLKQQTLQQLQRILTTRQTARALLVQTDYISRLRALSSLWLARPRE

SEQ ID NO: 10 - protein, 485 aa, Eucalyptus grandis
MQSFKAAPPTNPDIYCPASFFLRGGDVMSRNQTRFPDLGELEQSVAAFHHEDAVDLSPSSAFSLKPNNSIAFVANNLSYGGLNPCMSSSGVVTVSSGGGGGGGGGGGGCVEMGEIYEKGGGNGGSLGNVQGIENWADSGMAAPDNSQQTDTSTDVDTDDKRHFYRVQRGALVAVDSADQSKMRMSDVKTLRRLAQNREAAKKSRLRKKAYVQQLENSRFRLMQLEQELQRARQQGIFVSSGNPGDLSHNMAAIGNGAMAFDTDYARWLDEHQRLINDLRSGVNSQLNDNELRMRVDAVMVHHDQIFRLKNIGAKADVFHILSGMWKTPAERCFMWLGGFRSSELLKILGNHLEPLTDQQLMGICNLQQSSQQAEDALSQGMEALQQSLVDTLSSTTLSPTGSGNVAEYMGQMAIAMGKLATLENFVHQADLLRQQTLQQMHRILTTRQAARALLVINDYISRLRALSSLWLARPRTENICSAKLF

SEQ ID NO: 11 - protein, 224 aa, Gossypium raimondii
MRELVNLKQQGSIFSLKSNNFGVFSNTLHYGALNMSIGATMIVSSGTGCLDTGQFMYQKGATFGAPLGNGQIENWADSGLANNSQQTDTSTDVDTDHTKQLPRVQHGAVMVNTVDQPKSKNGDQKTLRRLAQNQEAARKSRLRKKAYVQQLESSRLRLTELEQELERTQQQGIFIASGLSGDHGHTVAGNAALAFDMEYGRWFDEHQRLKLCNNLIHRMINYGR

SEQ ID NO: 12 - protein, 363 aa, Gossypium raimondii
MEVVCRCGILLMRQEFLLKTSRLECPKFDGNDFREWWTKLEQYFEVEGIPEVNKQGSIFCLKSNNVGVVANTLHYGALNMSIGATAIVSSGTGCLDTGQFMYQKGVTFGAPLGNGQMENWPDSDNSQQTDTSTDVDTDYKKIASWSSTWSCNAARKSRLRKKAYVQQLESSRLRFTELEQELQRARQQGIFIASGLSGDHGHTVAGNVALAFDMEYGRWLDEHQRLINNLRSAVFRLKNTGAKADVFHMLSGMWKTPIDMCFMWLGGFCSKAYVFHMLYGMWKTPAERCFLWLGGFHSSELMKIVKNHLEPLADQQQGFGRIGYLVVRVALQSNDIELIAVNDPFITTDYMTYMFKYDSVHGQ

SEQ ID NO: 13 - protein, 192 aa, Gossypium raimondii
MPSWPTYIKSLQDRFGCKPFWDSIRELVNLKQEGSISSLKSNNVGVVANTLHYGALNTSIGATATVSSGMGCLDTGQFMYQKGATLGAFLGNGQIKNWADSGLADYSQQTDTSTDVDTNHKNQLHGVQHGAVMVKTIDQSKSKSNDQKTLRRLAQNREAARNSRLRKKTYVQQLESSRLRLTELEQELQRAQ

SEQ ID NO: 14 - protein, 459 aa, Gossypium raimondii
MQSFKAAATTNPDMFCHSSFFLRGDDCNSRNQTRFPDLGELDHPAAAFHHDDAFDLSPSSIFSLKSNNVGVVANTLHYGALNTSIGATAIVSSGTGCLDTGQFMYQKGATFGASLGNGHIENWADSGLADNSQQTDTSTDVDTDHKNQLHGVQLGAVMVDSVDQSKSKSGDQKTLRRLAQNREAARKSRLRKKAYVQQLESSRLRLTELEQELQRARQQGIFIASGLSGDHGHTVAGNAALAFDMEYGRWLDEHQRLINDLRSAVNSHMGDNELCILVGGVMAHYDEVFRLKSIGAKADVFHMLSGMWKTPAERCFMWLGGFRSSELLKILGNHLEPLTDQQLMGICNLQQSSQQAEDALSQGMEALQQSLVDTLSSASLGPSASGNVADYMGQMAIAMGKLATLENFLHQADLLRQQTLQQMHRILTTRQAARALLVFSDYTSRLRALSSLWLARPRN

SEQ ID NO: 15 - protein, 437 aa, Gossypium raimondii
MQSFKAAAPHTTPDMFCHSSFFLRGDDDFNPANAFHHDDSVDLSSSSIFSLKSSNVDVVGNSLHYDAFSTGIRAAEIVSSGTGCLDTGQFMYQKGTTLGNGHIENWGDSGLADNSQQTDTSTDVDTDDKNQLQHGAIITVDQSKLKTGDQKTLRRLAQNREAARKSRLRKKAYVQQLESSQLRLTQLEQELQKARQQGFFIASGLSGGHGLSVSGNAALAFDMDYAHWLNEHQRLINDLRSGLNSHLGDSELRILVESVMAHYDEVFKLKSIGVKADAFHMLSGMWKTPAERCFMWLGGFRSSELLKILGNHLEPLTDQQLMGICNLQQSSQQAEDALSQGMEALQQALVDTLPSACLGPTACGNVADYMSQMAIAMSKLATLENFLHQADLLRHQTLQQMHRILTTRQAARALLVFSDYNSRLRALSSLWLARPRN

SEQ ID NO: 16 - protein, 466 aa, Manihot esculenta
MEVMSSFVFSDKMKFLIHFPVSIFLWVLGNRGEDGSRNQTRFADLGELEQPAPAFHHDDAVDLSPSSMFSLKSGNVAVLSSNLQYDAVLNTSIGSAEIATTGTGCLDTGQYMYHKGTTIASSLGNAHCIENWGDSGMADNSQQTDTSTDVDTDDRNQLHGVQHGTVMVVDSMEKSKAKAGDQKTLRRLAQNREAARKSRLRKKAYVQQLESSRHRLAQLEQELQRARQQGIFVASGLSGDHGAGNGAVAFNMDYARWLEEHQRLISDLRSAVNSHMGDNELHVLVDAVMSHYDEIFRLKSIGTKADVFHMLSGMWKTPAERCFMWLGGFRSSELLKILGNHLEPLTDQQLMGICNLQQSSQQAEDALSQGMEALQQSLVETVSSTSLGPAGSGKVADYMGQMAIAMGKLATLENFIHQADLLRQQTLQQMNRILTTRQAARALLVISDYTSRLRALSSLWLARPRD

SEQ ID NO: 17 - protein, 452 aa, Manihot esculenta
MQSFKHLPPPEMYCHSSFFLRGEDGSRNQARFRYLGELDQTASAFHPDDAIDLSPNSMFSLKSGNVAVLSNNFNYDPALNTSIGSAEIATTGTGCLDTGQYMHPKGTTIVSSLGNGHSIENWGHSGMADNGQQTDTSTDVDTDDRNQLHGVQHGPVVVVDSRPKTGDQKTLRRLAQNREAARKSRLRKKAYVQQLETSRLRLAQLEQELQRAQQQGVFVATGFSGDHGSGNGVVVFDMEYAHWLEEHQRLISDLRAAVSSHMGDNELHLHVDGVMAHYDEIFRLKSIGTKADVFHMLSGMWKTPAERCFMWLGGFRSSELLKILGNHLEPLTDQQIMGICNLQQSSQQAEDALSQGMEALQQSLVDTLSSTSLGFAGSGNVADYMGQMAIAMGKLATLENFLHQADLLRQQTLQQMRRILTTRQAARALLVFGDYASRLRALSSLWLARPRD

SEQ ID NO: 18 - protein, 464 aa, Prunus persica
MQSFKAASTNPELYSHSSFYFRGDDTNRNQTRFTDLGELEQSATVFPHDDAVDLIPSSMFSLKANNVAVVPNNLHYGALNTSTGCLDIGSTITGTGGGCVETGHHQYMYHQQKGTTSSGNGHFENWGDSAMADNSQQTDDTSTDVDTDDKNQLHGIPHGALTVVDSMEQPRERSGDQKTLRRLAQNREAARKSRLRKKAYVQQLENSRLRLSHLEQELQRARQQGMFIATGLSGDNGHSMAGNGALAFDLEYARWLDEHQRLIHDMRSAVNSHTGDNELRILVDSVMTHYDEIFRLKSIAAKTDVFHMLSGMWKTPAERCFMWLGGFRSSELLKILGNQLEPLTDQQLMGICNLQQSSQQAEDALSQGMEALQQSLVDTLCSGTHGPTGSGNVADYMGQMAIAMGKLATLENFLHQADLLRQQTLQQMHRILTTRQAARALLVISDYFSRLRALSSLWLARPRD

SEQ ID NO: 19 - protein, 484 aa, Solanum lycopersicum
MQAFKEAAVINNNNNSNSNSNSSMYCHSSYYLRGDEDSRLAARFADIGELEQSTGFTQDDAVDLSRSTVYGEMRPNNVGIVSCNNNNLHFGELNTSIGSSETGVETGRFMLQKAQTGMVGGGGGGGGVVGGGVALGNVQHFENWGDSGIADHSQQTDTSTDVDTDERNHQGQGVQHGALIAVDSMDLSKVKLGDQKTLRRLAQNREAARKSRLRKKAYVQQLENSRLKLSQLEQELKRARQQGIFIANGYAGDQSLSAGGNGALAFDMDYARWLDEHQRLISDLRSAVNSHRGDNELRHLVDGVMSHYDEKFKLKSVGLKADVFHMLSGMWKTPVERCFMWLGGFRSSELLKILGNHLEPFTEQQLIGICNLQQSSQQAEDALSQGMEALQQLLIDTLSSTSLGPSGSGNVADYMGQMAIAMNQLATLENFLYQADLLRQRTLQQLHRILTTRQAARALLVISDYMSRLRALSSLWLARPKDQL

SEQ ID NO: 20 - protein, 377 aa, Hordeum vulgare
MAAPGMALAAAEADRFCLPRAAAQQFENWGDSVSGIVVSSPLTETSTDLGDSGEKHHALMGCGGGAGTGAGGGAHSQRRVGCVDSSERTGDEKTARRLAQNREAARKSRIRKKAYLSQLETSRSKLTHLEQELQRHLSQGIFIAGGGSGDCSSGGALAFDLEYARWLDDHQRHINNLRVAVIADISDDELRILVESVLLHHDEFFRLKNLATKADVFHVLSGMWMSPAERFFMWLGGFRSSEILKVLASHLEPLTDQQLMGICNLQQSSQQXXXXLSQGMEALQQALAETLAAAAGGGPLAGADNVTNYMGQMAIAMGKLSTLENFLRQGDLLRQQTLQQMHRILTTRQAARALLVISDYFSRLRALSSLWLARPRD

SEQ ID NO: 21 - protein, 377 aa, Hordeum vulgare
MAAPGMALAAAEADRFCLPRAAAQQFENWGDSVSGIVVSSPLTETSTDLGDSGEKHHALMGCGGGAGTGAGGGAHSQRRVGCVDSSERTGDEKTARRLAQNREAARKSRIRKKAYLSQLETSRSKLTHLEQELQRHLSQGIFIAGGGSGDCSSGGALAFDLEYARWLDDHQRHINNLRVAVIADISDDELRILVESVLLHHDEFFRLKNLATKADVFHVLSGMWMSPAERFFMWLGGFRSSEILKVLASHLEPLTDQQLMGICNLQQSSQQAEDALSQGMEALQQALAETLAAAAGGGPLAGADNVTNYMGQMAIAMGKLSTLENFLRQGDLLRQQTLQQMHRILTTRQAARALLVISDYFSRLRALSSLWLARPRD

SEQ ID NO: 22 - protein, 240 aa, Triticum aestivum
MPRAAAQQLENWGDSLSGIVVTSPLTETSTDLDDSGDKHHALMGGGGAGAGGGAHSQRRVGCADSSERTGDEKTARRLAQNREAARKSRIRKKAYLAQLENSRSKLTHLEQELQRARQQGIFIAGGGSGDCSSGGALAFDLEYARWLDDHQRHINNLRVAVIADMGDDELRILVESVLLHYDEFFRLKNLATKSDVFHVLSGMWMSPAERFFMWLGXXXXXDPQGRFFMATRTVLHLERP

SEQ ID NO: 23 - protein, 439 aa, Setaria italica
MHHPSHVFRAEQGAGGYHHHVGDGALLPELPRSPNYSSKSSSNLTVSTFVPPLAVAHGGVAPSFGMAPPGMAAAADDGRFCLPWAAPAGAAQFENWGDSGIVVTSPLTEATSTDVSGDKQQAQMGGAMTQSVAVHVDSCAVSKDGSSRDQKVQRRLAQNREAARKSRMRKKAYIVELESSRVKLAQLEQELQRARQQGMFIASGRAGDLGGSTGGASAFDLEYARWLDEHQRHMTDLRVALAAPQIGDDDLRVLVDGAMLHYEHMFRLKGAATRADVFHVLSGMWASPAERFFMWLGGFRSSELLKVLAGHVEPLTEQQLVGICSLRQSSQQAEDALSQGMEALQQALADTLAAAAAAGAGAGAADSVTNYMGQMAVAMGKLATVENFLRQADLLRQQTLQQMRRILTTRQAARALLVVSDYFSRLRALSSLWLTRPRD

SEQ ID NO: 24 - protein, 432 aa, Zea mays
MHRQPSPHAFSSSGSWAEQGAGGYRHGRDGATFLLPELLQRSPNPSSKSSSAATFVPPLAAAHGGGVAAPFGMAPLGVAAADEARFCMTPWSAAAHFENWGDSGIVVTSPLAETASTDVDMGGGGAMAQSVDGHDNSLPACKVEPRDHKAQRRLAQNREAARKSRMRKKAYIVELENSRSKLSHLEQELQRARQQGMFIASGRSGDHGCSTGGALAFDLEYARWLDEHQHHMNDLRVALSAQIGDDDLGVLVDGAMLHYDQMFRLKGVATRTDVFHVLSGMWMSPAERFFMWLGGFRSSELLKVLARHVEPLTEQQLVGICGLQQSLQQAEDALSQGMEALQQALGDTLAAAATPCAADSVTNYMGQMAVAMSKLATVENFLRQADLLRQQTLKQVRRILTTRQAARALLVISDYFSRLRALSSLWLTRPTD

SEQ ID NO: 25 - protein, 140 aa, Oryza sativa
MRSRILWMSHAERFFMWLGGFCCSELLKVLESHLEPLTNQQLMGICNLQQSSQQAEDALSQGMEALQQTLGDTLASAAATVVVGGVGADNVTNYMGQMAIAMAMLTTLENFLKLRSNINGELNCLLPWRDAMIFLCRLIC

SEQ ID NO: 26 - protein, 89 aa, Oryza sativa
MARLPLVPCGDVADADEETALLAADARVARRRPHPDDVVNGGGGANALMSEDELRVLVDAVMMHYDQVLESHLEPLTDQQLMGICNLQQ

SEQ ID NO: 27 - protein, 151 aa, Oryza sativa
MVLLIWMSPQPLLEMVRLPLVPCDGVADADEETALLAADARVLESHLEPLTDQLFMGICNLQQSSQQVEDALSQGMEALQQTLGDTLASAAATVVVGGIGADNVTNYMGQIAIAMAMLTTLENFLKLRSNINGELNCLLPWRDAMIFFCVG

SEQ ID NO: 28 - protein, 385 aa, Oryza sativa
MAAHQGMAAATAADRFCLPRMAAAAAAASQVENWGDSGVIVSSPFTDDTSTDLDDSADKHHLHALVGGGDGGDDAGEQRGADSSAVSKERRGDQKMQRRLAQNREAARKSRMRKKAYIQQLESSRSKLMHLEQELQRARQQGIFIATGGSGDHGHSIGGNGTLAFDLEYARWLDEHQRHINDLRVALNAQMSDDELCELVDAVMMHYDQVFRLKSFATKSDVFHVLSGMWMSPAERFFMWLGGFRSSELLKVLASHLEPLTDQQLMGICNLQQSSQQAEDALSQGMEALQQTLGDTLVSAAATVVSGGGGADNVTNYMGQMAIAMAKLTTLENFLRQADLLRHQTLQQMHRILTTRQAARALLVISDYFSRLRALSSLWLARPRD

SEQ ID NO: 29 - protein, 429 aa, Musa acuminata
MYSSHLYRGEQAPSGFNIGDAVLDLTRSPILSPKSSSNLTAVNSSPLHLHFGAINHASMASLDVAAPTAAAATHVDAGRMAFSRGQIENWGDSAMVVSSPRTDTSTDVEIEDRTKMFGGVQQGAIICADSSAMSKGKAGEQKTLRRLAQNREAARKSRLRKKAYVQQLENSRVKLIQLEQVLQRARQQGILIASGFMGDHSYSIGGNGALAFDLEYARWLEDHQRRINDLRSALNSQVNDDELRLTVETFMGHYDEVFRLKSIGIKSDVFHILSGMWTTPAERCFMWLGGFRSSELLKILVSHLEPLTDQQLMGLCNLQHSSQQAEDALSQGMEALQQSLSETLASASLVPAGGADNVSNYMGQMTIAMGKLSTLENFLRQADLLRQQTLQQMHRILTTRQAARALLTISDYFSRLRALSSLWLARPRD

SEQ ID NO: 30 - protein, 275 aa, Spirodela polyrhiza
MFSFLQTSAYVQQLESSRLKLLQLEQELQRARQQGVFVANGCLGEQGHSLGGSGSLTFDMEYSRWLYEHQRQIGGLRVAVNSHVSDNELSLLVDAVMAHYDEVFKLKSIGAKSNIFHMLSGMWTTPAERCFMWLGGFRSSELLKILASQVEPLTDHQLMGLCSLQQSSQQAEDALSQGMEALQQSLAETLASGSLCPAPGSDNVASYMGQMALAMGKLGTLENFLRQADLLRQQTLHQMQRILTTRQAARALLAINDYFSRLRALSSLWLARPRE

SEQ ID NO: 31 - protein, 373 aa, Lotus japonicus
MELEVRSDMEKIDDVMVMPGFRFHPTDEELVGYYLKRKVQQKSLPVDLIKQVDIYKYDPWDLPKLASTGEKEWYFYCPRDRKYRNSARPNRVTRAGFWKATGTDRPIYSSEGKCIGLKKSLVFYRGRAAKGMKTDWMMHEFRLPCMPSDSAAPPKKLSDKSFSATDSWAICRIFKKTNSLSMVQKALSHPWISELPGGMVSDLLTQQGANFNQFCSENITTPSAIQFCGNTKQEQLHLISNANITNFSVSASASDIPAYKPIINSNTVTKSQQIPVSDGDLADNLMFYTLEPTGPTNPDYIGFEEPSQNYGGFSISLPQDMQPNITQVQLNQWESTVERTIGFPFNLPSNEAWKSALASWDSPPCSTEMSTSF

SEQ ID NO: 32 - protein, 345 aa, Amborella trichopoda
MFYVREEMATIGEKEWYFYCPRDRKYRNSARPNRVTGAGFWKATGTDRPIYSSEGSKCIGLKKSLVFYKGRAAKGIKTDWMMHEFRLPSLTDSNSPKKPLDKNLPANDSWAICRIFKKTNSMTQRALSQSWVSPLPETNNQDIFSNNISYTNFSSENISCTSEAGPGTQFYSEILHASNPDFSHLNLASYKPINSTLSKPFPLTQTSAPEFAFSPLDMLGPAKSTMDIASMLLNLSPQILGDGGKASANVEFGQQLQNQSSSFGVNLQPQMNENSQNGEGEACLTKRENNSRDVSGNQWCNARAGGFPLNLPSALFDAWKPNWSWDCSSCPSEMSTNYSTSKSYT

SEQ ID NO: 33 - protein, 402 aa, Aquilegia coerulea
MDDKNDFDKVDDVMLPGFRFHPTDEELVGFYLKRKIQHRSLPIELIKQLDIYKYDPWDLPKLATTGEKEWYFYCPRDRKYRNSARPNRVTGAGFWKATGTDRPIYSSEGNKCIGLKKSLVFYRGRAAKGIKTDWMMHEFRLPSLTDSAPPKRPLDKSFPANDSWAICRIFKKTNSMAQRALSHSWVSPFPETTGTDTLPEGTHYTQFSPENLSCTTEARSVFQLCCNNDVYQSSTTGTTYPSIDAQSYKSYNTMVCKPNLPSTPNGELPSGLMFSGFDMSGPAKCTVDVTSLLANLSPAFLGDGGKTSENIDFAGAQQQSNGFSINLPQEMQGNMRMGEEETCLRKNSNASHINSHWGARSVGFPFNLPLDNPDAWKPNLPWDSPPCPSELSTSYSTNKCYT

SEQ ID NO: 34 - protein, 337 aa, Arabidopsis thaliana
MVLVMDDEESNNVERYDDVVLPGFRFHPTDEELVSFYLKRKVLHKSLPFDLIKKVDIYKYDPWDLPKLAAMGEKEWYFYCPRDRKYRNSTRPNRVTGGGFWKATGTDRPIYSLDSTRCIGLKKSLVFYRGRAAKGVKTDWMMHEFRLPSLSDSHHSSYPNYNNKKQHLNNNNNSKELPSNDAWAICRIFKKTNAVSSQRSIPQSWVYPTIPDNNQQSHNNTATLLASSDVLSHISTRQNFIPSPVNEPASFTESAASYFASQMLGVTYNTARNNGTGDALFLRNNGTGDALVLSNNENNYFNNLTGGLTHEVPNVRSMVMEETTGSEMSATSYSTNN

SEQ ID NO: 35 - protein, 383 aa, Solanum lycopersicum
MDDKSDVEKLDEVMLPGFRFHPTDEELVGFYLKRKIQQRSLPIELIKQVDIYKYDPWDLPKLASTGEKEWYFYCPRDRKYRNSARPNRVTGAGFWKATGTDRPIYSSDSTKCIGLKKSLVFYRGRAARGIKTDWMMHEFRLPTTNNESGPPKKFLEKNFPPNDSWAICRIFKKTNSMANRALSYAWGNPLSEVASPELFNQSANAHFVSENVSSITETGTALHLSNNNDLLQGASNVPSYKSLNAMASRPSYLSSPSTDISYNFMFSSPEVSGQNNRCLLDANSMFFNTSPALITDISKTSESIDFDGSNQQLKMQTSISMENDEAGLKSNRTSINDNTEWENMRSVGFPFSLAEEWKPSLSWDSPPCPSEISTSFSTNKCYN

SEQ ID NO: 36 - protein, 386 aa, Eucalyptus grandis
MEDKSDVEKVDDVMLPGFRFHPTDEELVGFYLKRKIQQRSLPIELIKQVDIYKYDPWDLPKLATTGEKEWYFYCPRDRKYRNSARPNRVTGSGFWKATGTDRPIYSSDGTKCIGLKKSLVFYRGRAAKGMKTDWMMHEFRLPSITDSAPPKKFLDKNIAPNDSWAICRIFKKTNSMAQRALSHSWVTQLPEPIGSDLLGHGAQYSQFSSENISCTTEIGSAINHPNSASFNSMDVQSYRPMLYPPVSKASTLTISNSDLPSAFVFSPLDMPPGPTKCTVDTSPMLMCPALMGGEVNKASESISFEGSHQFSGFSIGLPHEMQGNIGGEEEAAGLKKNPALVHANNQWPATTRSMGFPFSLPSNMPWDSPPCPSEMSTAFSTNKCYN

SEQ ID NO: 37 - protein, 332 aa, Gossypium raimondii
MDDVEKVEDVMLPGFRFHPTDEELVEFYLKNKIQQKSLPIQLITQLDIYKYEPWDLPKLAASGEKEWYFYCPRDRKYRNSARPNRVTRGGFWKATGTDRPIYSSDATKCIGLKKSLVFYRGRAAKGIKTDWMMHEFRLPSLSDKTLSANVMILIRSLISNDSWAICKIFRKTNCMAQRALSHTSIPSDIPHHYGPFNHFNEVQHAQQWTTSPLPVPNGDLHNNSMFLQCTVDTSSVVLSPDATMIADDVRNNSESIDFKGKQQSFNGFSIRLPQDMEGTRNDDGDWRSIPLMGFPFSLSPNVLDSWKPNLPWDTSLYPTEMSTTYSTDRYHT

SEQ ID NO: 38 - protein, 386 aa, Gossypium raimondii
MDEKNDVDKVDDVMLPGFRFHPTDEELVGFYLKKKIQQRPLPIELIKQLDIYKYEPWDLPKLAASGEKEWYFYCPRDRKYRNSARPNRVTGAGFWKATGTDRPIYSSDGTKCIGLKKSLVFYRGRAAKGMKTDWMMHEFRLPSMSDSIPPSKKLFDKTLPANDAWAICRIFKKTSTMAQRALSHTWISSLNHGAACSQLSSENISCTTEIGSTIPLCSNGELQQDCSAGFSALDILSYKSFHQTVYRPSPFHVSNGDLHNNMMFSQLDNMSGSSPKCTIDTSSMLLSPPLISNASASTDFRGQQHQYGNFSISSPQDMQGNGGGAESEEGTRKTSDSIPDYNHLSMGFPFRLSPNMPDSWKPNLPWDSPQCPSEMSTTYSTDKCYT

SEQ ID NO: 39 - protein, 397 aa, Manihot esculenta
MMEEKNDVDKIDDVMLPGFRFHPTDEELVGFYLKRKIQQRSLPIELIKQVDIYKYDPWDLPKLATTGEKEWYFYCPRDRKYRNSARPNRVTRAGFWKATGTDRPIYSSDGSKCIGLKKSLVFYRGRAAKGIKTDWMMHEFRLPSLAEPSPPKKLLERSLPPNDAWAICRIFKKTNSMAQRALNYSWISQLPEATASDVVNQGTHCTQFSSENISCTTESVFQICSNNLQQASSGNFRALDISSYKSITTTVDKPSLFPSSNGDLTNSFLFSSIEMSAPTKCPVDDSSMLLNSALIGNVSKTSESIDYEGSQHQFNGFSISLPQVTQDHMAADEDEACLRKNPSGVHENSQWETMRSIGFPFSLTSNLPDAWKSNLPWDSPPCPSEMSSTYSTNKCYT

SEQ ID NO: 40 - protein, 402 aa, Manihot esculenta
MKRIMEEKNDVDKIEDAMLPGFRFHPTDEELVGFYLKRKIQQQPLPIELIKQVDIYKYDPWDLPKELATTGEKEWYFYCPRDRKYRNSARPNRVTGAGFWKATGTDRPIYSSDGTKCIGLKKSLVFYRGRAAKGIKTDWMMHEFRLPTLAEPSPPKKLLDKSLPPNDAWAICRIFKKANSMAQRALNISWVSQLSDTAASDILNQGPQSTQFISENISCTTEIGSVFQMCSNDLQQASSANFSALDISSYKPITTTVDKPSLFPVSNGDLPNNFMFSPIEMPAPAKCTIDAPMLVNHDLMVDVNKASESMDYDGSQHQFNGFSISLHQDTQGSVGAEDGETGLRKNPSGFHDNSQWENMQSIGFPFSLPSNIPDDWKSNLPWDSPPCPSEMSSNYSANKCYT

SEQ ID NO: 41 - protein, 380 aa, Glycine max
MERNEMERIDDVMPGFRFHPTDEELVDFYLKRKIQQKSLPIELIKQVDIYKYDPWDLPKLAGTGEKEWYFYCPRDRKYRNSARPNRVTRAGFWKATGTDRPIYSSEGKCIGLKKSLVFYRGRAAKGMKTDWMMHEFRLPCISDSSPPKKLSDRSLPPNDSWAICRIFKKTNSLSLAQKALSLPLISQLPGGIVSDMFTQGLKCHNIGSPAIQFCGDKQDELHQVSNNAINNNFSASDIPTYKLINNSTVSKPSPQSPVSDGDLADNFIFYSSWPTKCCTLDPTSMLSPNPDYIEFEDPNQQHYSGFSINLPQDMKQNMSTETGTKEQDHQWETTIGMRTTGYPFSLSPPYDAWKASSVAWESRPCPSDMSTTYSTNKCYT

SEQ ID NO: 42 - protein, 367 aa, Glycine max
MEERNEMERIDDVMPGFRFHPTDEELVDFYLKRKIQQKSLPIELIKQVDIYKYDPWDLPKLAGTGEKEWYFYCPRDRKYRNSARPNRVTRAGFWKATGTDRPIYSSEGKCIGLKKSLVFYRGRAAKGMKTDWMMHEFRLPCISDSSPPKKLSDKSLPPSDSWAICRIFKKANSFSMAQKALSLPWISQLPGGMVSDMFTQGLNCHNIESPAIQFCGDKQDELHQVSNNAINTNFTASDIPTYKPINNSTASKSPVSDGDLAADNFIFYTGPTKCCSTLLDATNSMLSHPNPDYIAFEEPNQQHYSGFSINLPQDMQQNMSTETGTTGFPFSLSPPYDAAWKASSVAWDSPPCPSDMSTTYSTNKCYT

SEQ ID NO: 43 - protein, 398 aa, Prunus persica
MDEKNDMDKIDDVMLPGFRFHPTDEELVGFYLRRKIQQRLLPIELIKQVDIYKYDPWDLPKLASTGEKEWYFYCPRDRKYRNSARPNRVTGAGFWKATGTDRPIYSSEGSKCIGLKKSLVFYRGRAAKGIKTDWMMHEFRLPSLSESAPPPKKLLDKSLPANDAWAICRIFKKTNSMAQRALSHSWGSPFPEPTAASGLLNQGAHCTQFSSENMSCTTDIGSSIQFWSNNDLQQASNASYSAFDTLPYKPINPTGSKPSIFDADQFPNGFMFSPVEMSGPISPALIGDLSNTTGSIDFDGSQQQQFSGFSINLSQNQMQGNMETRENEGGFRKNLSSSTSHANNNNNNNNGQWGSIRPIAFPFSLASDDDAWKPTLPSDSPPCPSDMSTSYSTNKCYT

SEQ ID NO: 44 - protein, 363 aa, Aquilegia coerulea
MNEKHESDNIDEIMLPGFRFHPTDEELVSFYLKRKIQQRPLSIELIKQLDIYKYDPWDLPKELASTGEKEWYFYCPRDRKYRNSARPNRVTKAGFWKATGTDRPVYSSDGSKCIGLKKSLVFYQGRAAKGMKTDWMMHEFRLPSLTNSMALKRPIDKNLPANDAWAICRIFKKTNSVAQRALSQSWISSVSTTSTTETSSSTQLNCNNNDFKTSNVYSFPSTDIPSIKPIEQISSMPSILSIINGDFPSGFNMFSTLDMSPPSLLGDNMNTSNNLDFAGIQQLCDSFPIRSQNEIQAGNTMTSKEDVWFNSPKVSNVHNQLGNFESVEFPIPMFDEWRPNLLWDPLSFPTEFSIDYSANKCYI

SEQ ID NO: 45 - protein, 390 aa, Eucalyptus grandis
MEESVDCEKSMDEVMLPGFRFHPTDEELVGFYLKRKIQQRPLSIELIRQLDIYKYDPWDLPKLATTGEKEWYFYCPRDRKYRNSARPNRVTGAGFWKATGTDRPIYSSDGSKCIGLKKSLVFYQGRAAKGVKTDWMMHEFRLPSLTDLSSQNIPAHDSWAICRIFKKTNSTSHRSLAHSYVSTMLPTETTASETANSLFHSQLSPDTPSFSPSPTKTSSSVHIPPHNITPVFDSSSLVPWTNLDFAIISSNLDPNPNHFLFPPLQETTASDVSSILQHMSSSMFGDVGRSFESDLNDHQEQFNDGFPLLTLPLEMQVNGAVVGGSGGEEASSLLPRNPSRDETWEAVRFGGSPYGSDNYLNVGDAWKSNLGWDAMSDLSTYYYPTTKCFT

SEQ ID NO: 46 - protein, 442 aa, Glycine max
MMEERNNNDAAEKLDEVMLPGFRFHPTDEELVGFYLKRKIQQRPLTIELIKQLDIYKFDPWDLPKLATTGEKEWYFYCPRDRKYRNSARPNRVTGAGFWKATGTDRPIYSSEGSKCIGLKKSLVFYKGRAAKGVKTDWMMHEFRLPSLTDSLSPKYIDKITIPANESWAICRIFKKTNATAQRALSHSWVSSLPETRTSDHHILTKDQDNTTQFCSDNMSMTKKTTLASQYCSNNYNDSQPLTTNSSTLCPLDVASCKSIINPLLYKAFDHFPISNEDLSTSLLFSSPPFETSETNPKSTIMDVSSMLLNMSSSVLGDFNKTTSEDTNTNAASNFGVLQEHCSGYPIPFLRENMQTTFGNQQQQYCDNIYALVKGPNVNVPRVDDPELELETVVQRSIGFPLSVTMPLNIGDAWKSNNLLWDTSSFPCDVPSSYYSTTKCYT

SEQ ID NO: 47 - protein, 438 aa, Glycine max
MMEERNNDAEKLDEVMLPGFRFHPTDEELVGFYLKRKIQQRPLTIELIKQLDIYKFDPWDLPKLATTGEKEWYFYCPRDRKYRNSARPNRVTGAGFWKATGTDRPIYSSEGSKCIGLKKSLVFYKGRAAKGVKTDWMMHEFRLPSLTDSLSPKYIDKTIPANESWAICRIFKKINATAQRTLSQSWASSLPPDTRTSDHHIQTKDQDSTTQFCSDNMPMTNKTTLTSQFCNNNYNDSQPLPTNSSSSPLCPLDVASYKSIINPLLYKAFDHFPISNEDLRTSLLFSPPFETSDANPKSTMVDVSSMLLSLPSSVLGDFNKTTSEDTNTASNFGVLQEHCSGYPIPFLPEMQTTFGNQQQQYCDNINALVKGPNVNVPRVDDQELELETVVQRSIGFPLSATMPLNIVDAWKSNNLLWDTSSFPCDMPSSYYSTTKCYT

SEQ ID NO: 48 - protein, 413 aa, Glycine max
MEERNDNGEKLDEVMLPGFRFHPTDEELVGFYLKRKIQQRPLSIELIKQLDIYKYDPWDLPKMATTGEKEWYFYCPRDRKYRNSARPNRVTGAGFWKATGTDRPIYSSEGSKCIGLKKSLVFYKGRAAKGIKTDWMMHEFRLPSLTHPPSSLKKFMDKTIPANESWAICRIFKKTNATAQRALSHSWVSPLPETTTTNSDHMITTDHRDINQFCQANMPFTKKTSLASQFCTFNHNDTQHSSTTTSTTPCPLDVVASYNKPLMNPLMLYKSLDRLPFSNYGDLLTSGLIFSSPLETSTTTNAKSSMDASSLLLSTSSSVLGEFSKNSEGIITTNFGVLQEHGNDYQIPLLRDNMQGTFGNHDDNNVLVKIPNVNVVPRVGDQQLETVRSIGFPFNIGDAWKPNMLWDASSLSL

SEQ ID NO: 49 - protein, 401 aa, Glycine max
MEERNHDGEKLDEVMLPGFRFHPTDEELVGFYLKRKIQQRPLSIELIKQLDIYKYDPWDLSKMATTGEKEWYFFCPRDRKYRNSARPNRVTGAGFWKATGTDRPIYSSEGSKCIGLKKSLVFYKGRAAKGIKTDWMMHEFRLPSLTHPSSDPKKYMDKTIPANESWAICRIFKKTNATAQRALSHSWVSPLPETTTTSDHRDGNQLCQANMPFTKKTSLASQFYTFNHNESQHSTTTSTTPCPLDVVASYNKPLINPLMLYKPSDRLPFSNGDLISSGVIFSSLETSTTSAKSSTDASSLLLSTSSSVLGDFSKTSEGTITTNFGGLQEHGNGYQIPLLRDNMQGNFGNHDDNALGKIPNVNVVPRVGDQQLETVRSIGFPFNIGDAWKPNMLWDTSSLSL

SEQ ID NO: 50 - protein, 482 aa, Prunus persica
MDERSDMNIIGGDKMDEVMLPGFRFHPTDEELVGFYLKRKIQQRPLSIELIKQLDIYKFDPWDLPKLAASGEKEWYFYCPRDRKYRNSTRPNRVTGAGFWKATGTDRPIYSSEGACSSKCIGLKKSLVFYKGRAAKGVKTDWMMHEFRLPSLTDSLQTKRSSSFMDKTIPANDSWAICRIFKKTNSSHAHQRALSHHSWVSVLPETNTYDDHMLGSKAAHSAAHHHHQHEFSSERMPLTSKTNSTTNFCANNNNDIQISSTTSSMSPLDIYTSSYKPMNPLMMGCNIKLPYQYFPISNSNGDYFSSTPSFTFSSSPLETPSGPVAKCAVDVSSLLLNMSSSMLGDYNSSNSINKASAESTNFEFSTTTGSQDQCNGGFSSMPLPHHHEVAQGNNNMGNGHDTVVGLIKNNVSNVNSNLTEDADDQQWETNVRSNIGFPLMMSNSLPLPLNIGGGGGGDAWKSSLTWESSPCPSEMSTTRCYT

SEQ ID NO: 51 - protein, 380 aa, Gossypium raimondii
MKERSESDKMEEIMLPGFRFHPTDEELVGFYLKRKVQQRPPSMEFIKQLDIYKYDPWDLPKMATTGEKEWYFYCPRDRKYRNSARPNRVTGAGFWKATGTDRPIYSSEGNKCIGLKKSLVFYKGRATKGVKTDWMMHEFRLPSLADSAPPKIFLEKTLPPNDAWAICRIFKKTNSTAQRALSQSRVSQVPETSSSSISEVPSMPLTAKTSLNLNVDLLQTSNGAFSSLDFVPYKPPQHSMSNGDLTSLVFTPLDHTSTDSRAVDVTSMLLNMLSSILGDHYCGRATDGLHFGGGFTGTLPHELQGNNMMSGGDHESSLFKSLNVGSHGDDQWGGVRSIGFPISMPLPMNVMGDAWKPSLAWDSSCPSEMSTSFCSTKCYT

SEQ ID NO: 52 - protein, 386 aa, Gossypium raimondii
MDERVSDSDKMEEIMLPGFRFHPTDEELVGFYLKRKIQQRPLSVELIKQLDIYKYDPWDLPKLATTGEKEWYFYCPRDRKYRNSARPNRVTGAGFWKATGTDRPIYSSEGSKCIGLKKSLVFYKGRAAKGVKTDWMMHEFRLPSLTDSAATPPKRFLDKCLPANDSWAICRIFKKTNSTAQRALCHSWVSPIPETSSISDMLRGSDTPQLSSDNISLTPKTSSSPVQFNLISNTDLLQASSAAFSPLDFVPYKPLSQMAPQLPISNGDLTSLIFAPLDQTPSPAKSAVDVTSVLLNMSSSMLGEYDSTVHFHGGSQDHCNGFSSGTLPHGIQGNMVINNEDHENALLKNLNVTHVDDQWDGIRSIGFPFSLPLPMSVADAWKPSMP

SEQ ID NO: 53 - protein, 412 aa, Manihot esculenta
MDERNDVDKIDEVMLPGFRFHPTDEELVGFYLKRKIQQRPLSIELIKQLDIYKYDPWDLPKLATTGEKEWYFYCPRDRKYRNSARPNRVTGAGFWKATGTDRPIYSSEGNKCIGLKKSLVFYKGRAAKGIKTDWMMHEFRLPCLADSAPPKRFIDKTIPANDSWAICRIFKKTNSTAQRALSNPWVSPLPEPSASSLISKGSQTSHHHFSSQNMPLATETSTGIQLNYNNHEIQHSSITSFSPSDFSSFKPVNHNSSLPIKLSHHLPISNGDLTNNFFFSPIETLAPAKCTVDVSSVLLNMSSSMLEDIGYHKVATDSSLDFAGPQDHCNGFSLTCLPQVMQVNAGNGDDHAPMKNPNATHINDQWETVPSIGFPFSLPMSMGDTWKPNLVWDSSSCSSEMSTSFSTAECYT

SEQ ID NO: 54 - protein, 410 aa, Manihot esculenta
MDYRNDVDKIDEVMLPGFRFHPTDEELVGFYLKRKIQQRPLSIELIIKQLDIYKYDPWDLPKFATTGEKEWYFYCPRDRKYRNSARPNRVTGAGFWKATGTDRPIYSSEGNKCIGLKKSLVFYKGRAARGIKTEWMMHEFRLPSLTDSAPPKSFIDKTIPANDSWAICRIFKKTNSTAQRALSHSLVSPLPEPSTSPLLAKGSQVSSHQQFSSQNMPLTTETSSGIHLNYNNHEIQRSFVANLSALGFSTCKPMNNNSSPLASKLSQLPISNGDLTENFFLQPVGTLAHAAKCTVDASSVLLNMSSSMLGDFGDHKLASDESSTDFAGPQDQHCSGFSLTCLPQVMQANAGDNALIKNPNATHMDDQWETVRSIGLPFSLPVSVGDAWKPNLLWDSSSCPIEMSTSFSTN

SEQ ID NO: 55 - protein, 362 aa, Hordeum vulgare
MESRNDVKSDEILLPGFRFHPTDEELVSFYLKKKIQQKPISIELIRQLDIYKFDPWDLPKLASTRGETDWYFYCPRDRKYRNSVRPNRVTAAGFWKATGTDRPIYSSEDTRCIGLKKSLVFYKGRAARGIKTDWMMHEFRLPSLADTSLPKSRPIDKNIALNDSWTICRIFKKTSSTVAQRALSHAWGAPSPGATEQHLLSAMQPMQTSHFALESSTCSLQVAAVAPSNQFISFNGGPSPQVLKGPTPTILPFQTPLPSHKPVLRTTPPPFFFDAHFGQPEQITGFVTGSSADVNAGMSCRDQESSTTKHGGNFRSLNNEREAPGRLDFPFDLVADSSNDWECNIPWESFLSPTLPAEISQH

SEQ ID NO: 56 - protein, 362 aa, Hordeum vulgare; identical to SEQ ID NO: 55

SEQ ID NO: 57 - protein, 360 aa, Triticum aestivum
MEGRDDVKSDEILLPGFRFHPTDEELVSFYLKKKIQQKPISIELIMQQDIYKFDPWDLPKLASTSSETEWYFYCPRDRKYRNSARPNRVTAAGFWKATGTDRPIYSSEGTRCVGLKKSLVFYKGRAARGIKTDWMMHEFRLPSLTDPSLPKRPIDKNIPLNDSWTICRIFKKTGSMAAQRALSHAWGAPLPGATEQQHLLSAMQTGQASHFASESSTRSLQVAAVAPSNQFISFNCGPSPQVFKGPTILPFQTQAPSHKPVLRTAPPSLFFDAHFGQPEQIAGFVTGSSADVNASMSCRDQESSTTKHGGNFRSMNNEWEAPGRLDFPFGLVEDSSDDWECNIPWESFLSPTVPAEIPQH

SEQ ID NO: 58 - protein, 357 aa, Triticum aestivum
MEGRDDVKSDEILLPGFRFHPTDEELVSFYLKKKIQQKPISIELIRQQDIYKFDPWDLPKLASTSSETEWYFYCPRDRKYRKSTRPNRVTATGFWKATGTDRPIYSSEGTRCVGLKKSLVFYKGRAARGIKTDWMMHEFRLPSLTDPSLPKRPIDKNIPLNDSWTICRIFKKTSSMAAQRALSHAWGATLPGAPEEQHLLSAMQPVQASHFASESSTSSLQVAAVAPSNHFISFNCGPSQQVLKGPIILPFQTQAPSGKTMRTVPPPLFFDAHFGQPEQIAGFVTGSSVDVNSSMSCRDQESSTTKRGGNFRNNEWEAPERLDFPFGLVEDSSDDWECNIPWESFLSPTVPAEIPQH

SEQ ID NO: 59 - protein, 352 aa, Triticum aestivum
MEGRDDVKSDEILLPGFRFHPTDEELVSFYLKKKIQQKPISIELIKQQDIYKFDPWDLPKLASTSSETEWYFYCPRDRKYHNSARPNRVTAAGFWKATGTDRPIYSSEGTRCVGLKKSLVFYKGRAARGIKTDWMMHEFRLPSLADPSLPKRPIDKNIPLNDSWTICRIFKKPGSMAAQRALSHAWGAPLPGATEQPMQASHFASESSTRSLQVAAVAPSSQFISFNCGPSPQVLKGPTILPFQTQAPSHKPVLHTAPPPLFFDAHFGQPEQSAGFVTGSSADVNASMSCRDQESSTTKHGGKFRSMNNEWEAPGRLDFPFGLVADSSDDWECNIPWESFLSPTVPAEISQH

SEQ ID NO: 60 - protein, 387 aa, Hordeum vulgare
MESRNDIKSDEILLPGFRFHPTDEELVSFYLKKKIQQKPISIELIRQLDIYKFDPWDLPKLASTGGETEWYFYCPRDRKYRNSVRPNRVTAAGFWKATGTDRPIYSSVGTRCIGLKKSLVFYKGRAARGMKTDWMMHEFKLPSLTDPSLPKSRPIDKNIPLNDSWTICRIFKKTSSMAAQRALSHTWGLPLPGATEQDLFSALQPVQALHYASESSSRPLQVAAVAPSNPFNGNYGFQGQHQQFQKPSNTQEDGSSCKVISFNCRPSLQVLKGPIVLPFQTQPPSQKHVHIAPPLFFDTQFGQHEQTTGFVTGSSADVNAGMSCRDQESATTKHGNTFSMNNEREDPGLGRLNFPFNLEADSPDDWECNIPWESFLSPTLPAEISQY

SEQ ID NO: 61 - protein, 387 aa, Triticum aestivum
MESRNDVKSDEILMPGFRFHPTDEELVSFYLKKKIQQKPISIELIRQLDIYKFDPWDLPKLASTGGETESYFYCPRDRKYRNSARPNRVTAAGFWKATGTDRPIYSSEGTRCVGLKKSLVFYKGRAARGIKTDWMMHEFRLPSLADTSLPKSRPIDKNIPLNDSWTICRIFKKTSSMAAQRALSHTWGAPLPGETEQDLFSALQPVQALHFASESSSSSLQVAAALPSYQFNGKYGFQGQHQQFQKPSNTQENGSSCKVISFNCGPSLQVLKGPIILPFQTQPPSQRPMLHTSPPIFFDAQFGQPEQITGVVVTGSSEDVNADMSCREQESSTTKHDDTFSMKNELEAPGRLNFPFDLGADSSDDWECNIPWESFLSPTAPDEIIQY

SEQ ID NO: 62 - protein, 387 aa, Triticum aestivum
MESRDDVKSDEILMPGFRFHPTDEELVSFYLKKKIQQKPISIELIRQLDIYKFDPWDLPKLASTGGETEWYFYCPRDRKYRNSARPNRVTAAGFWKATGTDRPIYSSEDTRCVGLKKSLVFYKGRAARGIKTDWMMHEFRFPSLADTSLPKSRPIDKNIPLNDSWTICRIFKKTSSMAAQRALSHTWGAPLPGATEQDLFSALQPVQALHFASESSSSSLQVAAALPSNQFNGNYGFQGQHQQFLKPSNTREDGSSCKVISFNCGPSLQVLKGPIVLPFQTQPPSQKPVHVAPPLFFDTQSGQPEQITGFATGSSADVNADMSCRDQESATTKHGNTFSMNSEREDVGLGRLNFPFDLGAGSSDDWECNIPWESFLSPTVPAEITQY

SEQ ID NO: 63 - protein, 381 aa, Triticum aestivum
MEGINDVNMDKSEEIIMPGFRFHPTDEELVSFYLKKKIQQKPISIELIRQLDIYKFDPWDLPKLASTGETDWYFYCPRDRKYRNSARPNRVTAAGFWKATGTDRPIYSSEDTRCIGLKKSLVFYRGRAARGIKTDWMMHEFRLPSLTDPSLPKRPIDKNIPLNDSWTICKIFKKTSSMAQQMVLSHTWGPPLPGTTQQDLFSAMQPMQASHFASESSSRSLQVAEAPASQFNSKHGFQGQQQLQKPSNTQDGSSCKVISFNCSPSLELEGPTILPFQTQTSQKPVHTAPPLFNMQFGQPEQQITGFLADSSADVNAGMSCRNQESSTMKHGNTFSMNNELEAPGRLNFPFDLGADSSDDWKCNIPWESFLSPTVPAEMPQY

SEQ ID NO: 64 - protein, 381 aa, Triticum aestivum
MEGINDVNMDKSDEIIMPGFRFHPTDEELVSFYLKKKIQQKPISIELIRQLDIYKFDPWDLPKLASTGETDWYFYCPRDRKYRNSARPNRVTAAGFWKATGTDRPIYSSEGTRCIGLKKSLVFYRGRAARGIKTDWMMHEFRLPSLTDPSLPKRPIDKNIPLNDSWTICKIFKKTSSMAQQMALSHTWGPPLPGTTEQDLFSAMQPVQASHFASESSSRSLHVAEAPASQFNSKHGFQGQQQLQKPSNTQDGSSCKVISFNCNPSLELEGPTILPFQTQPSQKPEHTAPPLFNMQFGQPEQQITGFLADSSADVNAGMSCRNQESSTMKHGNTFSMNNELEAPGKLTFPFDLGADSSDDWKCNMPWESFLSPTVPAEMPQY

SEQ ID NO: 65 - protein, 385 aa, Oryza sativa
MEERNDVNMDKSDEILLPGFRFHPTDEELVSFYLKRKIQQKPISIELIRQLDIYKFDPWDLPKLASTGEKEWYFYCPRDRKYRNSVRPNRVTTAGFWKATGTDRPIYSTEGTKCIGLKKSLVFYKGRAARGIKTDWMMHEFRLPTLTDPSLPKKPIDKNIPLNDSWTICRIFKKTSSMAQRALCQTWGAQLPGTIDPDIFSTLQSVQASQFALESSSCSLQAAATAAHQITSKYALQGNNNNQQQQQQHKPSNPLDGSSCKVINFNCSQSAEVQNSQIILPFEAHTSQKTATPLLFDTQFGQPDQISRFVVDSSVNANGGGISNKSQDPSARKPGSGFSMNSDWDGVARINFPFDLGADSSEDWRSSIPWESFLSPTTVHAEMPN

SEQ ID NO: 66 - protein, 381 aa, Setaria italica
MEARNDINMDRSDEILMPGFRFHPTDEELVSFYLKRKIQQKPISIELIRQLDIYKYDPWDLPKLASTGEKEWYFYCPRDRKYRNSVRPNRVTAAGFWKATGTDRPIYSSEGTKCIGLKKSLVFYKGRAARGMKTDWMMHEFRLPSLNDPSLPKRPIDKTIPLNDSWTICRIFKKTSSMAQRALSHTWGPPLPGATEAEMFAAFQSVQASEFALESSSCSLQAAQPAPASQFTSRHGLQGHQQNKVNNPSLDGSSCKLINFNCSQSLEPQNFPISSFPFEVQTSQKTTAAAPMFFSTQPDHQLSGFVVDSSADVNGGIGSRSQDSSTRKPGNGFSMNSSDWEAVGRINFPFDLGADSAEDWRCNIPWESFLSPAAVQTELPH

SEQ ID NO: 67 - protein, 392 aa, Zea mays
MEERNGTNMEKSDEILMPGFRFHPTDEELVSFYLKRKVQQKPISIELIRQLDIYKYDPWDLPKLASTGEKEWYFYCPRDRKYRNSVRPNRVTAAGFWKATGTDRPIYSSEGTKCVGLKKSLVFYKGRAARGMKTDWMMHEFRLPSLADPSLPKRPIDKTIPLNDSWTICRIFKKTSSLAQRALSHTWGPPLLGATEADDMINALQSVQASEFALESSLQVAPAAPAAGQFSSRHGLHVHGQHQQKKGTNSPSLDGSSSCKLINFTSSSPSEEGRPQSLSFPVTFPFEVQGTHKTAAAAPVFFGAHPDHHLGGFVVDSSADVNGGIVGGRSQDSSTRKPSNNGFSTSSGDWETAGRVSFPFDLGADSSEEWRCNIPWESLLTPAAVQAELLPH

SEQ ID NO: 68 - protein, 381 aa, Zea mays
MEGERNDINVQRSDEILMPGFRFHPTDEELVSFYLRRKIQQKPISIELIRQLDIYKYDPWDLPKLASTGEKEWYFYCPRDRKYRNSVRPNRVTAAGFWKATGTDRPIYSSEGTKCVGLKKSLVFYRGRAARGMKTDWMMHEFRLPSLTDPKRPVDKTIPLNDSWTICRIFKKTSSMAQRALSHTWGPPLPGATAEADMFAALQSVQASELALERSLSLQAAPPAPAAGQFTSTHGLHSQHHHQQQKGVNNPSLDGASCRLINFTSSPSEEPENFPITFPFEVQAPHRAAAPVFFGAHPDNRLGGFVVDSSADVSGCIGGRSQDSSTGKPSSGFSANSSDWETAGRINFPFDLGADSSEEWRCSIPWESFLSPAAVQAELPR

SEQ ID NO: 69 - protein, 419 aa, Zea mays
MDDDRADTNKPDEVLLPGFRFHPTDEELISFYLRRKIQQRPLSVELIRQLDIYKYDPWDLPKLSSSGEKEWYFYCPRDRKYRNSARPNRVTGAGFWKATGTDRPVYSSEGAKCIGLKKSLVFYKGRAAKGIKTDWMMHEYRLPSLGDPSRPNKTPKDKNIPADDAWAICRVFKKPNSVAQRVLSRSWGPASIATADPELLPALQSIQASRFALETSTCSANRFNSQQQQNLSSSQDGSSCKVITFNRSPYLPSEKDIHSSSVVILPLEIQSQQKSSDVTSALLSMAPGILSSANEAIPNTEFGLLGPSNGYPIDWATDTSGGIVNSDDDPYKRKSGNGYSSGYECGIPRKIKFPFNLGNDMMFLMVHGGEVIIDGGTKSSMGGGRRAWNSSQQIWSDIAPYDLDGRLIESRWLEDVNSL

SEQ ID NO: 70 - protein, 401 aa, Spirodela polyrhiza
MDAKNDVEKADEGMLPGFRFHPTDEELVGFYLKRKVQKRPVSIELIRQLDIYKYDPWDLPRLAMAGEKEWYFYCPRDRKYRNSARPNRVTGAGFWKATGTDRAIYASEGAVCIGLKKSLVFYRGRAAKGIKTDWMMHEYRLPSPADPSSLPSKTVGKEISASDSWAICRIFRKTCSVTQRSLSHSWVPPLAEARDYPEEFPVSGNLHCPELASEGISSCPAETRRSTQPSGSTGHGPPTLSDYSSMDINPYILSSARLTSGITTHPLAEAAPSSRCPLDGAAAAAALLQYMQPQLVGDAERNSSSSMDFSQHRHHHQPQQLHAFSTIDLLLEMTGNLGRGGEEMGTRKSPDASLQASDGDSHWEALRALALPFAPPQGRSDAWKVGWEREVLFSIENVKNF

SEQ ID NO: 71 - protein, 385 aa, Musa acuminata
MDERSHDMEKGDEVILPGFRFHPTDEELVSFYLKRKVQQRPLSIELIRQLDIYKYDPWDLPKLATTGEKEWYFYCPRDRKYRNSTRPNRVTGAGFWKATGTDRPIYSSEGSKCIGLKKSLVFYKGRAAKGIKTDWMMHEFRLPSLTDSSLFKRPLDKNIPANDSWAICRIFKKTNSVAQRALSHSWVSPQPEATEPDLFSISRAAHSIHFGSEMVSCITEAGSAPAIQVGCSSQQQQQQQHNTIISYPPLDFSCYRPIDLGHCRPPPSVQMPTNFTFSPAEMQLTSKCTMDVTSMLLNLSPAILGDVEKASHMDFLQQPQQCNEFAKMVNACGLNSNRWGGSRKSSVDFPFGLPTMFSDEWKPNLPLNSSSYPSEISTSYSTNKC

SEQ ID NO: 72 - protein, 422 aa, Musa acuminata
MDERSDMEKGDDIMLPGFRFHPTDEELVGFYLNRKIQQKPPSIELIRQLDIYKYDPWDLPELATTTGEKEWYFYCPRDRKYKNSARPNRVTGAGFWKATGTDRPIYSSEGNKCIGLKKSLVFYRGRAAKGIKTEWMMHEFRLPSLAHKNISINVRTTDSWTICRIFKKPNTVAHRAAPPPSWVSPVPDPNGAADLFSILRTAQSTQLSSEMLRHPLESSCYKPELPNAQVAPTSFMFSPEHMQIPSKSAMDVASMLLNASPSIVGDVEEPLTSIDFGQQPMRSTASLANGEEGKICSLENSQWGFSLLPTISGELNLAWESPPYLLFLSLFYINWESSSFGEISRRADRIESILDSKGGKGYICCPPTAYLLACLPRYHRRPSSCASPSLGRLSSSFPTIAVAEEETICSQFYHLSTLLKFL

SEQ ID NO: 73 - protein, 375 aa, Spirodela polyrhiza
MDERNSDVTEKADEMILPGFRFHPTDEELVGFYLKRKVQQKPLSIELIRQLDIYKYDPWDLPKMATTGEKEWYFYCPRDRKYRNSARPNRVTGAGFWKATGTDRPIYSSEGAKCIGLKKSLVFYRGRAAKGVKTDWMMHEFRLPSHADSSLPRGQADKGVHPNDSWAICRIFKKTNSMARRAPPHPWASASPGTPAAADLFAHSRGLQFPHQLASKNDSCATEHGPQAQFGGHAGVLHHHQLQPLLDFTPSSSYRPIVGMGYSKPPVTVPSSAGFFFPPAEAAPSAGIDFGLQQRYQHPPPQQLFGSAPEIAVEISGHLGNGEPGSSHWGTLQTIAFPFSISSHPPDFLKSHQTWDSAACPSEMSTGYSVNKCYT

SEQ ID NO: 74 - protein, 202 aa, Lotus japonicus
MAAHRVIIIASLLFCLAGTCYGKVLFSSLKKTLDVTASPKHGQVVEAGLDTITVTWALNKTLPAGTDSSYKTIKVKLCYAPISQQDRAWRKTEDELSRDKTCQHKIVAKPYDASNKTVHTFEWLIERDVPEATYFVRAYALDSNDVQVGYGQTSDAKKTSNLFKIQAITGRHLSLDICSACFSAFSVVSLIFFFYIEKRKKH

SEQ ID NO: 75 - protein, 188 aa, Lotus japonicus
MTSVQCYKTCEQTCQQKNQHGSYGQKVADLFKGNHGHSKDKECHTGHNGHSNGAHTGHLVLGHSNGGHTNGGHTGHLGHSKDGYKVNHGHSKEGFEVKRCSKTEVVSQSAGTKAHKTELSCSKTQTTHAAGGTKHKGSTTRKHKKNLFGKIKDGLSGHSSDDNSSCSSSSESDSDDEKKCSRRKNICN

SEQ ID NO: 76 - protein, 373 aa, Lotus japonicus; identical to SEQ ID NO: 31

SEQ ID NO: 77 - protein, 282 aa, Lotus japonicus
MASATVVSQIQSLYIIKPRLSPPPPPHRQFRSIYFPTTRLLQQHRFRQMKSVVIVSATAAETARKRYPGESKGFVEEMRFVAMKLHTKDQAKEGEKEVTEPEEKAVTKWEPTVDGYLKFLVDSKVVYDTLEKIVQDATHPSYAEFRNTGLERSASLAKDLEWFKEQGYTIPQPSSPGLNYAQYLRDLSQNDPQAFICHFYNIYFAHSAGGRMIGKKIAGELLNNKGLEFYKWDGDLSQLLQNVRDKLNKVAEQWTREEKNHCLEETEKSFKWSGEILRLILS

SEQ ID NO: 78 - protein, 530 aa, Lotus japonicus
MVEIEGSPGSSMHGVTGREQTFIATVNSPSVPTDTTAKFDLPVDSEHKAKVFKLFSLANPHMRTFHLSWISFFTCFVSTFAAAPLVPIIRDNLNLTKSDIGNAGVASVSGSIFSRLTMGVICDLLGPRYGCAFLIMLSAPTVFCMSFVNDAAGYIVVRFMIGFSLATFVSCQYWMSTMFNTKIIGLVNGTAAGWGNMGGGATQLIMPIVYELIRRAGSTPFTAWRIAFFIPGFMHVFMGILVLTLGQDLPDGNLSALQKKGDVAKDKFSKVLWYAITNYRTWIFALLYGYSMGVELTTDNVIAEYFYDRFNLKLHTAGIIAASFGMANLVARPFGGYVSDVAARLFGMRGRLWTLWILQTLGGVFCIWLGRANSLPIAVLAMILFSVGAQAACGATFGIIPFISRRSLGIISGLTGAGGNFGSGLTQLVFFSTSRFSTAAGLSWMGVMIVGCTLPVTLVHFPQWGSMFLPPSKDIHKSSEEHYYTAEWDEEERKKGLHSQSLKFAENSRSERGKRISSAPTPPNTTPTHV

SEQ ID NO: 79 - protein, 586 aa, Lotus japonicus
MCGILAVLGCSDFTQAKRVRVLELSRRLKHRGPDWSGLHQHGDCYLAHQRLAIVDPASGDQPLFNEDKSIIVTVNGEIYNHEELRKQLPNHQFRTGSDCDVIAHLYEEHGENFMDMLDGIFSFVLLDTRDNTFIVARDAIGVTSLYIGWGLDGSVWISSEMKGLNDDCEHFEVFPPGHLYSSRERAFRRWYNPTWFSESIPSAPYDPLAVRHAFEKAVIKRLMTDVPFGVLLSGGLDSSLVASITSRYLATTKAAEQWGSKLHSFCVGLEGSPDLKAAKEVADYLGTVHHEFTFTVQDGIDAIEEVIYHVETYDVTTIRASTPMFLMSRKIKSLGVKWVISGEGSDEIFGGYLYFHKAPNKEEFHTETCRKIKALHQYDCLRANKSTFAWGLEARVPFLDKEFINVAMNIDPEYKMIKRDEGRIEKYILRRAFDDEEKPYLPKHILYRQKEQFSDGVGYSWIDGLKDHAAKHVTDKMILNAGNIFRHNTPLTKEAYYYRMIFERFFPQNSARLTVPGGPTVACSTAKAVEWDAAWSNNLDPSGRAALGVHLSAYDDKQNNLINNKPVEFEKLIPMEAPSLGVAIHS

SEQ ID NO: 80 - protein, 474 aa, Lotus japonicus
MQGFKTAQGNSHLYCHPSSFLLRGDDTSRNHTRFSDLGELQHSSNIFQQDDAVDLSSSNLPCSMYSVKSCNVVGGGSNMQYGTLNTNLGSAEIVSTGAGCLETGQQLMYHKEISMATMPMGNGHVENWADSSGMADNSQQTDDTSTDVDTDDKIQSNRDKRGTHMVVHSRDQTKSKPEDQKILRRLAQNREAAKKSRLRKKAYVQQLENSRLKLVQLEQELQRARQQGVFMASPGDQSHLAVGGANGASAFDMEYAHWVDEHQRLLTDLRSALNSQMGDNELHLLVDGVMAHYDELFRLKSLGAKADVFHMHSGLWKTPAERCFMWLGGFRSTELLKIVKNHLEPLTEPQLMGIDNLLRCAQQGEDALTQGMEALQQALVDTLSSTSLGPAGAGNVADYMGQMAIAMGKLATLESFLHSADLVKQQTLQQLQRILTTRQMARALLVRNDYISRLRALSSLWSSVMSSLPPSNIE

SEQ ID NO: 81 - protein, 459 aa, Vicia faba
MQTFKPAQSNSQLYCHQSFLLRGDDINRNQMRFSDLGDFHHSSPFFQQQDALDLTSTCMFNSVKSNNLAIGATNMHYGTTINTTVSSAEIASNGAGCSDAGQQQQQLTCNKGIASLPLGNSHVENWADSGMADNSQQTDDNSADIDIDSDDKHQSNRGNAHSKDQTEFRAKDHKTLRRLAQNREAARKSRLRKKAYVQQLESSRVKLVQLEQELQRARQQGIFIANPGDQGHSTVGNGALAFDLEYAHWVDEHQRLLNDLRSALNSQMGDNELHLLVDGVMAHYNELFRLKKIGAKADIFHMFYGLWKTPVERFFIWHGAFRSSELLKIVKNHLEPLTENQMMGICNLQQSTQQAEDALSQGMEVLKQSLLEIHSSTSSVPSTGSGDVADYMGQMAFAMSKLASLEEFLQKADLLKQESLQQLQRILTTRQTARALLVRNDYISRLRALSSLWLARPRE

SEQ ID NO: 82 - protein, 461 aa, Vigna unguiculata
MKNMQGFKTAPPSSQLYCHSSFLLRGNDGNRNPTTGFCDLGDLHHSSAVFHQDGVDLGSSSMFSVKSSNVVGGSNMQYGTLSTNVGSSEIGSGGGGCMDTGLQLMYHKGVAMQTLPLGNGHVDNWADSGIADNSQQTDDTSTDIDTDDRIQCNRGKNGSRMVMQSKDQTKIKPEDQKTLRRLAQNREAAKKSRLRKKAYVQQLENSRVKLVQLEQELQRARQQGIFIATEGNQGHLAVGNGALAFDLEYAHWVEEHQRLLNDLRLAVNSQMSDDELHILVEGVRAHYNELFRLKRIGAKADIFHILSGMWKTPVETRFMWLGGFRSSELLKIIKNHLEPLTEQQLLGICNLQQSSLQSEDALDKGMEALHQYLVETLSSTSLGPNGNGNVADYMDQMALAMAKLGELEGLLQQADLLKQHYLQQLQRILTVRQTARALLVQNDFISRLRALSSLWLARPKE

SEQ ID NO: 83 - protein, 364 aa, Lotus japonicus
MFQRGTIFNAVPLGNENWGDSGMADNSQQTEDTSTDVDTDDKNQCFSNVSWYNGVQQEPLVVVMDSKDQSKAKVKDEDQKTLRRLVQNREAARKSRLRKKAYVQQLENSRVRLAQMEQELQRVRQQDVSGITGDHAHSVVANGVLAFDMDYARWVDEHQHLINGIRSAINSQMVDKELHLLVDGVMAHYDELCRLKSIGAKADIFHMLSGMWKTPVERCFMWLGGYRSSELLKIVRNHLEQLTDQQLIGIYNLQQSSQQAEDALSQGMEALQQSLSETLSSTSLGPTCSGNVAEYMGQMAIAMAKLASLESFLHQADLLRQQTMHQLQRILTTNQAARALVVINDFISRLRALNSLWSACPKEY

SEQ ID NO: 84 - protein, 462 aa, Vigna unguiculata
MQSFNPTESESTSPLYSHSSFIHRGNDANPNPTRFSDLQHSFRQHDAIDLSSSSVFGAKSNNMAVVASNLQCGTFNTNLGGAEFASTVVGCAETDSQHMYQRGSSSATMPMGNQYVEKWADDSQHTEDTCTDIDTDDKNQCFSTVSWGTGVGDGALVVVDSQDQCKTKVKPEDQKTVRRLAQNREAARKSRLRKKAYVQQLENSRVRLAQLEQELQRARQQGVLVASGITADRGHSVVGNGALAFDMDYARWVDEHQRLINDLRSALNSRMGDNELHVLVDGVVAHYDELFRLKSIGAKADVFHILSGMWKTPAERCFIWLGGFRSSEILKIVRNQLEPLTEEQLMGIYNLQQSSQQAEDALSQGMEALQQSLSDTLSTPSQGPCGSGNVAEYMGQMAIALGKLATLESFLHQADLLRQQTLQQMRRILSTFQAARALLVINDYVSRLRALNSLWLACPREY

SEQ ID NO: 85 - DNA, 1317 bp, Lotus japonicus
  source 1..1317: mol_type = unassigned DNA
ggacatgagattgaagctccaaaattagctcttttttctgatgaatacttaatgctttgttgtattcacttgattaagtgctagaaatcatctttgcatgatcatagattaaatgaatttccagttggtgtgtggagagctattttgttatgctgacatctgcaatttgcagggcatctaatgattgtcatttcttaaattattattggttgtttccgtttctttaattatctgttttaatcttgcaggtcatacaaattaaaatactagccaccacccaagacatactaaatggggtagtagagggaagggtaaggtcgataaggatgactttttattctataaaatttaggagaatttgagcttaagtggcaaggcaaacgacattactatacgaattggctttgtaccagaaacagggaacaaataatattttacaaataagctattatcatgtcagctcatttgttcaactttgatttgattaaaaattaaatgaagttgaatttgttgagctgctttattatatatgccactggatgtttccgcattctaagtgcatgtttgaaaacatttctacaattgattacgaaggaaaaattaatcatggagagaagcttatgtgcgtagcttctgtatttctgaattgattctatctgtacagtagcatttagataatgaatgatcttggttctcgctaagcatcaaaccaatctctacccttttaaaattgcaagaattataagtcatgcattgacccaaatccttctgtggttatgccccttaaaaatccggcaagacatcaagttagttggtcattagggttccaccagctagctgacaccttgtacaacaactggccgtcctaaagttgggtaagcattacaatactaaatgccattttattatattttgcgcatggttatatacctaagtaggatttgtccacagtttctttgattcggaaaggaaaaaatatttagttgacactgacagaagcagattttatatacatatattatgaaatgactcctacatgagatacacgaatctcatccccatgagttgcagtttgacagagtacacacttatcaacttgctggaatataggaaagtctaaccaatgatgtcgatccgtattgccttaattttggtaaatttagtattacatgatcattattgatatactaaaccacaggatattttattgacaatgtgaatgttccatattttcaacaatgctgattccctctgataaagaacaagttccttttctctttccctgttaactatcatttgttccccacttcacaaaca

SEQ ID NO: 86 - DNA, 1135 bp, Medicago truncatula
  source 1..1135: mol_type = unassigned DNA
cgatccttcgttgtattcacttggtcaagtgcaagaaatcatctttacacaatcatataatgaatggacctttggttgatgtgttgagagctacattcagtcctctgctgacttctgcaacttgaggggaatctaatgattgtcttattgcaattaacaattccttttaattttttgacagaaagagttggttacaaaatctcatctaacatattgttgagttcctcttttttttttttttttttttttttgataaaagggtacatctgttcgcagctctgaagattaatccctcgaatagggttggataacatgaacgacaaatttgtttcttaagagttttaacactgtcatattcttatggcagaattcgaactcgaacccactggttaagctagaagtaagaagtgacccatgccatctcattcaatcgttcttggcttgttttcggtttagatgaatatgaagtacaaaaagctacgctcaagcaaaggagagcaggtaagaagggcatcttatttttttaatatattagatgtcaagcttacgtggcgatgtaaacactgtttctatccgaattagctatgtactagaaatagggaacaaataatataaatggtgagtttgtcaaggtgttttaatatattgaaaccaatgtaactggaagtgtcaacattgaaagctgttcattgcctgtattatattataattgcatatatataatggcattagcctttgttaaacgttaaaccatatcttttgctagtttgccccttaaaaatttggtaaaggcatcaagttagatggtctttaggtaccagccagctagttgacatcgtgtatggacaccaaattgggttataataccatattattcttaccatctttattatattctgcgcatgatttcatacttgtttgggatttgccaacagtgtctaagatttgaaaaagaaaaatagtagaactaatgacagagacatcagcatatatttttaatatcaaaccaaaagatatttgatgtccaaagaagagataaatataaagttagaagtatgacaataagtcttggttgttatttgccataagaaactctcttttctcttcccctcataatttgcatttcctcacaaca

SEQ ID NO: 87 - DNA, 2496 bp, Lotus japonicus
  source 1..2496: mol_type = unassigned DNA
tcgactatataaggggttcctccatacctaataagacatgtttgtttcatgtcgatctacttactctattttatggttttctcgtgagcaatttacatataaaatacacattaaattgcacccaataactaactagttacaagattagagcacataaatcatggaatgaaacaatagcctatgacttgggtattcggtccacaacatctctagaagttcgtacaacaacgatcaaagagaaagtactgactagtgatatgagaaatttggtcaggaacaatgggctggacacattacgaacttgagcctgaatagtagcagcatatgtctgccttcgcaatgacgaagaaggttggacggagctaaagttaaagctggaagggtgtgtcgtggcatttgaaatttagggttcaactgtagctttgtttgtctaactacccttctagctttaataaaaaaacgtaaatcaattaattaaagagaaatcacgccaacctgtctcaaacccgctctaacttgttggttaaggttatgtgttgtatttcaatttgcttgttgcttcaacttgactccttacctcctttcatgttttttcttgaaaattgtgtaaactcagttttttttttttgagaatgtatgagaatgtgatacaaattaaaatacaaacatacattgaatgagcggggctaaaatgactcaaggtacatatgagttaagtttttaccaacccatctctttgatgagttgggtttaattgggttagctcacgtgttaactatttcgacaattctaactttttggaataatgccatccaaccctaggtaaaactcaacatgacttctcttaaagaaatgaagagtgaaagtctacataaatcttaaacacttaaaacactacaacttggtagatcattaaaatatgctccagaaataccataatacttgaaaataaataatttataaatgtaaaatataaatttgttgctgcacccaagtctgagtgaggaatgaataaagataaataaaatatatatatgcgttgatactaacatatgatatgatgtgatagatgtcatatataatgaacagaatgatagaaattgttgagcgcatggaagcttcttgttcaataattcaagagctgcttgtggagggctgctgcagggttgttatggatgcaattatcaaatatcttgctggaaattgatccaaaagttgactattaccttatcaatgtccggtgtccttcaagccaccattcaattagtcaataaccttttgtttttgtttttttataattttggtgccaaataattttaaccatttatttttatcatatatattatataagatggcaagtctataatcatttaaaattaaattagtctaaatttttctaatatgctgtataaatttattatatataagaataaataatattaaaaatattgtttaattgctaccagttcaatcattaattaactattgactgaaaagaaaaatgtctaacaaattttgttacattctgtttgttttgagaaaaaaaaaattgtttttcattttctgaaaatatttatttttaaatttattaaatttagaaattatgtttgtttttacgctttgttttcatcttcaccgtcaccaccatcgctaccactgtcgtcactattactatataccactaccatcgttgttgccgttgtcattgtcaacaccattgctgtcgtcgttgttattatcgccccacaaccactatcatcatcactgctatcgtcatagctaccacgaacaccaccatcacttttcattgaaaatgaaaaatgtttatttatttattttgattatattttaaaaaatatttttaatcaaccaaatttctttttaactttttttttcttatgaaaaaaaataatatgaaaagaacaaaccaaacagatccttatattcttgtttccattctttatcacccatgagagtagctgtagcgcgttaactttctaatagaggcgggaatttaccgttaaccgttgggtcatcactcagcttactgagtcatcttcttcctactgtaggcggaacccacacgccactgcctctctattattacatcaaattctattttttttacatcatcatcaagataaataaattgcattattcagaaattaattcctgaatctccctatcttaactcaggtgtctctagctattaccacttaagctattattagaagtctacgtcaaattctataataaacacatattcttattcttattattatactaatacaaaccttgatttgaagccaaaaatgataaaacaattgcagactcgttcaaacaacagacaacatgtaccattaaattccaccccaaattctcaaagcaaattctgaattctccaactagtttccattctcctagtcaactcaactcaaaacccagatgagaatgagataattccaactttaacaaacacacacaattcatcattca

SEQ ID NO: 88 - DNA, 2000 bp, Medicago truncatula
  source 1..2000: mol_type = unassigned DNA
aaataatgtgtctaaaattaacaaagatattttatggttaatttattgagcttagttaaagtcattttctaaaacgagacattgtagttgttagataaataatttcctttaattttcatcaaagtttgtaaataattatagggcaggacacatatgtccatcaattgatatatatatatcttttgtaataacgatcttatagaagaatcaatatctacaaagaaagttatcacttgtctcaaattatgctttatgcaactggtattgtcatgtagattccattttggtttatatgatattgaagtcaacgaatcacagctgcttaggcgtggttcataatttaaaattgaaactatggtgtttgttggaattatgagaggacagactgtgaagtggttgcacagctaccgacagggttgccattactgatgcaaaggattaaacaatagaacattttcaatcaacttaatctatgtttgacatcacgctcgatgtgtcagaatcacgacgacccactgtaatttttaaaaaaattacattttataatttttgtagaatcacggtgtatcacagtgattttgacatacccactgtaataccaaacatgcacttgcattctttcttccgtttgatgccaaaaatggtattgtttataatacacttaaaataattcatatccaagttgttgtataaaaaaataaacaatgtgatttcccaaaaaaaaaaaataataagtgattggcatgcaacatcagattttgttgtccaagtggtcacatttgaaatatttgagtttacatttttctcactaatatctttatattgaacttcaatctgtctagctataatcaaatggttgactaaaatggttaataagcttcaattaaattcaaattatttgaaagaacttgaatttaaatcttaacttgaacataaccccgaagtggatgatttacccaaaaaaattacctaactactctaataatcattttaacaataagagtattttactcaaagaaatttatataatcacaaaatcctagtatactcaatgattttcttaaaaaactattataaccagaaatgacaatatgagacaaatataattattttaataaaaatatctaaattagaattatttgaaaatgtaaaactatattattttttgtagttttccaaactttattaagcatcgtttaattgcacacaaaatttactccaattttttttagtctctgaaagttgctttttggtccatgttttagttttggtaattataattaagtttgttcatattaattagttctaatgtgtgtactatatagatcatactaattttgttcccaaactcatcttagttaggatctttgtatctactataagattaatccttgtgccgatataagaatatttgtcatgttatcaactatcacaatattgtattggtgagatcatataagatttgatgtcaacatcttcgtaaaggtgtcagattcgattctccccggtatcaatttaagtgagctaatttagcttcttaaaaaataaaatcaaacaacttttacataaactcagtgaaaacttggatataaagtatccttatactactctttagtcttgattagtctctgcaaagatatttatatgtactttgtattatcataagaacattcattgacattttaagttaatgaattactaacatgtcaactcttattctagccaacagttactttgttccctccacattctctttgaaatagtcaaacgtatccaatcatgcatgtctgttctgatcataacagcaaaagcatgtgtatagaaaattgatagttgaattagagtcattttccataaaaaaatattcaataagtgtgacattatttttcgtatgaattaatccattttttgctgatttgagattctttctttctttgcttcttgctttccttcatcagccattttttttgttttctctttctctctctcttcttgattca

SEQ ID NO: 89 - DNA, 4171 bp, Lotus japonicus
  source 1..4171: mol_type = unassigned DNA
gcatgcatatagctctatttctttagtaatgttacacctgcacgatgtccataataatagaagtcataatacatatacagattaaaattaaataaacaatttctaatcaaatttaaaaatgtcaacttaatttcattattaaaatataacaatatgaataaccaaaaataaattaagacattcacccccccccccccgaaaagaaatttaagacaattacaattttttggtatatatattaaagacttccaattatggacataggatctcaacttagtaatcttcactttaggaaagtcttttccccacaagtcacaaccatctattaatatcaatacaaaatgaagtcaactcaataaaaagatccttttataggaaattgatgaataaaactgatatatatttcagttaaaattgttcaaacattagtgcaatggacagaagtatcctttgtgccctcatttgccaacaactggctcatcaagcaataaattaattcgccatttccaaacttttgcagttttaagtagaagatatccattcgttgaaactttcttcacaccaccaatttcctcctaaatgggttaacaaatgtgcaatgatcgaaatatatagttgaaacgatcaagatcctctcaatggtaaaagaatttgaccacgctaagttttattatctcactagctattaatttaattatcatttatcttttcaattattaaacacacaaataatcaatcctaaaatgatgaaacttgacatggtctatttttacaataacttaaccaaaaacttataagttagcaactttcaaaaacaggttttcccttgttaagaatagacaaatcaaatggagtgtgttaaatattgtgtttaaaatagtgtgttgcaagcatttctcttataaaaaatcagtataaatatgtttggaactgtttattttagtttatcttatattataaatacaaaacaagtgtttggtaaagctaatgaaaataacttaaaacatacctatggtttgtcggatgtgtcgcgggtggagctccttgccattttgtgtggcctttgtatgtgttgagatatggccatgggttattgtagaatccttcttcttttggattcttctttgttcctgtctctcattcaacgctcatgttacccatttcatccatgccaagttttttttatacatgcatctaaattttttccgccatatcttaattttgtttttattaaaattaaaaagaatatgattgaatgtcaatgtaaattttttttacacagacaatgcatatccattaaggtttgttagaattacactccaccccatttttatctaaaatctacatcccaccccattttatatagaggcaaatttagtgacgaaaaatattcttcattaataattagttattatttaaattgttaatcaataatttcaaaaaaaaattcaatataatccaataaatttaaaaatgaaaacatcacaatcctcctcattctctcaatcgcgttttacctccgtaaatttacaatgcaaattatgcaatagcacacctgcccgatttacaaaccatatttcgaacatagtgaaacatgcttgtgttttcatatttggtgataattcaattttaatcaaaataatctctttatacctccaattttcaaaattgggttgtaggccaaaaaagcaacacaaatgggtgaagaaaatagagaaacaaaattatgaaaatatgaagtggatctgaggttattagagcccaacgaggcggtggagcatcgtttttaacaaaatccaacaatatctttaggggtgaaatccaccaaccgagcgttcgctcacaacttaaaggggtgaaattcacaagaagtagtttaaaggggtgaaattcacaagaagtagttgaacaagtgactttaggaatgtgcgattcacgttctggggttcaggtcgcaacaaaactttgaggcatggtggtgccatgtggtttcacattgtcggacagtggagccgtgttaaagggagtaaaggcttggtggtggccgtttgtggtgaaaaatatgatttggagatagatgggacgtggacttaacagacagagtggatggtttttttttaaatttaattcagtaaaattatttttataaattaatgtatgatagtgtatatgcattaaatttatttaaatttttactaattagtaatttgtttttagtagtgacgaatttgtttttgtcactaaattttgcctttataaaaaatggggtggagtgtagatttttaaaaagaatggggtggagtgtcattcttgcaaatcttgaggggggtgagtgtattttactcaactcttaaaaaaattaggaattaattagttgtaaattataaaagtttatttcattgaataacataacaaattaaaggcaaaaaaatacaaaacttcattttatatgtatttcagaaaaattgcctactttcaattatgagaaactaaaattatgtttagtttaaaatgagcatagattcaaaaattaataaataatatatatagcaggatacatgcctatcaattaacatatcgtttgtccacgatgatgatcttattggaggatcaatatcttcaaattaacaaagttatcacttggctcttattggtcataatgcaataaaaaaattgcaattagtatcaaatcaaactgaaatttgcaactatatgctgctggtgttgtcgcgcagattcctttttgatttttatgggaatgaagtcaatgaagcaacagtttcacaggcgtgcttaaaaataaaaaaattggaaatttgatgtttgttaggattatgagaggacacaatgggaggatgtttcacaagctgcagacagggttgccacttcagatgcaaaggattaaataaacaaagccaaggtttgcaatcaacaagattccatcgtcgttttgcttcctttaatcgtattaatcaaaagcacaccaagtaaagcatcaatatataacatccaagaaatcacaacatgatagttgctcgtgtcgtctattaactatgatgtcaggagttcgatccccgctcatgtgaatggaagtcatttcgttgttagatgtttaccgtttaatgcaaatactcgcggtgagataataagtcattgttgtgggcgaataccctaaaataagaataaaattaaatatagcatccaagttattgcccaaatatataaacaatggtattgttgacattattaggcataaaagcagtaggtaagtgtattatatttatttaattttttaaaattttgaaattaattaataattgttaacataagtaaaccatttttagcaaaaactctacacttctattaccttaacaagtacatttttgatggtacaccttaacaattaacaagtcatatgattgacaaacatattttatatgctttacaatttattctaaaatcaaagtttatgggaagaagctcataaaagtagttcctgggtgttttttagaatagagaagttgatcatgttagaaattaagttaaaaatgagttgaaagtgatttatgtttgattatatttatgagaaaaatgaattgtctgatgtaatattgtaaaatctaacaattaattaagtaccacagaaactagaatttatagcttcaccttagaattgattttggagttaaaatcaattattaaaggagcaattattaaaggagacatccaaatacactagttaattttgacaatcaattctaacacttgcaaatgtgtaaccaaacttactatcagtaagtgaactaatgattcccaagtcaacttttgttctagctagccaaccgttactatgttccctccacaatacattctccttgaaactgtcaagtgtcaactgcacccaaacatccttgtttgtgatgaaaagatcgaaaacgtgtgcttatgaatttacatgtttacattcaccaaaaatcaaaagttacacctctatacttatcacatatgtttgagtcactttccatataaaatcccatagtctattaattatcagagtaagtgtgacattagtttcaagagaaaaataaatgatcaaaacctggtagagagtcctagaaattcaatgttctgatttctttcattcatctctgctgccattttgatttgcaca

SEQ ID NO: 90 - DNA, 24 bp, synthetic construct
  source 1..24: mol_type = other DNA
gaggtactgctagcattactacta

SEQ ID NO: 91 - DNA, 24 bp, synthetic construct
  source 1..24: mol_type = other DNA
atggccaccatgcctatggggaat

SEQ ID NO: 92 - DNA, 24 bp, synthetic construct
  source 1..24: mol_type = other DNA
agctgctgccctgtctccaagcac

SEQ ID NO: 93 - DNA, 24 bp, synthetic construct
  source 1..24: mol_type = other DNA
gaggtactgctagcattactacta

SEQ ID NO: 94 - DNA, 24 bp, synthetic construct
  source 1..24: mol_type = other DNA
ttgccgatgtcgcttttggtgaga

SEQ ID NO: 95 - DNA, 28 bp, synthetic construct
  source 1..28: mol_type = other DNA
tccgcttgatagatgaagtggccttaaa

SEQ ID NO: 96 - DNA, 24 bp, synthetic construct
  source 1..24: mol_type = other DNA
tcccttccattctgcattcccacc

SEQ ID NO: 97 - DNA, 23 bp, synthetic construct
  source 1..23: mol_type = other DNA
gcgtcagccacagttgtttccca

SEQ ID NO: 98 - DNA, 24 bp, synthetic construct
  source 1..24: mol_type = other DNA
tggttgcatgtcctgagggaggct

SEQ ID NO: 99 - DNA, 28 bp, synthetic construct
  source 1..28: mol_type = other DNA
tggatctctgagttaccaggaggcatgg

SEQ ID NO: 100 - DNA, 24 bp, synthetic construct
  source 1..24: mol_type = other DNA
gtggtggtcagttactttaggaga

SEQ ID NO: 101 - DNA, 27 bp, synthetic construct
  source 1..27: mol_type = other DNA
cccccattgaagcaattacctttttcc

SEQ ID NO: 102 - DNA, 24 bp, synthetic construct
  source 1..24: mol_type = other DNA
ttgccacccctcctccttcttgct

SEQ ID NO: 103 - DNA, 24 bp, synthetic construct
  source 1..24: mol_type = other DNA
gtggtggtcagttactttaggaga

SEQ ID NO: 104 - DNA, 24 bp, synthetic construct
  source 1..24: mol_type = other DNA
tagtgcgttgactgtggggcgaga

SEQ ID NO: 105 - DNA, 24 bp, synthetic construct
  source 1..24: mol_type = other DNA
ttgcagcgcggttgtttggtatga

SEQ ID NO: 106 - DNA, 24 bp, synthetic construct
  source 1..24: mol_type = other DNA
accgctttctcctcaggctccgtg

SEQ ID NO: 107 - DNA, 24 bp, synthetic construct
  source 1..24: mol_type = other DNA
agtcgcaaaagtgaagggccacca

SEQ ID NO: 108 - DNA, 27 bp, synthetic construct
  source 1..27: mol_type = other DNA
caccgcatatgaaaagattggtggtga

SEQ ID NO: 109 - DNA, 24 bp, synthetic construct
  source 1..24: mol_type = other DNA
tggttgcatgtcctgagggaggct

SEQ ID NO: 110 - DNA, 24 bp, synthetic construct
  source 1..24: mol_type = other DNA
ccttcacagtgctgatctcgtgaa

SEQ ID NO: 111 - DNA, 21 bp, synthetic construct
  source 1..21: mol_type = other DNA
aggccacttcatctatcaagc

SEQ ID NO: 112 - DNA, 23 bp, synthetic construct
  source 1..23: mol_type = other DNA
ctcatccttcctatgctgaattc

SEQ ID NO: 113 - DNA, 24 bp, synthetic construct
  source 1..24: mol_type = other DNA
ggaccttccaaaactggctagtac

SEQ ID NO: 114 - DNA, 23 bp, synthetic construct
  source 1..23: mol_type = other DNA
acatggacaagttgtggaagctg

SEQ ID NO: 115 - DNA, 22 bp, synthetic construct
  source 1..22: mol_type = other DNA
tctcgctacttggcaaccacaa

SEQ ID NO: 116 - DNA, 20 bp, synthetic construct
  source 1..20: mol_type = other DNA
atgtgcattttaagacaggg

SEQ ID NO: 117 - DNA, 24 bp, synthetic construct
  source 1..24: mol_type = other DNA
atcatttctgacaaggagagcacg

SEQ ID NO: 118 - DNA, 22 bp, synthetic construct
  source 1..22: mol_type = other DNA
gccagggaatccattaacattt

SEQ ID NO: 119 - DNA, 23 bp, synthetic construct
  source 1..23: mol_type = other DNA
agaggaaggttgaggaatggtgt

SEQ ID NO: 120 - DNA, 24 bp, synthetic construct
  source 1..24: mol_type = other DNA
cctgctcttgtcactctatttggc

SEQ ID NO: 121 - DNA, 23 bp, synthetic construct
  source 1..23: mol_type = other DNA
gtagcacagcttcacctttatgg

SEQ ID NO: 122 - DNA, 22 bp, synthetic construct
  source 1..22: mol_type = other DNA
gatcaggtgaaccctcaaggcc

SEQ ID NO: 123 - DNA, 21 bp, synthetic construct
  source 1..21: mol_type = other DNA
gaacgtagaagattgcctgaa

SEQ ID NO: 124 - DNA, 30 bp, synthetic construct
  source 1..30: mol_type = other DNA
acaagtagcttatgacgtacgacatatagt

SEQ ID NO: 125 - DNA, 30 bp, synthetic construct
  source 1..30: mol_type = other DNA
tctagattgtttgacgatagaatctccaat

SEQ ID NO: 126 - DNA, 30 bp, synthetic construct
  source 1..30: mol_type = other DNA
attattatgttgacggaaagacactacacc

SEQ ID NO: 127 - DNA, 30 bp, synthetic construct
  source 1..30: mol_type = other DNA
agatttaagttgacgtattaatgacaatga

SEQ ID NO: 128 - DNA, 30 bp, synthetic construct
  source 1..30: mol_type = other DNA
agatttaagtaataaatttaatgacaatga

SEQ ID NO: 129 - DNA, 30 bp, synthetic construct
  source 1..30: mol_type = other DNA
acaagtagcttaaataaatcgacatatagt

SEQ ID NO: 130 - DNA, 30 bp, synthetic construct
  source 1..30: mol_type = other DNA
tgtaataacttgtgagcgaagcagggatcc

SEQ ID NO: 131 - DNA, 30 bp, synthetic construct
  source 1..30: mol_type = other DNA
tcgagcctgccttgagcttgtcttctccat

SEQ ID NO: 132 - DNA, 30 bp, synthetic construct
  source 1..30: mol_type = other DNA
aggagaggtttgtgagcatcagcaaagtgt

SEQ ID NO: 133 - DNA, 30 bp, synthetic construct
  source 1..30: mol_type = other DNA
cgttcattaatatgacatcagaatttctct

SEQ ID NO: 134 - DNA, 30 bp, synthetic construct
  source 1..30: mol_type = other DNA
ggtttttatttttgacctcagaggggatcg

SEQ ID NO: 135 - DNA, 30 bp, synthetic construct
  source 1..30: mol_type = other DNA
agacctattttctgactcaatgtagagggt

SEQ ID NO: 136 - DNA, 30 bp, synthetic construct
  source 1..30: mol_type = other DNA
catgtgggcttttgacgaaggttcaagtag

SEQ ID NO: 137 - DNA, 30 bp, synthetic construct
  source 1..30: mol_type = other DNA
actatatgtcgtacgtcataagctacttgt

SEQ ID NO: 138 - DNA, 30 bp, synthetic construct; identical to SEQ ID NO: 125
  source 1..30: mol_type = other DNA

SEQ ID NO: 139 - DNA, 30 bp, synthetic construct
  source 1..30: mol_type = other DNA
ggtgtagtgtctttccgtcaacataataat

SEQ ID NO: 140 - DNA, 30 bp, synthetic construct
  source 1..30: mol_type = other DNA
tcattgtcattaatacgtcaacttaaatct

SEQ ID NO: 141 - DNA, 30 bp, synthetic construct
  source 1..30: mol_type = other DNA
actatatgtcgatttatttaagctacttgt

SEQ ID NO: 142 - DNA, 30 bp, synthetic construct
  source 1..30: mol_type = other DNA
tcattgtcattaaatttattacttaaatct

SEQ ID NO: 143 - DNA, 30 bp, synthetic construct
  source 1..30: mol_type = other DNA
ggatccctgcttcgctcacaagttattaca

SEQ ID NO: 144 - DNA, 30 bp, synthetic construct
  source 1..30: mol_type = other DNA
atggagaagacaagctcaaggcaggctcga

SEQ ID NO: 145 - DNA, 30 bp, synthetic construct
  source 1..30: mol_type = other DNA
acactttgctgatgctcacaaacctctcct

SEQ ID NO: 146 - DNA, 30 bp, synthetic construct
  source 1..30: mol_type = other DNA
agagaaattctgatgtcatattaatgaacg

SEQ ID NO: 147 - DNA, 30 bp, synthetic construct
  source 1..30: mol_type = other DNA
cgatcccctctgaggtcaaaaataaaaacc

SEQ ID NO: 148 - DNA, 30 bp, synthetic construct
  source 1..30: mol_type = other DNA
accctctacattgagtcagaaaataggtct

SEQ ID NO: 149 - DNA, 30 bp, synthetic construct; identical to SEQ ID NO: 136
  source 1..30: mol_type = other DNA

SEQ ID NO: 150 - protein, 488 aa, synthetic construct
MPFSSQRHSRIFLPSHTSLFLFSLMKNMQXFKTAQXSTSXLYCHSSFLLRGNDPSRNPTRFSDLGEXXHSSXXFHQEDAVDLSSSSMFSVKSSNVXVGGSNLQYGTXNTNLGCAEIGSXGGCXDTGQQLMYQKGTXXAAXPLGNGHVENWXDSGMADNSQQTXDTSTDIDTDDKXQCNGVKXGTLXVVXSKDQSKVKPEDQKTLRRLAQNREAARKSRLRKKAYVQQLENSRVRLAQLEQELQRARQQGXFIATGPGDQGHLAVGNGALAFDMDYARWVDEHQRLINDLRSAINSQMGDNELHLLVDGVMAHYDELFRLKSIGAKADVFHILSGMWKTPAERCFMWLGGFRSSELLKIVXNHLEPLTDQQLMGIYNLQQSSQQAEDALSQGMEALQQSLXETLSSTSLGPXGSGNVADYMGQMAIAMGKLATLESFLHQADLLRQQTLQQLQRILTTRQAARALLVINDYISRLRALSSLWLARPREX

(19) United States
(12) Patent Application Publication
(10) Pub. No.: US 2024/0271152 A1
(43) Pub. Date: Aug. 15, 2024

(54) ENHANCING NITROGEN FIXATION WITH FUN

(71) Applicant: Aarhus Universitet, Aarhus (DK)

(72) Inventors: Jieshun LIN, Aarhus (DK); Peter BJØRK, New York, NY (US); Jens Stougaard JENSEN, Aarhus (DK); Kasper Røjkjær ANDERSEN, Aarhus (DK); Dugald REID, Aarhus (DK)

(73) Assignee: Aarhus Universitet, Aarhus (DK)

(21) Appl. No.: 18/431,701

(22) Filed: Feb. 2, 2024

Related U.S. Application Data
(60) Provisional application No. 63/580,171, filed on Sep. 1, 2023.
(60) Provisional application No. 63/483,248, filed on Feb. 3, 2023.

(51) Int. Cl. C12N 15/82; C07K 14/415

(52) U.S. Cl. CPC C12N 15/8271; C07K 14/415; C12N 15/8266

(57) ABSTRACT

The present disclosure relates to enhancing nitrogen fixation in legumes grown under high nitrate or nitrate stress conditions. In particular, the present disclosure relates to genetically modified plants with altered level or expression of FUN or FUN downstream targets, and methods of producing and growing the same. The present disclosure further relates to nodule senescence controlled by FUN and its downstream targets, as well as the regulation of FUN activity or expression by cellular zinc.

### CROSS-REFERENCE TO RELATED APPLICATIONS

This application claims the benefit of U.S. Provisional Application No. 63/483,248, filed Feb. 3, 2023, and U.S. Provisional Application No. 63/580,171, filed Sep. 1, 2023, each of which are hereby incorporated by reference in their entirety.

### REFERENCE TO AN ELECTRONIC SEQUENCE LISTING

The contents of the electronic sequence listing (794542002200SEQLIST.xml; Size: 175,320 bytes; and Date of Creation: Feb. 1, 2024) is herein incorporated by reference in its entirety.

### TECHNICAL FIELD

The present disclosure relates to enhancing nitrogen fixation in legumes grown under high nitrate or nitrate stress conditions. In particular, the present disclosure relates to genetically modified plants with altered level or expression of FUN or FUN downstream targets, and methods of producing and growing the same. The present disclosure further relates to nodule senescence controlled by FUN and its downstream targets, as well as the regulation of FUN activity by cellular zinc.

### BACKGROUND

Plant growth and development depends on carbon dioxide and sunlight above ground, and water and mineral nutrients in the soul. The accessibility of nutrients in the soil depends on many factors, and nutrient availability varies spatially and temporally. Local nutrient sensing, as well as the perception of overall nutrient status, shape the plant's response to its nutrient environment, and act to coordinate plant development with microbial engagement to optimize nutrient capture and regulate plant growth. One of the principal nutrients that limits plant productivity is nitrogen (N).

Nitrogen fixation is critical to the sustainable and profitable production of legumes. The symbiosis between the legume plant and the nitrogen-fixing microbe is controlled by the plant in a number of ways, including through the number of root nodules that are allowed to form (Nishimura, R. et al. HAR1 mediates systemic regulation of symbiotic organ development. Nature 420, 426-429 (2002); Krusell, L., Madsen, L. H., Sato, S. & Aubert, G. Shoot control of root development and nodulation is mediated by a receptor-like kinase. Nature 420, 422-426 (2002); Searle, I. R. et al. Long-distance signaling in nodulation directed by a CLAVATA1-like receptor kinase. Science 299, 109-112 (2003); Tsikou, D. et al. Systemic control of legume susceptibility to rhizobial infection by a mobile microRNA. Science 362, 233-236 (2018)), and the function of the resulting organ. Nitrogen fixation in legumes can support all the nitrogen requirements of the plant and is balanced with the acquisition of nitrogen from available soil resources. The soil nitrogen supply fluctuates, however, and so does the demand for nitrogen by the plant.

In high-intensity agriculture, nitrogen can be applied at high concentrations in the form of inorganic fertilizers to promote crop productivity. These concentrations are generally higher than the amounts needed by plants or able to be stored in soil. This results in release of these nutrients into the environment, affecting ecosystems and biodiversity, and contributing to climate change (C. J. Stevens, Nitrogen in the environment. Science 363, 578-580 (2019); J. A Foley et al., Solutions for a cultivated planet. Nature 478, 337-342 (2011); J. Rockstrom et al., A safe operating space for humanity. Nature 461, 472-475 (2009)). The presence of high nitrate in the soil, however, has been shown to inhibit or suppress the ability of legumes to fix nitrogen.

There exists a general need to enhance the ability of legumes and other plants that engage in symbiosis with nitrogen fixing bacteria to fix nitrogen under high nitrate or nitrate stress conditions that would otherwise inhibit, suppress, or reduce nitrogen fixation. By way of example, the ability to modify a legume's or other plant's response to nitrate and the corresponding regulation of nitrogen fixation is needed. In particular, the identification of transcription factors able to regulate nitrogen fixation under high nitrate conditions is needed, as these provide a means of engineering legumes to fix nitrogen even when grown under high nitrate conditions. By way of further example, the ability to modify senescence of nodules in a legume or other plant in response to nitrate levels and/or nitrogen stress is needed. In particular, the identification of transcription factors able to regulate nodule senescence under high nitrate and/or nitrogen stress conditions is needed, as these provide a further means of engineering legumes to fix nitrogen even when grown under high nitrate conditions or under nitrogen stress.

### BRIEF SUMMARY

In order to meet these needs, the present disclosure provides the FUN transcription factor, which is a regulator of nitrogen fixation in legumes under high nitrate conditions. The present disclosure further provides downstream targets of FUN, which also act to regulate nitrogen fixation under high nitrate conditions. Mutating or downregulating either FUN or its downstream targets can be used to produce plants with nitrate resistant nitrogen fixation. This provides an opportunity to increase biological fixed nitrogen in fields or cropping conditions with high levels of soil nitrate.

An aspect of the disclosure includes a genetically modified plant or part thereof including one or more genetic alterations that result in decreased activity or expression of a FUN protein as compared to the activity or expression of a FUN protein in a control plant grown under the same conditions. In a further embodiment of this aspect, the FUN protein includes a polypeptide with at least 70% identity, at least 80% identity, at least 90% identity, at least 95% identity, or at least 99% identity to a protein selected from the group of SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, or SEQ ID NO: 82, or conserved domains thereof, or combinations thereof. In an additional embodiment of this aspect, the FUN protein includes SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, or SEQ ID NO: 82, or conserved domains thereof, or combinations thereof. In yet another embodiment of this aspect, the FUN protein includes SEQ ID NO: 1, SEQ ID NO: 8, or SEQ ID NO: 9, or conserved domains thereof, or combinations thereof.

A further aspect of the disclosure includes a genetically modified plant or part thereof including one or more genetic alterations that result in decreased activity or expression of one or more of a NRT3.1 protein, a bZIP28 protein, a NAC-domain containing protein, a HO1 protein, a NRT2.1 protein, or an AS1 protein as compared to the activity or expression of a NRT3.1 protein, a bZIP28 protein, a NAC-domain containing protein, a HO1 protein, a NRT2.1 protein, or an AS1 protein in a control plant grown under the same conditions. In a further embodiment of this aspect, wherein the protein is the NRT3.1 protein, and wherein the NRT3.1 protein includes a polypeptide with at least 70% identity, at least 80% identity, at least 90% identity, at least 95% identity, or at least 99% identity to SEQ ID NO: 74 or conserved domains thereof; wherein the protein is the bZIP28 protein, and wherein the bZIP28 protein includes a polypeptide with at least 70% identity, at least 80% identity, at least 90% identity, at least 95% identity, or at least 99% identity to SEQ ID NO: 75 or conserved domains thereof; wherein the protein is the NAC-domain containing protein, and wherein the NAC-domain containing protein includes a polypeptide with at least 70% identity, at least 80% identity, at least 90% identity, at least 95% identity, or at least 99% identity to a protein selected from the group of SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, or conserved domains thereof, or combinations thereof; wherein the protein is the HO1 protein, and wherein the HO1 protein includes a polypeptide with at least 70% identity, at least 80% identity, at least 90% identity, at least 95% identity, or at least 99% identity to SEQ ID NO: 77 or conserved domains thereof; wherein the protein is the NRT2.1 protein, and wherein the NRT2.1 protein includes a polypeptide with at least 70% identity, at least 80% identity, at least 90% identity, at least 95% identity, or at least 99% identity to SEQ ID NO: 78 or conserved domains thereof; or wherein the protein is the AS1 protein, and wherein the AS1 protein includes a polypeptide with at least 70% identity, at least 80% identity, at least 90% identity, at least 95% identity, or at least 99% identity to SEQ ID NO: 79 or conserved domains thereof. In an additional embodiment of this aspect, the NRT3.1 protein includes SEQ ID NO: 74 or conserved domains thereof; wherein the bZIP28 protein includes SEQ ID NO: 75 or conserved domains thereof; wherein the NAC-domain containing protein includes SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, or SEQ ID NO: 73, or conserved domains thereof; wherein the HO1 protein includes SEQ ID NO: 77 or conserved domains thereof; wherein the NRT2.1 protein includes SEQ ID NO: 78 or conserved domains thereof, or wherein the AS1 protein includes SEQ ID NO: 79. In still another embodiment of this aspect, the NAC-domain containing protein includes SEQ ID NO: 31, SEQ ID NO: 41, or SEQ ID NO: 42, or conserved domains thereof, or combinations thereof.

Another aspect of the disclosure includes a genetically modified plant including one or more genetic alterations that result in decreased activity or expression of one or more of a FUN protein, a FUN-like protein, a NRT3.1 protein, a bZIP28 protein, a NAC-domain containing protein, a HO1 protein, a NRT2.1 protein, or an AS1 protein as compared to the activity or expression of a FUN protein, a FUN-like protein, a NRT3.1 protein, a bZIP28 protein, a NAC-domain containing protein, a HO1 protein, a NRT2.1 protein, or an AS1 protein in a control plant grown under the same conditions, wherein the FUN protein, the FUN-like protein, the NRT3.1 protein, the bZIP28 protein, the NAC-domain containing protein, the HO1 protein, the NRT2.1 protein, or the AS1 protein is selected from the group of a polypeptide with at least 70% identity, at least 80% identity, at least 90% identity, at least 95% identity, or at least 99% identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, or SEQ ID NO: 84, or conserved domains thereof, or combinations thereof, and wherein the FUN protein, the FUN-like protein, the NRT3.1 protein, the bZIP28 protein, the NAC-domain containing protein, the HO1 protein, the NRT2.1 protein, or the AS1 protein has enhanced expression in a root nodule absent the one or more genetic alterations.

In an additional embodiment of this aspect, which may be combined with any of the preceding embodiments, the decrease is at least 10%, at least 20%, at least 30%, at least 40%, at least 50%, at least 60%, at least 70%, at least 80%, at least 90%, or at least 100%. In yet another embodiment of this aspect, which may be combined with any of the preceding embodiments, the decrease is due to knock-out of a gene for the protein, introduction of a premature stop codon in the coding sequence of the gene for the protein, RNAi silencing, knock-out of a domain of the protein, introduction of a transcriptional repressor protein binding site, or knock-out of a binding site in the promoter region of the gene and/or the genetic alterations include knock-out of a gene for the protein, introduction of a premature stop codon in the coding sequence of the gene for the protein, RNAi silencing, knock-out of a domain of the protein, introduction of a transcriptional repressor protein binding site, or knock-out of a binding site in the promoter region of the gene and/or the genetic alterations include knock-out of a gene for the protein, introduction of a premature stop codon in the coding sequence of the gene for the protein, RNAi silencing, knock-out of a domain of the protein, introduction of a transcriptional repressor protein binding site, or knock-out of a binding site in the promoter region of the gene, preferably the binding site is a transcriptional activator protein binding site or a TATA-box. In a further embodiment of this aspect, which may be combined with any of the preceding embodiments, the growth conditions include a moderate nitrate level, a high nitrate level, or a nitrate level around the plant that reduces or suppresses nitrogen fixation. In yet another embodiment of this aspect, the nitrate level is between about 10 mM and about 250 mM nitrate or includes at least about 10 mM nitrate, at least about 20 mM nitrate, at least about 30 mM nitrate, at least about 40 mM nitrate, at least about 50 mM nitrate, at least about 100 mM nitrate, at least about 150 mM nitrate, at least about 200 mM nitrate, or at least about 250 mM nitrate. In still another embodiment of this aspect, the genetically modified plant has increased nitrogen fixation as compared to the control plant when grown under the same growth conditions. In a further embodiment of this aspect, the nitrogen fixation is increased at least 10%, at least 20%, at least 30%, at least 40%, at least 50%, at least 60%, at least 70%, at least 80%, at least 90%, at least 100%, at least 150%, at least 200%, at least 300%, at least 400%, or at least 500%. In yet another embodiment of this aspect, which may be combined with any of the preceding embodiments, the plant forms nodules. In an additional embodiment of this aspect, the number of nodules is increased, hemoglobin content is increased, or the acetylene reduction assay (ARA) activity is increased compared to the control plant when grown under the same conditions.

An additional aspect of the disclosure includes methods of cultivating a genetically altered plant with increased nitrogen fixation under conditions including a nitrate level around the plant roots that suppresses nitrogen fixation, including: (a) providing the genetically altered plant, wherein the plant or a part thereof includes one or more genetic alterations that result in decreased activity or expression of a FUN protein, a NRT3.1 protein, a bZIP28 protein, a NAC-domain containing protein, a HO1 protein, a NRT2.1 protein, or an AS1 protein, or any combination thereof as compared to an activity or expression of a FUN protein, a NRT3.1 protein, a bZIP28 protein, a NAC-domain containing protein, a HO1 protein, a NRT2.1 protein, or an AS1 protein in a control plant grown under the same conditions, and wherein the one or more genetic alterations reduce the nitrate level suppression of nitrogen fixation; and (b) cultivating the genetically altered plant under the nitrate level around the plant roots, wherein the genetically modified plant has increased nitrogen fixation as compared to the control plant grown under the same conditions. In a further embodiment of this aspect, the FUN protein includes a polypeptide with at least 70% identity, at least 80% identity, at least 90% identity, at least 95% identity, or at least 99% identity to a protein selected from the group of SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, or SEQ ID NO: 82, or conserved domains thereof, or combinations thereof. In another embodiment of this aspect, the FUN protein includes SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, or SEQ ID NO: 82, or conserved domains thereof, or combinations thereof. In still another embodiment of this aspect, the FUN protein includes SEQ ID NO: 1, SEQ ID NO: 8, or SEQ ID NO: 9, or conserved domains thereof, or combinations thereof. In yet another embodiment of this aspect, wherein the protein is the NRT3.1 protein, and wherein the NRT3.1 protein includes a polypeptide with at least 70% identity, at least 80% identity, at least 90% identity, at least 95% identity, or at least 99% identity to SEQ ID NO: 74 or conserved domains thereof; wherein the protein is the bZIP28 protein, and wherein the bZIP28 protein includes a polypeptide with at least 70% identity, at least 80% identity, at least 90% identity, at least 95% identity, or at least 99% identity to SEQ ID NO: 75 or conserved domains thereof; wherein the protein is the NAC-domain containing protein, and wherein the NAC-domain containing protein includes a polypeptide with at least 70% identity, at least 80% identity, at least 90% identity, at least 95% identity, or at least 99% identity to a protein selected from the group of SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, or conserved domains thereof, or combinations thereof; wherein the protein is the HO1 protein, and wherein the HO1 protein includes a polypeptide with at least 70% identity, at least 80% identity, at least 90% identity, at least 95% identity, or at least 99% identity to SEQ ID NO: 77 or conserved domains thereof; wherein the protein is the NRT2.1 protein, and wherein the NRT2.1 protein includes a polypeptide with at least 70% identity, at least 80% identity, at least 90% identity, at least 95% identity, or at least 99% identity to SEQ ID NO: 78 or conserved domains thereof; or wherein the protein is the AS1 protein, and wherein the AS1 protein includes a polypeptide with at least 70% identity, at least 80% identity, at least 90% identity, at least 95% identity, or at least 99% identity to SEQ ID NO: 79 or conserved domains thereof. In a further embodiment of this aspect, wherein the NRT3.1 protein includes SEQ ID NO: 74 or conserved domains thereof; wherein the bZIP28 protein includes SEQ ID NO: 75 or conserved domains thereof; wherein the NAC-domain containing protein includes SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, or SEQ ID NO: 73, or conserved domains thereof, or combinations thereof; wherein the HO1 protein includes SEQ ID NO: 77 or conserved domains thereof; wherein the NRT2.1 protein includes SEQ ID NO: 78 or conserved domains thereof, or wherein the AS1 protein includes SEQ ID NO: 79 or conserved domains thereof. In still another embodiment of this aspect, the NAC-domain containing protein includes SEQ ID NO: 31, SEQ ID NO: 41, or SEQ ID NO: 42, or conserved domains thereof, or combinations thereof. Additional embodiments of this aspect, which may be combined with any of the preceding embodiments, include the nitrate level in step (c) being between about 10 mM and about 250 mM nitrate at least about 10 mM nitrate, at least about 20 mM nitrate, at least about 30 mM nitrate, at least about 40 mM nitrate, at least about 50 mM nitrate, at least about 100 mM nitrate, at least about 150 mM nitrate, at least about 200 mM nitrate, or at least about 250 mM nitrate. Further embodiments of this aspect, which may be combined with any of the preceding embodiments, include the nitrogen fixation being increased at least 10%, at least 20%, at least 30%, at least 40%, at least 50%, at least 60%, at least 70%, at least 80%, at least 90%, at least 100%, at least 150%, at least 200%, at least 300%, at least 400%, or at least 500%. In a further embodiment of this aspect, the number of nodules is increased or the hemoglobin content is increased compared to the control plant when grown under the same growth conditions. In additional embodiments of this aspect, increased nitrogen fixation is measured using a method selected from the group of measuring the number of pink nodules per plant as compared to a control plant, measuring the amount of acetylene (C₂H₂) reduced to ethylene (C₂H₄) per hour (acetylene reduction assay (ARA)) as compared to a control plant, or measuring the micrograms of hemoglobin per plant as compared to a control plant.

A further aspect of the disclosure includes methods of cultivating a genetically altered plant able to fix nitrogen when grown in nitrogen-fertilized conditions, including: (a) providing the genetically altered plant, wherein the plant or a part thereof includes one or more genetic alterations that result in decreased activity or expression of a FUN protein, a NRT3.1 protein, a bZIP28 protein, a NAC-domain containing protein, a HO1 protein, a NRT2.1 protein, or an AS1 protein, or any combination thereof as compared to an activity or expression of a FUN protein, a NRT3.1 protein, a bZIP28 protein, a NAC-domain containing protein, a HO1 protein, a NRT2.1 protein, or an AS1 protein in a control plant grown under the same conditions, and wherein the one or more genetic alterations reduce the nitrate level suppression of nitrogen fixation; (b) cultivating the plant under conditions including a standard nitrate level around the plant roots; and (c) applying nitrogen fertilizer, thereby generating conditions including a nitrate level around the plant roots that suppresses nitrogen fixation, wherein the genetically modified plant has increased nitrogen fixation as compared to the control plant grown under the same conditions. In a further embodiment of this aspect, the FUN protein includes a polypeptide with at least 70% identity, at least 80% identity, at least 90% identity, at least 95% identity, or at least 99% identity to a protein selected from the group of SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, or SEQ ID NO: 82, or conserved domains thereof, or combinations thereof. In another embodiment of this aspect, the FUN protein includes SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, or SEQ ID NO: 82, or conserved domains thereof, or combinations thereof. In still another embodiment of this aspect, the FUN protein includes SEQ ID NO: 1, SEQ ID NO: 8, or SEQ ID NO: 9, or conserved domains thereof, or combinations thereof. In yet another embodiment of this aspect, wherein the protein is the NRT3.1 protein, and wherein the NRT3.1 protein includes a polypeptide with at least 70% identity, at least 80% identity, at least 90% identity, at least 95% identity, or at least 99% identity to SEQ ID NO: 74 or conserved domains thereof; wherein the protein is the bZIP28 protein, and wherein the bZIP28 protein includes a polypeptide with at least 70% identity, at least 80% identity, at least 90% identity, at least 95% identity, or at least 99% identity to SEQ ID NO: 75 or conserved domains thereof; wherein the protein is the NAC-domain containing protein, and wherein the NAC-domain containing protein includes a polypeptide with at least 70% identity, at least 80% identity, at least 90% identity, at least 95% identity, or at least 99% identity to a protein selected from the group of SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, or conserved domains thereof, or combinations thereof; wherein the protein is the HO1 protein, and wherein the HO1 protein includes a polypeptide with at least 70% identity, at least 80% identity, at least 90% identity, at least 95% identity, or at least 99% identity to SEQ ID NO: 77 or conserved domains thereof; wherein the protein is the NRT2.1 protein, and wherein the NRT2.1 protein includes a polypeptide with at least 70% identity, at least 80% identity, at least 90% identity, at least 95% identity, or at least 99% identity to SEQ ID NO: 78 or conserved domains thereof; or wherein the protein is the AS1 protein, and wherein the AS1 protein includes a polypeptide with at least 70% identity, at least 80% identity, at least 90% identity, at least 95% identity, or at least 99% identity to SEQ ID NO: 79 or conserved domains thereof. In a further embodiment of this aspect, wherein the NRT3.1 protein includes SEQ ID NO: 74 or conserved domains thereof; wherein the bZIP28 protein includes SEQ ID NO: 75 or conserved domains thereof; wherein the NAC-domain containing protein includes SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, or SEQ ID NO: 73, or conserved domains thereof, or combinations thereof; wherein the HO1 protein includes SEQ ID NO: 77 or conserved domains thereof; wherein the NRT2.1 protein includes SEQ ID NO: 78 or conserved domains thereof, or wherein the AS1 protein includes SEQ ID NO: 79 or conserved domains thereof. In still another embodiment of this aspect, the NAC-domain containing protein includes SEQ ID NO: 31, SEQ ID NO: 41, or SEQ ID NO: 42, or conserved domains thereof, or combinations thereof. Additional embodiments of this aspect, which may be combined with any of the preceding embodiments, include the nitrate level in step (c) being between about 10 mM and about 250 mM nitrate or at least about 10 mM nitrate, at least about 20 mM nitrate, at least about 30 mM nitrate, at least about 40 mM nitrate, at least about 50 mM nitrate, at least about 100 mM nitrate, at least about 150 mM nitrate, at least about 200 mM nitrate, or at least about 250 mM nitrate. Further embodiments of this aspect, which may be combined with any of the preceding embodiments, include the nitrogen fixation being increased at least 10%, at least 20%, at least 30%, at least 40%, at least 50%, at least 60%, at least 70%, at least 80%, at least 90%, at least 100%, at least 150%, at least 200%, at least 300%, at least 400%, or at least 500%. In a further embodiment of this aspect, the number of nodules is increased or the hemoglobin content is increased compared to the control plant when grown under the same growth conditions. In additional embodiments of this aspect, increased nitrogen fixation is measured using a method selected from the group of measuring the number of pink nodules per plant as compared to a control plant, measuring the amount of acetylene (C₂H₂) reduced to ethylene (C₂H₄) per hour (acetylene reduction assay (ARA)) as compared to a control plant, or measuring the micrograms of hemoglobin per plant as compared to a control plant. In further embodiments of this aspect, which may be combined with any of the preceding embodiments, the genetically altered plant is grown in an intercropping system with a plant that does not fix nitrogen or in a sequential system after a plant that does not fix nitrogen.

An additional aspect of the disclosure includes methods of delaying nodule senescence, including: (a) providing a genetically altered plant, wherein the plant or a part thereof includes one or more genetic alterations that result in decreased activity or expression of a FUN protein, a NRT3.1 protein, a bZIP28 protein, a NAC-domain containing protein, a HO1 protein, a NRT2.1 protein, or an AS1 protein, or any combination thereof as compared to an activity or expression of a FUN protein, a NRT3.1 protein, a bZIP28 protein, a NAC-domain containing protein, a HO1 protein, a NRT2.1 protein, or an AS1 protein in a control plant grown under the same conditions, and wherein the one or more genetic alterations delay nodule senescence; and (b) cultivating the genetically altered plant under stress conditions, wherein the genetically altered plant has delayed nodule senescence as compared to the control plant grown under the same conditions. A further embodiment of this aspect includes the stress conditions being selected from the group of a moderate nitrate level, a high nitrate level, a nitrate level around the plant that promotes nodule senescence, a moderate heat level, a high heat level, a heat level around the plant that promotes nodule senescence, a moderate water deficit level, a high water deficit level, a water deficit level around the plant that promotes nodule senescence, a moderate waterlogging level, a high waterlogging level, or a waterlogging level around the plant that promotes nodule senescence. In a further embodiment of this aspect, which may be combined with any of the preceding embodiments. the FUN protein includes a polypeptide with at least 70% identity, at least 80% identity, at least 90% identity, at least 95% identity, or at least 99% identity to a protein selected from the group of SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, or SEQ ID NO: 82, or conserved domains thereof, or combinations thereof. In another embodiment of this aspect, the FUN protein includes SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, or SEQ ID NO: 82, or conserved domains thereof, or combinations thereof. In still another embodiment of this aspect, the FUN protein includes SEQ ID NO: 1, SEQ ID NO: 8, or SEQ ID NO: 9, or conserved domains thereof, or combinations thereof. In yet another embodiment of this aspect, which may be combined with any of the preceding embodiments that have a NAC-domain containing protein, wherein the protein is the NRT3.1 protein, and wherein the NRT3.1 protein includes a polypeptide with at least 70% identity, at least 80% identity, at least 90% identity, at least 95% identity, or at least 99% identity to SEQ ID NO: 74 or conserved domains thereof; wherein the protein is the bZIP28 protein, and wherein the bZIP28 protein includes a polypeptide with at least 70% identity, at least 80% identity, at least 90% identity, at least 95% identity, or at least 99% identity to SEQ ID NO: 75 or conserved domains thereof; wherein the protein is the NAC-domain containing protein, and wherein the NAC-domain containing protein includes a polypeptide with at least 70% identity, at least 80% identity, at least 90% identity, at least 95% identity, or at least 99% identity to a protein selected from the group of SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, or conserved domains thereof, or combinations thereof; wherein the protein is the HO1 protein, and wherein the HO1 protein includes a polypeptide with at least 70% identity, at least 80% identity, at least 90% identity, at least 95% identity, or at least 99% identity to SEQ ID NO: 77 or conserved domains thereof; wherein the protein is the NRT2.1 protein, and wherein the NRT2.1 protein includes a polypeptide with at least 70% identity, at least 80% identity, at least 90% identity, at least 95% identity, or at least 99% identity to SEQ ID NO: 78 or conserved domains thereof; or wherein the protein is the AS1 protein, and wherein the AS1 protein includes a polypeptide with at least 70% identity, at least 80% identity, at least 90% identity, at least 95% identity, or at least 99% identity to SEQ ID NO: 79 or conserved domains thereof. In a further embodiment of this aspect, wherein the NRT3.1 protein includes SEQ ID NO: 74 or conserved domains thereof; wherein the bZIP28 protein includes SEQ ID NO: 75 or conserved domains thereof; wherein the NAC-domain containing protein includes SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, or SEQ ID NO: 73, or conserved domains thereof, or combinations thereof; wherein the HO1 protein includes SEQ ID NO: 77 or conserved domains thereof; wherein the NRT2.1 protein includes SEQ ID NO: 78 or conserved domains thereof, or wherein the AS1 protein includes SEQ ID NO: 79 or conserved domains thereof. In still another embodiment of this aspect, the NAC-domain containing protein includes SEQ ID NO: 31, SEQ ID NO: 41, or SEQ ID NO: 42, or conserved domains thereof, or combinations thereof. In a further embodiment of this aspect, the nodule senescence is delayed at least 10%, at least 20%, at least 30%, at least 40%, at least 50%, at least 60%, at least 70%, at least 80%, at least 90%, at least 100%, at least 150%, at least 200%, at least 300%, at least 400%, or at least 500%.

Yet further aspects of the disclosure include methods of inducing filamentation of a FUN protein, including: (a) providing the FUN protein; and (b) increasing an amount of zinc or manganese in an environment of the FUN protein, wherein the increased amount of zinc or manganese induces filamentation as compared to the control FUN protein in an environment without the increased amount of zinc or manganese. In an additional embodiment of this aspect, the filamentation is induced under high nitrate conditions. In yet another embodiment of this aspect, which may be combined with any of the preceding embodiments, the method is performed in vitro.

Still further aspects of the disclosure include methods of inducing filamentation, including: (a) providing a plant including a FUN protein; and (b) cultivating the plant under increased zinc or manganese conditions, wherein filamentation of the FUN protein in the plant is induced as compared a FUN protein in a control plant grown under conditions without increased zinc or manganese. In another embodiment of this aspect, the plant comprises genetic alteration. In an additional embodiment of this aspect, the filamentation is induced under high nitrate conditions. In yet another embodiment of this aspect, which may be combined with any of the preceding embodiments where the plant comprises a genetic alteration, the genetic alteration decreases the activity of the FUN protein without eliminating the activity of the FUN protein. In a further embodiment of this aspect, the induction of filamentation results in increased nitrogen fixation in the genetically altered plant as compared to the control plant grown under the same conditions or reduces the activity or inactivates the FUN protein. In still another embodiment of this aspect, which may be combined with any of the preceding embodiments or aspects that have methods of inducing filamentation, the number of nodules is increased, hemoglobin content is increased, or the acetylene reduction assay (ARA) activity is increased compared to the control plant when grown under the same conditions.

Yet further aspects of the disclosure include methods of tuning nodule function to the amount of available nitrogen in the soil, including: a) providing a genetically altered plant comprising a FUN protein with altered activation by nitrate; and b) cultivating the genetically altered plant under nitrate concentration conditions, wherein the genetically altered plant has reduced activity or expression of FUN and/or reduced active form of FUN as compared to a WT plant grown under the same nitrate conditions. In an additional embodiment of this aspect, altering FUN protein activation by nitrate includes downregulating FUN, reducing FUN activity, knocking out FUN by mutation, knocking down FUN expression, knocking out promoter elements of FUN, or a combination thereof. In a further embodiment of this aspect, altering FUN protein activation by nitrate includes manipulating a level of environmental or cellular zinc, wherein this manipulation results in the FUN protein being maintained in inactive filament form. In still another embodiment of this aspect, altering FUN protein activation by nitrate comprises genetically modifying the FUN protein sequence to alter sensitivity to zinc.

In a further embodiment of this aspect, which may be combined with any of the preceding embodiments or aspects, the FUN protein includes a polypeptide with at least 70% identity, at least 80% identity, at least 90% identity, at least 95% identity, or at least 99% identity to a protein selected from the group of SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, SEQ ID NO: 82, or conserved domains thereof, or combinations thereof. In another embodiment of this aspect, the FUN protein includes SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, or SEQ ID NO: 82 or conserved domains thereof, or combinations thereof. In still another embodiment of this aspect, the FUN protein includes SEQ ID NO: 1, SEQ ID NO: 8, or SEQ ID NO: 9, or conserved domains thereof, or combinations thereof.

A further aspect of the disclosure includes a method of making a genetically altered plant with increased nitrogen fixation under conditions including a nitrate level around the plant roots that suppresses nitrogen fixation, including introducing into the plant or a part thereof one or more genetic alterations that decrease activity or expression of a FUN protein as compared to the activity or expression of a FUN protein in a control plant grown under the same conditions. In an additional embodiment of this aspect, the FUN protein includes a polypeptide with at least 70% identity, at least 80% identity, at least 90% identity, at least 95% identity, or at least 99% identity to a protein selected from the group of SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, or SEQ ID NO: 82, or conserved domains thereof, or combinations thereof; wherein the FUN protein includes SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, or SEQ ID NO: 82, or conserved domains thereof, or combinations thereof; or wherein the FUN protein includes SEQ ID NO: 1, SEQ ID NO: 8, or SEQ ID NO: 9, or conserved domains thereof, or combinations thereof.

An additional aspect of the disclosure includes methods of making a genetically altered plant with increased nitrogen fixation under conditions including a nitrate level around the plant roots that suppresses nitrogen fixation, including introducing into the plant or a part thereof one or more genetic alterations that decrease activity or expression of one or more of a NRT3.1 protein, a bZIP28 protein, a NAC-domain containing protein, a HO1 protein, a NRT2.1 protein, or an AS1 protein as compared to the activity or expression of a NRT3.1 protein, a bZIP28 protein, a NAC-domain containing protein, a HO1 protein, a NRT2.1 protein, or an AS1 protein in a control plant grown under the same conditions. In yet another embodiment of this aspect, wherein the protein is the NRT3.1 protein, and wherein the NRT3.1 protein includes a polypeptide with at least 70% identity, at least 80% identity, at least 90% identity, at least 95% identity, or at least 99% identity to SEQ ID NO: 74 or conserved domains thereof; wherein the protein is the bZIP28 protein, and wherein the bZIP28 protein includes a polypeptide with at least 70% identity, at least 80% identity, at least 90% identity, at least 95% identity, or at least 99% identity to SEQ ID NO: 75 or conserved domains thereof; wherein the protein is the NAC-domain containing protein, and wherein the NAC-domain containing protein includes a polypeptide with at least 70% identity, at least 80% identity, at least 90% identity, at least 95% identity, or at least 99% identity to a protein selected from the group of SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, or conserved domains thereof, or combinations thereof; wherein the protein is the HO1 protein, and wherein the HO1 protein includes a polypeptide with at least 70% identity, at least 80% identity, at least 90% identity, at least 95% identity, or at least 99% identity to SEQ ID NO: 77 or conserved domains thereof; wherein the protein is the NRT2.1 protein, and wherein the NRT2.1 protein includes a polypeptide with at least 70% identity, at least 80% identity, at least 90% identity, at least 95% identity, or at least 99% identity to SEQ ID NO: 78 or conserved domains thereof; or wherein the protein is the AS1 protein, and wherein the AS1 protein includes a polypeptide with at least 70% identity, at least 80% identity, at least 90% identity, at least 95% identity, or at least 99% identity to SEQ ID NO: 79 or conserved domains thereof; wherein the NRT3.1 protein includes SEQ ID NO: 74 or conserved domains thereof; wherein the bZIP28 protein includes SEQ ID NO: 75 or conserved domains thereof; wherein the NAC-domain containing protein includes SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, or SEQ ID NO: 73, or conserved domains thereof, or combinations thereof; wherein the HO1 protein includes SEQ ID NO: 77 or conserved domains thereof; wherein the NRT2.1 protein includes SEQ ID NO: 78 or conserved domains thereof, or wherein the AS1 protein includes SEQ ID NO: 79; or wherein the NAC-domain containing protein includes SEQ ID NO: 31, SEQ ID NO: 41, or SEQ ID NO: 42, or conserved domains thereof, or combinations thereof.

Yet another aspect of the disclosure includes methods of making a genetically altered plant with increased nitrogen fixation under conditions including a nitrate level around the plant roots that suppresses nitrogen fixation, including introducing into the plant or a part thereof one or more genetic alterations that decrease activity or expression of one or more of a FUN protein, a FUN-like protein, a NRT3.1 protein, a bZIP28 protein, a NAC-domain containing protein, a HO1 protein, a NRT2.1 protein, or an AS1 protein as compared to the activity or expression of a FUN protein, a FUN-like protein, a NRT3.1 protein, a bZIP28 protein, a NAC-domain containing protein, a HO1 protein, a NRT2.1 protein, or an AS1 protein in a control plant grown under the same conditions. In some embodiments of this aspect, the FUN protein, the FUN-like protein, the NRT3.1 protein, the bZIP28 protein, the NAC-domain containing protein, the HO1 protein, the NRT2.1 protein, or the AS1 protein is selected from the group of a polypeptide with at least 70% identity, at least 80% identity, at least 90% identity, at least 95% identity, or at least 99% identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, or SEQ ID NO: 84, or conserved domains thereof, or combinations thereof, and wherein the FUN protein, the FUN-like protein, the NRT3.1 protein, the bZIP28 protein, the NAC-domain containing protein, the HO1 protein, the NRT2.1 protein, or the AS1 protein has enhanced expression in a root nodule absent the one or more genetic alterations.

An additional aspect of the disclosure includes methods of making the genetically modified plant or part thereof of any of the above embodiments, including: introducing a genetic alteration to the plant cell that reduces or knocks out activity or expression of a FUN protein, a FUN-like protein, a NAC-domain containing protein, a NRT3.1 protein, a bZIP28 protein, a NAC-domain containing protein, a HO1 protein, a NRT2.1 protein, or an AS1 protein. In a further embodiment of this aspect, the genetic alteration includes a first nucleic acid sequence able to reduce or knock out a second nucleic acid sequence encoding a FUN protein, a FUN-like protein, a NAC-domain containing protein, a NRT3.1 protein, a bZIP28 protein, a NAC-domain containing protein, a HO1 protein, a NRT2.1 protein, or an AS1 protein operably linked to a promoter. In an additional embodiment of this aspect, which may be combined with any of the preceding embodiments, the genetically altered plant is selected from one or more of the group consisting of alfalfa, Bambara groundnut, bean (e.g., kidney beans, black beans, etc.), black currant, chickpea, clover, cowpea, forage legumes, legume trees, lentil, lotus, lupin, Medicago spp., pea, peanut, pigeon pea, soybean, Parasponia, alder trees, and elm trees. In another embodiment of this aspect, the nucleic acid includes a RNA silencing associated short RNA, an antisense RNA, a siRNA, a miRNA, a dsRNA, a tasiRNA, or a secondary siRNA. In yet another embodiment of this aspect, the promoter is a nodule specific promoter, a root specific promoter, an inducible promoter, a constitutive promoter, or a combination thereof. In a further embodiment of this aspect, the promoter is a constitutive promoter, and wherein the promoter is selected from the group including of a CaMV35S promoter, a derivative of the CaMV35S promoter, a maize ubiquitin promoter, a polyubiquitin promoter, a vein mosaic cassava virus promoter, or an Arabidopsis UBQ10 promoter. In still another embodiment of this aspect, the nucleic acid sequence is inserted into the genome of the plant so that the nucleic acid sequence is operably linked to an endogenous promoter. In a further embodiment of this aspect, the endogenous promoter is a nodule specific promoter or a root specific promoter.

A further aspect of the disclosure includes methods of making the genetically modified plant or part thereof of any of the preceding embodiments, including genetically modifying the plant cell by transforming the plant cell with one or more gene editing components that target an endogenous nuclear genome sequence encoding a FUN protein, a FUN-like protein, a NAC-domain containing protein, a NRT3.1 protein, a bZIP28 protein, a NAC-domain containing protein, a HO1 protein, a NRT2.1 protein, or an AS1 protein, wherein the endogenous nuclear genome sequence or a part thereof is knocked out. In another embodiment of this aspect, the one or more gene editing components include a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence.

Yet another aspect of the disclosure includes an expression vector or isolated DNA molecule including (i) one or more nucleotide sequences encoding a FUN protein, a FUN-like protein, a HO1 protein, a NAC-domain containing protein, a bZIP28 protein, a NRT2.1 protein, a NRT3.1 protein, an AS1 protein, or a combination thereof, wherein the one or more nucleotide sequences are operably linked to at least one expression control sequence, (ii) one or more nucleotide sequences able to reduce or knock out a nucleic acid sequence encoding a FUN protein, a FUN-like protein, a HO1 protein, a NAC-domain containing protein, a bZIP28 protein, a NRT2.1 protein, a NRT3.1 protein, an AS1 protein, or a combination thereof, wherein the one or more nucleotide sequences are operably linked to at least one expression control sequence, or (iii) one or more nucleotide sequences including a mutation in a gene for a FUN protein, a FUN-like protein, a HO1 protein, a NAC-domain containing protein, a bZIP28 protein, a NRT2.1 protein, a NRT3.1 protein, an AS1 protein, or a combination thereof, wherein the mutation reduces or knocks out the activity or expression of the protein and the one or more nucleotide sequences are operably linked to at least one homologous nucleic acid sequence that hybridizes adjacent to the mutation site in the gene. In a further embodiment of this aspect, the protein is a FUN protein, and wherein the FUN protein includes a polypeptide with at least 70% identity, at least 80% identity, at least 90% identity, at least 95% identity, or at least 99% identity to a protein selected from the group of SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, and SEQ ID NO: 82; wherein the FUN protein includes SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, SEQ ID NO: 82, or conserved domains thereof, or combinations thereof; or wherein the FUN protein includes SEQ ID NO: 1, SEQ ID NO: 8, or SEQ ID NO: 9, or conserved domains thereof, or combinations thereof; wherein the protein is a FUN-like protein, and wherein the FUN-like protein includes a polypeptide with at least 70% identity, at least 80% identity, at least 90% identity, at least 95% identity, or at least 99% identity to a protein selected from the group of SEQ ID NO: 4, SEQ ID NO: 6, SEQ ID NO: 7, SEQ ID NO: 83, and SEQ ID NO: 84, or conserved domains thereof, or combinations thereof; wherein the FUN-like protein includes SEQ ID NO: 4, SEQ ID NO: 6, SEQ ID NO: 7, SEQ ID NO: 83, or SEQ ID NO: 84, or conserved domains thereof, or combinations thereof; and/or wherein the protein is the NRT3.1 protein, and wherein the NRT3.1 protein includes a polypeptide with at least 70% identity, at least 80% identity, at least 90% identity, at least 95% identity, or at least 99% identity to SEQ ID NO: 74 or conserved domains thereof; wherein the protein is the bZIP28 protein, and wherein the bZIP28 protein includes a polypeptide with at least 70% identity, at least 80% identity, at least 90% identity, at least 95% identity, or at least 99% identity to SEQ ID NO: 75 or conserved domains thereof; wherein the protein is the NAC-domain containing protein, and wherein the NAC-domain containing protein includes a polypeptide with at least 70% identity, at least 80% identity, at least 90% identity, at least 95% identity, or at least 99% identity to a protein selected from the group of SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, or conserved domains thereof, or combinations thereof; wherein the protein is the HO1 protein, and wherein the HO1 protein includes a polypeptide with at least 70% identity, at least 80% identity, at least 90% identity, at least 95% identity, or at least 99% identity to SEQ ID NO: 77 or conserved domains thereof; wherein the protein is the NRT2.1 protein, and wherein the NRT2.1 protein includes a polypeptide with at least 70% identity, at least 80% identity, at least 90% identity, at least 95% identity, or at least 99% identity to SEQ ID NO: 78 or conserved domains thereof; or wherein the protein is the AS1 protein, and wherein the AS1 protein includes a polypeptide with at least 70% identity, at least 80% identity, at least 90% identity, at least 95% identity, or at least 99% identity to SEQ ID NO: 79 or conserved domains thereof; wherein the NRT3.1 protein includes SEQ ID NO: 74 or conserved domains thereof; wherein the bZIP28 protein includes SEQ ID NO: 75 or conserved domains thereof; wherein the NAC-domain containing protein includes SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, or SEQ ID NO: 73, or conserved domains thereof, or combinations thereof; wherein the HO1 protein includes SEQ ID NO: 77 or conserved domains thereof; wherein the NRT2.1 protein includes SEQ ID NO: 78, or wherein the AS1 protein includes SEQ ID NO: 79 or conserved domains thereof; or wherein the NAC-domain containing protein includes SEQ ID NO: 31, SEQ ID NO: 41, or SEQ ID NO: 42, or conserved domains thereof, or combinations thereof.

Some aspects of the present disclosure relate to a bacterial cell or an Agrobacterium cell including the expression vector or isolated DNA molecule of any of the preceding embodiments.

Additional aspects of the present disclosure relate to genetically modified plant, plant part, plant cell, or seed including the expression vector or isolated DNA molecule of any of the preceding embodiments.

Further aspects of the present disclosure relate to a kit including the expression vector or isolated DNA molecule of any of the preceding embodiments or the bacterial cell or the Agrobacterium cell of the preceding embodiments.

Still further aspects of the present disclosure relate to methods of increasing nitrogen fixation, delaying nodule senescence, or inducing FUN filamentation in a plant, including: (a) introducing a genetic alteration via an expression vector or isolated DNA molecule of any of the preceding embodiments; and optionally (b) treating the plant with zinc or manganese or growing the plant under high zinc, high manganese, or high nitrate conditions.

An additional aspect of the present disclosure relates to a genetically altered plant genome including (i) the one or more genetic alterations in the genetically modified plant or part thereof of any one of the preceding embodiments, or (ii) the one or more genetic alterations in the genetically modified plant or part produced by the method of any one of the preceding embodiments.

Yet further aspects of the disclosure relate to a non-regenerable part or cell of the genetically modified plant or part thereof of any one of the preceding embodiments.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1V show the nodule phenotype and nitrogen fixation activity of fun mutant Lotus plants in restrictive nitrate conditions, as well as that Fun is expressed specifically in root nodules. FIG. 1A shows representative pictures of the nodule phenotype of wild type (WT; Gifu) Lotus plants under KCl conditions (control; top left), WT Lotus plants under 10 mM KNO₃ conditions (top middle), and fun mutant Lotus plants under 10 mM KNO₃ conditions (top right and bottom row), the fun mutants including fun (top right), fun-2 (bottom left), fun-3 (bottom middle), and fun-4 (bottom right). Scale bar=1 cm. FIG. 1B shows a diagram of the FUN gene and LORE1 insertions of each genetic background phenotyped in FIG. 1A. In fun and fun-4, LORE1 is inserted in the promoter regions (light grey line). In fun-2, LORE1 is inserted at the end of the fourth intron (introns represented by the black line). In fun-3 (30099638), LORE1 is inserted at the end of the seventh intron. Arrows represent insertion points for LORE1 along the gene. FIG. 1C shows the total number of nodules (white boxes, labeled “total”) and the number of pink functional nodules (colored boxes, labeled “pink”) formed on wild type (WT; Gifu) Lotus plants and fun mutant Lotus plants under 2 weeks of 10 mM KNO₃ exposure. FIG. 1D shows nitrogen fixation activity quantified using an acetylene reduction assay (ARA; nmol C₂H₂/h/plant) for wild type (WT; Gifu) Lotus plants and fun mutant Lotus plants under 2 weeks of 10 mM KNO₃ exposure. FIG. 1E displays the ARA measurements of FIG. 1D (vertical axis) tracked over the course of 14 days (top) and up to 20 mM KNO₃(bottom). The light grey line with blue circles represents the ARA results for WT plants. On the top, the medium grey line with orange circles represents the ARA results for fun mutant plants, and the black line with medium grey circles represents the ARA results for fun-3 mutant plants. On the bottom, the black line with orange circles represents the ARA results for fun mutant plants. FIG. 1F shows the total number of nodules (white boxes, labeled “total”) and the number of pink functional nodules (grey boxes, labeled “pink”) formed on wild type (WT; Gifu) Lotus plants, fun mutant Lotus plants, fun-2 mutant Lotus plants, fun-3 mutant Lotus plants, and fun-4 mutant Lotus plants under 2 weeks of 10 mM KNO₃ exposure. FIG. 1G shows nitrogen fixation activity quantified using an acetylene reduction assay (ARA; nmol C₂H₂/h/plant) for wild type (WT; Gifu) Lotus plants, fun mutant Lotus plants, fun-2 mutant Lotus plants, fun-3 mutant Lotus plants, and fun-4 mutant Lotus plants under 2 weeks of 10 mM KNO₃ exposure. FIG. 1H shows the number of pink nodules per plant for wild type (WT), fun, fun-3, and fun-4 mutants with 5 mM nitrate application prior to inoculation (grey boxes, labeled “5 mM”) and without nitrate application prior to inoculation (white boxes, labeled “0 mM”). Plants were grown on plates with 0 or 5 mM KNO₃ and then inoculated with rhizobia; pink nodules were measured 3 weeks post inoculation. FIG. 1I shows the total number of nodules per plant for wild type (WT),fun,fun-3, and fun-4 mutants with 5 mM nitrate application prior to inoculation (grey boxes, labeled “5 mM”) and without nitrate application prior to inoculation (white boxes, labeled “0 mM”). Plants were grown on plates with 0 or 5 mM KNO₃ and then inoculated with rhizobia; nodules were measured 3 weeks post inoculation. FIG. 1J shows nitrogen fixation activity quantified using an acetylene reduction assay (ARA; nmol C₂H₂/h/plant) for wild type (WT), fun, fun-3, and fun-4 mutants with 5 mM nitrate application prior to inoculation (grey boxes, labeled “5 mM”) and without nitrate application prior to inoculation (white boxes, labeled “0 mM”). Plants were grown on plates with 0 or 5 mM KNO₃ and then inoculated with rhizobia; the ARA was performed 3 weeks post inoculation. For FIGS. 1F-1J, letters indicate significant differences (p<0.05) between compared groups of plants. FIG. 1K shows the leghemoglobin content (μg leghemoglobin per plant) of wild type (WT; Gifu) Lotus plants and fun mutant Lotus plants under 2 weeks of 10 mM KNO₃ exposure. FIG. 1L shows the complementation of fun mutants grown under 2 weeks of 10 mM KNO₃ exposure, with grey boxes indicating empty vector (labeled “EV”) transformed into a WT background (left) or into a fun mutant background (middle), and the white box (right) indicating proUbi:FUN-GFP transformed into a fun mutant background. Letters represent significant differences between compared groups of plants. FIG. 1M shows a representative image of plant roots with nodules of a plant expressing the proFun:GUS reporter construct used for in situ visualization of Fun gene expression. The roots are stained with 5-bromo-4-chloro-3-indolyl glucuronide (X-Gluc). Blue staining indicates expression of the GUS reporter; scale bar=2 cm. FIG. 1N is a median longitudinal section of a nodule imaged with light microscopy. Blue staining indicates expression of the GUS reporter; scale bar=200 μm. FIG. 1O is a magnification of FIG. 1N, with ‘ic’ indicating infected cells, ‘uc’ indicating uninfected cells, and ‘nc’ indicating nodule cortex; scale bar=200 μm. In FIGS. 1M-1O, proFun:GUS is a construct where the native Fun promoter sequence (proFUN) was fused to the coding sequence of β-glucuronidase (GUS) followed by the native Fun terminator sequence (tFUN). FIG. 1P is a schematic diagram of the FUN protein, showing the bZIP DNA binding domain (grey oval) and the sensor domain (dark grey star). FIG. 1Q shows a bar plot comparing normalized RNA measurements for FUN transcripts across different plant tissues of Lotus japonicus Gifu, as calculated in Kamal et al. (2020). Insights into the evolution of symbiosis gene copy number and distribution from a chromosome-scale Lotus japonicus Gifu genome sequence.” DNA Res. 27(3). The L. japonicus Gifu tissues are listed along the vertical axis. “Nodule 21d” and “Nodule 10d” refer to measurements taken from nodules 21 days and 10 days, respectively, post inoculation with Mesorhizobium loti R7A. FIG. 1R shows a bar plot of significantly differentially expressed (DE) genes that are upregulated (top) or downregulated (bottom) in either WT plants (black boxes) or fun mutant plants (grey boxes). The number of DE genes is plotted along the horizontal axis. FIG. 1S shows bar plots of the level of upregulated differential expression (in log 2 fold-change relative to WT plant expression, horizontal axis) of select genes (vertical axis) in fun mutant plants (black boxes) and fun-3 mutant plants (grey boxes). FIG. 1T shows bar plots of the level of downregulated differential expression (in log 2 fold-change relative to WT plant expression, horizontal axis) of select genes (vertical axis) in fun mutant plants (black boxes) and fun-3 mutant plants (grey boxes). FIG. 1U shows ontology groups identified by GO-MWU as enriched among upregulated genes (in red) and downregulated genes (in blue). Text size and boldness indicate the p value. FIG. 1V shows relative expressions of downstream targets of FUN with identified TGA motifs within the promoter that are differentially expressed in fun relative to wild type. Relative expressions are shown in an RNAseq timeseries from Wang et al. (Wang, L. et al. A transcription factor of the NAC family regulates nitrate-induced legume nodule senescence. New Phytol. (2023) doi:10.1111/nph.18896). In FIGS. 1C-1D and 1F-1L, circles represent individual plants. In FIGS. 1C-1E and 1K-1L, asterisks denote significant differences between compared groups; “**” refers to a p-value <0.01 and “*” refers to a p-value <0.05.

FIGS. 2A-2S show that FUN controls the expression of the downstream genes Nrt2.1, Ho1, NAC094, Nrt3.1, and AS1 to regulate nitrate signaling and nitrogen fixation in root nodules. FIG. 2A shows expression levels of Nrt2.1 (left), Ho1 (center), and NAC094 (right) genes in the nodules of wild type Lotus plants (Gifu; white),fun mutant Lotus plants (grey), and fun-3 mutant Lotus plants (dark grey) after 0 hours, 3 hours and 24 hours of 10 mM KNO₃ nitrate treatment. FIG. 2B shows expression levels of Nrt3.1 (left), and AS1 (right) genes in the nodules of wild type Lotus plants (Gifu; white), fun mutant Lotus plants (grey), and fun-3 mutant Lotus plants (dark grey) after 0 hours, 3 hours and 24 hours of 10 mM KNO₃ nitrate treatment. FIG. 2C shows a schematic diagram of the promoter of Nrt2.1 (proNRT2.1; top), Ho1 (proHO1; second from top), NAC094 (proNAC094; middle) Nrt3.1 (proNRT3.1, second from bottom), and AS1 (proAS1, bottom). The promoter of Nrt2.1 has four putative FUN binding sites (FBSs) designated p1, p2, p3, and p4; the promoter of Ho1 has two putative FBSs designated p1 and p2; the promoter of NAC094 has one putative FBS designated p1; the promoter of Nrt3.1 has three putative FBSs designated p1, p2, and p3; and the promoter of AS1 has one putative FBS designated p1. FIG. 2D shows a gel image from an EMSA assay demonstrating the binding of FUN protein to DNA probes containing the FBSs p1, p2, p3, and p4 from the promoter of Nrt2.1 (on left), to DNA probes containing the FBSs p1 and p2 from the promoter of Ho1 (center), and to DNA probes containing the FBS p1 from the promoter of NAC094 (on right). FIG. 2E shows a gel image from an EMSA assay demonstrating the binding of FUN protein to DNA probes containing the FBS p1 from the promoter of Nrt2.1, the FBSs p1, p2, and p3 from the promoter of Nrt3.1, and the FBS p1 from the promoter of AS1. FIG. 2F shows a gel image from EMSAs targeting p1 (top) and p4 (bottom) FBSs from the promoter of Nrt2.1 in a competition assay. Competition DNA is 50-, 150-, and 500-times concentration of WT DNA without the tagging placed on the probes. The label “m” corresponds to DNA probes with mutation of TGACG, the core binding site. FIG. 2G shows the results of a transactivation assay of the promoter of Nrt2.1 (on left), Ho1 (center), and NAC094 (on right) by FUN in N. benthamiana leaves, with the white bar representing GFP and the grey bar representing pro35S:FUN-GFP. Pro35S:FUN-GFP was expressed as the effector, and GUS was driven by either the promoter of Nrt2.1, the promoter of Ho1, or the promoter of NAC094 as the reporter. FIG. 2H shows the results of a transactivation assay of the promoter of Nrt3.1 (on left) and AS1 (on right) by FUN in N. benthamiana leaves, with the white bar representing GFP and the grey bar representing pro35S:FUN-GFP. Pro35S:FUN-GFP was expressed as the effector, and the GUS reporter was driven by either the promoter of Nrt3.1 or the promoter of AS1. FIG. 2I shows representative pictures of the nodule phenotype of WT (Gifu) Lotus plants, nrt2.1-3 mutant Lotus plants, ho1-4 mutant Lotus plants, or nac094-3 mutant Lotus plants under 2 weeks of 10 mM KNO₃ exposure. Scale bars are 1 cm for all four pictures. FIG. 2J shows the total number of nodules (grey boxes, labeled “total”) and the number of pink functional nodules (white boxes, labeled “pink”) formed on wild type (WT; Gifu) Lotus plants, nrt2.1-3 mutant Lotus plants, and nrt2.1-4 mutant Lotus plants under 2 weeks of 10 mM KNO₃ exposure. FIG. 2K shows nitrogen fixation activity quantified using an acetylene reduction assay (ARA; nmol C₂H₂/h/plant) for wild type (WT; Gifu) Lotus plants, nrt2.1-3 mutant Lotus plants, and nrt2.1-4 mutant Lotus plants under 2 weeks of 10 mM KNO₃ exposure. FIG. 2L shows the leghemoglobin content (μg leghemoglobin per plant) of wild type (WT; Gifu) Lotus plants, nrt2.1-3 mutant Lotus plants, and nrt2.1-4 mutant Lotus plants under 2 weeks of 10 mM KNO₃ exposure. FIG. 2M shows the total number of nodules (white boxes, labeled “total”) and the number of pink functional nodules (grey boxes, labeled “pink”) formed on wild type (WT; Gifu) Lotus plants, ho1-4 mutant Lotus plants, and ho1-5 mutant Lotus plants under 2 weeks of 10 mM KNO₃ exposure. FIG. 2N shows the leghemoglobin content (μg leghemoglobin per plant) of wild type (WT; Gifu) Lotus plants, nac094-3 mutant Lotus plants, and nac094-4 mutant Lotus plants under 2 weeks of 10 mM KNO₃ exposure. FIG. 2O shows the total number of nodules (white boxes, labeled “total”) and the number of pink functional nodules (grey boxes, labeled “pink”) formed on wild type (WT; Gifu) Lotus plants, nac094-3 mutant Lotus plants, and nac094-4 mutant Lotus plants under 2 weeks of 10 mM KNO₃ exposure. FIG. 2P shows nitrogen fixation activity quantified using an acetylene reduction assay (ARA; nmol C₂H₂/h/plant) for wild type (WT; Gifu) Lotus plants, ho1-4 mutant Lotus plants, and ho1-5 mutant Lotus plants under 2 weeks of 10 mM KNO₃ exposure. FIG. 2Q shows the leghemoglobin content (μg leghemoglobin per plant) of wild type (WT; Gifu) Lotus plants, ho1-4 mutant Lotus plants, and ho1-5 mutant Lotus plants under 2 weeks of 10 mM KNO₃ exposure. FIG. 2R shows nitrogen fixation activity quantified using an acetylene reduction assay (ARA; nmol C₂H₂/h/plant) for wild type (WT; Gifu) Lotus plants, nac094-3 mutant Lotus plants, and nac094-4 mutant Lotus plants under 2 weeks of 10 mM KNO₃ exposure. FIG. 2S shows the normalized read counts of downstream targets of FUN with identified TGA motifs within the promoters, for wild type plants receiving mock treatment (labeled “WT mock”) and for wild type, fun, and fun-3 mutant plants 24 after exposure to nitrate (labeled “WT 24 h,” “fun 24 h,” and “fun-3 24 h” respectively). Graphs from left to right show normalized read count for NRT2.1, HO1, NAC094, NRT3.1, and AS1. In FIGS. 2A-2B, 2G-2H, and 2J-2R, circles represent individual plants. In FIGS. 2A-2B, 2G-2H, and 2J-2R, asterisks denote significant differences between compared groups; “**” refers to a p-value <0.01 and “*” refers to a p-value <0.05.

FIGS. 3A-3L show that the sensor domain of FUN forms filament structures in the presence of physiological concentrations of zinc (Zn). FIG. 3A shows the results of dynamic light scattering (DLS) analyses on the FUN sensor domain with an exposure to 4 mM of MgCl₂, CaCl₂), MnCl₂, ZnCl₂, NH₄Cl, KNO₃, KNO₂, KCl, or a blank sample (“FUN sensor” alone, control). FIG. 3B shows the results of DLS analysis on FUN (bZIP) with MnCl₂ at a concentration of 8 mM, 4 mM, 2 mM, 1 mM, 500 μM, 250 μM, 62.5 μM, 15.6 μM, 3.9 μM, or 0 μM. FIG. 3C shows the results of DLS analysis on FUN (bZIP) with ZnCl₂ at a concentration of 125 μM, 62.5 μM, 31.3 μM, 15.6 μM, 7.8 μM, 3.9 μM, 2.0 μM, 0 μM, or 8 mM without FUN (8 mM no bZIP; control). FIG. 3D shows the results of DLS analysis on the FUN bZIP alone (“Fun sensor”), FUN with 100 μM ZnCl₂ (“FUN sensor+Zn”), or FUN with 100 μM ZnCl₂ and 5 mM ethylenediaminetetraacetic acid (EDTA) (“FUN sensor+Zn+EDTA”). FIG. 3E shows a plot of SAXS analysis, with scattering intensity on the vertical axis as “I(q)”, in cm⁻¹, as a function of q(Å⁻¹) on the horizontal axis. Scattering is plotted for the FUN sensor alone (bZIP only) (“FUN sensor”, grey), the FUN bZIP bound to Zn (“FUN sensor+Zn”, green), or the FUN bZIP with Zn removed using EDTA (“FUN sensor+Zn+EDTA”, black). FIG. 3F shows the plotted histogram of distances between pairs of points within particles, based on the analysis of FIG. 3E, with the pair distance distribution model p(r) plotted on the vertical axis and distance in angstroms, r(Å), plotted on the horizontal axis. “394 Å” with an arrow represents the maximum diameter (Dmax) of 394 Å for the zinc-bound FUN bZIP (“FUN sensor+Zn”). “118 Å” with an arrow represents the Dmax of 118 Å for the measurements of only the FUN bZIP (“FUN sensor”) or the FUN bZIP with Zn removed (“FUN sensor+Zn+EDTA”). FIG. 3G shows Guinier plots calculated from FIGS. 3E and 3F, wherein the radius of gyration is calculated through the scattering intensity as a function of the scattering vector q (the vertical axis vs. the horizontal axis). Closed circles are data used in the fit and open circles are data points omitted. The p(r) function shows radii of gyration of 39±1 Å for the pure FUN sensor sample and the EDTA+zinc-containing sample, whereas it is 125±1 Å for the zinc-bound sample. The values were slightly lower for all samples in the Guinier analysis. FIG. 3H shows representative electron microscopy images of the FUN sensor domain alone (bZIP1 sensor; top), the FUN sensor domain with 300 μM ZnCl₂ (bZIP1 sensor+300 μM Zn; middle); and FUN with 300 μM ZnCl₂ and 5 mM EDTA (bZIP1 sensor+300 μM Zn+5 mM EDTA; bottom). FIG. 3I shows the relative expression (vertical axis) of Fun over time (horizontal axis) in 3-week-old nodules exposed to 10 mM KNO 3 for 0, 0.5, and 3 hours (left), and 0, 1, 3, and 7 days (right).

FIG. 3J shows the purification and thermostability of the FUN sensor domain. The left shows a chromatogram from size exclusion chromatography (Superdex 200 increase 10/300) of the FUN sensor domain, plotting the absorption (vertical axis) across elution volumes (horizontal axis). The right shows a sodium dodecyl sulphate-polyacrylamide gel electrophoresis (SDS-PAGE) analysis of SEC fractions. Fractions 14-17 were pooled and saved as indicated by the dashed lines on the chromatogram and horizontal line above the SDS-PAGE. FIG. 3K shows the thermostability of the purified FUN sensor domain through measuring inflection temperatures (Ti) (° C.) either alone (“FUN sensor only”) or with different ions at a concentration of 4 mM, namely MgCl₂, CaCl₂), MnCl₂, ZnCl₂, NH₄Cl, KNO₃, KNO₂, or KCl (vertical axis). FIG. 3L shows the results of DLS analysis of the FUN protein containing zipper and sensor domains in the absence (grey line) and presence of 100 μM ZnCl₂ (green line). The zinc-induced change in hydrodynamic radius is reversed with 5 mM EDTA (black line). In FIGS. 3C-3G, “FUN sensor” in grey represents measurements of the FUN sensor alone, “FUN sensor+Zn” in green represents measurements of the FUN sensor treated with 100 μM ZnCl₂, and “FUN sensor+Zn+EDTA” in black represents the FUN sensor treated with 100 μM ZnCl₂ and subsequently treated with 5 mM EDTA to remove Zn. In FIGS. 3C-3G, the four-pointed star labeled “apo” represents the FUN sensor in the apostructural form (“apo”). The chain of overlapping four-pointed stars labeled “Zn-bound” represents the combination of FUN sensors bound to Zn in a larger-size oligomer.

FIGS. 4A-4H show that zinc regulates the subcellular location and function of FUN. FIG. 4A shows a representative image of pro35S:FUN-GFP subcellular location in N. benthamiana leaves. FIG. 4B shows the fluorescence distribution of FUN-GFP subcellular location in N. benthamiana leaves. FIG. 4C shows the ratio of dots nucleus (dark grey) to total nucleus (homo; light grey) of FUN-GFP subcellular location in N. benthamiana leaves. In FIGS. 4A-4C, the results were obtained under 500 μM MgCl₂ (mock), MnCl₂ (Mn), and ZnCl₂ (Zn) conditions. FIG. 4D shows the results of a transactivation assay of the promoter of Nrt2.1 by FUN in N. benthamiana leaves after 500 μM MgCl₂ (mock) and ZnCl₂ (Zn) treatments, with the white bars representing GFP and the grey bars representing pro35S:FUN-GFP. FUN-GFP was expressed as the effector, and GUS was driven by the promoter of Nrt2.1 (proNrt2.1:GUS) as the reporter. FIG. 4E shows nitrogen fixation activity quantified using an acetylene reduction assay (ARA; nmol C₂H₂/h/plant) for wild type (WT; Gifu) Lotus plants exposed to 500 μM MgCl₂ (mock; white) and ZnCl₂ (Zn; dark grey) in combination with KCl (left) or 10 mM KNO₃(center and right). FIG. 4F shows nitrogen fixation activity quantified using an acetylene reduction assay (ARA; nmol C₂H₂/h/plant) for wild type (WT; Gifu) Lotus plants and fun mutant Lotus plants exposed to 500 μM MgCl₂(mock; white) and ZnCl₂ (Zn; dark grey) together with 10 mM KNO₃. FIG. 4G shows the leghemoglobin content (μg leghemoglobin per plant) of wild type (WT; Gifu) Lotus plants and fun mutant Lotus plants exposed to 500 μM MgCl₂ (mock; white) and ZnCl₂ (Zn; dark grey) together with 10 mM KNO₃. FIG. 4H shows the leghemoglobin content (μg leghemoglobin per plant) of wild type (WT; Gifu) Lotus plants after 2 weeks of 10 mM KCl exposure (left), 10 mM KNO₃ exposure (center), or 10 mM KNO₃ in addition to ZnCl₂ treatment (right). In FIGS. 4D-4H, circles represent individual plants.

FIGS. 5A-5H show that nitrate facilitates export of zinc from nodule cells using the zinc-sensitive dye Zinpyr-1. FIG. 5A shows that expression of the two putative zinc transporter genes Zip2 and Zip4 is induced in nodules after being treated for 24 hours with 10 mM KNO₃ (nitrate). FIG. 5B depicts mechanisms of FUN-regulated nodule function. On the left is the mechanism acting under low soil nitrate levels, wherein zinc accumulates in nodules, retaining FUN in inactive filaments and allowing continued nitrogen fixation; on the right is the mechanism acting under high soil nitrate levels, wherein cellular zinc levels decrease, liberating active FUN from filaments and increasing expression of target genes. Arrows denote the directional effect of these conditions. “NAC094”, “HO1”, and “NRT2.1” represent the activity of these three target genes inducing nodule senescence. Pink shows the presence of inactive FUN filaments and represents the corresponding phenotype of pink nodules. Green shows the presence of active FUN. FIG. 5C shows cellular zinc levels within nodules as indicated by the Zinpyr-1 fluorescent dye at 24 hours post mock treatment (left, KCl) and nitrate treatment (right, KNO₃). Scale bar=200 μm. FIG. 5D shows the average intensity of the fixation zones indicated with the dashed circles in FIG. 5C. FIG. 5E shows images of sections of nodules from X-Ray Fluorescent (XRF) Microscopy taken 24 hours post mock treatment (left, KCl) or post nitrate treatment (right, KNO₃). Scale bar=20 μm. FIG. 5F shows the images from FIG. 5E with white boxes enclosing the regions analyzed for quantification of zinc. Scale bar=20 μm. FIG. 5G shows images of the fluorescence produced by FUN-GFP constructs in Lotus roots with control (left, labeled “MgCl₂”) and zinc (right, labeled “ZnCl₂”) treatments. Scale bar=20 μm. Values for chi-squared testing are shown below; ***=p value <0.01. FIG. 5H shows confocal images of N. benthamiana leaves expressing a FUN-GFP construct and co-infiltrated with either MgCl₂ (left) or ZnCl₂ (right) two days before confocal observation. Scale bar=5 μm. Values for chi-squared testing are shown below; *=p value <0.05.

FIGS. 6A-6E show phylogenetic trees of FUN proteins and the relative expression pattern of soybean FUN orthologues. FIG. 6A shows a phylogeny of FUN orthologous genes that were identified using shoot.bio as well as FUN and LjFUN-like. Support values for the tree are plotted at bifurcation points. FIG. 6B shows the relative expression (vertical axis) pattern of the closest FUN soybean orthologues, Glyma.02G097900 and Glyma.01G084200, across various tissues (horizontal axis). Expression levels of Glyma.02G097900 are shown in blue and expression levels of Glyma.01G084200 are shown in orange. FIG. 6C shows a schematic diagram of the LjFUN protein. The DNA binding bZIP domain is shown in blue, while the zinc sensor domain is shown in red. FIG. 6D shows the first half of a protein alignment of selected orthologues of FUN and FUN-like proteins. FIG. 6E shows the second half of a protein alignment of selected orthologues of FUN and FUN-like proteins. In FIGS. 6A, 6D, and 6E, plant species names correspond to the abbreviations used as follows: Prunus persica: Prupe; Lotus japonicus: Lj; Glycine max: Glyma; Manihot esculenta: Manes; Gossypium raimondii: Gorai; Eucalyptus grandis: Eucgr; Brassica oleracea: Bol; Arabidopsis thaliana: AT; Solanum lycopersicum: Solyc; Aquilegia coerulea: Aqcoe; Amborella trichopoda: AmTr; Spirodela polyrhiza: Spipo; Musa acuminata: GSMUA; Zea mays: GRMZM; Setaria italica: Seita; Triticum Aestivum: Traes; Hordeum vulgare: HORVU; and Oryza sativa: Os. In FIGS. 6D-6E, from top to bottom, the aligned protein sequences are the consensus sequence (SEQ ID NO: 150), Lotus japonicus LjFUN (SEQ ID NO: 1), Glycine max Glyma.02G097900.1 (SEQ ID NO: 8), Glycine max Glyma.01G084200.1 (SEQ ID NO: 9), Glycine max Glyma.10G276100 (SEQ ID NO: 6), Glycine max Glyma.20G113600.1 (SEQ ID NO: 7), Lotus japonicus LjFUNL (SEQ ID NO: 83), and Arabidopsis thaliana AT1G68640.1 (SEQ ID NO: 4).

FIG. 7 shows a phylogenetic tree of the Lotus japonicus NAC-domain containing protein Nac094 (blue; labelled “LjNAC094”) and orthologous NAC-domain containing proteins in other species. Species names and protein identifiers are indicated at the end of the branches. Support values for the tree are plotted at bifurcation points.

FIGS. 8A-8C show evidence that FUN plays a role in drought and heat tolerance. FIG. 8A shows RNAseq counts of NAC094 (left graph) and HO1 (right graph) in nodules of Medicago truncatula under the following conditions, from left to right: watered, 2 days of drought, and 4 days of drought. FIG. 8B shows RNAseq counts of NAC094 (left graph) and HO1 (right graph) in nodules of Lotus japonicus under the following conditions, from left to right: watered, 2 days of drought, and 4 days of drought. FIG. 8C shows nitrogen fixation activity quantified using an acetylene reduction assay (ARA; nmol C2H2/h/plant) for wild type plant (left) and fun-3 mutant plants (right) before heat stress (grey boxes) and after 7 days of heat stress (white boxes). ‘ns’ indicates non-significant differences.

### DETAILED DESCRIPTION

The following description sets forth exemplary methods, parameters, and the like. It should be recognized, however, that such description is not intended as a limitation on the scope of the present disclosure but is instead provided as a description of exemplary embodiments.

### FUN Proteins

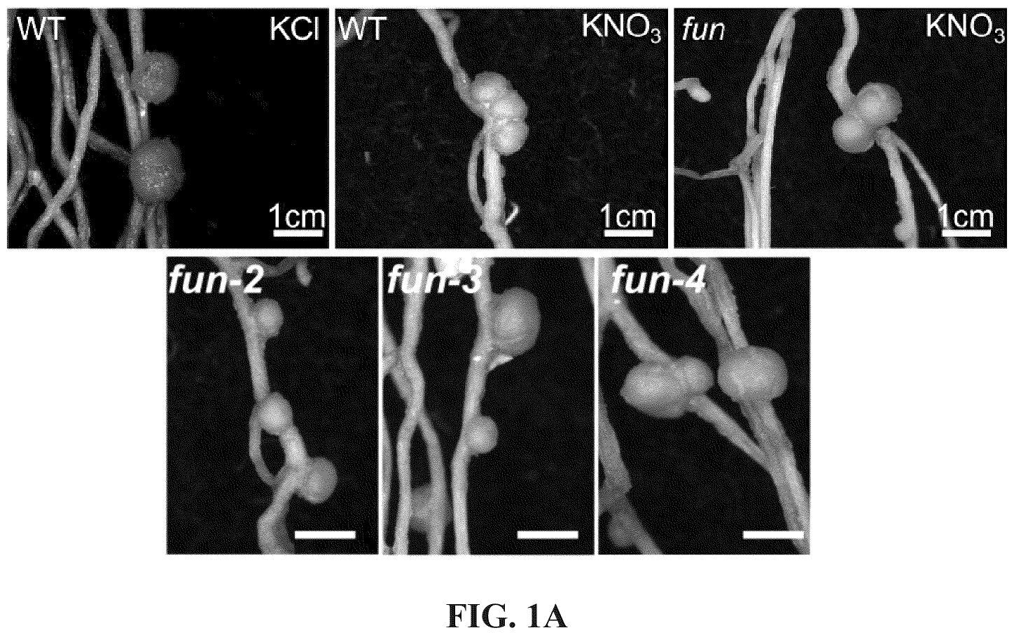

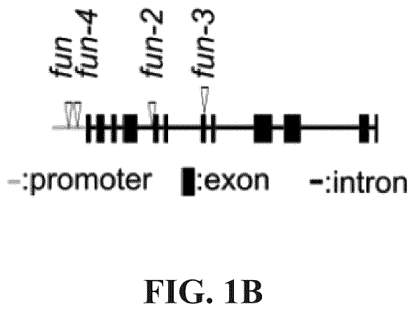

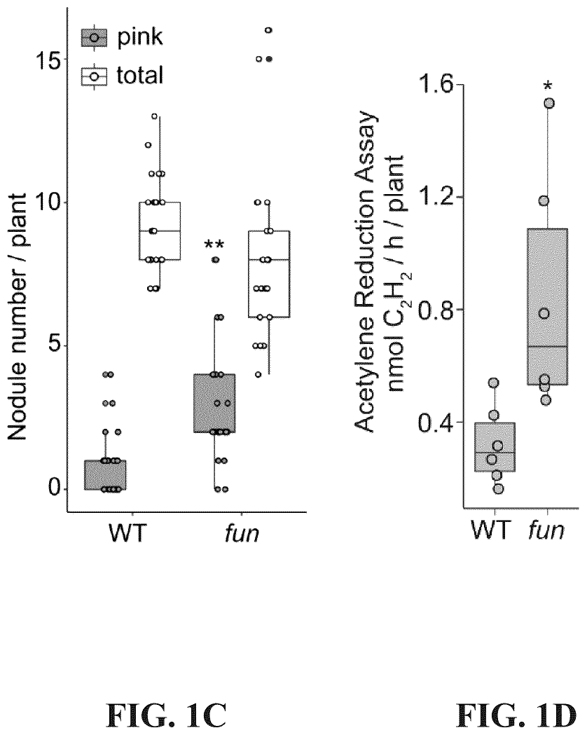

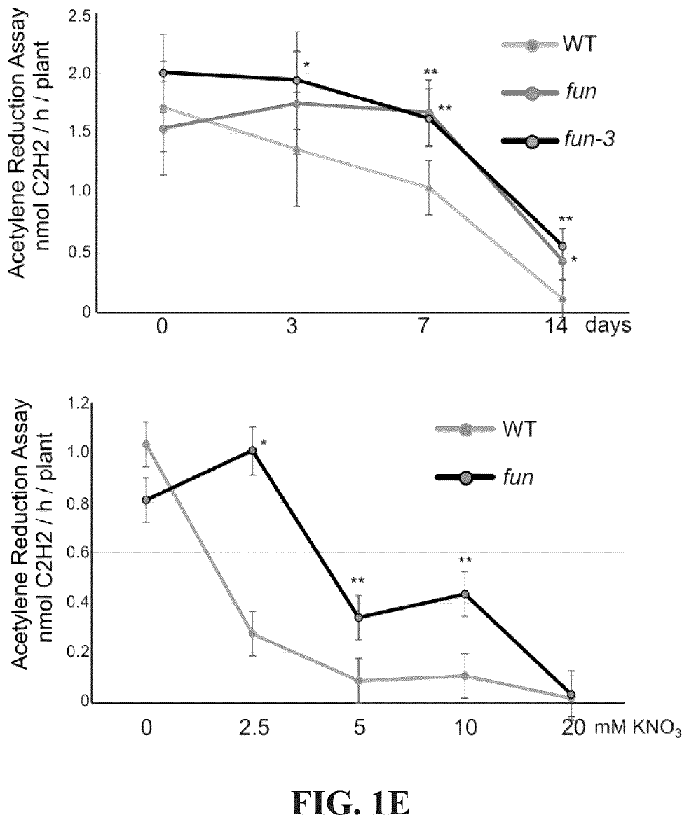

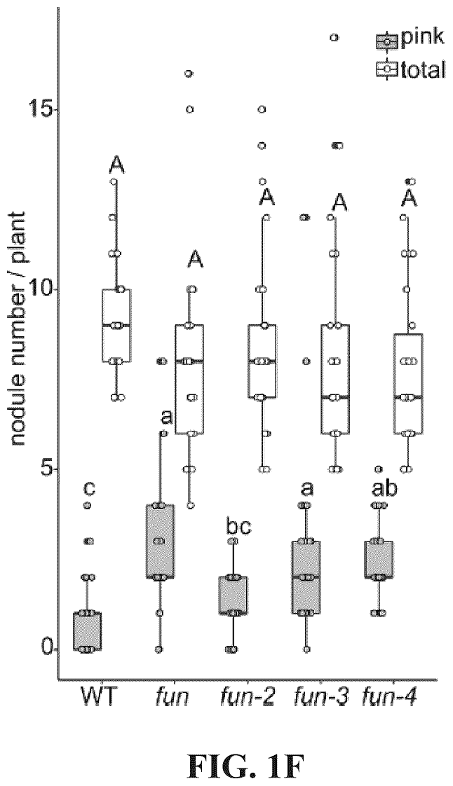

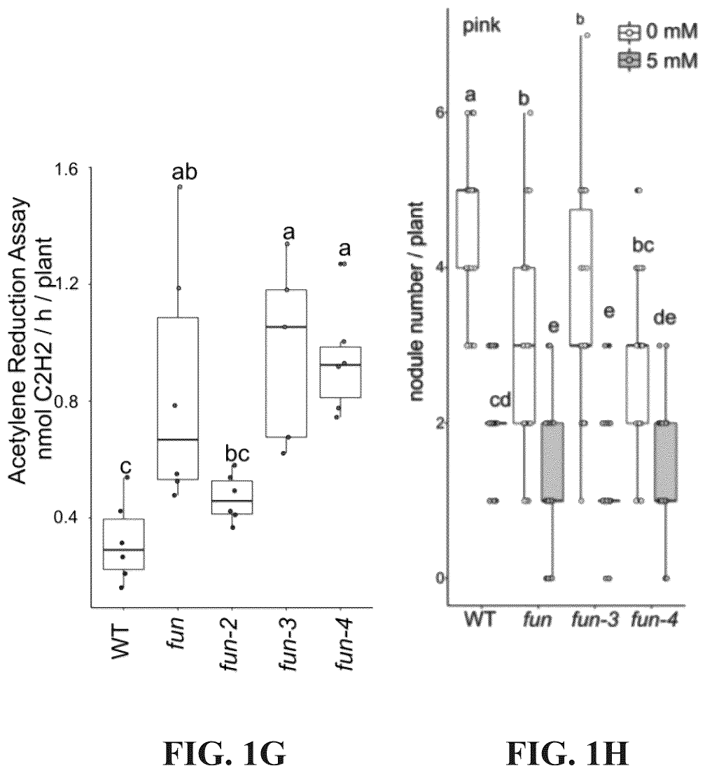

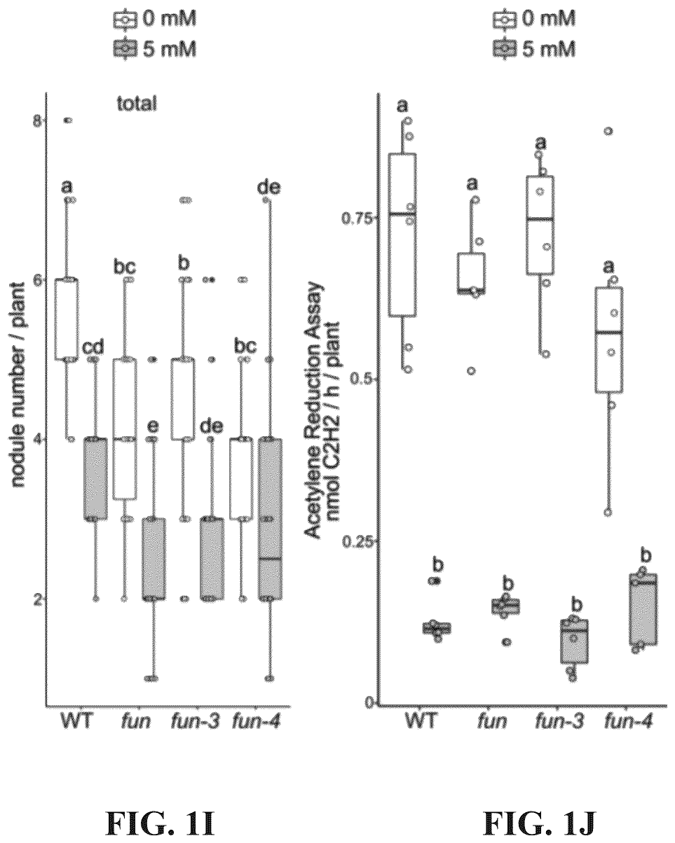

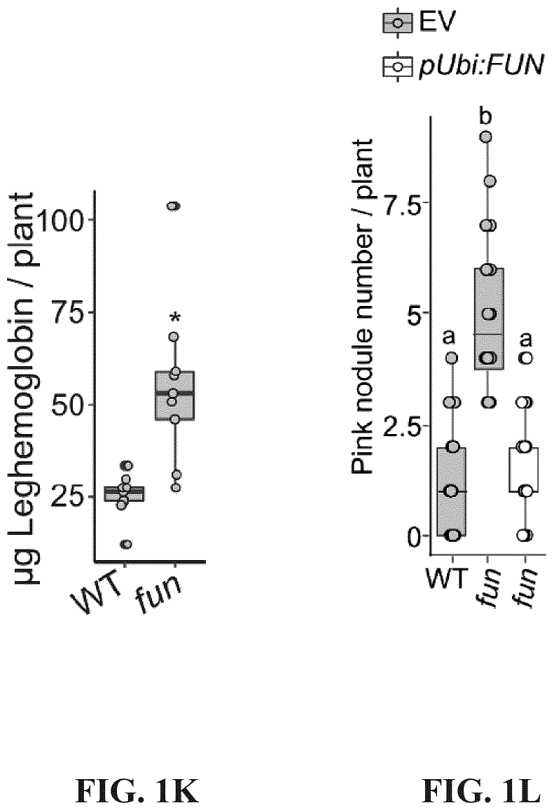

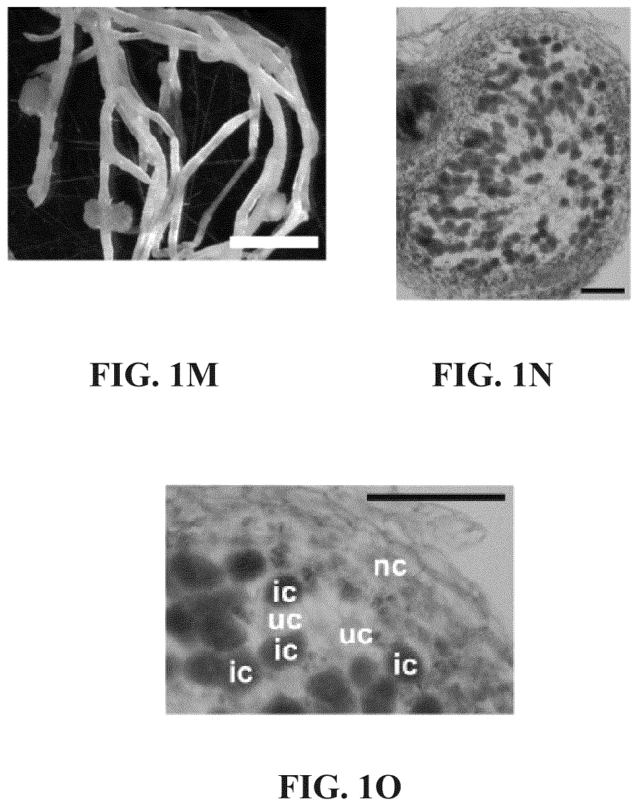

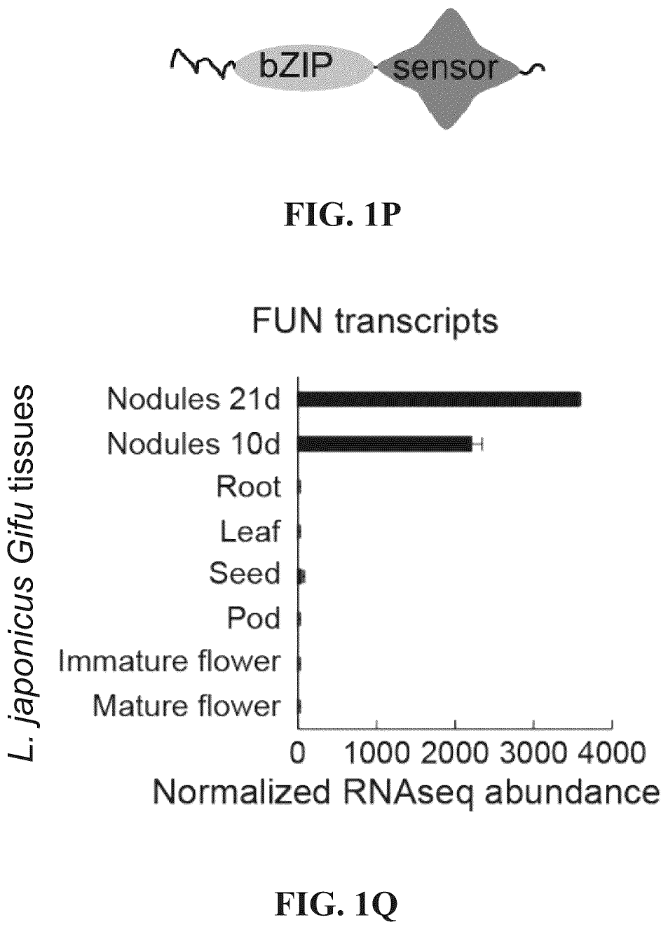

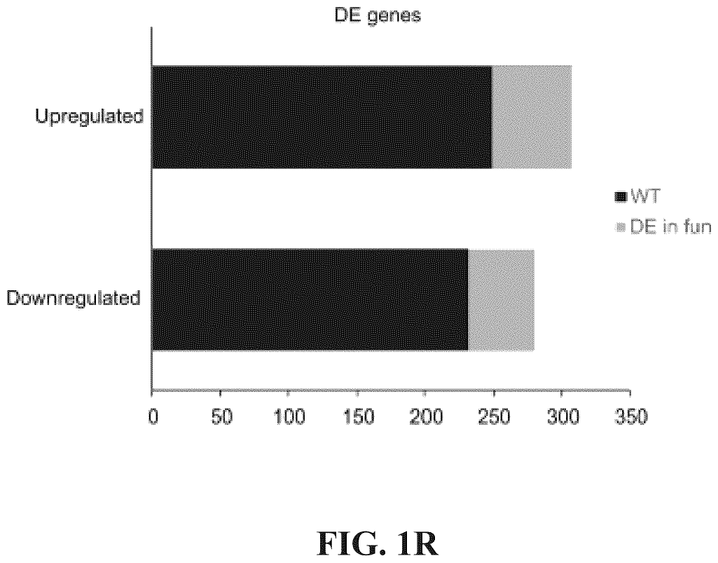

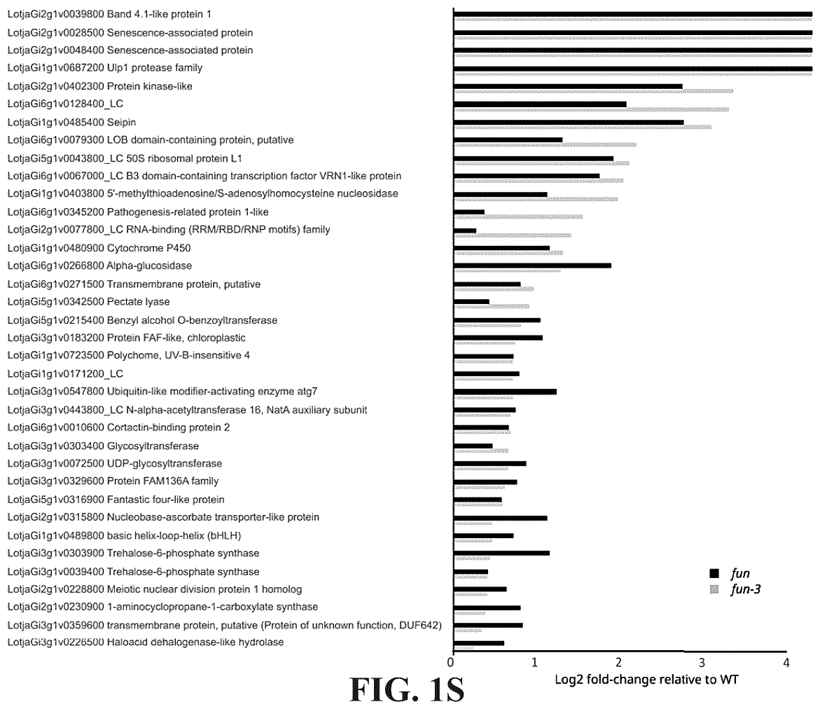

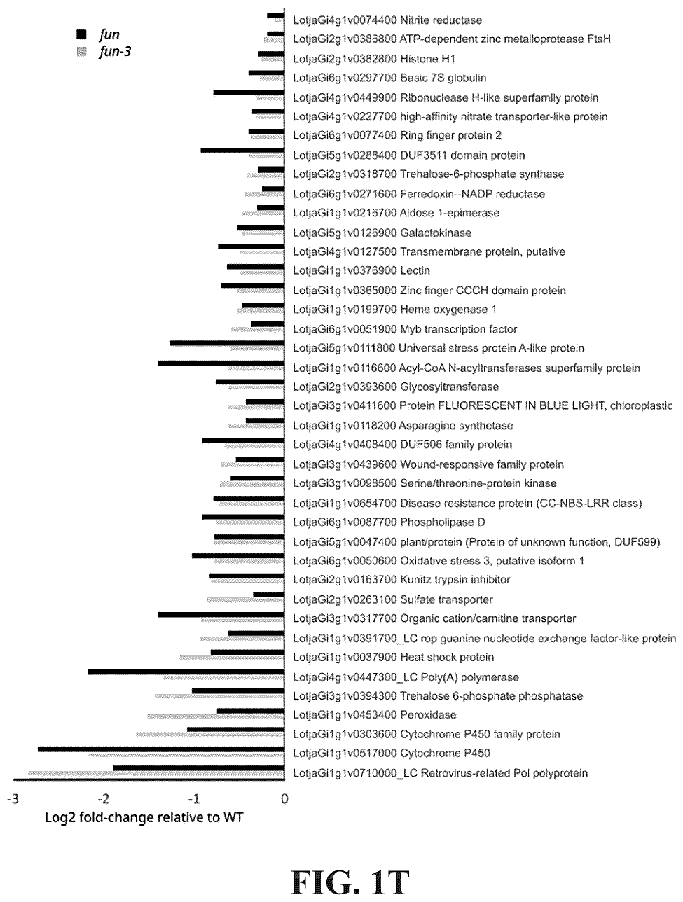

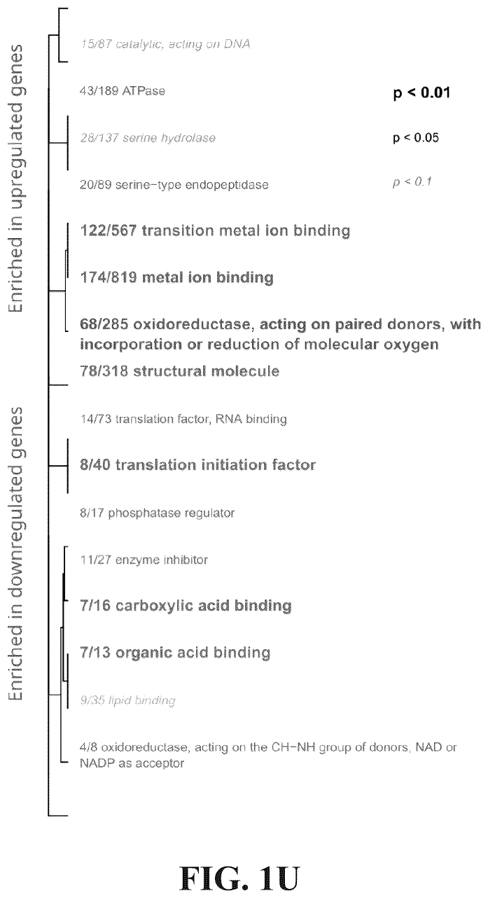

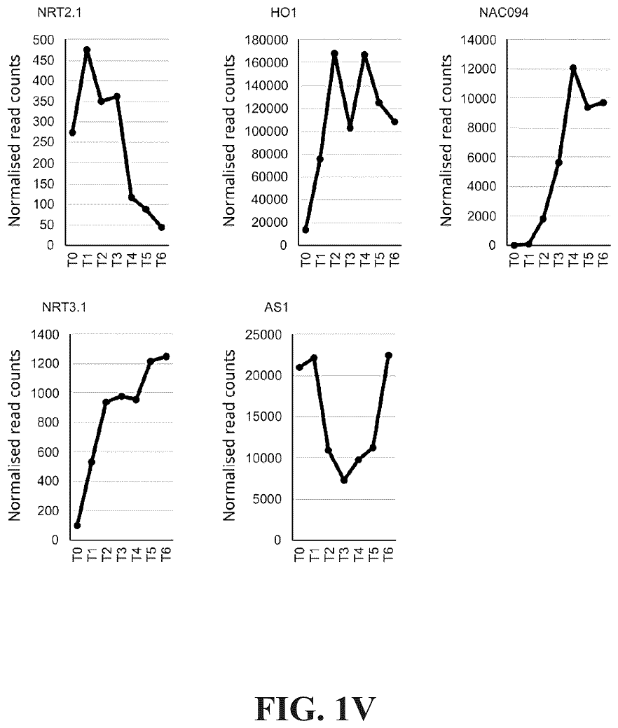

One aspect of the disclosure includes a FUN (fixation under nitrate) protein with associated uses disclosed throughout, wherein FUN is a transcriptional regulator that is expressed in nodules. Example 1 describes the identification of a basic leucine zipper transcription factor, FUN, as a novel master regulator of nitrogen fixation in legumes. Examples of FUN proteins include, without limitation, the originally identified Lotus japonicus FUN protein (LotjaGi2glv0279100; SEQ ID NO: 1) as well as orthologs of the Lotus japonicus FUN protein, (which are also expressed in the respective plant's nodules) including, without limitation, Soybean FUNa protein and FUNb protein (Glyma.02G097900; SEQ ID NO: 8, and Glyma.01G084200; SEQ ID NO: 9), Vicia faba FUN protein (Vfaba.Hedin2.R1.1g203360.1; SEQ ID NO: 81), and V. unguiculata (Cowpea) FUN (VigunO2g036100.1.p; SEQ ID NO: 82). FUN proteins that show activity, expression, or enhanced activity or enhanced expression in root nodules can be readily distinguished from FUN-like proteins, paralogs of FUN proteins, that can be distinguished by their lack of enhanced expression or activity in the nodule. FUN-like proteins also form an independent paralogous branch on the phylogenetic tree. Exemplary FUN-like proteins include Lotus FUN-like protein (LotjaGi5glvO341400; SEQ ID NO: 83); Cowpea FUN-like protein (Vigun07g272100.1.p; SEQ ID NO: 84); Soybean FUN-likeA (G. max Wm82.a2.vl|Glyma.10G276100.1.p; SEQ ID ON: 6); Soybean FUN-likeB (G. max Wm82.a2.vl|Glyma.20Gl13600.1.p; SEQ ID NO: 7), and Arabidopsis PAN (A. thaliana AraportlI11|AT1G68640.1; SEQ ID NO: 4). FUN proteins can be those that overexpress in the nodule. A FUN or FUN-like protein can be in an inactive, filamentous form (e.g., large filaments) or an active, oligomeric, non-filamentous form. The FUN or FUN-like gene encodes a protein of the TGA family of transcription factors. TGA transcription factors belong to the bZIP family of transcription factors, wherein the bZIP family of transcription factors can be characterized by the presence oaf leucine zipper (bZIP) DNA-binding domain in the N-terminus and a DOG1 domain at the C-terminus, wherein the DOG1 domain is referred to as the sensor domain in FIG. 1P (Tomaz̆, S̆., Gruden, K. & Col, A. TGA transcription factors-Structural characteristics as basis for functional variability. Front. Plant Sci. 13, 935819 (2022)). Protein domains and detailed positions were analyzed and listed in Table 1 for the FUN and FUN-like protein sequences disclosed herein.

TABLE 1
Protein domains and positions identified for FUN, FUN-like,
NAC, NRT3.1, NRT2.1, AS1, and HO1 protein sequences.

| SEQ | | | | |
|---|---|---|---|---|
| ID NO | Gene | Pfam | Position(E-value) | Description |
| 1 | FUN | DOG1 | 266 . . . 340(1.2e−29) | PF14144, Seed dormancy control |
| 1 | FUN | bZIP_1 | 181 . . . 221(2e−06) | PF00170, bZIP transcription factor |
| 1 | FUN | bZIP_2 | 179 . . . 227(0.00079) | PF07716, Basic region leucine zipper |
| 1 | FUN | bZIP_Maf | 170 . . . 230(0.035) | PF03131, bZIP Maf transcription factor |
| 2 | FUN | DOG1 | 242 . . . 317(5.9e−31) | PF14144, Seed dormancy control |
| 2 | FUN | bZIP_1 | 160 . . . 200(1.3e−06) | PF00170, bZIP transcription factor |
| 2 | FUN | bZIP_2 | 158 . . . 205(2.1e−06) | PF07716, Basic region leucine zipper |
| 2 | FUN | bZIP_Maf | 152 . . . 205(0.014) | PF03131, bZIP Maf transcription factor |
| 2 | FUN | Arena_RNA_pol | 238 . . . 285(0.3) | PF06317, Arenavirus RNA polymerase |
| 2 | FUN | End3 | 119 . . . 251(0.045) | PF12761, Actin cytoskeleton-regulatory |
| | | | | complex protein END3 |
| 3 | FUN | DOG1 | 151 . . . 225(5.1e−29) | PF14144, Seed dormancy control |
| 3 | FUN | bZIP_1 | 64 . . . 105(4.1e−06) | PF00170, bZIP transcription factor |
| 3 | FUN | bZIP_2 | 63 . . . 113(3.1e−05) | PF07716, Basic region leucine zipper |
| 3 | FUN | DnaJ- | 62 . . . 170(0.087) | PF11875, DnaJ-like protein C11, C- |
| | | like_C11_C | | terminal |
| 5 | FUN | DOG1 | 241 . . . 314(2.1e−27) | PF14144, Seed dormancy control |
| 5 | FUN | bZIP_1 | 155 . . . 195(2.5e−07) | PF00170, bZIP transcription factor |
| 5 | FUN | bZIP_2 | 153 . . . 197(1.2e−05) | PF07716, Basic region leucine zipper |
| 8 | FUN | DOG1 | 266 . . . 340(1.5e−28) | PF14144, Seed dormancy control |
| 8 | FUN | bZIP_1 | 183 . . . 219(2.6e−07) | PF00170, bZIP transcription factor |
| 8 | FUN | bZIP_2 | 181 . . . 229(2e−06) | PF07716, Basic region leucine zipper |
| 8 | FUN | bZIP_Maf | 183 . . . 228(0.01) | PF03131, bZIP Maf transcription factor |
| 9 | FUN | DOG1 | 285 . . . 359(4e−29) | PF14144, Seed dormancy control |
| 9 | FUN | bZIP_1 | 202 . . . 240(4.8e−07) | PF00170, bZIP transcription factor |
| 9 | FUN | bZIP_2 | 200 . . . 248(3.3e−06) | PF07716, Basic region leucine zipper |
| 9 | FUN | bZIP_Maf | 202 . . . 248(0.019) | PF03131, bZIP Maf transcription factor |
| 9 | FUN | BAAT_C | 115 . . . 212(0.25) | PF08840, BAAT/Acyl-CoA thioester |
| | | | | hydrolase C terminal |
| 10 | FUN | DOG1 | 275 . . . 348(2e−27) | PF14144, Seed dormancy control |
| 10 | FUN | bZIP_1 | 187 . . . 217(2.7e−07) | PF00170, bZIP transcription factor |
| 10 | FUN | bZIP_2 | 186 . . . 234(3.1e−06) | PF07716, Basic region leucine zipper |
| 10 | FUN | bZIP_Maf | 183 . . . 233(0.026) | PF03131, bZIP Maf transcription factor |
| 11 | FUN | bZIP_1 | 125 . . . 165(7.6e−07) | PF00170, bZIP transcription factor |
| 11 | FUN | bZIP_2 | 123 . . . 170(8.6e−06) | PF07716, Basic region leucine zipper |
| 12 | FUN | DOG1 | 233 . . . 268(7.7e−12), | PF14144, Seed dormancy control |
| | | | 270 . . . 306(1.8e−13) | |
| 12 | FUN | Gp_dh_N | 318 . . . 363(1.3e−12) | PF00044, Glyceraldehyde 3-phosphate |
| | | | | dehydrogenase, NAD binding domain |
| 12 | FUN | bZIP_1 | 153 . . . 180(0.022) | PF00170, bZIP transcription factor |
| 12 | FUN | PcRGLX_1st | 100 . . . 163(0.058) | PF19501, PcRGLX-like N-terminal RIFT |
| | | | | barrel domain |
| 12 | FUN | bZIP_2 | 153 . . . 188(0.092) | PF07716, Basic region leucine zipper |
| 12 | FUN | TgMIC1 | 59 . . . 112(0.23) | PF11476, Toxoplasma gondii micronemal |
| | | | | protein 1 TgMIC1 |
| 13 | FUN | bZIP_1 | 148 . . . 186(7.6e−07) | PF00170, bZIP transcription factor |
| 13 | FUN | bZIP_2 | 146 . . . 190(7.7e−06) | PF07716, Basic region leucine zipper |
| 13 | FUN | bZIP_Maf | 143 . . . 190(0.28) | PF03131, bZIP Maf transcription factor |
| 14 | FUN | DOG1 | 258 . . . 331(4.9e−30) | PF14144, Seed dormancy control |
| 14 | FUN | bZIP_2 | 171 . . . 215(6e−07) | PF07716, Basic region leucine zipper |
| 14 | FUN | bZIP_1 | 173 . . . 213(1.3e−06) | PF00170, bZIP transcription factor |
| 15 | FUN | DOG1 | 236 . . . 309(4e−29) | PF14144, Seed dormancy control |
| 15 | FUN | bZIP_1 | 151 . . . 193(6.3e−07) | PF00170, bZIP transcription factor |
| 15 | FUN | bZIP_2 | 149 . . . 193(2.8e−06) | PF07716, Basic region leucine zipper |
| 15 | FUN | FRG1 | 129 . . . 200(0.55) | PF06229, FRG1-like domain |
| 16 | FUN | DOG1 | 265 . . . 338(6.9e−31) | PF14144, Seed dormancy control |
| 16 | FUN | bZIP_1 | 183 . . . 222(3.9e−07) | PF00170, bZIP transcription factor |
| 16 | FUN | bZIP_2 | 181 . . . 229(1.6e−06) | PF07716, Basic region leucine zipper |
| 16 | FUN | PRCC | 139 . . . 227(0.11) | PF10253, Mitotic checkpoint regulator, |
| | | | | MAD2B-interacting |
| 16 | FUN | Arena_RNA_pol | 259 . . . 307(0.09) | PF06317, Arenavirus RNA polymerase |
| 16 | FUN | FapA | 139 . . . 232(0.35) | PF03961, Flagellar Assembly Protein A |
| | | | | beta solenoid domain |
| 17 | FUN | DOG1 | 251 . . . 324(2.2e−30) | PF14144, Seed dormancy control |
| 17 | FUN | bZIP_1 | 169 . . . 206(7e−07) | PF00170, bZIP transcription factor |
| 17 | FUN | bZIP_2 | 167 . . . 214(2.1e−06) | PF07716, Basic region leucine zipper |
| 18 | FUN | DOG1 | 263 . . . 336(1.1e−31) | PF14144, Seed dormancy control |
| 18 | FUN | bZIP_1 | 178 . . . 214(1.2e−06) | PF00170, bZIP transcription factor |
| 18 | FUN | bZIP_2 | 176 . . . 224(2.6e−06) | PF07716, Basic region leucine zipper |
| 19 | FUN | DOG1 | 281 . . . 354(4.1e−30) | PF14144, Seed dormancy control |
| 19 | FUN | bZIP_1 | 196 . . . 237(1.6e−06) | PF00170, bZIP transcription factor |
| 19 | FUN | bZIP_2 | 194 . . . 237(4.6e−05) | PF07716, Basic region leucine zipper |
| 19 | FUN | bZIP_Maf | 194 . . . 242(0.031) | PF03131, bZIP Maf transcription factor |
| 19 | FUN | FRG1 | 193 . . . 245(0.2) | PF06229, FRG1-like domain |
| 20 | FUN | DOG1 | 173 . . . 249(9.9e−27) | PF14144, Seed dormancy control |
| 20 | FUN | bZIP_1 | 93 . . . 138(1e−07) | PF00170, bZIP transcription factor |
| 20 | FUN | bZIP_2 | 91 . . . 138(5.4e−07) | PF07716, Basic region leucine zipper |
| 20 | FUN | Arteri_nucleo | 101 . . . 175(0.014) | PF01481, Arterivirus nucleocapsid protein |
| 20 | FUN | bZIP_Maf | 93 . . . 138(0.01) | PF03131, bZIP Maf transcription factor |
| 21 | FUN | DOG1 | 173 . . . 249(9.9e−27) | PF14144, Seed dormancy control |
| 21 | FUN | bZIP_1 | 93 . . . 138(1e−07) | PF00170, bZIP transcription factor |
| 21 | FUN | bZIP_2 | 91 . . . 138(5.4e−07) | PF07716, Basic region leucine zipper |
| 21 | FUN | Arteri_nucleo | 101 . . . 175(0.014) | PF01481, Arterivirus nucleocapsid protein |
| 21 | FUN | bZIP_Maf | 93 . . . 138(0.01) | PF03131, bZIP Maf transcription factor |
| 22 | FUN | DOG1 | 153 . . . 216(2.8e−21) | PF14144, Seed dormancy control |
| 22 | FUN | bZIP_2 | 71 . . . 115(7.1e−07) | PF07716, Basic region leucine zipper |
| 22 | FUN | bZIP_1 | 73 . . . 114(1.1e−06) | PF00170, bZIP transcription factor |
| 22 | FUN | Arteri_nucleo | 81 . . . 155(0.00018) | PF01481, Arterivirus nucleocapsid protein |
| 22 | FUN | Spectrin_2 | 152 . . . 187(0.032) | PF18373, Spectrin like domain |
| 22 | FUN | bZIP_Maf | 73 . . . 118(0.03) | PF03131, bZIP Maf transcription factor |
| 23 | FUN | DOG1 | 233 . . . 308(2.7e−27) | PF14144, Seed dormancy control |
| 23 | FUN | bZIP_1 | 149 . . . 187(4.4e−07) | PF00170, bZIP transcription factor |
| 23 | FUN | bZIP_2 | 149 . . . 193(2.6e−05) | PF07716, Basic region leucine zipper |
| 24 | FUN | DOG1 | 231 . . . 306(2.3e−27) | PF14144, Seed dormancy control |
| 24 | FUN | bZIP_1 | 148 . . . 190(7.6e−08) | PF00170, bZIP transcription factor |
| 24 | FUN | bZIP_2 | 148 . . . 194(3.4e−05) | PF07716, Basic region leucine zipper |
| 24 | FUN | bZIP_Maf | 149 . . . 194(0.022) | PF03131, bZIP Maf transcription factor |
| 24 | FUN | Herpes_PAP | 339 . . . 393(0.37) | PF03325, Herpesvirus polymerase |
| | | | | accessory protein |
| 25 | FUN | DOG1 | 7 . . . 32(2.5e−05) | PF14144, Seed dormancy control |
| 25 | FUN | Cucumopine_C | 54 . . . 128(0.072) | PF18631, Cucumopine synthase C- |
| | | | | terminal helical bundle domain |
| 25 | FUN | Big_3_3 | 47 . . . 92(0.24) | PF13750, Bacterial Ig-like domain (group |
| | | | | 3) |
| 26 | FUN | DOG1 | 47 . . . 70(0.0049) | PF14144, Seed dormancy control |
| 27 | FUN | Cucumopine_C | 75 . . . 141(0.16) | PF18631, Cucumopine synthase C- |
| | | | | terminal helical bundle domain |
| 28 | FUN | DOG1 | 179 . . . 255(4.5e−30) | PF14144, Seed dormancy control |
| 28 | FUN | bZIP_1 | 95 . . . 135(1.9e−07) | PF00170, bZIP transcription factor |
| 28 | FUN | bZIP_2 | 93 . . . 137(5.6e−07) | PF07716, Basic region leucine zipper |
| 28 | FUN | bZIP_Maf | 86 . . . 139(0.015) | PF03131, bZIP Maf transcription factor |
| 29 | FUN | DOG1 | 227 . . . 302(8e−30) | PF14144, Seed dormancy control |
| 29 | FUN | bZIP_1 | 141 . . . 176(3.7e−08) | PF00170, bZIP transcription factor |
| 29 | FUN | bZIP_2 | 140 . . . 187(1.1e−05) | PF07716, Basic region leucine zipper |
| 30 | FUN | DOG1 | 73 . . . 148(2.6e−29) | PF14144, Seed dormancy control |
| 80 | FUN | DOG1 | 266 . . . 340(1.2e−29) | PF14144, Seed dormancy control |
| 80 | FUN | bZIP_1 | 181 . . . 221(1.9e−06) | PF00170, bZIP transcription factor |
| 80 | FUN | bZIP_2 | 179 . . . 227(0.00073) | PF07716, Basic region leucine zipper |
| 80 | FUN | bZIP_Maf | 170 . . . 230(0.034) | PF03131, bZIP Maf transcription factor |
| 81 | FUN | DOG1 | 257 . . . 331(1.7e−25) | PF14144, Seed dormancy control |
| 81 | FUN | bZIP_1 | 171 . . . 212(1.9e−07) | PF00170, bZIP transcription factor |
| 81 | FUN | bZIP_2 | 171 . . . 220(9e−06) | PF07716, Basic region leucine zipper |
| 81 | FUN | FAN1_HTH | 410 . . . 439(0.17) | PF21315, FAN1, HTH domain |
| 81 | FUN | Translin | 340 . . . 446(0.08) | PF01997, Translin family |
| 82 | FUN | DOG1 | 260 . . . 334(1.6e−26) | PF14144, Seed dormancy control |
| 82 | FUN | bZIP_1 | 176 . . . 216(2e−07) | PF00170, bZIP transcription factor |
| 82 | FUN | bZIP_2 | 175 . . . 223(1.2e−05) | PF07716, Basic region leucine zipper |
| 82 | FUN | SprA-related | 133 . . . 224(0.065) | PF12118, SprA-related family |
| 82 | FUN | bZIP_Maf | 173 . . . 223(0.063) | PF03131, bZIP Maf transcription factor |
| 4 | FUN-like | DOG1 | 251 . . . 324(1.5e−27) | PF14144, Seed dormancy control |
| 4 | FUN-like | bZIP_1 | 166 . . . 206(6.7e−07) | PF00170, bZIP transcription factor |
| 4 | FUN-like | bZIP_2 | 163 . . . 212(4.9e−06) | PF07716, Basic region leucine zipper |
| 4 | FUN-like | FRG1 | 154 . . . 217(0.025) | PF06229, FRG1-like domain |
| 4 | FUN-like | CCDC32 | 173 . . . 214(0.73) | PF14989, Coiled-coil domain containing |
| | | | | 32 |
| 6 | FUN-like | DOG1 | 254 . . . 328(5.5e−30) | PF14144, Seed dormancy control |
| 6 | FUN-like | bZIP_1 | 168 . . . 208(6.6e−07) | PF00170, bZIP transcription factor |
| 6 | FUN-like | bZIP_2 | 167 . . . 215(3.7e−06) | PF07716, Basic region leucine zipper |
| 6 | FUN-like | Gastrin | 154 . . . 253(0.21) | PF00918, Gastrin/cholecystokinin family |
| 7 | FUN-like | DOG1 | 255 . . . 327(5.3e−28) | PF14144, Seed dormancy control |
| 7 | FUN-like | bZIP_1 | 167 . . . 207(1.3e−06) | PF00170, bZIP transcription factor |
| 7 | FUN-like | bZIP_2 | 166 . . . 213(3.6e−06) | PF07716, Basic region leucine zipper |
| 7 | FUN-like | Gastrin | 152 . . . 247(0.11) | PF00918, Gastrin/cholecystokinin family |
| 7 | FUN-like | TMP-TENI | 409 . . . 443(0.47) | PF02581, Thiamine monophosphate |
| | | | | synthase |
| 83 | FUN-like | DOG1 | 163 . . . 236(1.3e−27) | PF14144, Seed dormancy control |
| 83 | FUN-like | bZIP_1 | 79 . . . 110(3.8e−07) | PF00170, bZIP transcription factor |
| 83 | FUN-like | bZIP_2 | 77 . . . 121(1.2e−05) | PF07716, Basic region leucine zipper |
| 83 | FUN-like | IclR | 89 . . . 149(0.062) | PF01614, Bacterial transcriptional |
| | | | | regulator |
| 83 | FUN-like | NAM-associated | 26 . . . 133(0.46) | PF14303, No apical meristem-associated |
| | | | | C-terminal domain |
| 83 | FUN-like | FUSC | 69 . . . 168(0.69) | PF04632, Fusaric acid resistance protein |
| | | | | family |
| 84 | FUN-like | DOG1 | 260 . . . 334(4e−30) | PF14144, Seed dormancy control |
| 84 | FUN-like | bZIP_1 | 175 . . . 214(4.3e−07) | PF00170, bZIP transcription factor |
| 84 | FUN-like | bZIP_2 | 173 . . . 220(7.9e−06) | PF07716, Basic region leucine zipper |
| 31 | NAC | NAM | 19 . . . 143(5.9e−49) | PF02365, No apical meristem (NAM) |
| | | | | protein |
| 32 | NAC | NAM | 9 . . . 87(6e−25) | PF02365, No apical meristem (NAM) |
| | | | | protein |
| 33 | NAC | NAM | 16 . . . 141(9e−48) | PF02365, No apical meristem (NAM) |
| | | | | protein |
| 34 | NAC | NAM | 22 . . . 147(9.9e−49) | PF02365, No apical meristem (NAM) |
| | | | | protein |
| 35 | NAC | NAM | 16 . . . 141(6.9e−48) | PF02365, No apical meristem (NAM) |
| | | | | protein |
| 36 | NAC | NAM | 16 . . . 141(3.4e−48) | PF02365, No apical meristem (NAM) |
| | | | | protein |
| 37 | NAC | NAM | 13 . . . 138(4.9e−48) | PF02365, No apical meristem (NAM) |
| | | | | protein |
| 37 | NAC | BH4 | 22 . . . 38(0.19) | PF02180, Bcl-2 homology region 4 |
| 38 | NAC | NAM | 16 . . . 141(6e−48) | PF02365, No apical meristem (NAM) |
| | | | | protein |
| 38 | NAC | RPEL | 32 . . . 48(0.15) | PF02755, RPEL repeat |
| 39 | NAC | NAM | 17 . . . 142(4.8e−48) | PF02365, No apical meristem (NAM) |
| | | | | protein |
| 40 | NAC | NAM | 20 . . . 146(3e−48) | PF02365, No apical meristem (NAM) |
| | | | | protein |
| 41 | NAC | NAM | 14 . . . 138(3.2e−48) | PF02365, No apical meristem (NAM) |
| | | | | protein |
| 42 | NAC | NAM | 15 . . . 139(2.9e−48) | PF02365, No apical meristem (NAM) |
| | | | | protein |
| 43 | NAC | NAM | 16 . . . 141(8.8e−48) | PF02365, No apical meristem (NAM) |
| | | | | protein |
| 44 | NAC | NAM | 16 . . . 142(8.3e−48) | PF02365, No apical meristem (NAM) |
| | | | | protein |
| 45 | NAC | NAM | 17 . . . 142(9.3e−48) | PF02365, No apical meristem (NAM) |
| | | | | protein |
| 46 | NAC | NAM | 20 . . . 145(7.8e−48) | PF02365, No apical meristem (NAM) |
| | | | | protein |
| 47 | NAC | NAM | 18 . . . 143(7.6e−48) | PF02365, No apical meristem (NAM) |
| | | | | protein |
| 48 | NAC | NAM | 17 . . . 142(5.2e−48) | PF02365, No apical meristem (NAM) |
| | | | | protein |
| 49 | NAC | NAM | 17 . . . 142(5.6e−48) | PF02365, No apical meristem (NAM) |
| | | | | protein |
| 50 | NAC | NAM | 21 . . . 149(1.4e−47) | PF02365, No apical meristem (NAM) |
| | | | | protein |
| 50 | NAC | Ly49 | 178 . . . 252(0.24) | PF08391, Ly49-like protein, N-terminal |
| | | | | region |
| 51 | NAC | NAM | 16 . . . 141(2.5e−48) | PF02365, No apical meristem (NAM) |
| | | | | protein |
| 51 | NAC | Maf1 | 173 . . . 239(0.4) | PF09174, Maf1 regulator |
| 52 | NAC | NAM | 17 . . . 142(3e−48) | PF02365, No apical meristem (NAM) |
| | | | | protein |
| 53 | NAC | NAM | 16 . . . 141(5.9e−48) | PF02365, No apical meristem (NAM) |
| | | | | protein |
| 54 | NAC | NAM | 16 . . . 142(4.3e−47) | PF02365, No apical meristem (NAM) |
| | | | | protein |
| 55 | NAC | NAM | 15 . . . 141(2.7e−46) | PF02365, No apical meristem (NAM) |
| | | | | protein |
| 56 | NAC | NAM | 15 . . . 141(2.7e−46) | PF02365, No apical meristem (NAM) |
| | | | | protein |
| 57 | NAC | NAM | 15 . . . 141(3.5e−46) | PF02365, No apical meristem (NAM) |
| | | | | protein |
| 58 | NAC | NAM | 15 . . . 141(2.1e−46) | PF02365, No apical meristem (NAM) |
| | | | | protein |
| 59 | NAC | NAM | 15 . . . 141(1.7e−46) | PF02365, No apical meristem (NAM) |
| | | | | protein |
| 60 | NAC | NAM | 15 . . . 141(2.3e−46) | PF02365, No apical meristem (NAM) |
| | | | | protein |
| 61 | NAC | NAM | 15 . . . 141(1.2e−45) | PF02365, No apical meristem (NAM) |
| | | | | protein |
| 62 | NAC | NAM | 15 . . . 140(1e−46) | PF02365, No apical meristem (NAM) |
| | | | | protein |
| 63 | NAC | NAM | 18 . . . 143(8.5e−47) | PF02365, No apical meristem (NAM) |
| | | | | protein |
| 64 | NAC | NAM | 18 . . . 143(6.9e−47) | PF02365, No apical meristem (NAM) |
| | | | | protein |
| 65 | NAC | NAM | 18 . . . 143(1.2e−47) | PF02365, No apical meristem (NAM) |
| | | | | protein |
| 66 | NAC | NAM | 18 . . . 143(8.7e−48) | PF02365, No apical meristem (NAM) |
| | | | | protein |
| 67 | NAC | NAM | 18 . . . 143(8.5e−48) | PF02365, No apical meristem (NAM) |
| | | | | protein |
| 68 | NAC | NAM | 19 . . . 144(9.9e−48) | PF02365, No apical meristem (NAM) |
| | | | | protein |
| 69 | NAC | NAM | 17 . . . 142(1.7e−48) | PF02365, No apical meristem (NAM) |
| | | | | protein |
| 70 | NAC | NAM | 16 . . . 141(3.4e−47) | PF02365, No apical meristem (NAM) |
| | | | | protein |
| 71 | NAC | NAM | 17 . . . 142(3.8e−48) | PF02365, No apical meristem (NAM) |
| | | | | protein |
| 72 | NAC | NAM | 16 . . . 142(1.7e−46) | PF02365, No apical meristem (NAM) |
| | | | | protein |
| 73 | NAC | NAM | 18 . . . 143(4.9e−48) | PF02365, No apical meristem (NAM) |
| | | | | protein |
| 76 | NAC | NAM | 19 . . . 143(5.9e−49) | PF02365, No apical meristem (NAM) |
| | | | | protein |
| 74 | NRT3.1 | NAR2 | 26 . . . 170(5.7e−65) | PF16974, High-affinity nitrate transporter |
| | | | | accessory, Ig-like domain |
| 78 | NRT2.1 | MFS_1 | 69 . . . 425(2e−23) | PF07690, Major Facilitator Superfamily |
| 79 | AS1 | Asn_synthase | 210 . . . 361(1.4e−50), | PF00733, Asparagine synthase |
| | | | 359 . . . 511(3.6e−34) | |
| 79 | AS1 | GATase_7 | 48 . . . 165(1.3e−38) | PF13537, Glutamine amidotransferase |
| | | | | domain |
| 79 | AS1 | GATase_6 | 33 . . . 159(1e−33) | PF13522, Glutamine amidotransferase |
| | | | | domain |
| 79 | AS1 | DUF3700 | 113 . . . 194(6.9e−09) | PF12481, Aluminium induced protein |
| 79 | AS1 | NAD_synthase | 228 . . . 290(0.077) | PF02540, NAD synthase |
| 77 | HO1 | Heme_oxygenase | 110 . . . 276(2.1e−15) | PF01126, Heme oxygenase |

Protein domains and motifs are both conserved sequence patterns. A domain is an independently-folding unit of a protein. A motif is a small region or set of small regions of three-dimensional structure or nucleotide sequence, or amino acid sequence that are shared among proteins. Conserved domains or motifs are recurring units in molecular evolution, the extents of which can be determined by sequence and structure analysis. Conserved domains or motifs can contain conserved sequence patterns or sequence motifs, which allow for detection of the domain or motif in polypeptide sequences.

### Downstream Targets of FUN Proteins

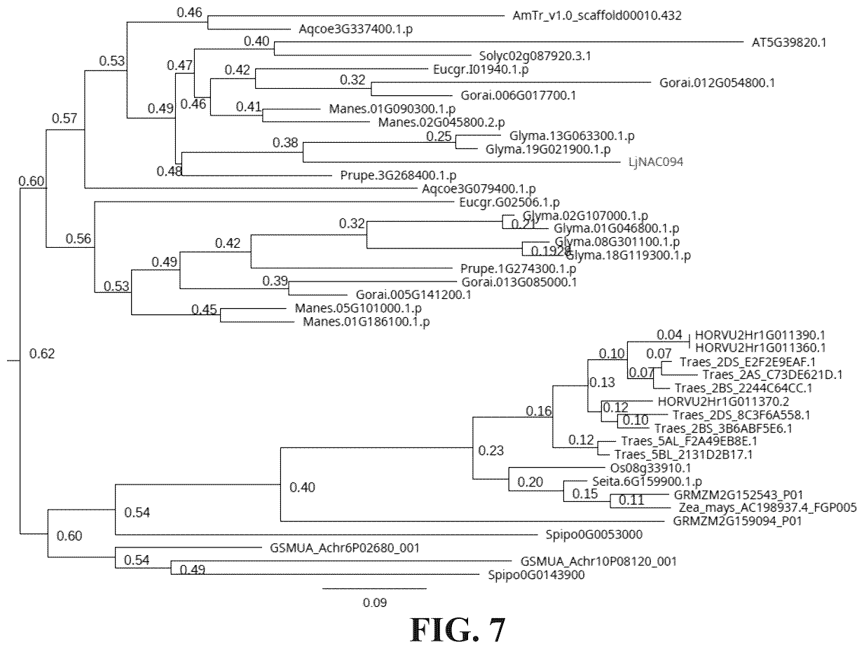

FUN is a transcriptional regulator that controls the expression of downstream genes including High-affinity Nitrate Transporter 2.1 (NRT2.1), Heme Oxygenase (HO1), NAC domain containing protein 94 (NAC transcription factor 94, or NAC094), High-affinity Nitrate Transporter 3.1 (NRT3.1), basic leucine zipper transcription factor 28 (bZIP28), and Asparagine Synthetase 1 (AS1) to regulate nitrate signaling and nitrogen fixation in root nodules. An exemplary NRT2.1 protein includes SEQ ID NO: 78. Exemplary HO1 protein includes SEQ ID NO: 77. An exemplary NRT3.1 protein includes SEQ ID NO: 74. An exemplary bZIP28 protein includes SEQ ID NO: 75. An exemplary AS1 protein includes SEQ ID NO: 79. L. japonicus NAC094 (SEQ ID NO: 31; also referred to as a FEZ protein), which is a downstream target of FUN (FIG. 7). Exemplary NAC094 homologues (NAC-domain containing protein) include SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, and SEQ ID NO: 76.

### Genetically Modified Plants and Related Methods

An aspect of the disclosure includes a genetically modified plant or part thereof including one or more genetic alterations that result in decreased activity or expression of a FUN protein as compared to the activity or expression of a FUN protein in a control plant grown under the same conditions. In a further embodiment of this aspect, the FUN protein includes a polypeptide with at least 70% identity, at least 75% identity, at least 80% identity, at least 85% identity, at least 90% identity, at least 95% identity, or at least 99% identity to a protein selected from the group of SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, or SEQ ID NO: 82, or a polypeptide with at least 70% identity, at least 75% identity, at least 80% identity, at least 85% identity, at least 90% identity, at least 95% identity, or at least 99% identity to domains thereof, for example, at least 70% identity, at least 75% identity, at least 80% identity, at least 85% identity, at least 90% identity, at least 95% identity, or at least 99% identity to the respective domains thereof recited in Table 1, for example, bZIP1, bZIP2, or DOG1 domains thereof, or combinations thereof. In an additional embodiment of this aspect, the FUN protein includes SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, or SEQ ID NO: 82, or respective domains thereof recited in Table 1, or combinations thereof. In yet another embodiment of this aspect, the FUN protein includes SEQ ID NO: 1, SEQ ID NO: 8, or SEQ ID NO: 9, or respective domains thereof recited in Table 1, or combinations thereof.

A further aspect of the disclosure includes a genetically modified plant or part thereof including one or more genetic alterations that result in decreased activity or expression of one or more of a NRT3.1 protein, a bZIP28 protein, a NAC-domain containing protein (also referred to as a FEZ protein), a HO1 protein, a NRT2.1 protein, or an AS1 protein as compared to the activity or expression of a NRT3.1 protein, a bZIP28 protein, a NAC-domain containing protein, a HO1 protein, a NRT2.1 protein, or an AS1 protein in a control plant grown under the same conditions. In a further embodiment of this aspect, wherein the protein is the NRT3.1 protein, and wherein the NRT3.1 protein includes a polypeptide with at least 70% identity, at least 75% identity, at least 80% identity, at least 85% identity, at least 90% identity, at least 95% identity, or at least 99% identity to SEQ ID NO: 74; wherein the protein is the bZIP28 protein, and wherein the bZIP28 protein includes a polypeptide with at least 70% identity, at least 75% identity, at least 80% identity, at least 85% identity, at least 90% identity, at least 95% identity, or at least 99% identity to SEQ ID NO: 75; wherein the protein is the NAC-domain containing protein, and wherein the NAC-domain containing protein includes a polypeptide with at least 70% identity, at least 75% identity, at least 80% identity, at least 85% identity, at least 90% identity, at least 95% identity, or at least 99% identity to a protein selected from the group of SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, and SEQ ID NO: 73; wherein the protein is the HO1 protein, and wherein the HO1 protein includes a polypeptide with at least 70% identity, at least 75% identity, at least 80% identity, at least 85% identity, at least 90% identity, at least 95% identity, or at least 99% identity to SEQ ID NO: 77; wherein the protein is the NRT2.1 protein, and wherein the NRT2.1 protein includes a polypeptide with at least 70% identity, at least 75% identity, at least 80% identity, at least 85% identity, at least 90% identity, at least 95% identity, or at least 99% identity to SEQ ID NO: 78; or wherein the protein is the AS1 protein, and wherein the AS1 protein includes a polypeptide with at least 70% identity, at least 75% identity, at least 80% identity, at least 85% identity, at least 90% identity, at least 95% identity, or at least 99% identity to SEQ ID NO: 79, or a polypeptide with at least 70% identity, at least 75% identity, at least 80% identity, at least 85% identity, at least 90% identity, at least 95% identity, or at least 99% identity to domains thereof, for example, at least 70% identity, at least 75% identity, at least 80% identity, at least 85% identity, at least 90% identity, at least 95% identity, or at least 99% identity to respective domains thereof recited in Table 1, or combinations thereof. In an additional embodiment of this aspect, the NRT3.1 protein includes SEQ ID NO: 74; wherein the bZIP28 protein includes SEQ ID NO: 75; wherein the NAC-domain containing protein includes SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, or SEQ ID NO: 73; wherein the HO1 protein includes SEQ ID NO: 77; wherein the NRT2.1 protein includes SEQ ID NO: 78, or wherein the AS1 protein includes SEQ ID NO: 79, or respective domains thereof recited in Table 1, or combinations thereof. In still another embodiment of this aspect, the NAC-domain containing protein includes SEQ ID NO: 31, SEQ ID NO: 41, or SEQ ID NO: 42, or respective domains thereof recited in Table 1, or combinations thereof. Protein domains and detailed positions were analyzed and listed in Table 1 for the NAC-domain containing NRT3.1, NRT2.1, AS1, and HO1 protein sequences disclosed herein.

An additional aspect of the present disclosure relates to a genetically altered plant genome including (i) the one or more genetic alterations in the genetically modified plant or part thereof of any one of the preceding embodiments, or (ii) the one or more genetic alterations in the genetically modified plant or part produced by the method of any one of the preceding embodiments. In yet another embodiment of this aspect, the FUN protein includes a polypeptide with at least 70% identity, at least 75% identity, at least 80% identity, at least 85% identity, at least 90% identity, at least 95% identity, or at least 99% identity to a protein selected from the group of SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, or SEQ ID NO: 82, or a polypeptide with at least 70% identity, at least 75% identity, at least 80% identity, at least 85% identity, at least 90% identity, at least 95% identity, or at least 99% identity to domains thereof, for example, at least 70% identity, at least 75% identity, at least 80% identity, at least 85% identity, at least 90% identity, at least 95% identity, or at least 99% identity to respective domains thereof recited in Table 1, or combinations thereof. In another embodiment of this aspect, the FUN protein includes SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, or SEQ ID NO: 82, or respective domains thereof recited in Table 1, or combinations thereof. In a further embodiment of this aspect, wherein the protein is the NRT3.1 protein, and wherein the NRT3.1 protein includes a polypeptide with at least 70% identity, at least 75% identity, at least 80% identity, at least 85% identity, at least 90% identity, at least 95% identity, or at least 99% identity to SEQ ID NO: 74; wherein the protein is the bZIP28 protein, and wherein the bZIP28 protein includes a polypeptide with at least 70% identity, at least 75% identity, at least 80% identity, at least 85% identity, at least 90% identity, at least 95% identity, or at least 99% identity to SEQ ID NO: 75; wherein the protein is the NAC-domain containing protein, and wherein the NAC-domain containing protein includes a polypeptide with at least 70% identity, at least 75% identity, at least 80% identity, at least 85% identity, at least 90% identity, at least 95% identity, or at least 99% identity to a protein selected from the group of SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, and SEQ ID NO: 73; wherein the protein is the HO1 protein, and wherein the HO1 protein includes a polypeptide with at least 70% identity, at least 75% identity, at least 80% identity, at least 85% identity, at least 90% identity, at least 95% identity, or at least 99% identity to SEQ ID NO: 77; wherein the protein is the NRT2.1 protein, and wherein the NRT2.1 protein includes a polypeptide with at least 70% identity, at least 75% identity, at least 80% identity, at least 85% identity, at least 90% identity, at least 95% identity, or at least 99% identity to SEQ ID NO: 78; or wherein the protein is the AS1 protein, and wherein the AS1 protein includes a polypeptide with at least 70% identity, at least 75% identity, at least 80% identity, at least 85% identity, at least 90% identity, at least 95% identity, or at least 99% identity to SEQ ID NO: 79, or a polypeptide with at least 70% identity, at least 75% identity, at least 80% identity, at least 85% identity, at least 90% identity, at least 95% identity, or at least 99% identity to domains thereof, for example, at least 70% identity, at least 75% identity, at least 80% identity, at least 85% identity, at least 90% identity, at least 95% identity, or at least 99% identity to respective domains thereof recited in Table 1, or combinations thereof. In an additional embodiment of this aspect, the NRT3.1 protein includes SEQ ID NO: 74; wherein the bZIP28 protein includes SEQ ID NO: 75; wherein the NAC-domain containing protein includes SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, or SEQ ID NO: 73; wherein the HO1 protein includes SEQ ID NO: 77; wherein the NRT2.1 protein includes SEQ ID NO: 78, or wherein the AS1 protein includes SEQ ID NO: 79, or respective domains thereof recited in Table 1, or combinations thereof. In still another embodiment of this aspect, the NAC-domain containing protein includes SEQ ID NO: 31, SEQ ID NO: 41, or SEQ ID NO: 42, or respective domains thereof recited in Table 1, or combinations thereof.

In an additional embodiment of this aspect, which may be combined with any of the preceding embodiments, the decrease is at least 10%, at least 20%, at least 30%, at least 40%, at least 50%, at least 60%, at least 70%, at least 80%, at least 90%, or at least 100%. In yet another embodiment of this aspect, which may be combined with any of the preceding embodiments, the decrease is due to knock-out of a gene for the protein, introduction of a premature stop codon in the coding sequence of the gene for the protein, RNAi silencing, knock-out of a domain of the protein, introduction of a transcriptional repressor protein binding site, or knock-out of a binding site in the promoter region of the gene and/or the genetic alterations include knock-out of a gene for the protein, introduction of a premature stop codon in the coding sequence of the gene for the protein, RNAi silencing, knock-out of a domain of the protein, introduction of a transcriptional repressor protein binding site, or knock-out of a binding site in the promoter region of the gene and/or the genetic alterations include knock-out of a gene for the protein, introduction of a premature stop codon in the coding sequence of the gene for the protein, RNAi silencing, knock-out of a domain of the protein, introduction of a transcriptional repressor protein binding site, or knock-out of a binding site in the promoter region of the gene, preferably the binding site is a transcriptional activator protein binding site or a TATA-box. In a further embodiment of this aspect, which may be combined with any of the preceding embodiments, the growth conditions include a moderate nitrate level, a high nitrate level, or a nitrate level around the plant that reduces or suppresses nitrogen fixation. In yet another embodiment of this aspect, the nitrate level is between about 10 mM and about 250 mM nitrate or includes at least about 10 mM nitrate, at least about 20 mM nitrate, at least about 30 mM nitrate, at least about 40 mM nitrate, at least about 50 mM nitrate, at least about 100 mM nitrate, at least about 150 mM nitrate, at least about 200 mM nitrate, or at least about 250 mM nitrate. In still another embodiment of this aspect, the genetically modified plant has increased nitrogen fixation as compared to the control plant when grown under the same growth conditions. In a further embodiment of this aspect, the nitrogen fixation is increased at least 10%, at least 20%, at least 30%, at least 40%, at least 50%, at least 60%, at least 70%, at least 80%, at least 90%, at least 100%, at least 150%, at least 200%, at least 300%, at least 400%, or at least 500%. In yet another embodiment of this aspect, which may be combined with any of the preceding embodiments, the plant forms nodules. In an additional embodiment of this aspect, the number of nodules is increased, hemoglobin content is increased, or the acetylene reduction assay (ARA) activity is increased compared to the control plant when grown under the same conditions.

In certain embodiments of any of the preceding aspects and their various embodiments, the plant part may be a seed, pod, fruit, leaf, flower, stem, root, any part of the foregoing or a cell thereof, or a non-regenerable part or cell of a genetically modified plant part. As used in this context, a “non-regenerable” part or cell of a genetically modified plant or part thereof is a part or cell that itself cannot be induced to form a whole plant or cannot be induced to form a whole plant capable of sexual and/or asexual reproduction. In certain embodiments, the non-regenerable part or cell of the plant part is a part of a transgenic seed, pod, fruit, leaf, flower, stem or root or is a cell thereof. In other embodiments, the non-regenerable part or cell of the plant part is part of a processed plant product.

Processed plant products that contain a detectable amount of a nucleotide segment, expressed RNA, and/or protein comprising a genetic modification disclosed herein are also provided. Such processed products include, but are not limited to, plant biomass, oil, meal, animal feed, flour, flakes, bran, lint, hulls, and processed seed. The processed product may be non-regenerable. The plant product can comprise commodity or other products of commerce derived from a transgenic plant or transgenic plant part, where the commodity or other products can be tracked through commerce by detecting a nucleotide segment, expressed RNA, and/or protein that comprises distinguishing portions of a genetic modification disclosed herein.

An additional aspect of the disclosure includes methods of cultivating a genetically altered plant with increased nitrogen fixation under conditions including a nitrate level around the plant roots that suppresses nitrogen fixation, including: (a) providing the genetically altered plant, wherein the plant or a part thereof includes one or more genetic alterations that result in decreased activity or expression of a FUN protein, a NRT3.1 protein, a bZIP28 protein, a NAC-domain containing protein (also referred to as a FEZ protein), a HO1 protein, a NRT2.1 protein, or an AS1 protein, or any combination thereof as compared to an activity or expression of a FUN protein, a NRT3.1 protein, a bZIP28 protein, a NAC-domain containing protein, a HO1 protein, a NRT2.1 protein, or an AS1 protein in a control plant grown under the same conditions, and wherein the one or more genetic alterations reduce the nitrate level suppression of nitrogen fixation; and (b) cultivating the genetically altered plant under the nitrate level around the plant roots, wherein the genetically modified plant has increased nitrogen fixation as compared to the control plant grown under the same conditions. In yet another embodiment of this aspect, the decreased activity or expression is due to knock-out of a gene for the protein, introduction of a premature stop codon in the coding sequence of the gene for the protein, RNAi silencing, knock-out of a domain of the protein, introduction of a transcriptional repressor protein binding site, or knock-out of a binding site in the promoter region of the gene and/or the genetic alterations include knock-out of a gene for the protein, introduction of a premature stop codon in the coding sequence of the gene for the protein, RNAi silencing, knock-out of a domain of the protein, introduction of a transcriptional repressor protein binding site, or knock-out of a binding site in the promoter region of the gene and/or the genetic alterations include knock-out of a gene for the protein, introduction of a premature stop codon in the coding sequence of the gene for the protein, RNAi silencing, knock-out of a domain of the protein, introduction of a transcriptional repressor protein binding site, or knock-out of a binding site in the promoter region of the gene, preferably the binding site is a transcriptional activator protein binding site or a TATA-box. In a further embodiment of this aspect, the FUN protein includes a polypeptide with at least 70% identity, at least 75% identity, at least 80% identity, at least 85% identity, at least 90% identity, at least 95% identity, or at least 99% identity to a protein selected from the group of SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, or SEQ ID NO: 82, or a polypeptide with at least 70% identity, at least 75% identity, at least 80% identity, at least 85% identity, at least 90% identity, at least 95% identity, or at least 99% identity to domains thereof, for example, at least 70% identity, at least 75% identity, at least 80% identity, at least 85% identity, at least 90% identity, at least 95% identity, or at least 99% identity to the respective domains thereof recited in Table 1, for example, bZIP1, bZIP2, or DOG1 domains thereof, or combinations thereof. In another embodiment of this aspect, the FUN protein includes SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, or SEQ ID NO: 82, or respective domains thereof recited in Table 1, or combinations thereof. In still another embodiment of this aspect, the FUN protein includes SEQ ID NO: 1, SEQ ID NO: 8, or SEQ ID NO: 9, or respective domains thereof recited in Table 1, or combinations thereof. In yet another embodiment of this aspect, wherein the protein is the NRT3.1 protein, and wherein the NRT3.1 protein includes a polypeptide with at least 70% identity, at least 75% identity, at least 80% identity, at least 85% identity, at least 90% identity, at least 95% identity, or at least 99% identity to SEQ ID NO: 74; wherein the protein is the bZIP28 protein, and wherein the bZIP28 protein includes a polypeptide with at least 70% identity, at least 75% identity, at least 80% identity, at least 85% identity, at least 90% identity, at least 95% identity, or at least 99% identity to SEQ ID NO: 75; wherein the protein is the NAC-domain containing protein, and wherein the NAC-domain containing protein includes a polypeptide with at least 70% identity, at least 75% identity, at least 80% identity, at least 85% identity, at least 90% identity, at least 95% identity, or at least 99% identity to a protein selected from the group of SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, and SEQ ID NO: 73; wherein the protein is the HO1 protein, and wherein the HO1 protein includes a polypeptide with at least 70% identity, at least 75% identity, at least 80% identity, at least 85% identity, at least 90% identity, at least 95% identity, or at least 99% identity to SEQ ID NO: 77; wherein the protein is the NRT2.1 protein, and wherein the NRT2.1 protein includes a polypeptide with at least 70% identity, at least 75% identity, at least 80% identity, at least 85% identity, at least 90% identity, at least 95% identity, or at least 99% identity to SEQ ID NO: 78; or wherein the protein is the AS1 protein, and wherein the AS1 protein includes a polypeptide with at least 70% identity, at least 75% identity, at least 80% identity, at least 85% identity, at least 90% identity, at least 95% identity, or at least 99% identity to SEQ ID NO: 79, or a polypeptide with at least 70% identity, at least 75% identity, at least 80% identity, at least 85% identity, at least 90% identity, at least 95% identity, or at least 99% identity to domains thereof, for example, at least 70% identity, at least 75% identity, at least 80% identity, at least 85% identity, at least 90% identity, at least 95% identity, or at least 99% identity to respective domains thereof recited in Table 1, or combinations thereof. In a further embodiment of this aspect, wherein the NRT3.1 protein includes SEQ ID NO: 74; wherein the bZIP28 protein includes SEQ ID NO: 75; wherein the NAC-domain containing protein includes SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, or SEQ ID NO: 73; wherein the HO1 protein includes SEQ ID NO: 77; wherein the NRT2.1 protein includes SEQ ID NO: 78, or wherein the AS1 protein includes SEQ ID NO: 79, or respective domains thereof recited in Table 1, or combinations thereof. In still another embodiment of this aspect, the NAC-domain containing protein includes SEQ ID NO: 31, SEQ ID NO: 41, or SEQ ID NO: 42, or respective domains thereof recited in Table 1, or combinations thereof. Additional embodiments of this aspect, which may be combined with any of the preceding embodiments, include the nitrate level in step (c) being between about 10 mM and about 250 mM nitrate or at least about 10 mM nitrate, at least about 20 mM nitrate, at least about 30 mM nitrate, at least about 40 mM nitrate, at least about 50 mM nitrate, at least about 100 mM nitrate, at least about 150 mM nitrate, at least about 200 mM nitrate, or at least about 250 mM nitrate. Further embodiments of this aspect, which may be combined with any of the preceding embodiments, include the nitrogen fixation being increased at least 10%, at least 20%, at least 30%, at least 40%, at least 50%, at least 60%, at least 70%, at least 80%, at least 90%, at least 100%, at least 150%, at least 200%, at least 300%, at least 400%, or at least 500%. In a further embodiment of this aspect, the number of nodules is increased or the hemoglobin content is increased compared to the control plant when grown under the same growth conditions. In additional embodiments of this aspect, increased nitrogen fixation is measured using a method selected from the group of measuring the number of pink nodules per plant as compared to a control plant, measuring the amount of acetylene (C₂H₂) reduced to ethylene (C₂H₄) per hour (acetylene reduction assay (ARA)) as compared to a control plant, or measuring the micrograms of hemoglobin per plant as compared to a control plant (e.g., as described in Example 1).

A further aspect of the disclosure includes methods of cultivating a genetically altered plant able to fix nitrogen when grown in nitrogen-fertilized conditions, including: (a) providing the genetically altered plant, wherein the plant or a part thereof includes one or more genetic alterations that result in decreased activity or expression of a FUN protein, a NRT3.1 protein, a bZIP28 protein, a NAC-domain containing protein (also referred to as a FEZ protein), a HO1 protein, a NRT2.1 protein, or an AS1 protein, or any combination thereof as compared to an activity or expression of a FUN protein, a NRT3.1 protein, a bZIP28 protein, a NAC-domain containing protein, a HO1 protein, a NRT2.1 protein, or an AS1 protein in a control plant grown under the same conditions, and wherein the one or more genetic alterations reduce the nitrate level suppression of nitrogen fixation; (b) cultivating the plant under conditions including a standard nitrate level around the plant roots; and (c) applying nitrogen fertilizer, thereby generating conditions including a nitrate level around the plant roots that suppresses nitrogen fixation, wherein the genetically modified plant has increased nitrogen fixation as compared to the control plant grown under the same conditions. In yet another embodiment of this aspect, the decreased activity or expression is due to knock-out of a gene for the protein, introduction of a premature stop codon in the coding sequence of the gene for the protein, RNAi silencing, knock-out of a domain of the protein, introduction of a transcriptional repressor protein binding site, or knock-out of a binding site in the promoter region of the gene and/or the genetic alterations include knock-out of a gene for the protein, introduction of a premature stop codon in the coding sequence of the gene for the protein, RNAi silencing, knock-out of a domain of the protein, introduction of a transcriptional repressor protein binding site, or knock-out of a binding site in the promoter region of the gene and/or the genetic alterations include knock-out of a gene for the protein, introduction of a premature stop codon in the coding sequence of the gene for the protein, RNAi silencing, knock-out of a domain of the protein, introduction of a transcriptional repressor protein binding site, or knock-out of a binding site in the promoter region of the gene, preferably the binding site is a transcriptional activator protein binding site or a TATA-box. In a further embodiment of this aspect, the FUN protein includes a polypeptide with at least 70% identity, at least 75% identity, at least 80% identity, at least 85% identity, at least 90% identity, at least 95% identity, or at least 99% identity to a protein selected from the group of SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, or SEQ ID NO: 82, or a polypeptide with at least 70% identity, at least 75% identity, at least 80% identity, at least 85% identity, at least 90% identity, at least 95% identity, or at least 99% identity to domains thereof, for example, at least 70% identity, at least 75% identity, at least 80% identity, at least 85% identity, at least 90% identity, at least 95% identity, or at least 99% identity to respective domains thereof recited in TABLE X, or combinations thereof. In another embodiment of this aspect, the FUN protein includes SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, or SEQ ID NO: 82, or respective domains thereof recited in Table 1, or combinations thereof. In still another embodiment of this aspect, the FUN protein includes SEQ ID NO: 1, SEQ ID NO: 8, or SEQ ID NO: 9, or respective domains thereof recited in Table 1, or combinations thereof. In yet another embodiment of this aspect, wherein the protein is the NRT3.1 protein, and wherein the NRT3.1 protein includes a polypeptide with at least 70% identity, at least 75% identity, at least 80% identity, at least 85% identity, at least 90% identity, at least 95% identity, or at least 99% identity to SEQ ID NO: 74; wherein the protein is the bZIP28 protein, and wherein the bZIP28 protein includes a polypeptide with at least 70% identity, at least 75% identity, at least 80% identity, at least 85% identity, at least 90% identity, at least 95% identity, or at least 99% identity to SEQ ID NO: 75; wherein the protein is the NAC-domain containing protein, and wherein the NAC-domain containing protein includes a polypeptide with at least 70% identity, at least 75% identity, at least 80% identity, at least 85% identity, at least 90% identity, at least 95% identity, or at least 99% identity to a protein selected from the group of SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, and SEQ ID NO: 73; wherein the protein is the HO1 protein, and wherein the HO1 protein includes a polypeptide with at least 70% identity, at least 75% identity, at least 80% identity, at least 85% identity, at least 90% identity, at least 95% identity, or at least 99% identity to SEQ ID NO: 77; wherein the protein is the NRT2.1 protein, and wherein the NRT2.1 protein includes a polypeptide with at least 70% identity, at least 75% identity, at least 80% identity, at least 85% identity, at least 90% identity, at least 95% identity, or at least 99% identity to SEQ ID NO: 78; or wherein the protein is the AS1 protein, and wherein the AS1 protein includes a polypeptide with at least 70% identity, at least 75% identity, at least 80% identity, at least 85% identity, at least 90% identity, at least 95% identity, or at least 99% identity to SEQ ID NO: 79. In a further embodiment of this aspect, wherein the NRT3.1 protein includes SEQ ID NO: 74; wherein the bZIP28 protein includes SEQ ID NO: 75; wherein the NAC-domain containing protein includes SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, or SEQ ID NO: 73; wherein the HO1 protein includes SEQ ID NO: 77; wherein the NRT2.1 protein includes SEQ ID NO: 78, or wherein the AS1 protein includes SEQ ID NO: 79, or respective domains thereof recited in Table 1, or combinations thereof. In still another embodiment of this aspect, the NAC-domain containing protein includes SEQ ID NO: 31, SEQ ID NO: 41, or SEQ ID NO: 42, or respective domains thereof recited in Table 1, or combinations thereof. Additional embodiments of this aspect, which may be combined with any of the preceding embodiments, include the nitrate level in step (c) being between about 10 mM and about 250 mM nitrate or at least about 10 mM nitrate, at least about 20 mM nitrate, at least about 30 mM nitrate, at least about 40 mM nitrate, at least about 50 mM nitrate, at least about 100 mM nitrate, at least about 150 mM nitrate, at least about 200 mM nitrate, or at least about 250 mM nitrate. Further embodiments of this aspect, which may be combined with any of the preceding embodiments, include the nitrogen fixation being increased at least 10%, at least 20%, at least 30%, at least 40%, at least 50%, at least 60%, at least 70%, at least 80%, at least 90%, at least 100%, at least 150%, at least 200%, at least 300%, at least 400%, or at least 500%. In a further embodiment of this aspect, the number of nodules is increased or the hemoglobin content is increased compared to the control plant when grown under the same growth conditions. In additional embodiments of this aspect, increased nitrogen fixation is measured using a method selected from the group of measuring the number of pink nodules per plant as compared to a control plant, measuring the amount of acetylene (C₂H₂) reduced to ethylene (C₂H₄) per hour (acetylene reduction assay (ARA)) as compared to a control plant, or measuring the micrograms of hemoglobin per plant as compared to a control plant (e.g., as described in Example 1). In further embodiments of this aspect, which may be combined with any of the preceding embodiments, the genetically altered plant is grown in an intercropping system with a plant that does not fix nitrogen or in a sequential system after a plant that does not fix nitrogen.

An additional aspect of the disclosure includes methods of delaying nodule senescence, including: (a) providing a genetically altered plant, wherein the plant or a part thereof includes one or more genetic alterations that result in decreased activity or expression of a FUN protein, a NRT3.1 protein, a bZIP28 protein, a NAC-domain containing protein, a HO1 protein, a NRT2.1 protein, or an AS1 protein, or any combination thereof as compared to an activity or expression of a FUN protein, a NRT3.1 protein, a bZIP28 protein, a NAC-domain containing protein, a HO1 protein, a NRT2.1 protein, or an AS1 protein in a control plant grown under the same conditions, and wherein the one or more genetic alterations delay nodule senescence; and (b) cultivating the genetically altered plant under stress conditions, wherein the genetically altered plant has delayed nodule senescence as compared to the control plant grown under the same conditions. In yet another embodiment of this aspect, the decreased activity or expression is due to knock-out of a gene for the protein, introduction of a premature stop codon in the coding sequence of the gene for the protein, RNAi silencing, knock-out of a domain of the protein, introduction of a transcriptional repressor protein binding site, or knock-out of a binding site in the promoter region of the gene and/or the genetic alterations include knock-out of a gene for the protein, introduction of a premature stop codon in the coding sequence of the gene for the protein, RNAi silencing, knock-out of a domain of the protein, introduction of a transcriptional repressor protein binding site, or knock-out of a binding site in the promoter region of the gene and/or the genetic alterations include knock-out of a gene for the protein, introduction of a premature stop codon in the coding sequence of the gene for the protein, RNAi silencing, knock-out of a domain of the protein, introduction of a transcriptional repressor protein binding site, or knock-out of a binding site in the promoter region of the gene, preferably the binding site is a transcriptional activator protein binding site or a TATA-box. A further embodiment of this aspect includes the stress conditions being selected from the group of a moderate nitrate level, a high nitrate level, a nitrate level around the plant that promotes nodule senescence, a moderate heat level, a high heat level, a heat level around the plant that promotes nodule senescence, a moderate water deficit (i.e., drought) level, a high water deficit level, a water deficit level around the plant that promotes nodule senescence, a moderate waterlogging level, a high waterlogging level, or a waterlogging level around the plant that promotes nodule senescence. For each of these conditions, a stress level is considered to be a level sufficient to inhibit nitrogen fixation in the particular plant species. In a further embodiment of this aspect, which may be combined with any of the preceding embodiments. the FUN protein includes a polypeptide with at least 70% identity, at least 75% identity, at least 80% identity, at least 85% identity, at least 90% identity, at least 95% identity, or at least 99% identity to a protein selected from the group of SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, or SEQ ID NO: 82, or a polypeptide with at least 70% identity, at least 75% identity, at least 80% identity, at least 85% identity, at least 90% identity, at least 95% identity, or at least 99% identity to domains thereof, for example, at least 70% identity, at least 75% identity, at least 80% identity, at least 85% identity, at least 90% identity, at least 95% identity, or at least 99% identity to respective domains thereof recited in Table 1, or combinations thereof. In another embodiment of this aspect, the FUN protein includes SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, or SEQ ID NO: 82, or respective domains thereof recited in Table 1, or combinations thereof. In still another embodiment of this aspect, the FUN protein includes SEQ ID NO: 1, SEQ ID NO: 8, or SEQ ID NO: 9, or respective domains thereof recited in Table 1, or combinations thereof. In yet another embodiment of this aspect, which may be combined with any of the preceding embodiments that have a NAC-domain containing protein, wherein the protein is the NRT3.1 protein, and wherein the NRT3.1 protein includes a polypeptide with at least 70% identity, at least 75% identity, at least 80% identity, at least 85% identity, at least 90% identity, at least 95% identity, or at least 99% identity to SEQ ID NO: 74; wherein the protein is the bZIP28 protein, and wherein the bZIP28 protein includes a polypeptide with at least 70% identity, at least 75% identity, at least 80% identity, at least 85% identity, at least 90% identity, at least 95% identity, or at least 99% identity to SEQ ID NO: 75; wherein the protein is the NAC-domain containing protein, and wherein the NAC-domain containing protein includes a polypeptide with at least 70% identity, at least 75% identity, at least 80% identity, at least 85% identity, at least 90% identity, at least 95% identity, or at least 99% identity to a protein selected from the group of SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, and SEQ ID NO: 73; wherein the protein is the HO1 protein, and wherein the HO1 protein includes a polypeptide with at least 70% identity, at least 75% identity, at least 80% identity, at least 85% identity, at least 90% identity, at least 95% identity, or at least 99% identity to SEQ ID NO: 77; wherein the protein is the NRT2.1 protein, and wherein the NRT2.1 protein includes a polypeptide with at least 70% identity, at least 75% identity, at least 80% identity, at least 85% identity, at least 90% identity, at least 95% identity, or at least 99% identity to SEQ ID NO: 78; or wherein the protein is the AS1 protein, and wherein the AS1 protein includes a polypeptide with at least 70% identity, at least 75% identity, at least 80% identity, at least 85% identity, at least 90% identity, at least 95% identity, or at least 99% identity to SEQ ID NO: 79, or a polypeptide with at least 70% identity, at least 75% identity, at least 80% identity, at least 85% identity, at least 90% identity, at least 95% identity, or at least 99% identity to domains thereof, for example, at least 70% identity, at least 75% identity, at least 80% identity, at least 85% identity, at least 90% identity, at least 95% identity, or at least 99% identity to respective domains thereof recited in Table 1, or combinations thereof. In a further embodiment of this aspect, wherein the NRT3.1 protein includes SEQ ID NO: 74; wherein the bZIP28 protein includes SEQ ID NO: 75; wherein the NAC-domain containing protein includes SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, or SEQ ID NO: 73; wherein the HO1 protein includes SEQ ID NO: 77; wherein the NRT2.1 protein includes SEQ ID NO: 78, or wherein the AS1 protein includes SEQ ID NO: 79, or respective domains thereof recited in Table 1, or combinations thereof. In still another embodiment of this aspect, the NAC-domain containing protein includes SEQ ID NO: 31, SEQ ID NO: 41, or SEQ ID NO: 42, or respective domains thereof recited in Table 1, or combinations thereof. In a further embodiment of this aspect, the nodule senescence is delayed at least 10%, at least 20%, at least 30%, at least 40%, at least 50%, at least 60%, at least 70%, at least 80%, at least 90%, at least 100%, at least 150%, at least 200%, at least 300%, at least 400%, or at least 500%.

Yet further aspects of the disclosure include methods of inducing filamentation of a FUN protein, including: (a) providing the FUN protein; and (b) increasing an amount of zinc or manganese in an environment of the FUN protein, wherein the increased amount of zinc or manganese induces filamentation as compared to the control FUN protein in an environment without the increased amount of zinc or manganese. In an additional embodiment of this aspect, the filamentation is induced under high nitrate conditions. In yet another embodiment of this aspect, which may be combined with any of the preceding embodiments, the method is performed in vitro.

Still further aspects of the disclosure include methods of inducing filamentation, including: (a) providing a plant including a FUN protein; and (b) cultivating the plant under increased zinc or manganese conditions, wherein filamentation of the FUN protein in the plant is induced as compared a FUN protein in a control plant grown under conditions without increased zinc or manganese. In another embodiment of this aspect, the plant comprises genetic alteration. In an additional embodiment of this aspect, the filamentation is induced under high nitrate conditions. In yet another embodiment of this aspect, which may be combined with any of the preceding embodiments where the plant comprises a genetic alteration, the genetic alteration reduces the activity of the FUN protein without eliminating the activity of the FUN protein. In a further embodiment of this aspect, the induction of filamentation results in increased nitrogen fixation in the genetically altered plant as compared to the control plant grown under the same conditions or reduces the activity or inactivates the FUN protein. In still another embodiment of this aspect, which may be combined with any of the preceding embodiments or aspects that have methods of inducing filamentation, the number of nodules is increased, hemoglobin content is increased, or the acetylene reduction assay (ARA) activity is increased compared to the control plant when grown under the same conditions.

Yet further aspects of the disclosure include methods of tuning nodule function to the amount of available nitrogen in the soil, including: a) providing a genetically altered plant comprising a FUN protein with altered activation by nitrate; and b) cultivating the genetically altered plant under nitrate concentration conditions, wherein the genetically altered plant has reduced activity or expression of FUN and/or reduced active form of FUN as compared to a WT plant grown under the same nitrate conditions. In an additional embodiment of this aspect, altering FUN protein activation by nitrate includes downregulating FUN, reducing FUN activity, knocking out FUN by mutation, knocking down FUN expression, knocking out promoter elements of FUN, or a combination thereof. In a further embodiment of this aspect, altering FUN protein activation by nitrate includes manipulating a level of environmental or cellular zinc, wherein this manipulation results in the FUN protein being maintained in inactive filament form. In still another embodiment of this aspect, altering FUN protein activation by nitrate comprises genetically modifying the FUN protein sequence to alter sensitivity to zinc.

In a further embodiment of this aspect, which may be combined with any of the preceding embodiments and aspects, the FUN protein includes a polypeptide with at least 70% identity, at least 75% identity, at least 80% identity, at least 85% identity, at least 90% identity, at least 95% identity, or at least 99% identity to a protein selected from the group of SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, or SEQ ID NO: 82, or a polypeptide with at least 70% identity, at least 75% identity, at least 80% identity, at least 85% identity, at least 90% identity, at least 95% identity, or at least 99% identity to domains thereof, for example, at least 70% identity, at least 75% identity, at least 80% identity, at least 85% identity, at least 90% identity, at least 95% identity, or at least 99% identity to respective domains thereof recited in Table 1, or combinations thereof. In another embodiment of this aspect, the FUN protein includes SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, or SEQ ID NO: 82, or respective domains thereof recited in Table 1, or combinations thereof. In still another embodiment of this aspect, the FUN protein includes SEQ ID NO: 1, SEQ ID NO: 8, or SEQ ID NO: 9, or respective domains thereof recited in Table 1, or combinations thereof.

A further aspect of the disclosure includes a method of making a genetically altered plant with increased nitrogen fixation under conditions including a nitrate level around the plant roots that suppresses nitrogen fixation, including introducing into the plant or a part thereof one or more genetic alterations that decrease activity or expression of a FUN protein as compared to the activity or expression of a FUN protein in a control plant grown under the same conditions. In yet another embodiment of this aspect, the decreased activity or expression is due to knock-out of a gene for the protein, introduction of a premature stop codon in the coding sequence of the gene for the protein, RNAi silencing, knock-out of a domain of the protein, introduction of a transcriptional repressor protein binding site, or knock-out of a binding site in the promoter region of the gene and/or the genetic alterations include knock-out of a gene for the protein, introduction of a premature stop codon in the coding sequence of the gene for the protein, RNAi silencing, knock-out of a domain of the protein, introduction of a transcriptional repressor protein binding site, or knock-out of a binding site in the promoter region of the gene and/or the genetic alterations include knock-out of a gene for the protein, introduction of a premature stop codon in the coding sequence of the gene for the protein, RNAi silencing, knock-out of a domain of the protein, introduction of a transcriptional repressor protein binding site, or knock-out of a binding site in the promoter region of the gene, preferably the binding site is a transcriptional activator protein binding site or a TATA-box. In an additional embodiment of this aspect, the FUN protein includes a polypeptide with at least 70% identity, at least 75% identity, at least 80% identity, at least 85% identity, at least 90% identity, at least 95% identity, or at least 99% identity to a protein selected from the group of SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, or SEQ ID NO: 82; wherein the FUN protein includes SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, or SEQ ID NO: 82; or wherein the FUN protein includes SEQ ID NO: 1, SEQ ID NO: 8, or SEQ ID NO: 9, or a polypeptide with at least 70% identity, at least 75% identity, at least 80% identity, at least 85% identity, at least 90% identity, at least 95% identity, or at least 99% identity to domains thereof, for example, at least 70% identity, at least 75% identity, at least 80% identity, at least 85% identity, at least 90% identity, at least 95% identity, or at least 99% identity to respective domains thereof recited in Table 1, or combinations thereof.

An additional aspect of the disclosure includes methods of making a genetically altered plant with increased nitrogen fixation under conditions including a nitrate level around the plant roots that suppresses nitrogen fixation, including introducing into the plant or a part thereof one or more genetic alterations that decrease activity or expression of one or more of a NRT3.1 protein, a bZIP28 protein, a NAC-domain containing protein (also referred to as a FEZ protein), a HO1 protein, a NRT2.1 protein, or an AS1 protein as compared to the activity or expression of a NRT3.1 protein, a bZIP28 protein, a NAC-domain containing protein, a HO1 protein, a NRT2.1 protein, or an AS1 protein in a control plant grown under the same conditions. In yet another embodiment of this aspect, the decreased activity or expression is due to knock-out of a gene for the protein, introduction of a premature stop codon in the coding sequence of the gene for the protein, RNAi silencing, knock-out of a domain of the protein, introduction of a transcriptional repressor protein binding site, or knock-out of a binding site in the promoter region of the gene and/or the genetic alterations include knock-out of a gene for the protein, introduction of a premature stop codon in the coding sequence of the gene for the protein, RNAi silencing, knock-out of a domain of the protein, introduction of a transcriptional repressor protein binding site, or knock-out of a binding site in the promoter region of the gene and/or the genetic alterations include knock-out of a gene for the protein, introduction of a premature stop codon in the coding sequence of the gene for the protein, RNAi silencing, knock-out of a domain of the protein, introduction of a transcriptional repressor protein binding site, or knock-out of a binding site in the promoter region of the gene, preferably the binding site is a transcriptional activator protein binding site or a TATA-box. In yet another embodiment of this aspect, wherein the protein is the NRT3.1 protein, and wherein the NRT3.1 protein includes a polypeptide with at least 70% identity, at least 75% identity, at least 80% identity, at least 85% identity, at least 90% identity, at least 95% identity, or at least 99% identity to SEQ ID NO: 74; wherein the protein is the bZIP28 protein, and wherein the bZIP28 protein includes a polypeptide with at least 70% identity, at least 75% identity, at least 80% identity, at least 85% identity, at least 90% identity, at least 95% identity, or at least 99% identity to SEQ ID NO: 75; wherein the protein is the NAC-domain containing protein, and wherein the NAC-domain containing protein includes a polypeptide with at least 70% identity, at least 75% identity, at least 80% identity, at least 85% identity, at least 90% identity, at least 95% identity, or at least 99% identity to a protein selected from the group of SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, and SEQ ID NO: 73; wherein the protein is the HO1 protein, and wherein the HO1 protein includes a polypeptide with at least 70% identity, at least 75% identity, at least 80% identity, at least 85% identity, at least 90% identity, at least 95% identity, or at least 99% identity to SEQ ID NO: 77; wherein the protein is the NRT2.1 protein, and wherein the NRT2.1 protein includes a polypeptide with at least 70% identity, at least 75% identity, at least 80% identity, at least 85% identity, at least 90% identity, at least 95% identity, or at least 99% identity to SEQ ID NO: 78; or wherein the protein is the AS1 protein, and wherein the AS1 protein includes a polypeptide with at least 70% identity, at least 75% identity, at least 80% identity, at least 85% identity, at least 90% identity, at least 95% identity, or at least 99% identity to SEQ ID NO: 79; wherein the NRT3.1 protein includes SEQ ID NO: 74; wherein the bZIP28 protein includes SEQ ID NO: 75; wherein the NAC-domain containing protein includes SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, or SEQ ID NO: 73; wherein the HO1 protein includes SEQ ID NO: 77; wherein the NRT2.1 protein includes SEQ ID NO: 78, or wherein the AS1 protein includes SEQ ID NO: 79; or wherein the NAC-domain containing protein includes SEQ ID NO: 31, SEQ ID NO: 41, or SEQ ID NO: 42, or a polypeptide with at least 70% identity, at least 75% identity, at least 80% identity, at least 85% identity, at least 90% identity, at least 95% identity, or at least 99% identity to domains thereof, for example, at least 70% identity, at least 75% identity, at least 80% identity, at least 85% identity, at least 90% identity, at least 95% identity, at least 99% or 100% identity to respective domains thereof recited in Table 1, or combinations thereof.

Yet another aspect of the disclosure includes methods of making a genetically altered plant with increased nitrogen fixation under conditions including a nitrate level around the plant roots that suppresses nitrogen fixation, including introducing into the plant or a part thereof one or more genetic alterations that decrease activity or expression of one or more of a FUN protein, a FUN-like protein, a NRT3.1 protein, a bZIP28 protein, a NAC-domain containing protein (also referred to as a FEZ protein), a HO1 protein, a NRT2.1 protein, or an AS1 protein as compared to the activity or expression of a FUN protein, a FUN-like protein, a NRT3.1 protein, a bZIP28 protein, a NAC-domain containing protein, a HO1 protein, a NRT2.1 protein, or an AS1 protein in a control plant grown under the same conditions. In yet another embodiment of this aspect, the decreased activity or expression is due to knock-out of a gene for the protein, introduction of a premature stop codon in the coding sequence of the gene for the protein, RNAi silencing, knock-out of a domain of the protein, introduction of a transcriptional repressor protein binding site, or knock-out of a binding site in the promoter region of the gene and/or the genetic alterations include knock-out of a gene for the protein, introduction of a premature stop codon in the coding sequence of the gene for the protein, RNAi silencing, knock-out of a domain of the protein, introduction of a transcriptional repressor protein binding site, or knock-out of a binding site in the promoter region of the gene and/or the genetic alterations include knock-out of a gene for the protein, introduction of a premature stop codon in the coding sequence of the gene for the protein, RNAi silencing, knock-out of a domain of the protein, introduction of a transcriptional repressor protein binding site, or knock-out of a binding site in the promoter region of the gene, preferably the binding site is a transcriptional activator protein binding site or a TATA-box. In some embodiments of this aspect, the FUN protein, the FUN-like protein, the NRT3.1 protein, the bZIP28 protein, the NAC-domain containing protein, the HO1 protein, the NRT2.1 protein, or the AS1 protein is selected from the group of a polypeptide with at least 70% identity, at least 75% identity, at least 80% identity, at least 85% identity, at least 90% identity, at least 95% identity, or at least 99% identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, or SEQ ID NO: 84, or respective domains thereof recited in Table 1, or combinations thereof, and wherein the FUN protein, the FUN-like protein, the NRT3.1 protein, the bZIP28 protein, the NAC-domain containing protein, the HO1 protein, the NRT2.1 protein, or the AS1 protein has enhanced expression in a root nodule absent the one or more genetic alterations.

An additional aspect of the disclosure includes methods of making the genetically modified plant or part thereof of any of the above embodiments, including: introducing a genetic alteration to the plant cell that reduces or knocks out activity or expression of a FUN protein, a FUN-like protein, a NAC-domain containing protein (also referred to as a FEZ protein), a NRT3.1 protein, a bZIP28 protein, a NAC-domain containing protein, a HO1 protein, a NRT2.1 protein, or an AS1 protein. In a further embodiment of this aspect, the genetic alteration includes a first nucleic acid sequence able to reduce or knock out a second nucleic acid sequence encoding a FUN protein, a FUN-like protein, a NAC-domain containing protein, a NRT3.1 protein, a bZIP28 protein, a NAC-domain containing protein, a HO1 protein, a NRT2.1 protein, or an AS1 protein operably linked to a promoter. In an additional embodiment of this aspect, which may be combined with any of the preceding embodiments, the genetically altered plant is selected from one or more of the group consisting of alfalfa, Bambara groundnut, bean (e.g., kidney beans, black beans, etc.), black currant, chickpea, clover, cowpea, forage legumes, legume trees, lentil, lotus, lupin, Medicago spp., pea, peanut, pigeon pea, soybean, Parasponia, alder trees, and elm trees. In another embodiment of this aspect, the nucleic acid includes a RNA silencing associated short RNA, an antisense RNA, a siRNA, a miRNA, a dsRNA, a tasiRNA, or a secondary siRNA. In yet another embodiment of this aspect, the promoter is a nodule specific promoter, a root specific promoter, an inducible promoter, a constitutive promoter, or a combination thereof. In an additional embodiment of this aspect, the promoter is a root specific promoter, and wherein the promoter is selected from the group consisting of a NFR1 promoter, a NFR5 promoter, a LYK3 promoter, a CERK6 promoter, a NFP promoter, a Lotus japonicus NFR5 promoter (SEQ ID NO: 85), a Lotus japonicus NFR1 promoter (SEQ ID NO: 89), a Lotus japonicus CERK6 promoter (SEQ ID NO: 87), a Medicago truncatula NFP promoter (SEQ ID NO: 86), a Medicago truncatula LYK3 promoter (SEQ ID NO: 88), a maize metallothionein promoter, a chitinase promoter, a maize ZRP2 promoter, a tomato LeExt1 promoter, a glutamine synthetase soybean root promoter, a RCC3 promoter, a rice antiquitin promoter, a LRR receptor kinase promoter, and an Arabidopsis pCO2 promoter. In a further embodiment of this aspect, the promoter is a constitutive promoter, and wherein the promoter is selected from the group including of a CaMV35S promoter, a derivative of the CaMV35S promoter, a maize ubiquitin promoter, a polyubiquitin promoter, a vein mosaic cassava virus promoter, or an Arabidopsis UBQ10 promoter. In still another embodiment of this aspect, the nucleic acid sequence is inserted into the genome of the plant so that the nucleic acid sequence is operably linked to an endogenous promoter. In a further embodiment of this aspect, the endogenous promoter is a nodule specific promoter or a root specific promoter.

A further aspect of the disclosure includes methods of making the genetically modified plant or part thereof of any of the preceding embodiments, including genetically modifying the plant cell by transforming the plant cell with one or more gene editing components that target an endogenous nuclear genome sequence encoding a FUN protein, a FUN-like protein, a NAC-domain containing protein (also referred to as a FEZ protein), a NRT3.1 protein, a bZIP28 protein, a NAC-domain containing protein, a HO1 protein, a NRT2.1 protein, or an AS1 protein, wherein the endogenous nuclear genome sequence or a part thereof is knocked out. In another embodiment of this aspect, the one or more gene editing components include a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence.

A control as described herein can be a control sample or a reference sample from a wild-type, an azygous, or a null-segregant plant, species, or sample or from populations thereof. A control plant as described herein can also be a plant that is the same as a genetically altered or genetically modified plant, without the alteration or modification, or a wild type plant under the same growing conditions, soil, and/or growth medium. A reference value can be used in place of a control or reference sample, which was previously obtained from a wild-type, azygous, or null-segregant plant, species, or sample or from populations thereof or a group of a wild-type, azygous, or null-segregant plant, species, or sample. A control sample or a reference sample can also be a sample with a known amount of a detectable composition or a spiked sample.

Recitation of each discrete value described herein is understood to include ranges between each value. Recitation of ranges of values as described herein is understood to include discrete values within the range.

### Expression Vectors or Isolated DNA Molecules, Cells or Kits Including the Same, and Related Methods

Yet another aspect of the disclosure includes an expression vector or isolated DNA molecule including (i) one or more nucleotide sequences encoding a FUN protein, a FUN-like protein, a HO1 protein, a NAC-domain containing protein (also referred to as a FEZ protein), a bZIP28 protein, a NRT2.1 protein, a NRT3.1 protein, an AS1 protein, or a combination thereof, wherein the one or more nucleotide sequences are operably linked to at least one expression control sequence, (ii) one or more nucleotide sequences able to reduce or knock out a nucleic acid sequence encoding a FUN protein, a FUN-like protein, a HO1 protein, a NAC-domain containing protein, a bZIP28 protein, a NRT2.1 protein, a NRT3.1 protein, an AS1 protein, or a combination thereof, wherein the one or more nucleotide sequences are operably linked to at least one expression control sequence, or (iii) one or more nucleotide sequences including a mutation in a gene for a FUN protein, a FUN-like protein, a HO1 protein, a NAC-domain containing protein, a bZIP28 protein, a NRT2.1 protein, a NRT3.1 protein, an AS1 protein, or a combination thereof, wherein the mutation reduces or knocks out the activity or expression of the protein and the one or more nucleotide sequences are operably linked to at least one homologous nucleic acid sequence that hybridizes adjacent to the mutation site in the gene. In another embodiment of this aspect, the expression control sequence includes a nodule specific promoter, a root specific promoter, an inducible promoter, a constitutive promoter, or a combination thereof. In still another embodiment of this aspect, wherein the promoter is a root specific promoter, and wherein the promoter is selected from the group consisting of a NFR1 promoter, a NFR5 promoter, a LYK3 promoter, a CERK6 promoter, a NFP promoter, a Lotus japonicus NFR5 promoter (SEQ ID NO: 85), a Lotus japonicus NFR1 promoter (SEQ ID NO: 89), a Lotus japonicus CERK6 promoter (SEQ ID NO: 87), a Medicago truncatula NFP promoter (SEQ ID NO: 86), a Medicago truncatula LYK3 promoter (SEQ ID NO: 88), a maize metallothionein promoter, a chitinase promoter, a maize ZRP2 promoter, a tomato LeExt1 promoter, a glutamine synthetase soybean root promoter, a RCC3 promoter, a rice antiquitin promoter, a LRR receptor kinase promoter, and an Arabidopsis pCO2 promoter; or wherein the promoter is a constitutive promoter, and wherein the promoter is selected from the group consisting of a CaMV35S promoter, a derivative of the CaMV35S promoter, a maize ubiquitin promoter, a polyubiquitin promoter, a vein mosaic cassava virus promoter, and an Arabidopsis UBQ10 promoter. In a further embodiment of this aspect, the protein is a FUN protein, and wherein the FUN protein includes a polypeptide with at least 70% identity, at least 75% identity, at least 80% identity, at least 85% identity, at least 90% identity, at least 95% identity, or at least 99% identity to a protein selected from the group of SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, and SEQ ID NO: 82; wherein the FUN protein includes SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, and SEQ ID NO: 82; or wherein the FUN protein includes SEQ ID NO: 1, SEQ ID NO: 8, or SEQ ID NO: 9; wherein the protein is a FUN-like protein, and wherein the FUN-like protein includes a polypeptide with at least 70% identity, at least 75% identity, at least 80% identity, at least 85% identity, at least 90% identity, at least 95% identity, or at least 99% identity to a protein selected from the group of SEQ ID NO: 4, SEQ ID NO: 6, SEQ ID NO: 7, SEQ ID NO: 83, and SEQ ID NO: 84; wherein the FUN-like protein includes SEQ ID NO: 4, SEQ ID NO: 6, SEQ ID NO: 7, SEQ ID NO: 83, or SEQ ID NO: 84; and/or wherein the protein is the NRT3.1 protein, and wherein the NRT3.1 protein includes a polypeptide with at least 70% identity, at least 75% identity, at least 80% identity, at least 85% identity, at least 90% identity, at least 95% identity, or at least 99% identity to SEQ ID NO: 74; wherein the protein is the bZIP28 protein, and wherein the bZIP28 protein includes a polypeptide with at least 70% identity, at least 75% identity, at least 80% identity, at least 85% identity, at least 90% identity, at least 95% identity, or at least 99% identity to SEQ ID NO: 75; wherein the protein is the NAC-domain containing protein, and wherein the NAC-domain containing protein includes a polypeptide with at least 70% identity, at least 75% identity, at least 80% identity, at least 85% identity, at least 90% identity, at least 95% identity, or at least 99% identity to a protein selected from the group of SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, and SEQ ID NO: 73; wherein the protein is the HO1 protein, and wherein the HO1 protein includes a polypeptide with at least 70% identity, at least 75% identity, at least 80% identity, at least 85% identity, at least 90% identity, at least 95% identity, or at least 99% identity to SEQ ID NO: 77; wherein the protein is the NRT2.1 protein, and wherein the NRT2.1 protein includes a polypeptide with at least 70% identity, at least 75% identity, at least 80% identity, at least 85% identity, at least 90% identity, at least 95% identity, or at least 99% identity to SEQ ID NO: 78; or wherein the protein is the AS1 protein, and wherein the AS1 protein includes a polypeptide with at least 70% identity, at least 80% identity, at least 90% identity, at least 95% identity, or at least 99% identity to SEQ ID NO: 79; wherein the NRT3.1 protein includes SEQ ID NO: 74; wherein the bZIP28 protein includes SEQ ID NO: 75; wherein the NAC-domain containing protein includes SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, or SEQ ID NO: 73; wherein the HO1 protein includes SEQ ID NO: 77; wherein the NRT2.1 protein includes SEQ ID NO: 78, or wherein the AS1 protein includes SEQ ID NO: 79; or wherein the NAC-domain containing protein includes SEQ ID NO: 31, SEQ ID NO: 41, or SEQ ID NO: 42, or a polypeptide with at least 70% identity, at least 75% identity, at least 80% identity, at least 85% identity, at least 90% identity, at least 95% identity, or at least 99% identity to domains thereof, for example, at least 70% identity, at least 75% identity, at least 80% identity, at least 85% identity, at least 90% identity, at least 95% identity, or at least 99% identity to respective domains thereof recited in Table 1, or combinations thereof.

Some aspects of the present disclosure relate to a bacterial cell or an Agrobacterium cell including the expression vector or isolated DNA molecule of any of the preceding embodiments.

Additional aspects of the present disclosure relate to genetically modified plant, plant part, plant cell, or seed including the expression vector or isolated DNA molecule of any of the preceding embodiments.

Further aspects of the present disclosure relate to a kit including the expression vector or isolated DNA molecule of any of the preceding embodiments or the bacterial cell or the Agrobacterium cell of the preceding embodiments.

Still further aspects of the present disclosure relate to methods of increasing nitrogen fixation, delaying nodule senescence, or inducing FUN filamentation in a plant, including: (a) introducing a genetic alteration via an expression vector or isolated DNA molecule of any of the preceding embodiments; and optionally (b) treating the plant with zinc or manganese or growing the plant under high zinc, high manganese, or high nitrate conditions. High manganese, high zinc, or high nitrate conditions can be a level that is higher than the ambient conditions of the soil in which the plant is growing, higher than optimal for plant growth, in any amount that is not natural or not naturally present, or in an amount to induce or sustain filamentation or aggregation of a FUN protein. In some embodiments, the high level can be 10%, 20%, 30%, 40%, 50%, 75%, 100%, 150%, 250%, or 500% higher than such ambient conditions, such optimal conditions, or such natural amount present.

### Plant Breeding Methods

Plant breeding begins with the analysis of the current germplasm, the definition of problems and weaknesses of the current germplasm, the establishment of program goals, and the definition of specific breeding objectives. The next step is the selection of germplasm that possess the traits to meet the program goals. The selected germplasm is crossed in order to recombine the desired traits and through selection, varieties or parent lines are developed. The goal is to combine in a single variety or hybrid an improved combination of desirable traits from the parental germplasm. These important traits may include higher yield, field performance, improved fruit and agronomic quality, resistance to biological stresses, such as diseases and pests, and tolerance to environmental stresses, such as drought and heat.

Each breeding program should include a periodic, objective evaluation of the efficiency of the breeding procedure. Evaluation criteria vary depending on the goal and objectives, but should include gain from selection per year based on comparisons to an appropriate standard, overall value of the advanced breeding lines, and number of successful cultivars produced per unit of input (e.g., per year, per dollar expended, etc.). Promising advanced breeding lines are thoroughly tested and compared to appropriate standards in environments representative of the commercial target area(s) for three years at least. The best lines are candidates for new commercial cultivars; those still deficient in a few traits are used as parents to produce new populations for further selection. These processes, which lead to the final step of marketing and distribution, usually take five to ten years from the time the first cross or selection is made.

The choice of breeding or selection methods depends on the mode of plant reproduction, the heritability of the trait(s) being improved, and the type of cultivar used commercially (e.g., F₁ hybrid cultivar, inbred cultivar, etc.). For highly heritable traits, a choice of superior individual plants evaluated at a single location will be effective, whereas for traits with low heritability, selection should be based on mean values obtained from replicated evaluations of families of related plants. The complexity of inheritance also influences the choice of the breeding method. Backcross breeding is used to transfer one or a few genes for a highly heritable trait into a desirable cultivar (e.g., for breeding disease-resistant cultivars), while recurrent selection techniques are used for quantitatively inherited traits controlled by numerous genes, various recurrent selection techniques are used. Commonly used selection methods include pedigree selection, modified pedigree selection, mass selection, and recurrent selection.

Pedigree selection is generally used for the improvement of self-pollinating crops or inbred lines of cross-pollinating crops. Two parents which possess favorable, complementary traits are crossed to produce an F₁. An F₂ population is produced by selfing one or several F₁s or by intercrossing two F₁s (sib mating). Selection of the best individuals is usually begun in the F₂ population; then, beginning in the F₃, the best individuals in the best families are selected. Replicated testing of families, or hybrid combinations involving individuals of these families, often follows in the F₄ generation to improve the effectiveness of selection for traits with low heritability. At an advanced stage of inbreeding (i.e., F₆ and F₇), the best lines or mixtures of phenotypically similar lines are tested for potential release as new cultivars.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding (i.e., recurrent selection) may be used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In addition to phenotypic observations, the genotype of a plant can also be examined. There are many laboratory-based techniques available for the analysis, comparison and characterization of plant genotype; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs, which are also referred to as Microsatellites), Fluorescently Tagged Inter-simple Sequence Repeats (ISSRs), Single Nucleotide Polymorphisms (SNPs), Genotyping by Sequencing (GbS), and Next-generation Sequencing (NGS).

Molecular markers, or “markers”, can also be used during the breeding process for the selection of qualitative traits. For example, markers closely linked to alleles or markers containing sequences within the actual alleles of interest can be used to select plants that contain the alleles of interest. The use of markers in the selection process is often called genetic marker enhanced selection or marker-assisted selection. Methods of performing marker analysis are generally known to those of skill in the art.

Mutation breeding may also be used to introduce new traits into plant varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development: Theory and Technique, Walter Fehr (1991), Agronomy Books, 1 (https://lib.dr.iastate.edu/agron_books/1).

The production of double haploids can also be used for the development of homozygous lines in a breeding program. Double haploids are produced by the doubling of a set of chromosomes from a heterozygous plant to produce a completely homozygous individual. For example, see Wan, et al., Theor. Appl. Genet., 77:889-892, 1989.

Additional non-limiting examples of breeding methods that may be used include, without limitation, those found in Principles of Plant Breeding, John Wiley and Son, pp. 115-161 (1960); Principles of Cultivar Development: Theory and Technique, Walter Fehr (1991), Agronomy Books, 1 (https://lib.dr.iastate.edu/agron_books/1), which are herewith incorporated by reference.

### Molecular Biological Methods to Produce Genetically Modified Plant Cells, Plant Parts, and Plants

One aspect of the present disclosure provides genetically altered or modified plants or parts thereof including one or more genetic alterations that result in decreased activity or expression of a FUN protein. Another aspect of the disclosure includes genetically modified plants or parts thereof including one or more genetic alterations that result in decreased activity or expression of one or more of a NRT3.1 protein, a bZIP28 protein, a NAC-domain containing protein (also referred to as a FEZ protein), a HO1 protein, a NRT2.1 protein, or an AS1 protein.

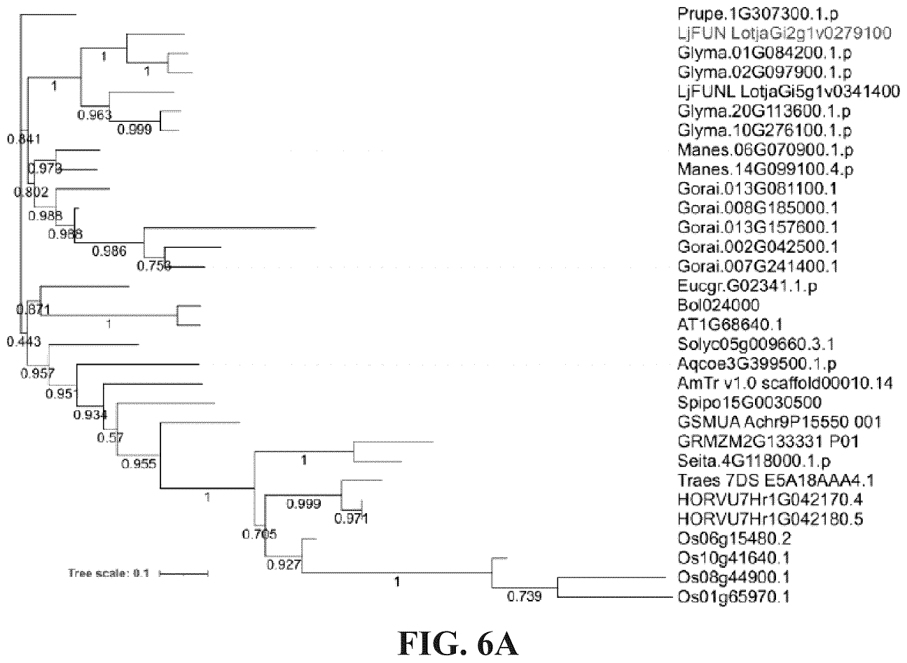

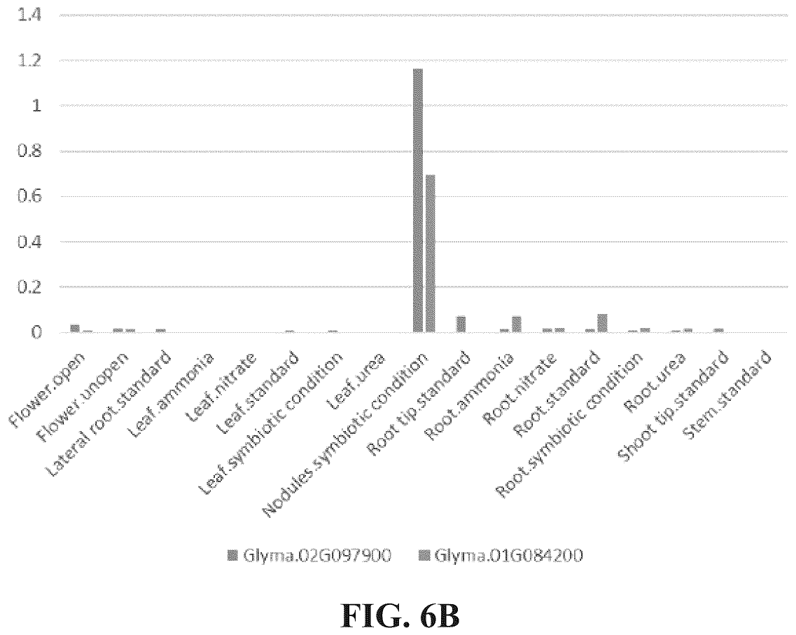

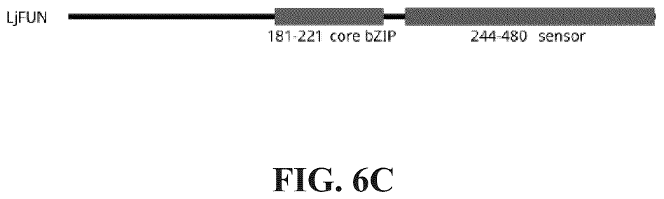

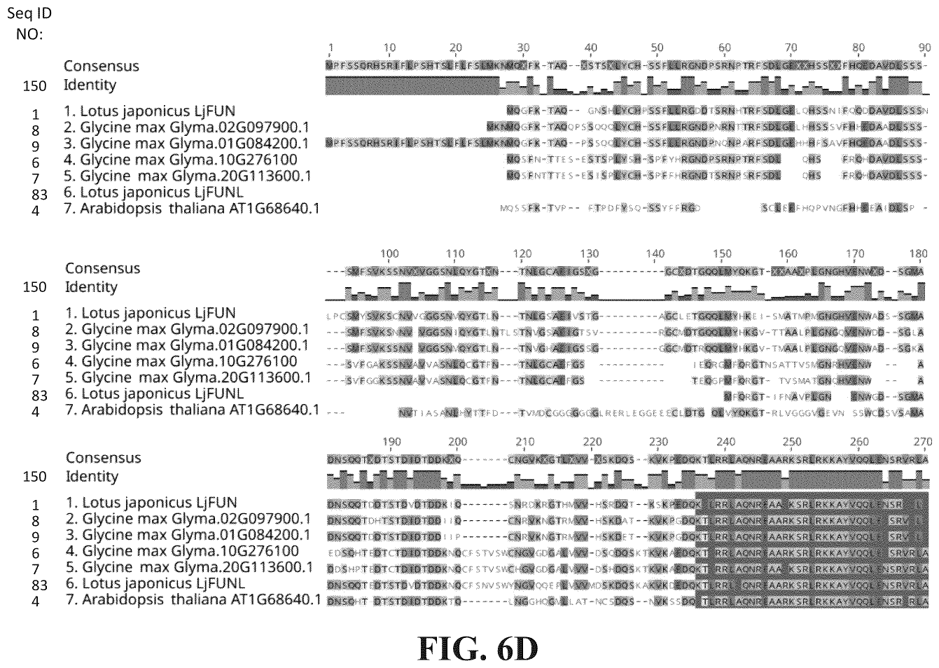

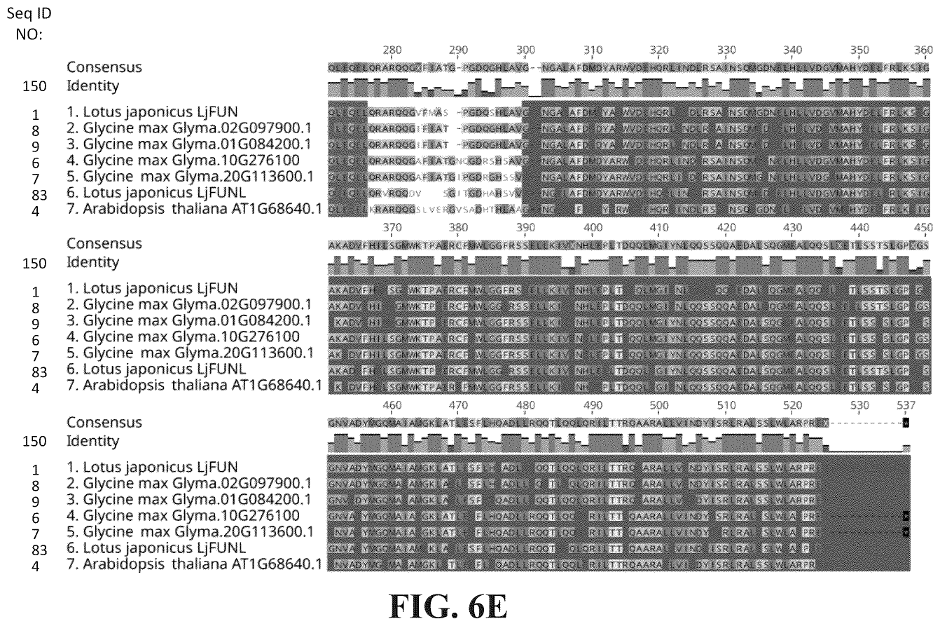

FUN is a member of the TGA transcription factor family. TGA transcription factors can be characterized by a DNA-binding bZIP domain at the N-terminus, and a DOG1 domain at the C-terminus (Tomaz̆, S̆., Gruden, K. & Coll, A. TGA transcription factors-Structural characteristics as basis for functional variability. Front. Plant Sci. 13, 935819 (2022)). The present disclosure redefines the DOG1 domain as the sensor domain (FIG. 1P), as in L. japonicus FUN (SEQ ID NO: 1), this domain senses zinc. FUN is highly conserved in legumes, as evidenced by all analyzed legumes carrying both a FUN protein and a FUN-like paralogue protein in the PAN orthogroup (FIG. 6A). Exemplary FUN homologues include SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30. Exemplary FUN-like homologues include SEQ ID NO: 4, SEQ ID NO: 6, SEQ ID NO: 7, SEQ ID NO: 80, SEQ ID NO: 81, SEQ ID NO: 82, SEQ ID NO: 83, and SEQ ID NO: 84. Additional TGA transcription factors related to FUN proteins are FUN-like proteins. FUN-like proteins can be distinguished from FUN proteins by the lack of enhanced expression in the nodule (there may be some expression generally in the root, potentially including the nodule) and the fact that they form an independent paralogous branch on the phylogenetic tree.

L. japonicus NAC094 (SEQ ID NO: 31; also referred to as a FEZ protein) is a downstream target of FUN (FIG. 7). Exemplary NAC094 homologues include SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, and SEQ ID NO: 73.

Transformation and generation of genetically altered monocotyledonous and dicotyledonous plant cells is well known in the art. See, e.g., Weising, et al., Ann. Rev. Genet. 22:421-477 (1988); U.S. Pat. No. 5,679,558; Agrobacterium Protocols, ed: Gartland, Humana Press Inc. (1995); Wang, et al. Acta Hort. 461:401-408 (1998), and Broothaerts, et al. Nature 433:629-633 (2005). The choice of method varies with the type of plant to be transformed, the particular application, and/or the desired result. The appropriate transformation technique is readily chosen by the skilled practitioner.

Any methodology known in the art to delete, insert or otherwise modify the cellular DNA (e.g., genomic DNA and organelle DNA) can be used in practicing the compositions, methods, and processes disclosed herein. As an example, the CRISPR/Cas-9 system and related systems (e.g., TALEN, ZFN, ODN, etc.) may be used to insert a heterologous gene to a targeted site in the genomic DNA or substantially edit an endogenous gene to express the heterologous gene or to modify the promoter to increase or otherwise alter expression of an endogenous gene through, for example, removal of repressor binding sites or introduction of enhancer binding sites. For example, a disarmed Ti plasmid, containing a genetic construct for deletion or insertion of a target gene, in Agrobacterium tumefaciens can be used to transform a plant cell, and thereafter, a transformed plant can be regenerated from the transformed plant cell using procedures described in the art, for example, in EP 0116718, EP 0270822, PCT publication WO 84/02913 and published European Patent application (“EP”) 0242246. Ti-plasmid vectors each contain the gene between the border sequences, or at least located to the left of the right border sequence, of the T-DNA of the Ti-plasmid. Of course, other types of vectors can be used to transform the plant cell, using procedures such as direct gene transfer (as described, for example in EP 0233247), pollen mediated transformation (as described, for example in EP 0270356, PCT publication WO 85/01856, and U.S. Pat. No. 4,684,611), plant RNA virus-mediated transformation (as described, for example in EP 0 067 553 and U.S. Pat. No. 4,407,956), liposome-mediated transformation (as described, for example in U.S. Pat. No. 4,536,475), and other methods such as the methods for transforming certain lines of corn (e.g., U.S. Pat. No. 6,140,553; Fromm et al., Bio/Technology (1990) 8, 833-839); Gordon-Kamm et al., The Plant Cell, (1990) 2, 603-618), rice (Shimamoto et al., Nature, (1989) 338, 274-276; Datta et al., Bio/Technology, (1990) 8, 736-740), and the method for transforming monocots generally (PCT publication WO 92/09696). For cotton transformation, the method described in PCT patent publication WO 00/71733 can be used. For soybean transformation, reference is made to methods known in the art, e.g., Hinchee et al. (Bio/Technology, (1988) 6, 915) and Christou et al. (Trends Biotech, (1990) 8, 145) or the method of WO 00/42207.

Genetically altered plants of the present disclosure can be used in a conventional plant breeding scheme to produce more genetically altered plants with the same characteristics, or to introduce the genetic alteration(s) in other varieties of the same or related plant species. Seeds, which are obtained from the altered plants, preferably contain the genetic alteration(s) as a stable insert in chromosomal DNA or as modifications to an endogenous gene or promoter. Plants including the genetic alteration(s) in accordance with this disclosure include plants including, or derived from, root stocks of plants including the genetic alteration(s) of this disclosure, e.g., fruit trees or ornamental plants. Hence, any non-transgenic grafted plant parts inserted on a transformed plant or plant part are included in this disclosure.

Genetic alterations of the disclosure, including in an expression vector or expression cassette, which result in the expression of an introduced gene or altered expression of an endogenous gene will typically utilize a plant-expressible promoter. A ‘plant-expressible promoter’ as used herein refers to a promoter that ensures expression of the genetic alteration(s) of this disclosure in a plant cell. Examples of constitutive promoters that are often used in plant cells are the cauliflower mosaic (CaMV) 35S promoter (Kay et al. Science, 236, 4805, 1987), the minimal CaMV 35S promoter (Benfey & Chua, Science, (1990) 250, 959-966), various other derivatives of the CaMV 35S promoter, the figwort mosaic virus (FMV) promoter (Richins, et al., Nucleic Acids Res. (1987) 15:8451-8466) the maize ubiquitin promoter (Christensen & Quail, Transgenic Res, 5, 213-8, 1996), the polyubiquitin promoter (Ljubql, Maekawa et al. Mol Plant Microbe Interact. 21, 375-82, 2008), the vein mosaic cassava virus promoter (International Application WO 97/48819), and the Arabidopsis UBQ10 promoter, Norris et al. Plant Mol. Biol. 21, 895-906, 1993).

Additional examples of promoters directing constitutive expression in plants are known in the art and include: the strong constitutive 35S promoters (the “35S promoters”) of the cauliflower mosaic virus (CaMV), e.g., of isolates CM 1841 (Gardner et al., Nucleic Acids Res, (1981) 9, 2871-2887), CabbB S (Franck et al., Cell (1980) 21, 285-294) and CabbB JI (Hull and Howell, Virology, (1987) 86, 482-493); promoters from the ubiquitin family (e.g., the maize ubiquitin promoter of Christensen et al., Plant Mol Biol, (1992) 18, 675-689), the gos2 promoter (de Pater et al., The Plant J (1992) 2, 834-844), the emu promoter (Last et al., Theor Appl Genet, (1990) 81, 581-588), actin promoters such as the promoter described by An et al. (The Plant J, (1996) 10, 107), the rice actin promoter described by Zhang et al. (The Plant Cell, (1991) 3, 1155-1165); promoters of the figwort mosaic virus (FMV) (Richins, et al., Nucleic Acids Res. (1987) 15:8451-8466), promoters of the Cassava vein mosaic virus (WO 97/48819; Verdaguer et al., Plant Mol Biol, (1998) 37, 1055-1067), the pPLEX series of promoters from Subterranean Clover Stunt Virus (WO 96/06932, particularly the S4 or S7 promoter), an alcohol dehydrogenase promoter, e.g., pAdh1S (GenBank accession numbers X04049, X00581), and the TR1′ promoter and the TR2′ promoter (the “TR1′ promoter” and “TR2′ promoter”, respectively) which drive the expression of the 1′ and 2′ genes, respectively, of the T DNA (Velten et al., EMBO J, (1984) 3, 2723-2730).

Alternatively, a plant-expressible promoter can be a tissue-specific promoter, i.e., a promoter directing a higher level of expression in some cells or tissues of the plant, e.g., in root epidermal cells or root cortex cells. In preferred embodiments, LysM receptor promoters will be used. Non-limiting examples include NFR1 promoters, NFR5 promoters, LYK3 promoters, NFP promoters, the Lotus japonicus NFR5 promoter (SEQ ID NO: 27), the Lotus japonicus NFR1 promoter (SEQ ID NO: 27), the Medicago truncatula NFP promoter (SEQ ID NO: 29), the Lotus japonicus CERK6 promoter (SEQ ID NO: 46), and the Medicago truncatula LYK3 promoter (SEQ ID NO: 28). In additional preferred embodiments, root specific promoters will be used. Non-limiting examples include the promoter of the maize metallothionein (De Framond et al, FEBS 290, 103.-106, 1991 Application EP 452269), the chitinase promoter (Samac et al. Plant Physiol 93, 907-914, 1990), the glutamine synthetase soybean root promoter (Hirel et al. Plant Mol. Biol. 20, 207-218, 1992), the RCC3 promoter (PCT Application WO 2009/016104), the rice antiquitin promoter (PCT Application WO 2007/076115), the LRR receptor kinase promoter (PCT application WO 02/46439), the maize ZRP2 promoter (U.S. Pat. No. 5,633,363), the tomato LeExt1 promoter (Bucher et al. Plant Physiol. 128, 911-923, 2002), and the Arabidopsis pCO2 promoter (Heidstra et al, Genes Dev. 18, 1964-1969, 2004). These plant promoters can be combined with enhancer elements, they can be combined with minimal promoter elements, or can include repeated elements to ensure the expression profile desired.

Examples of constitutive promoters that are often used in plant cells are the cauliflower mosaic (CaMV) 35S promoter (Kay et al. Science, 236, 4805, 1987), and various derivatives of the promoter, virus promoter vein mosaic cassava (International Application WO 97/48819), the maize ubiquitin promoter (Christensen & Quail, Transgenic Res, 5, 213-8, 1996), polyubiquitin (Ljubql, Maekawa et al. Mol Plant Microbe Interact. 21, 375-82, 2008) and Arabidopsis UBQ10 (Norris et al. Plant Mol. Biol. 21, 895-906, 1993).

In some embodiments, further genetic alterations to increase expression in plant cells can be utilized. For example, an intron at the 5′ end or 3′ end of an introduced gene, or in the coding sequence of the introduced gene, e.g., the hsp70 intron. Other such genetic elements can include, but are not limited to, promoter enhancer elements, duplicated or triplicated promoter regions, 5′ leader sequences different from another transgene or different from an endogenous (plant host) gene leader sequence, 3′ trailer sequences different from another transgene used in the same plant or different from an endogenous (plant host) trailer sequence.

An introduced gene of the present disclosure can be inserted in host cell DNA so that the inserted gene part is upstream (i.e., 5′) of suitable 3′ end transcription regulation signals (i.e., transcript formation and polyadenylation signals). This is preferably accomplished by inserting the gene in the plant cell genome (nuclear or chloroplast). Preferred polyadenylation and transcript formation signals include those of the nopaline synthase gene (Depicker et al., J. Molec Appl Gen, (1982) 1, 561-573), the octopine synthase gene (Gielen et al., EMBO J, (1984) 3:835-845), the SCSV or the Malic enzyme terminators (Schunmann et al., Plant Funct Biol, (2003) 30:453-460), and the T DNA gene 7 (Velten and Schell, Nucleic Acids Res, (1985) 13, 6981-6998), which act as 3′ untranslated DNA sequences in transformed plant cells. In some embodiments, one or more of the introduced genes are stably integrated into the nuclear genome. Stable integration is present when the nucleic acid sequence remains integrated into the nuclear genome and continues to be expressed (i.e., detectable mRNA transcript or protein is produced) throughout subsequent plant generations. Stable integration into the nuclear genome can be accomplished by any known method in the art (e.g., microparticle bombardment, Agrobacterium-mediated transformation, CRISPR/Cas9, electroporation of protoplasts, microinjection, etc.).

The term recombinant or modified nucleic acids refers to polynucleotides which are made by the combination of two otherwise separated segments of sequence accomplished by the artificial manipulation of isolated segments of polynucleotides by genetic engineering techniques or by chemical synthesis. In doing so, one may join together polynucleotide segments of desired functions to generate a desired combination of functions.

As used herein, the term “overexpression” refers to increased expression (e.g., of mRNA, polypeptides, etc.) relative to expression in a wild type organism (e.g., plant) as a result of genetic modification and can refer to expression of heterologous genes at a sufficient level to achieve the desired result such as increased yield. In some embodiments, the increase in expression is a slight increase of about 10% more than expression in wild type. In some embodiments, the increase in expression is an increase of 50% or more (e.g., 60%, 70%, 80%, 100%, etc.) relative to expression in wild type. In some embodiments, an endogenous gene is upregulated. In some embodiments, an exogenous gene is upregulated by virtue of being expressed. Upregulation of a gene in plants can be achieved through any known method in the art, including but not limited to, the use of constitutive promoters with inducible response elements added, inducible promoters, high expression promoters (e.g., PsaD promoter) with inducible response elements added, enhancers, transcriptional and/or translational regulatory sequences, codon optimization, modified transcription factors, and/or mutant or modified genes that control expression of the gene to be upregulated in response to a stimulus such as cytokinin signaling.

Where a recombinant nucleic acid is intended for expression, cloning, or replication of a particular sequence, DNA constructs prepared for introduction into a host cell will typically include a replication system (e.g., vector) recognized by the host, including the intended DNA fragment encoding a desired polypeptide, and can also include transcription and translational initiation regulatory sequences operably linked to the polypeptide-encoding segment. Additionally, such constructs can include cellular localization signals (e.g., plasma membrane localization signals). In preferred embodiments, such DNA constructs are introduced into a host cell's genomic DNA, chloroplast DNA or mitochondrial DNA.

In some embodiments, a non-integrated expression system can be used to induce expression of one or more introduced genes. Expression systems (expression vectors) can include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides can also be included where appropriate from secreted polypeptides of the same or related species, which allow the protein to cross and/or lodge in cell membranes, cell wall, or be secreted from the cell.

Selectable markers useful in practicing the methodologies disclosed herein can be positive selectable markers. Typically, positive selection refers to the case in which a genetically altered cell can survive in the presence of a toxic substance only if the recombinant polynucleotide of interest is present within the cell. Negative selectable markers and screenable markers are also well known in the art and are contemplated by the present disclosure. One of skill in the art will recognize that any relevant markers available can be utilized in practicing the compositions, methods, and processes disclosed herein.

Screening and molecular analysis of recombinant strains of the present disclosure can be performed utilizing nucleic acid hybridization techniques. Hybridization procedures are useful for identifying polynucleotides, such as those modified using the techniques described herein, with sufficient homology to the subject regulatory sequences to be useful as taught herein. The particular hybridization techniques are not essential to this disclosure. As improvements are made in hybridization techniques, they can be readily applied by one of skill in the art. Hybridization probes can be labeled with any appropriate label known to those of skill in the art. Hybridization conditions and washing conditions, for example temperature and salt concentration, can be altered to change the stringency of the detection threshold. See, e.g., Sambrook et al. (1989) vide infra or Ausubel et al. (1995) Current Protocols in Molecular Biology, John Wiley & Sons, NY, N.Y., for further guidance on hybridization conditions.

Additionally, screening and molecular analysis of genetically altered strains, as well as creation of desired isolated nucleic acids can be performed using Polymerase Chain Reaction (PCR). PCR is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki et al. (1985) Science 230:1350-1354). PCR is based on the enzymatic amplification of a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Because the extension product of each primer can serve as a template for the other primer, each cycle essentially doubles the amount of DNA template produced in the previous cycle. This results in the exponential accumulation of the specific target fragment, up to several million-fold in a few hours. By using a thermostable DNA polymerase such as the Taq polymerase, which is isolated from the thermophilic bacterium Thermus aquaticus, the amplification process can be completely automated. Other enzymes which can be used are known to those skilled in the art.

Nucleic acids and proteins of the present disclosure can also encompass homologues of the specifically disclosed sequences. Homology (e.g., sequence identity) can be 50%-100%. In some instances, such homology is greater than 80%, greater than 85%, greater than 90%, or greater than 95%. The degree of homology or identity needed for any intended use of the sequence(s) is readily identified by one of skill in the art. As used herein percent sequence identity of two nucleic acids is determined using an algorithm known in the art, such as that disclosed by Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the BLASTN, BLASTP, and BLASTX, programs of Altschul et al. (1990) J. Mol. Biol. 215:402-410. BLAST nucleotide searches are performed with the BLASTN program, score=100, wordlength=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped BLAST programs, the default parameters of the respective programs (BLASTN and BLASTX) are used. See www.ncbi.nih.gov. One of skill in the art can readily determine in a sequence of interest where a position corresponding to amino acid or nucleic acid in a reference sequence occurs by aligning the sequence of interest with the reference sequence using the suitable BLAST program with the default settings (e.g., for BLASTP: Gap opening penalty: 11, Gap extension penalty: 1, Expectation value: 10, Word size: 3, Max scores: 25, Max alignments: 15, and Matrix: blosum62; and for BLASTN: Gap opening penalty: 5, Gap extension penalty:2, Nucleic match: 1, Nucleic mismatch −3, Expectation value: 10, Word size: 11, Max scores: 25, and Max alignments: 15).

Preferred host cells are plant cells. Recombinant host cells, in the present context, are those which have been genetically modified to contain an isolated nucleic molecule, contain one or more deleted or otherwise non-functional genes normally present and functional in the host cell, or contain one or more genes to produce at least one recombinant protein. The nucleic acid(s) encoding the protein(s) of the present disclosure can be introduced by any means known to the art which is appropriate for the particular type of cell, including without limitation, transformation, lipofection, electroporation or any other methodology known by those skilled in the art.

“Isolated”, “isolated DNA molecule” or an equivalent term or phrase is intended to mean that the DNA molecule or other moiety is one that is present alone or in combination with other compositions, but altered from or not within its natural environment. For example, nucleic acid elements such as a coding sequence, intron sequence, untranslated leader sequence, promoter sequence, transcriptional termination sequence, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” from its natural setting within the scope of this disclosure so long as the element is not within the genome of the organism in which it is naturally found, the element is altered from its natural form, or the element is not at the location within the genome in which it is naturally found. Similarly, a nucleotide sequence encoding a protein or any naturally occurring variant of that protein would be an isolated nucleotide sequence so long as the nucleotide sequence was not within the DNA of the organism from which the sequence encoding the protein is naturally found in its natural location or if that nucleotide sequence was altered from its natural form. A synthetic nucleotide sequence encoding the amino acid sequence of the naturally occurring protein would be considered to be isolated for the purposes of this disclosure. For the purposes of this disclosure, any transgenic nucleotide sequence, i.e., the nucleotide sequence of the DNA inserted into the genome of the cells of a plant, alga, fungus, or bacterium, or present in an extrachromosomal vector, would be considered to be an isolated nucleotide sequence whether it is present within the plasmid or similar structure used to transform the cells, within the genome of the plant or bacterium, or present in detectable amounts in tissues, progeny, biological samples or commodity products derived from the plant or bacterium.

### Plants

Compositions and methods described herein can be utilized with a plant, plant cell, plant part, or progeny thereof, such as plants capable of nodulation and have endogenous FUN (e.g., legumes). Legumes are plants that belong to the family Fabaceae (Leguminosae) and can be characterized by their ability to fix nitrogen in the soil through a symbiotic relationship with nitrogen-fixing bacteria in their root nodules. As such, the plant, plant cell, plant part, or progeny thereof as described herein can be selected from the group of alfalfa, Bambara groundnut, bean (e.g., kidney beans, black beans, etc.), black currant, chickpea, clover, cowpea, forage legumes, legume trees, lentil, lotus, lupin, Medicago spp., pea, peanut, pigeon pea, soybean, Parasponia, alder trees, or elm trees.

Plants having identified FUN orthologs can be Prunus persica (peach), Lotus japonicus (e.g., Japanese lotus or bird's-Foot trefoil), Glycine max (soybean), Manihot esculenta (cassava), Gossypium raimondii (wild cotton, Eucalyptus grandis (e.g., flooded gum or Rose Gum), Brassica oleracea (e.g., wild cabbage, species can include various cultivated forms like broccoli, cauliflower, cabbage, etc.), Arabidopsis thaliana (thale cress), Solanum lycopersicum (tomato), Aquilegia coerulea (Colorado blue columbine), Amborella trichopoda, Spirodela polyrhiza (greater duckweed), Musa acuminata (banana), Zea mays (maize, corn), Setaria italica (foxtail millet), Triticum aestivum (common wheat), Hordeum vulgare (barley), or Oryza sativa (rice).

Cover crops and combinations of cover crops can be used to add nitrogen to soil and can benefit from decreased expression of FUN or its downstream targets by increasing nitrogen content. For example, a cover crop can be a legume. In some embodiments, a legume can be soybean, cowpea, clover (e.g., red clover, white clover, crimson clover, balansa clover, berseem clover, bersian clover, arrowleaf clover, ball clover, subterranean clovers), vetch (e.g., common vetch, hairy vetch), or peas (e.g., Austrian winter peas, field peas). As another example, a cover crop can be a grass. In some embodiments, the grass can be rye (e.g., winter rye, cereal rye, Italian ryegrass), triticale, fescue (e.g., tall fescue, meadow fescue), or sudangrass (e.g., sudangrass, sorghum-sudangrass hybrids), or alfalfa. As yet another example, a cover crop can be a Brassica. In some embodiments, the Brassica can be mustard (e.g., white mustard), radish (e.g., Daikon radishes, oilseed radish), turnips (e.g., purple top turnips, forage turnips), or rapeseed (e.g., canola), Phacelia, sunflower, sunn hemp, kale, or a cereal (e.g., oats, buckwheat, or millet (e.g., pearl millet)).

Having generally described the compositions, methods, and processes of this disclosure, the same will be better understood by reference to certain specific examples, which are included herein to further illustrate the disclosure and are not intended to limit the scope of the invention as defined by the claims.

### EXAMPLES

The present disclosure is described in further detail in the following examples which are not in any way intended to limit the scope of the disclosure as claimed. The attached figures are meant to be considered as integral parts of the specification and description of the disclosure. The following example is offered to illustrate, but not to limit the claimed disclosure.

### Example 1: FUN Controls Suppression of Nitrogen Fixation by Nitrate

The following example describes the identification of the Fun gene as a regulator of nitrogen fixation in Lotus japonicus. Further, experiments to characterize the expression pattern of the Fun gene are described.

### Materials and Methods

### Mutagenesis Screen

The Lotus japonicus Gifu ecotype background was used for a forward genetic screen to identify mutants that maintained nitrogen fixation despite restrictive nitrate conditions. Functional nodules were allowed to form before restrictive nitrate conditions were applied, which meant that the screen specifically identified mutants impaired in regulation of nodule function. The distinctive color of nitrogen-fixing nodules (functional nodules are pink) was used to screen for mutants that retained nodule function when watered with water containing 10 mM KNO₃ for two weeks. Most nodules on wild type plants became green and senescent under these growing conditions, but the fixation under nitrate (fun) mutant plants continued to form pink nodules even under these high concentrations of nitrate.

### Plant Lines and Growth Conditions

The Lotus japonicus Gifu ecotype was used as the wild type (WT). LORE1 insertion mutants were ordered through LotusBase (lotus[dot]au[dot]dk) and homozygotes were isolated for phenotyping and generation of higher order mutants as described (Emms, D. M. & Kelly, S. SHOOT: phylogenetic gene search and ortholog inference. Genome Biol. 23, 85 (2022)). The mutant lines fun, fun-2, fun-3, and fun-4 were tested, and the specific insertions generating the mutant genotypes are illustrated in FIG. 1B. The line numbers and genotyping primers used are provided in Table 2, below.

TABLE 2
Line numbers and genotyping primers.

| | Gene | | ID in | | |
|---|---|---|---|---|---|
| Gene | ID | mutants | Lotusbase | primer F | primer R |
| FUN | LotjaGi2g1v | fun | N/A | GAGGTACTGCTAGC | GTGGTGGTCAGTTACTT |
| | 279100 | | | ATTACTACTA | TAGGAGA |
| | | | | (SEQ ID NO: 90) | (SEQ ID NO: 100) |
| | | fun-2 | 30099638 | ATGGCCACCATGCC | CCCCCATTGAAGCAATT |
| | | | | TATGGGGAAT | ACCTTTTTCC |
| | | | | (SEQ ID NO: 91) | (SEQ ID NO: 101) |
| | | fun-3 | 30089287 | AGCTGCTGCCCTGT | TTGCCACCCCTCCTCCT |
| | | | | CTCCAAGCAC | TCTTGCT |
| | | | | (SEQ ID NO: 92) | (SEQ ID NO: 102) |
| | | fun-4 | 30072479 | GAGGTACTGCTAGC | GTGGTGGTCAGTTACTT |
| | | | | ATTACTACTA | TAGGAGA |
| | | | | (SEQ ID NO: 93) | (SEQ ID NO: 103) |
| NRT2.1 | LotjaGi3g1v | nrt2.1-3 | 30128053 | TTGCCGATGTCGCT | TAGTGCGTTGACTGTGG |
| | 487600 | | | TTTGGTGAGA | GGCGAGA |
| | | | | (SEQ ID NO: 94) | SEQ ID NO: 104) |
| | | nrt2.1-4 | 30010185 | TCCGCTTGATAGAT | GGTATGA |
| | | | | GAAGTGGCCTTAAA | TTGCAGCGCGGTTGTTT |
| | | | | (SEQ ID NO: 95) | (SEQ ID NO: 105) |
| HO1 | LotjaGi1g1v | ho1-4 | 30128287 | TCCCTTCCATTCTG | ACCGCTTTCTCCTCAGG |
| | 199700 | | | CATTCCCACC | CTCCGTG |
| | | | | (SEQ ID NO: 96) | SEQ ID NO: 106) |
| | | ho1-5 | 30116204 | GCGTCAGCCACAGT | AGTCGCAAAAGTGAAG |
| | | | | TGTTTCCCA | GGCCACCA |
| | | | | (SEQ ID NO: 97) | (SEQ ID NO: 107) |
| NAC094 | LotjaGi2g1v | nac094-3 | 30165542 | TGGTTGCATGTCCT | CACCGCATATGAAAAG |
| | 259200 | | | GAGGGAGGCT | ATTGGTGGTGA |
| | | | | (SEQ ID NO: 98) | (SEQ ID NO: 108) |
| | | nac094-4 | 30007808 | TGGATCTCTGAGTT | TGGTTGCATGTCCTGAG |
| | | | | ACCAGGAGGCATG | GGAGGCT |
| | | | | G | (SEQ ID NO: 109) |
| | | | | (SEQ ID NO: 99) | |

All plants were grown at 21° C. under 16 hour light/8 hour dark conditions. For germination, Lotus seeds were scarified with sandpaper and surface sterilized for 10 minutes with 1% sodium hypochlorite. Seedlings were washed with sterile water 5 times and were germinated on wet filter paper (AGF 651; Frisenette ApS) in an upright position in sterile square Petri dishes at 21° C. for two days. Then, seedlings were transferred into the substrate mixture (leca:vermiculite=3:1).

### Mutant Screening and Sequence Analysis

A LORE1 mutant pool, in which there were random LORE1 insertions in the genome of each individual, were germinated in substrate mixture (leca:vermiculite 3:1) and inoculated with M. loti NZP2235. Four weeks post inoculation, plants were watered with 10 mM KNO₃ for three weeks. Most nodules became green or black, and plants with pink nodules were isolated for rescreening in subsequent generations. DNA from mutant plants was isolated and LORE1 flanking sequences were sequenced to identify LORE1 insertion positions as previously described (Urbaiiski, D. F., Malolepszy, A., Stougaard, J. & Andersen, S. U. Genome-wide LORE1 retrotransposon mutagenesis and high-throughput insertion detection in Lotus japonicus. Plant J. 69, 731-741 (2012)).

### Bacterial Strains and Culture Conditions

Chemically competent E. coli TOP10 (ThermoFisherScientific) were used for molecular cloning and were grown in LB medium at 37° C.

Agrobacterium rhizogenes strain AR1193 (Stougaard, J. Methods Mol Biol 1995 49:49-61) was used for all hairy root transformation experiments and cultured in LB medium at 28° C.

### Generation of Plant Expression Vectors

To validate the function of the Fun gene, an expression construct was generated to express FUN fused to green fluorescent protein (GFP) under the control of the ubiquitin promoter (pUbi), and the construct was designated pUbi:FUN, proUbi:FUN-GFP, or FUN-GFP. For the tobacco assay, the 35S promoter (pro35S) was used, and the construct was designated pro35S:FUN, pro35S:FUN-GFP, or FUN-GFP.

To study the expression pattern of the Fun gene in situ, the coding sequence for b-glucuronidase (GUS) was assembled with the native Fun promoter sequence (proFUN) and the native Fun terminator sequence (tFUN), and the construct was designated proFun:GUS.

### Hairy Root Transformation

For hairy root transformation of L. japonicus, the pIV10 expression vector (Hansen, J. et al. Plant Cell Rep 1989 8: 12-15) was used. This expression vector contains a sequence encoding triple YFP fused to a nuclear localization signal (pIV10_tYFP-NLS) that serves as a transformation control. In addition, the Lotus ubiquitin promoter and the 35S terminator were cloned into the pIV10 expression vector.

L. japonicus seeds were scarified with sulfuric acid for 15 minutes, respectively, washed 5 times in ddH₂O and dispersed on wet filter paper for germination. 3 day old seedlings were transferred to square plates containing solid ½ B5 medium. A. rhizogenes AR1193 strains (Stougaard, 1987 #432) carrying the construct of interest were grown for two days on LB Agar containing Ampicillin, Rifampicin, and Spectinomycin. For each construct the cells grown on one plate were resuspended in 4 ml YMB media. The bacterial suspension was then used to transform the hypocotyl of 6-day old seedlings using a 1 ml syringe with a needle (Sterican® Ø 0.40×20 mm), punching the hypocotyl and placing a droplet on the wound. Square plates containing the transformed seedlings were sealed and left in the dark for two days and then moved to 21° C. under 16/8-hour light/dark conditions. After three weeks, non-transformed roots were removed, and seedlings were transferred to the substrate mixture described above or onto ¼× B&D plates. After transformation, plants were inoculated with rhizobia and watered with nitrate as described above. All plants were grown at 21° C. under 16/8-hour light/dark conditions.

### Nodulation Assay

The number of pink, functional nodules and total nodules per plant were counted after 2 weeks of 10 mM KNO₃ exposure. Pictures were acquired with a Leica M165FC Fluorescent Stereo Microscope equipped with the Leica DFC310 FX digital color camera. Means between treatment groups were compared using ANOVA and Tukey post-hoc testing.

### Nitrogen Fixation Assay

Nitrogen fixation activity was quantified using an acetylene reduction assay (ARA) that measures the amount of acetylene (C₂H₂) reduced to ethylene (C₂H₄) per hour per Lotus plant, after 2 weeks of 10 mM KNO₃ exposure as described previously (Reid, D. E., Heckmann, A. B., Novák, O., Kelly, S. & Stougaard, J. CYTOKININ OXIDASE/DEHYDROGENASE3 maintains cytokinin homeostasis during root and nodule development in Lotus japonicus. Plant Physiol. 170, 1060-1074 (2016)). The nodulated root from single plants was placed in a 5 ml glass GC vial. A syringe was used to replace 500 μl air in the vial with 2% acetylene. Samples were incubated at room temperature for 30 min before ethylene quantification using a SensorSense (Nijmegen, NL) ETD-300 ethylene detector operating in sample mode with 2.5 L/h flow rate and 6-min detection time. The curve was integrated using the SensorSense valve controller software to calculate the total ethylene production per sample. Means between treatment groups were compared using ANOVA and Tukey post-hoc testing.

### Leghemoglobin Content Assay

Leghemoglobin content was assayed after 2 weeks of 10 mM KNO₃ exposure as described previously (Du, M., Gao, Z., Li, X., and Liao, H. (2020). Excess nitrate induces nodule greening and reduces transcript and protein expression levels of soybean leghemoglobin. Ann. Bot. 126: 61-72)). Fresh nodules from each individual plant were first ground and homogenized in 16-fold volumes of 0.1 M precooled PBS (Na₂HPO₄—NaH₂PO₄ buffer at 5° C., pH 6.8). The resulting slurry was then centrifuged at 12,000 g for 15 min prior to assaying the supernatant by spectrophotometry at a wavelength of 540, 520, and 560 nm. The Leghemoglobin content was calculated from a standard curve using Bovine Hb as a protein standard. Means between treatment groups were compared using ANOVA and Tukey post-hoc testing.

### GUS Staining

Three weeks post inoculation, hairy roots were put into GUS staining buffer, which contains 0.5 mg/mi 5-bromo-4-chloro-3-indolyl-β-D-glucuronic acid (X-Gluc), 100 mM potassium phosphate buffer (pH 7.0), 10 mM EDTA (pH 8.0), 1 mM potassium ferricyanide, 1 mM potassium ferrocyanide, and 0.1% Triton X-100. The roots were incubated at 37° C. overnight. Roots were washed with 70% ethanol twice before image acquisition.

### Statistical Analysis

Means between treatment groups were compared using ANOVA and Tukey post-hoc testing.

### Results

### FUN Controls Suppression of Nitrogen Fixation by Nitrate

To identify environmental regulators of nodulation, it was reasoned that by applying restrictive conditions after functional root nodules were formed, mutants with specific impairments in regulating nodule function could be identified. Using the distinctive pink color of nitrogen-fixing nodules as opposed to green senescent nodules, a population of LORE1 (Fukai, E. et al. Establishment of a Lotus japonicus gene tagging population using the exon-targeting endogenous retrotransposon LORE1. Plant J. 69, 720-730 (2012); Urbanski, D. F., Malolepszy, A., Stougaard, J. & Andersen, S. U. Genome-wide LORE1 retrotransposon mutagenesis and high-throughput insertion detection in Lotus japonicus. Plant J. 69, 731-741 (2012); Malolepszy, A. et al. The LORE1 insertion mutant resource. Plant J. 88, 306-317 (2016)) insertion mutants in the model legume Lotus japonicus (Lotus) were screened to identify genotypes retaining nodule function despite suppressive nitrate conditions. A mutant was identified that retained a higher number of pink nodules relative to wild-type (WT) and named fixation under nitrate (fun) (FIGS. 1A and 1C). Additional LORE1 insertion mutants were also identified (FIG. 1B). When the amount of functional pink nodules and the total number of nodules per plant were assayed for fun mutant Lotus plants and compared to WT (Gifu) Lotus plants, it was found that fun, fun-2, fun-3, and fun-4 plants retained a significantly higher number of pink nodules relative to WT (FIGS. 1C and 1F).

The function of these pink nodules was confirmed by elevated nitrogen fixation rates when assayed by an acetylene reduction assay (ARA) (FIGS. 1D, 1E, and 1G). In addition, the fun mutant Lotus plants had increased leghemoglobin content compared to WT Lotus plants (FIG. 1K). Analysis of the abundance of FUN transcripts from an available expression atlas (Kamal et al., DNA Res. 7 (2020)) for Lotus japonicus Gifu showed high upregulation of FUN activity localized to the nodules where nitrogen fixation takes place (FIG. 1Q).

### FUN Protein Structure

A LORE1 retrotransposon insertion was identified in the promoter region of a bZIP-type transcription factor, which was named FUN as described above. As shown in FIG. 1B, additional LORE1 insertions were subsequently identified in the promoter and gene region of FUN. The FUN gene encoded a protein of the TGA family of transcription factors, with greatest similarity to the Arabidopsis PERIANTHIA (PAN) transcription factor (Running, M. P. & Meyerowitz, E. M. Mutations in the PERIANTHIA gene of Arabidopsis specifically alter floral organ number and initiation pattern. Development 122, 1261-1269 (1996); Maier, A. T., Stehling-Sun, S., Offenburger, S.-L. & Lohmann, J. U. The bZIP Transcription Factor PERIANTHIA: A Multifunctional Hub for Meristem Control. Front. Plant Sci. 2, 79 (2011)). The TGA family belongs to group D bZIP transcription factors (Dröge-Laser, W., Snoek, B. L., Snel, B. & Weiste, C. The Arabidopsis bZIP transcription factor family—an update. Curr. Opin. Plant Biol. 45, 36-49 (2018)) and is characterized by the presence of a basic leucine zipper (bZIP) DNA-binding domain in the N-terminus, and a DOG1 domain of unknown function in the C-terminus (Tomaz̆, S̆., Gruden, K. & Coll, A. TGA transcription factors-Structural characteristics as basis for functional variability. Front. Plant Sci. 13, 935819 (2022)). As described in more detail below, the DOG1 domain is referred to as the sensor domain for FUN. The protein structure of FUN is shown in FIG. 1P, which depicts the bZIP domain and the sensor domain.

### Fun is Expressed Specifically in Root Nodules

In Lotus, Fun transcripts were detected at high levels in nodules (FIG. 1Q), and promoter activity was evident in the nodules (FIGS. 1M, 1N, and 1O). To assess Fun expression in situ, a GUS transcriptional reporter construct driven by the native Fun promoter was transformed into wild type (Gifu) Lotus plants (FIGS. 1M-1O). After GUS staining of nodule-bearing Lotus roots, it was found that Fun was exclusively expressed in nodules (FIG. 1M). Stained nodules were sectioned and imaged with light microscopy to determine the cellular localization of Fun expression. This showed that Fun was dominantly expressed in uninfected cells in nodules (FIGS. 1N-1O).

### Complementation of Fun Mutant Lotus Plants

FUN was validated as the causative gene by complementing the fun mutation with a constitutively expressed FUN (FIG. 1L), and by confirming that the nodulation phenotype was consistent in three independent LORE1 mutant alleles that reduced gene expression via promoter insertion (fun and fun-4) or by interrupting function via exonic insertion (fun-3) (FIGS. 1A and 1E-1G). An intronic insertion allele (fun-2) was not impaired relative to wild type (FIG. 1F-1G). FUN regulation was restricted to mature functional nodules since application of nitrate prior to inoculation inhibited nodulation in fun mutant plants to the same degree as in wild type plants (FIG. 1H-1J). The Fun promoter was also shown to be sufficient to complement the fun mutation when cloned with the Fun genomic sequence (data not shown).

These results demonstrated that the Fun gene was specifically expressed in nodules, and controlled suppression of nitrogen fixation by nitrate.

### Example 2: FUN Initiates Nodule Senescence Via Multiple Pathways

The following example describes the identification of FUN target genes and FUN binding sites in FUN target gene promoters. In addition, the phenotypes for loss of function mutants of FUN targets in Lotus are described.

### Materials and Methods

### Gene Expression

For RNA-seq, three weeks post inoculation, plants were acclimated prior to treatment by submerging in ¼ Long Ashton liquid medium overnight, then treated with 0 or 10 mM KNO₃ for 24h. Mature nodules were harvested. mRNA was isolated using the NucleoSpin RNA Plant kit (Macherey-Nagel) and RNA sequencing (PE-150 bp Illumina sequencing) was conducted by Novogene. RNAseq analysis was performed by mapping reads to the reference transcriptome using Salmon 39 and quantification performed using DEseq2 (Love, M. I., Huber, W. & Anders, S. Moderated estimation of fold change and dispersion for RNA-seq data with DESeq2. Genome Biol. 15, 550 (2014). A publicly available timeseries of nitrate treated nodules (Wang, L. et al. A transcription factor of the NAC family regulates nitrate-induced legume nodule senescence. New Phytol. (2023) doi: 10.1111/nph.18896) was obtained from GEO using accession number GSE197362. GO enrichment was performed using GO_MWU with GO terms obtained from lotus[dot]au[dot]dk.

For the expression of target genes, RevertAid Reverse Transcriptase (Thermo) was used for the synthesis of first strand cDNA. LightCycler480 instrument and LightCycler480 SYBR Green I master (Roche Diagnostics) were used for the qRT-PCR. Ubiquitin-conjugating enzyme was used as a reference. The cDNA concentration of target genes was calculated using amplicon PCR efficiency calculations using LinRegPCR (Ramakers, C., Ruijter, J. M., Deprez, R. H. L. & Moorman, A. F. M. Assumption-free analysis of quantitative real-time polymerase chain reaction (PCR) data. Neurosci. Lett. 339, 62-66 (2003)). Target genes were compared to the reference for each of 5 biological repetitions (each consisting of 8 to 10 nodules). At least two technical repetitions were performed in each analysis. Primers used are listed below, in Table 3.

TABLE 3
Primers used in qPCR.

| Gene | Gene ID | primer F | primer R |
|---|---|---|---|
| FUN | LotjaGi2g1 | CCTTCAC | ATCATTT |
| | v0279100 | AGTGCTG | CTGACAA |
| | | ATCTCG | GGAGA |
| | | TGAA | GCACG |
| | | (SEQ ID | (SEQ ID |
| | | NO: 110) | NO: 117) |
| NRT2.1 | LotjaGi3g1 | AGGCCAC | GCCAGGG |
| | v0487600 | TTCATCT | AATCCAT |
| | | ATCAAG | TAACA |
| | | C | TTT |
| | | (SEQ ID | (SEQ ID |
| | | NO: 111) | NO: 118) |
| HO1 | LotjaGi1g1 | CTCATCC | AGAGGAA |
| | v0199700 | TTCCTAT | GGTTGAG |
| | | GCTGAA | GAATG |
| | | TTC | GTGT |
| | | (SEQ ID | (SEQ ID |
| | | NO: 112) | NO: 119) |
| NAC094 | LotjaGi2g1 | GGACCTT | CCTGCTC |
| | v0259200 | CCAAAAC | TTGTCAC |
| | | TGGCT | TCTATTT |
| | | AGTAC | GGC |
| | | (SEQ ID | (SEQ ID |
| | | NO: 113) | NO: 120) |
| NRT3.1 | LotjaGi4g1 | ACATGGA | GTAGCAC |
| | v0227700 | CAAGTIG | AGCTTCA |
| | | TGGAA | CCTTTA |
| | | GCTG | TGG |
| | | (SEQ ID | (SEQ ID |
| | | NO: 114) | NO: 121) |
| AS1 | LotjaGilg1 | TCTCGCT | GATCAGG |
| | v0118200 | ACTTGGC | TGAACCC |
| | | AACCAC | TCAAG |
| | | AA | GCC |
| | | (SEQ ID | (SEQ ID |
| | | NO: 115) | NO: 122) |
| Ubi | | atgtgca | gaacgta |
| | | ttttaag | gaagatt |
| | | acaggg | gcctgaa |
| | | (SEQ ID | (SEQ ID |
| | | NO: 116) | NO: 123) |

### Plant Lines and Growth Conditions

The plant lines and growth conditions were as described in Example 1.

### Bacterial Strains and Culture Conditions

The bacterial strains and culture conditions were as described in Example 1.

### Hairy Root Transformation

Hairy root transformation was as described in Example 1.

### Nodulation Assay

Nodulation assays were conducted as described in Example 1.

### Nitrogen Fixation Assay

Nitrogen fixation activity was quantified as described in Example 1.

### Leghemoglobin Content Assay

Leghemoglobin content was assayed as described in Example 1.

### Electrophoretic Mobility Shift Assay (EMSA)

The DNA probes with 6-FAM-label at the 5′ end were synthesized by Eurofins and are provided in Table 4, below. The purified FUN DNA binding domain (residues 178-237) was incubated with the probes at 37° C. for 60 min in EMSA buffer (25 mM Tris-HCl pH8.0, 80 mM NaCl, 35 mM KCl, 5 mM MgCl₂). After incubation, the reaction mixture was electrophoresed in 6% native polyacrylamide gel and then labelled DNA was detected with the Typhoon scanner (Fujifilm). Probes without 6-FAM-label served as competitors, while probes with mutation in the core binding sites (TGACG) served as mutants.

TABLE 4
Probes for EMSA.

| Probe name | forward labelled with 6FAM | reverse |
|---|---|---|
| NRT2.1 P1 | ACAAGTAGCTTATGACGTACGAC | ACTATATGTCGTACGTCATAAG |
| | ATATAGT (SEQ ID NO: 124) | CTACTTGT (SEQ ID NO: 137) |
| NRT2.1 P2 | TCTAGATTGTTTGACGATAGAAT | TCTAGATTGTTTGACGATAGAA |
| | CTCCAAT (SEQ ID NO: 125) | TCTCCAAT (SEQ ID NO: 138) |
| NRT2.1 P3 | ATTATTATGTTGACGGAAAGACA | GGTGTAGTGTCTTTCCGTCAAC |
| | CTACACC (SEQ ID NO: 126) | ATAATAAT (SEQ ID NO: 139) |
| NRT2.1 P4 | AGATTTAAGTTGACGTATTAATG | TCATTGTCATTAATACGTCAAC |
| | ACAATGA (SEQ ID NO: 127) | TTAAATCT (SEQ ID NO: 140) |
| NRT2.1 P1m | AGATTTAAGTaataaatTTAATGACA | ACTATATGTCGatttattTAAGCTAC |
| | ATGA (SEQ ID NO: 128) | TTGT (SEQ ID NO: 141) |
| NRT2.1 P4m | ACAAGTAGCTTAaataaatCGACATA | TCATTGTCATTAAatttattACTTAA |
| | TAGT (SEQ ID NO: 129) | ATCT (SEQ ID NO: 142) |
| HO1 P1 | TGTAATAACTTGTGAGCGAAGCA | GGATCCCTGCTTCGCTCACAAG |
| | GGGATCC (SEQ ID NO: 130) | TTATTACA (SEQ ID NO: 143) |
| HO1 P2 | TCGAGCCTGCCTTGAGCTTGTCT | ATGGAGAAGACAAGCTCAAGG |
| | TCTCCAT (SEQ ID NO: 131) | CAGGCTCGA (SEQ ID NO: 144) |
| NAC094 P1 | AGGAGAGGTTTGTGAGCATCAG | ACACTTTGCTGATGCTCACAAA |
| | CAAAGTGT (SEQ ID NO: 132) | CCTCTCCT (SEQ ID NO: 145) |
| NRT3.1 P1 | CGTTCATTAATATGACATCAGAA | AGAGAAATTCTGATGTCATATT |
| | TTTCTCT (SEQ ID NO: 133) | AATGAACG (SEQ ID NO: 146) |
| NRT3.1 P2 | GGTTTTTATTTTTGACCTCAGAG | CGATCCCCTCTGAGGTCAAAAA |
| | GGGATCG (SEQ ID NO: 134) | TAAAAACC (SEQ ID NO: 147) |
| NRT3.1 P3 | AGACCTATTTTCTGACTCAATGT | ACCCTCTACATTGAGTCAGAAA |
| | AGAGGGT (SEQ ID NO: 135) | ATAGGTCT (SEQ ID NO: 148) |
| AS1 P1 | CATGTGGGCTTTTGACGAAGGTT | CATGTGGGCTTTTGACGAAGGT |
| | CAAGTAG (SEQ ID NO: 136) | TCAAGTAG (SEQ ID NO: 149) |

### Transient Activation Assay

Promoters of FUN candidate target genes (NRT2.1, HO1, NAC094, NRT3.1, and AS1), the glucuronidase (GUS) CDS, and the 35S terminator were cloned into compatible Golden Gate vectors as reporters; while the 35S promoter, FUN CDS, eGFP, and 35S terminator were cloned as the effector. The reporters and effector were cloned into the p50507 Golden Gate binary vector. These constructs were then transformed into A. tumefaciens strain AGL1. These A. tumefaciens were diluted into OD₆₀₀=0.2 and were infiltrated into N. benthamiana leaves. Three days after infiltration, samples of about 20 mg were collected for protein extraction. GUS activities were measured with 4-methylumbelliferyl-β-D-glucuronide as substrate (Sigma-Aldrich) using a Thermo Scientific Varioskan flash. For Zn treatment, 2 days after A. tumefaciens infiltration, N. benthamiana leaves were infiltrated with 500 μM MgCl₂ (mock), 500 μM ZnCl₂, or 2.5 mM EDTA. GUS activities were measured 1 day after treatments.

### Results

### FUN is a Master Regulator of Nodule Senescence

Since FUN was a transcriptional regulator, RNAseq was conducted to search for gene targets associated with nitrate signaling or nodule function that may be directly regulated. RNAseq analysis identified 587 genes with greater than 2-fold expression change in WT nodules exposed to nitrate. Comparison with fun mutants showed that 106 of these genes were regulated differently in fun nodules (FIGS. 1R-1T). RNAseq analysis identified multiple downstream targets of FUN, some of which were upregulated and others of which were downregulated, with several gene ontology groups detected in both up- and down-regulated gene groups by GO-MWU (Nielsen, R. et al. A scan for positively selected genes in the genomes of humans and chimpanzees. PLoS Biol. 3, e170 (2005)) (FIG. 1U). Of these, 29 upregulated and 22 downregulated genes predicted as targets of FUN are provided in Tables 5A and 5B, below.

TABLE 5A
Upregulated downstream targets of FUN

| Gene ID | Gene annotation |
|---|---|
| LotjaGi6g1v0087700 | nitric oxide synthase-interacting protein |
| LotjaGi1g1v0118200 | glutamine-dependent asparagine synthase 1 |
| LotjaGi1g1v0654700 | Disease resistance protein (CC-NBS-LRR class) family |
| | protein |
| LotjaGi6g1v0050600 | Oxidative stress 3 |
| LotjaGi4g1v0227700 | High-affinity nitrate transporter 3.1 |
| LotjaGi2g1v0163700 | Kunitz trypsin inhibitor |
| LotjaGi4g1v0408400 | DNA-directed RNA polymerase subunit beta-beta protein |
| LotjaGi3g1v0411600 | Tetratricopeptide repeat (TPR)-like superfamily protein |
| LotjaGi1g1v0465800 | cytochrome P450, family 76, subfamily C, polypeptide 4 |
| LotjaGi3g1v0394300 | Trehalose 6-phosphate phosphatase |
| LotjaGi1g1v0517000 | Cytochrome P450 family protein |
| LotjaGi4g1v0339500 | Late embryogenesis abundant protein Lea5 |
| LotjaGi3g1v0113400 | FMN-dependent NADPH-azoreductase |
| LotjaGi3g1v0499400_LC | Pentatricopeptide repeat-containing protein family |
| LotjaGi3g1v0176800 | Myb family transcription factor APL |
| LotjaGi1g1v0415600 | Rhamnogalacturonate lyase |
| LotjaGi5g1v0186000 | Sieve element occlusion protein |
| LotjaGi1g1v0710000_LC | Retrovirus-related Pol polyprotein from transposon |
| LotjaGi3g1v0047400 | Transmembrane protein, putative |
| LotjaGi6g1v0212400 | Probable inositol transporter |
| LotjaGi4g1v0433900 | Basic-leucine zipper (bZIP) transcription factor family |
| | protein |
| LotjaGi1g1v0197200_LC | Phloem specific protein |
| LotjaGi2g1v0259200 | NAC domain-containing protein |
| LotjaGi1g1v0453400 | Peroxidase |
| LotjaGi3g1v0113400 | FMN-dependent NADPH-azoreductase |
| LotjaGi6g1v0271600 | Ferredoxin--NADP reductase |
| LotjaGi1g1v0199700 | Heme oxygenase 1 |
| LotjaGi3g1v0487600 | High-affinity nitrate transporter 2.2 |
| LotjaGi1g1v0118200 | Asparagine synthetase |

TABLE 5B
Downregulated downstream targets of FUN

| Gene ID | Gene annotation |
|---|---|
| LotjaGi1g1v0049800 | MLP-like protein |
| LotjaGi4g1v0362400 | Protein-tyrosine sulfotransferase-like protein |
| LotjaGi3g1v0087300 | cytochrome P450, family 82, subfamily G, polypeptide 1 |
| LotjaGi6g1v0067000_LC | B3 domain-containing transcription factor VRN1-like protein |
| LotjaGi2g1v0240500_LC | Plasma membrane fusion protein prm1 |
| LotjaGi1g1v0501300 | Leucine-rich repeat receptor-like protein kinase family |
| | protein |
| LotjaGi1g1v0480900 | Cytochrome P450 |
| LotjaGi1g1v0403800 | 5′-methylthioadenosine/S-adenosylhomocysteine |
| | nucleosidase |
| LotjaGi5g1v0043800_LC | 50S ribosomal protein L1 |
| LotjaGi1g1v0643500 | 5′-adenylylsulfate reductase-like 5 |
| LotjaGi2g1v0048400 | Senescence-associated protein |
| LotjaGi2g1v0028500 | Senescence-associated protein |
| LotjaGi1g1v0723500 | Polychome, UV-B-insensitive 4 |
| LotjaGi3g1v0443800_LC | N-alpha-acetyltransferase 16, NatA auxiliary subunit |
| LotjaGi3g1v0039400 | Trehalose-6-phosphate synthase |
| LotjaGi3g1v0303400 | Glycosyltransferase |
| LotjaGi6g1v0271500 | Transmembrane protein, putative |
| LotjaGi5g1v0215400 | Benzyl alcohol O-benzoyltransferase |
| LotjaGi3g1v0329600 | Protein FAM136A family |
| LotjaGi1g1v0171200_LC | |
| LotjaGi5g1v0316900 | Fantastic four-like protein |
| LotjaGi3g1v0183200 | Protein FAF-like, chloroplastic |

From this longer list, six upregulated genes were selected for investigation in more detail. These six genes are provided in Table 6, below. Particularly notable among these genes were the Hemne Oxygenase HO1, which degrades leghemoglobin during nodule senescence (Wang, L. et al. CRISPR/Cas9 knockout of leghemoglobin genes in Lotus japonicus uncovers their synergistic roles in symbiotic nitrogen fixation. New Phytol. 224, 818-832 (2019); Zhou, Y. et al. Heme catabolism mediated by heme oxygenase in uninfected interstitial cells enables efficient symbiotic nitrogen fixation in Lotus japonicus nodules. New Phytol. (2023) doi: 10.1111/nph.19074), a nitrate transporter, NRT3.1 and the Asparagine Synthetase 1 (AS1) gene, which is important for nitrogen assimilation (FIG. 1V). In addition, a number of putative TGA-type binding motifs (TGACG; Bartlett, A. et al. Mapping genome-wide transcription-factor binding sites using DAP-seq. Nat. Protoc. 12, 1659-1672 (2017)) were identified in the promoter regions of two genes showing similar phenotypes to fun when mutated: the nitrate transporter NRT2.1 (Misawa, F. et al. Nitrate transport via NRT2.1 mediates NIN-LIKE PROTEIN-dependent suppression of root nodulation in Lotus japonicus. Plant Cell 34, 1844-1862 (2022)), and a NAC transcription factor NAC094, which triggers nodule senescence (Wang, L. et al. A transcription factor of the NAC family regulates nitrate-induced legume nodule senescence. New Phytol. (2023) doi: 10.1111/nph.18896).

TABLE 6
Six genes investigated in more detail.

| Gene ID | Gene annotation | Gene name | Sequence |
|---|---|---|---|
| LotjaGi4g1v0227700 | High-affinity nitrate transporter | Nrt3.1 | SEQ ID NO: 74 |
| | 3.1 | | |
| LotjaGi4g1v0433900 | Basic-leucine zipper (bZIP) | Bzip28 | SEQ ID NO: 75 |
| | transcription factor family protein | | |
| LotjaGi2g1v0259200 | NAC domain-containing protein | Nac094 | SEQ ID NO: 76 |
| LotjaGi1g1v0199700 | Heme oxygenase 1 | Ho1 | SEQ ID NO: 77 |
| LotjaGi3g1v0487600 | High-affinity nitrate transporter | Nrt2.1 | SEQ ID NO: 78 |
| | 2.2 | | |
| LotjaGi1g1v0118200 | Asparagine synthetase | As1 | SEQ ID NO: 79 |

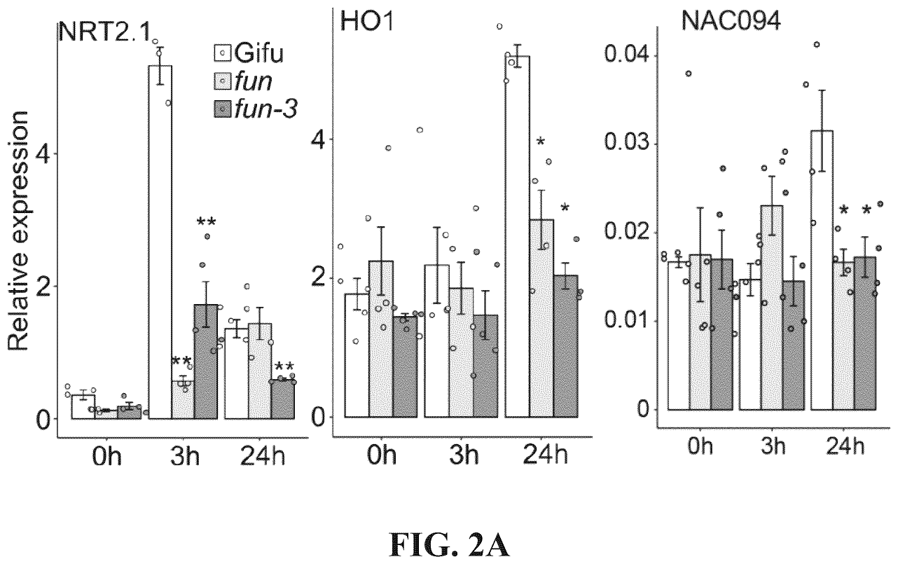

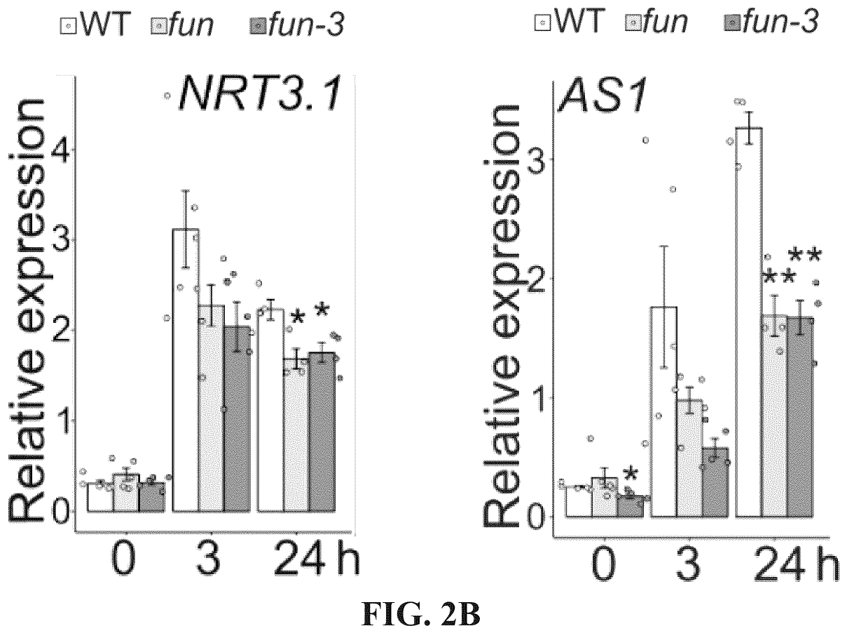

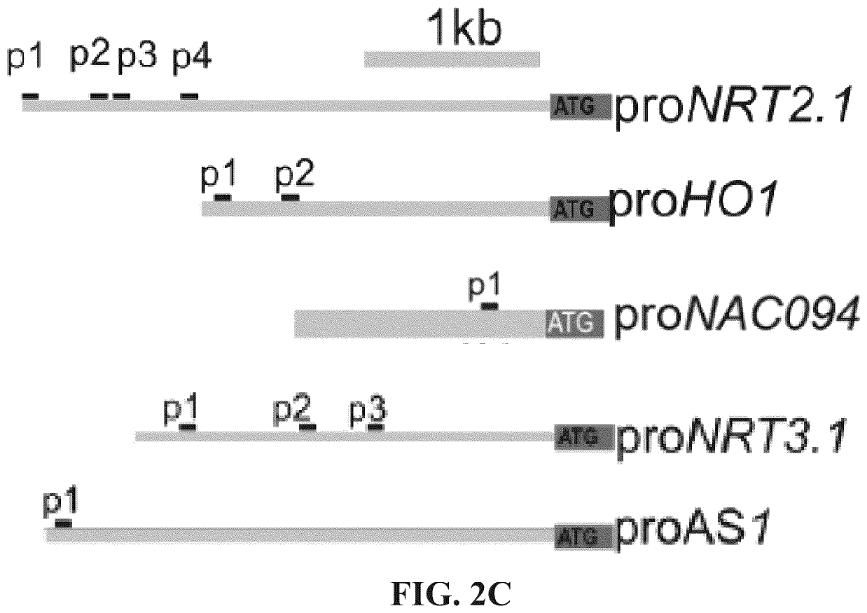

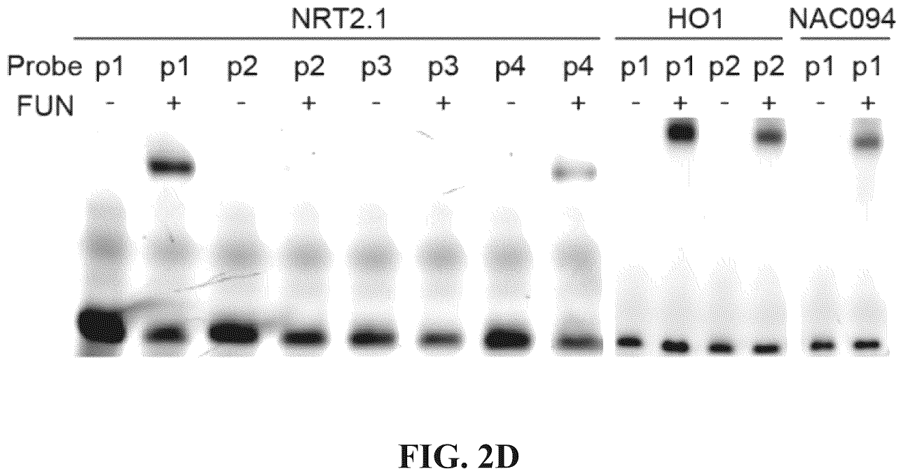

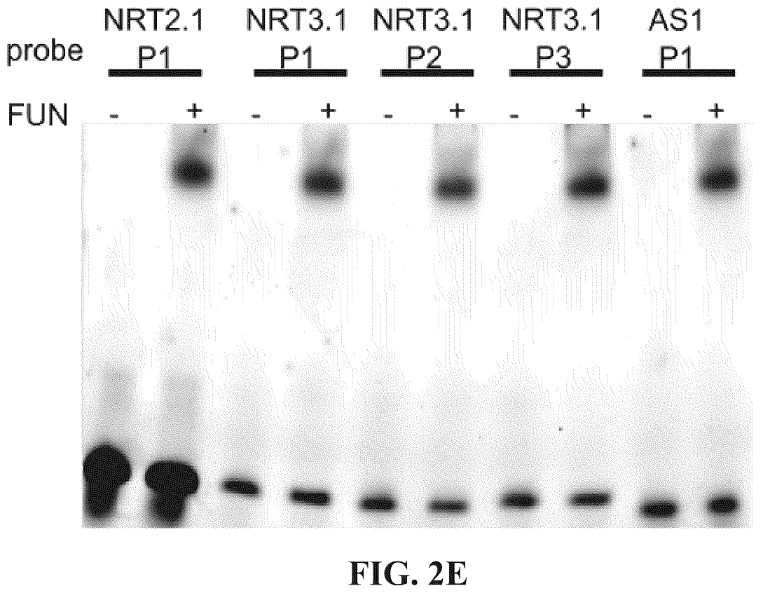

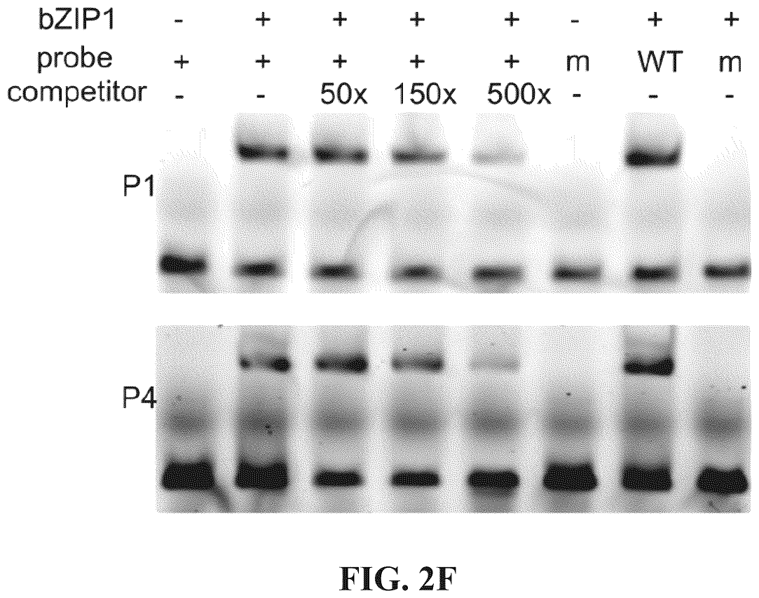

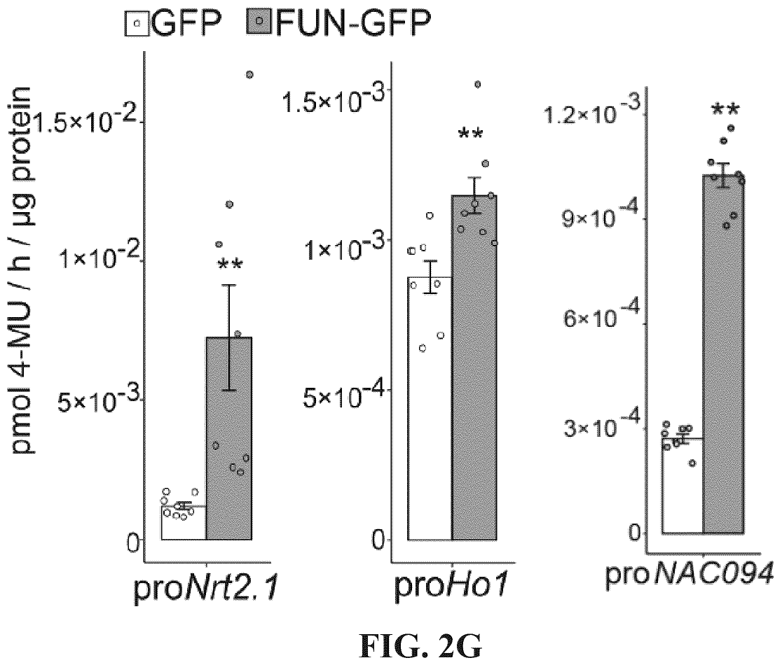

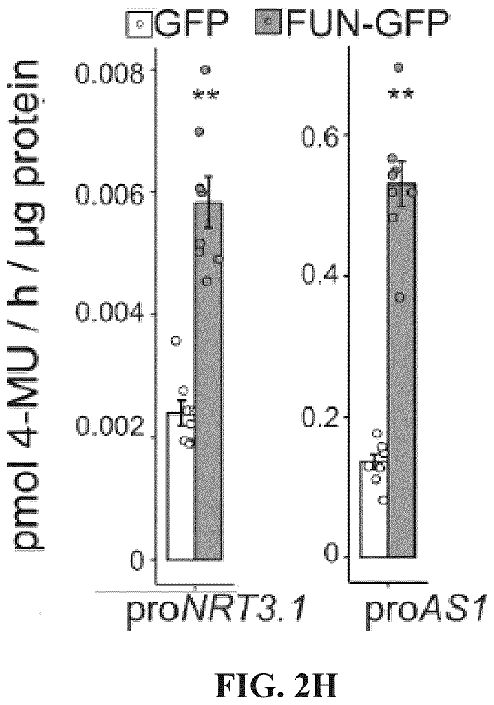

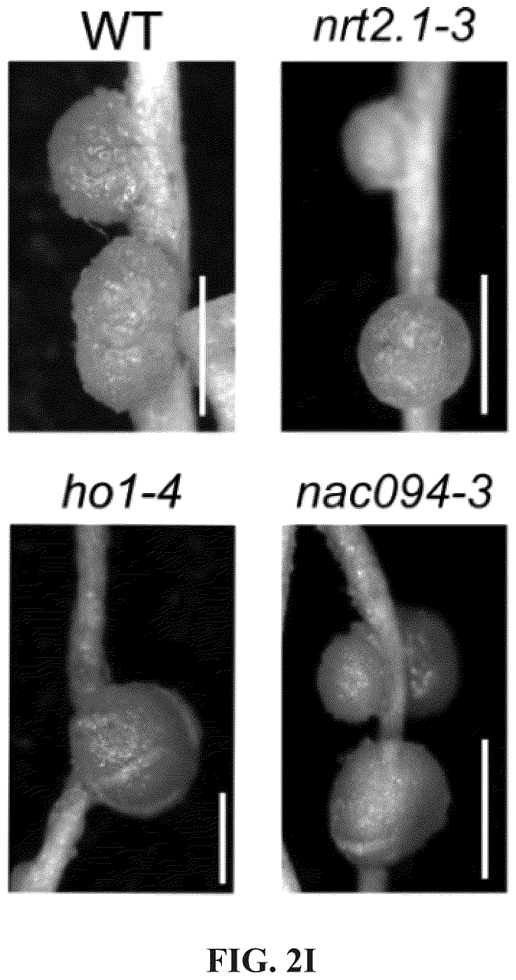

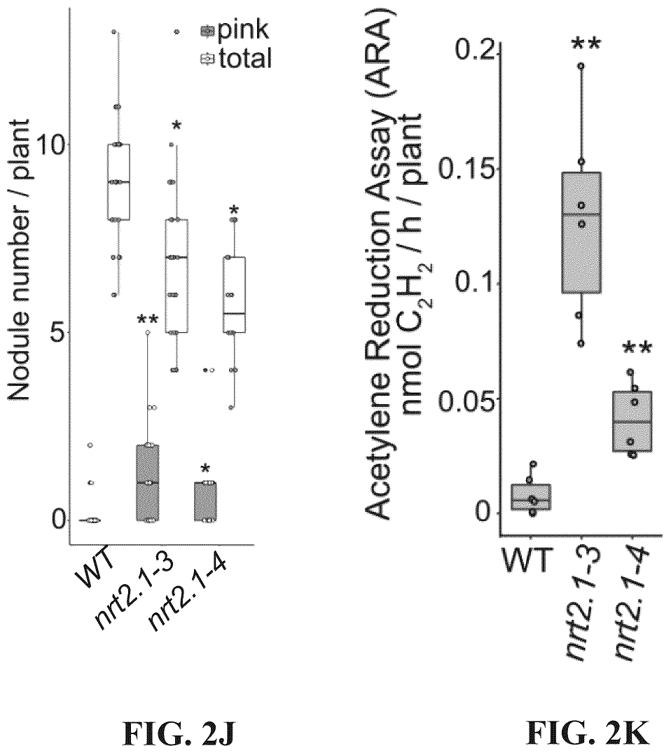

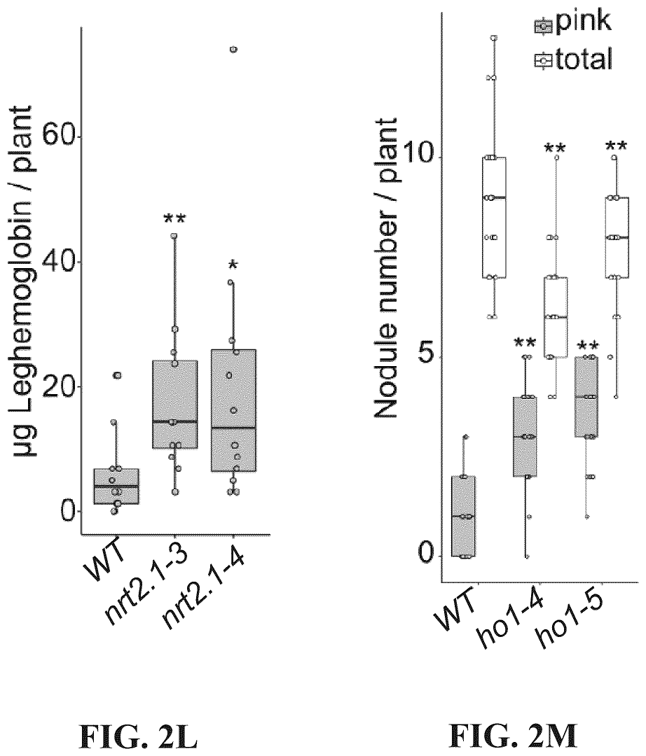

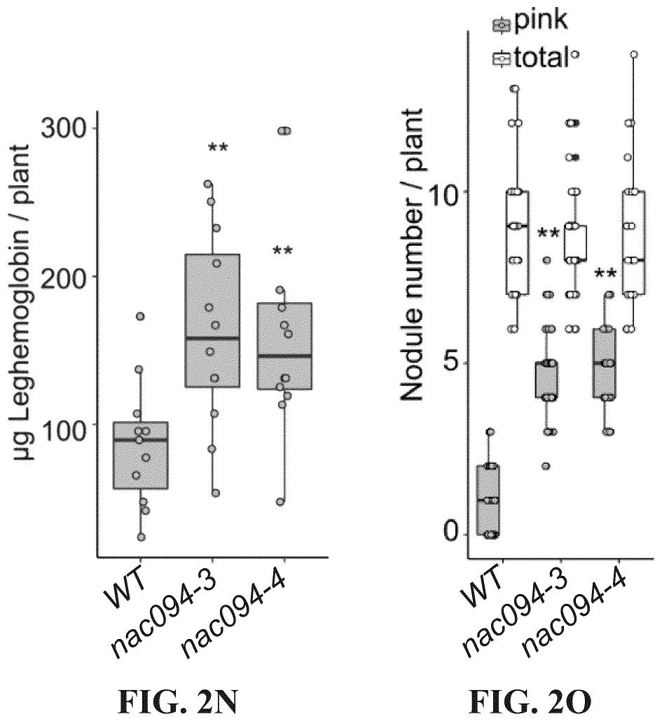

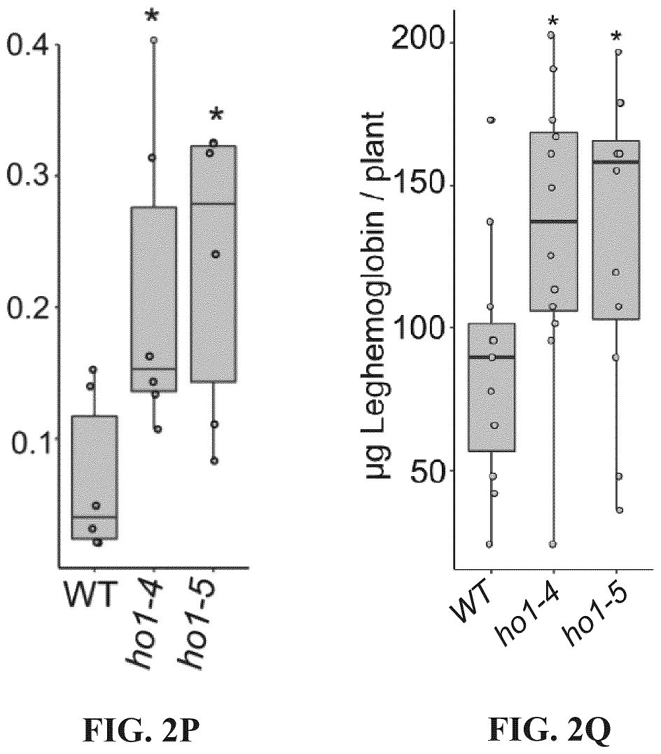

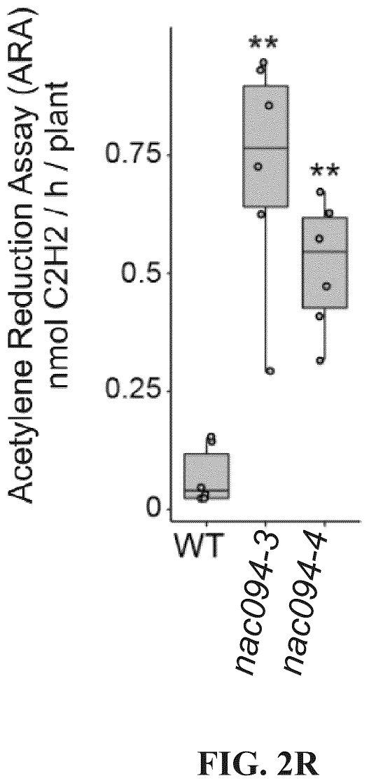

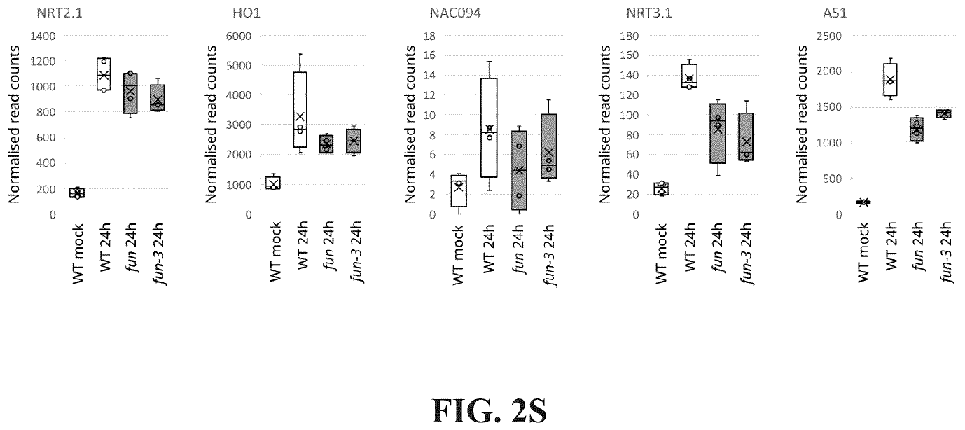

In order to test whether expression of these genes was controlled by FUN, the relative expression in nodules of fun mutants was examined following nitrate treatment. Induction of all of these genes by nitrate was attenuated in fun mutants analyzed by qRT-PCR (FIGS. 2A-2B) and by RNAseq (FIG. 2S). FUN was coexpressed in uninfected cells with NAC094 and HO1, while nitrate regulation of NAC094 that also occurred in infected cells (Wang, L. et al. A transcription factor of the NAC family regulates nitrate-induced legume nodule senescence. New Phytol. (2023) doi: 10.1111/nph.18896) may require additional regulators.

The promoter region of Nrt2.1 had four putative FBSs (P1-P4), the promoter region of Ho1 had two FBSs (P1 and P2), the promoter region of NAC094 had one FBS (P1), the promoter region of Nrt3.1 had three FBSs (P1, P2, and P3), and the promoter region of AS1 had one FBS (P1), all of which are illustrated in FIG. 2C. EMSA was conducted to test whether the FUN DNA-binding domain bound probes representing the FBSs. As can be seen in FIG. 2D, the FUN DNA-binding domain bound P1 and P4 in the Nrt2.1 promoter region, P1 and P2 in the Ho1 promoter region, and P1 in the NAC094 promoter region. As can be seen in FIG. 2E, the FUN DNA-binding domain additionally bound P1, P2, and P3 in the Nrt3.1 promoter region, as well as P1 in the AS1 promoter region. Competition assays with excess unlabeled probe demonstrated the specificity of this interaction (FIG. 2F).

To validate the relevance of the EMSA binding results in vivo, transient activation experiments in N. benthamiana were conducted for the NRT2.1, HO1, NAC094, NRT3.1, and AS1 promoters and showed that all the promoters coupled to the GUS reporter were significantly induced by FUN in this system. The FUN-GFP construct was expressed as the effector, and GUS driven by the promoters was expressed as the reporter. As shown in FIG. 2G, each of the Nrt2.1, Ho1, and NAC094 promoters coupled to the GUS reporter were significantly induced by FUN in this system. The same effect was shown for Nrt3.1 and AS1 in FIG. 2H.

Further supporting the view that FUN is a master regulator controlling these pathways, mutants obtained in nrt2.1, ho1 and nac094 showed similar nodule phenotypes to the original fun mutant, including enhanced nitrogen-fixation and leghemoglobin content. FIG. 2I shows the nodulation phenotype of the nrt2.1-3, ho1-4, and nac094-3 mutants as compared to WT. FIGS. 2J-2L show the results of nodule number, ARA activity and leghemoglobin content assays for the nrt2.1-3 and nrt2.1-4 mutants as compared to WT. FIGS. 2M-2Q show the results of nodule number, ARA activity and leghemoglobin content assays of the ho1-4 and ho1-5 mutants, as well as nac094-3 and nac094-4 mutants, as compared to WT. FIG. 2R shows the results of the ARA activity assay of the nac094-3 and nac094-4 mutants as compared to WT. Together, these data indicate that FUN targets nodule senescence and nitrate signaling pathways to modulate nodule function in relation to the environment. Regulation of the nitrate signaling pathway by FUN in this way may serve to alter the sensitivity of the nodule to nitrate relative to other root tissues.

### Example 3: The Oligomeric State of FUN is Regulated by Zinc

The following example describes the structural characterization of the FUN sensor domain and experiments assessing the contribution of manganese and zinc as sensor domain ligands.

### Materials and Methods

### Protein Production and Purification

The FUN sensor domain (residues 244-480) with a 3C cleavable N-terminal tag consisting of 10×Histidines, 7×Arginines and a SUMO tag was ordered from GenScript together with a construct of the FUN sensor with the zipper domain (residues 178-480) N-terminally tagged with 7×-Histidines and a GB1 tag. The plasmids were transformed into E. coli LOBSTR cells (Andersen, K. R., Leksa, N. C. & Schwartz, T. U. Optimized E. coli expression strain LOBSTR eliminates common contaminants from His-tag purification. Proteins 81, 1857-1861 (2013)). The expression culture was grown to OD₆₀₀=0.6 in LB media with 0.1 mg/mL ampicillin and 0.034 mg/mL chloramphenicol at 37° C. and 110 rpm. Cells were cold shocked on ice for 30 min before expression was induced with 0.4 mM IPTG at 18° C. overnight. The cells were pelleted (4400 g, 4° C., 10 min), resuspended in lysis buffer (50 mM Tris-HCl pH 8.0, 500 mM NaCl, 10% glycerol, 10 mM imidazole, 5 mM β-mercaptoethanol and 1 mM benzamidine) and lysed by sonication. The lysate was cleared by centrifugation (30600 g, 4° C., 30 min), and the proteins were purified from the cleared lysate using a Protino Ni-NTA 5 mL column (Machery-Nagel). The protein was eluted with a high-imidazole buffer (50 mM Tris-HCl pH 8.0, 250 mM NaCl, 5% glycerol, 500 mM imidazole, 5 mM β-mercaptoethanol). The FUN sensor with zipper was not purified further, while the FUN sensor was dialyzed overnight against 50 mM Tris-HCl pH 8.0, 250 mM NaCl, 5% glycerol, 5 mM β-mercaptoethanol with 3C protease in a 1:50 molar ratio. The cleaved tag and the protease were subsequently removed by a second Ni-IMAC step. The FUN sensor was further purified by SEC on a Superdex 200 Increase 10/300 GL (GE Healthcare) in minimal buffer (10 mM Tris-HCl pH 8.0, 150 mM NaCl, 5 mM β-mercaptoethanol).

For SAXS analysis, the FUN sensor was further purified on a ResourceQ 1 mL (GE Healthcare) and eluted with a linear gradient of 10-500 mM NaCl and 1OmM Tris-HCl pH 8.0 and 5 mM β-mercaptoethanol. Eluted fractions were pooled and dialyzed against minimal buffer.

Dynamic Light Scattering (DLS) and Nano Differential Scanning Fluorimetry (nanoDSF) Analyses

The FUN protein was analyzed on a Prometheus Panta instrument (NanoTemper Technologies) for alterations in thermal unfolding (nanoDSF) and size (DLS) upon addition of ligands. 0.8 mg/mL of the purified protein was incubated with 4 mM of different potential ligands or a 0-4 mM ZnCl₂ series for 20 min whereupon 5 mM EDTA was added to samples analyzed for reversible filamentation. Before addition, ZnCl₂ was filtered using VivaSpin MWCO 5 kDa and immediately added to the protein samples. 10 consecutive DLS measurements were performed for each sample at 25° C. with 100% laser power and followed by a nanoDSF experiment measured at a temperature slope of 1° C./min from 25-90 degrees with 100% excitation power. All measurements were performed in triplicates.

### Small-Angle X-Ray Scattering (SAXS) Analyses

SAXS measurements were performed on a NanoSTAR instrument (Pedersen, J. S. A flux- and background-optimized version of the NanoSTAR small-angle X-ray scattering camera for solution scattering. J. Appl. Crystallogr. 37, 369-380 (2004); Lyngss, J. & Pedersen, J. S. A high-flux automated laboratory small-angle X-ray scattering instrument optimized for solution scattering. J. Appl. Crystallogr. 54, 295-305 (2021)). The instrument uses a Cu rotating anode, has a scatterless pinhole in front of the sample and employs a two-dimensional position-sensitive gas detector (Vantec 500, Bruker AXS). The samples and buffer were measured in a homebuilt flow-through capillary. The intensity I(q) is displayed as a function of the modulus of the scattering vector, Q (and Q=4π·sin(2θ)/λ), where 2θ is the scattering angle and λ is the X-ray wavelength. The buffer scattering was subtracted from the scattering from the samples and the intensities were converted to an absolute scale and corrected for variations in detector efficiency by normalizing to the scattering of pure water (Pedersen, J. S. A flux- and background-optimized version of the NanoSTAR small-angle X-ray scattering camera for solution scattering. J. Appl. Crystallogr. 37, 369-380 (2004)). The data were plotted in Guinier of 1n(I(q)) versus q² to determine the radius of gyration, R_{g}, and an indirect Fourier transformation (IFT) (Glatter, O. A new method for the evaluation of small-angle scattering data. J. Appl. Crystallogr. 10, 415-421 (1977); Pedersen, J. S., Hansen, S. & Bauer, R. The aggregation behavior of zinc-free insulin studied by small-angle neutron scattering. Eur. Biophys. J. 22, 379-389 (1994)) was performed to obtain the pair distance-distribution function p(r), which is a histogram of distances between pair of points within the particles weighted by the excess scattering length density at the points. Note that the resolution of the SAXS data is about 400 Å and therefore the overall length of the fibrils induced by zinc is not resolved. The p(r) function is in this case related to the cross-section structure of the filaments.

### Negative Stain Electron Microscopy

For electron microscopy, 0.1 mg/mL of the purified FUN sensor domain was incubated 20 min at room temperature with or without 100 μM ZnCl₂ and with or without 5 mM EDTA. Samples for negative staining were prepared on 400 copper mesh grids that were manually covered with a collodion support film coated with carbon using a Leica EM SCD 500 High Vacuum Sputter Coater. Before staining, the grids were glow discharged with negative polarity, 25 mA for 45 s, using a PELCO easiGlow glow discharge system. 3 μL of the FUN sensor was deposited on the grid, incubated 30 s, and excess sample was removed from the grid using Whatman paper. After the blotting, the grid was floated 3 times on 2% uranyl formate solution for 15 s and then dried. Negative staining micrographs were recorded using a Tecnai G2 Spirit microscope operating at 120 kV, equipped with a TemCam-F416 (4k×4k) TVIPS CMOS camera and a Veleta (2k×2k) CCD camera, at a cryo-EM facility. Micrographs were recorded at a magnification of 42000× and 52000×.

### Microscope and Confocal Imaging

For the FUN expression pattern, the roots after GUS staining were observed by Leica M165FC Fluorescence stereomicroscope. Nodules were embedded in 3% agarose and sectioned in 100 μm slices using a vibratome. Nodule slices were observed by Zeiss Axioplan 2 light microscope. For FUN subcellular locations, Lotus hairy roots and N. benthamiana leaves expressing FUN-GFP were treated with 500 μM ZnCl₂ (Zn) or MgCl₂ (mock) for 3 days, and fluorescence was observed using a 491-535 nm filter on a Zeiss LSM 710 confocal microscope. For zinc biosensor (eCALWY and eCALWYnls) assays, nodules were embedded in 3% agarose and sectioned in 75 μm slices using a vibratome. Cerulean was excited at 458 nm and the citrine fluorophore captured on a 514-550 nm filter on a Zeiss LSM 710 confocal microscope. M. loti DsRed were detected using a 587-665 nm filter.

### Zinpyr-1 Imaging and Quantification

Plants with pink nodules (3 wpi) were acclimated prior to treatment by submerging in ¼ Long Ashton liquid medium overnight, then treated with 0 or 10 mM KNO₃ for 24 hours. Mature nodules were embedded in 3% agarose and section in 80 μm slices using a vibratome. Sliders were stained with 5 μM Zinpyr-1 for 3 hours and rinsed three times by water. Fluorescence was observed by Zeiss LSM 710 confocal microscope, using excitation at 488 nm and emission from 505-550 nm. Fluorescence densities were quantified by ImageJ.

### Micro-X-Ray Fluorescence Microscopy

Micro-X-ray (mXRF) images were acquired with a scanning X-ray microscope equipped with a liquid nitrogen passively cooled cryogenic stage (Cotte, M. et al. The ID21 X-ray and infrared microscopy beamline at the ESRF: status and recent applications to artistic materials. J. Anal. At. Spectrom. 32, 477-493 (2017)). Samples were prepared as described in Escudero et al. (Escudero, V. et al. Medicago truncatula Ferroportin2 mediates iron import into nodule symbiosomes. New Phytol. 228, 194-209 (2020).) Briefly, nodules were embedded in OCT medium and cryo-fixed by plunging them into liquid nitrogen-chilled isopentane. 20 mm sections of frozen samples were obtained using a Leica LN22 cryo-microtome and mounted in a liquid nitrogen-cooled sample holder between two Ultralene (Spex SamplePrep, Rickmansworth, UK) foils. The beam was focused to 0.9×0.6 mm² using Kirkpatrick-Baez mirror optics. The emitted fluorescence signal was detected with an energy-dispersive, large area (80 mm²) SDD detector equipped with a beryllium window (XFlash SGX, RaySpec, High Wycombe, UK). Images were acquired at a fixed energy of 9.8 keV by raster-scanning the sample with a step of 2×2 mm² and a 220 ms dwell time. Elemental distribution was calculated with the PyMca software package (Sold, V. A. et al. A multiplatform code for the analysis of energy-dispersive X-ray fluorescence spectra. Spectrochim. Acta Part B At. Spectrosc. 62, 63-68 (2007)).

### Results

### The Sensor Domain of FUN Forms Filamentous Structures in the Presence of Physiological Concentrations of Zinc

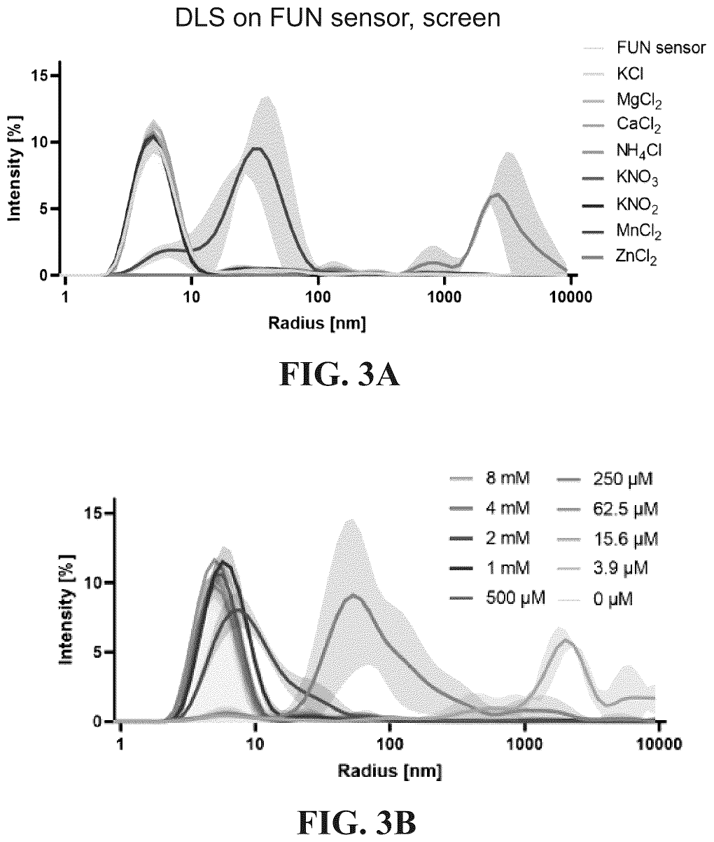

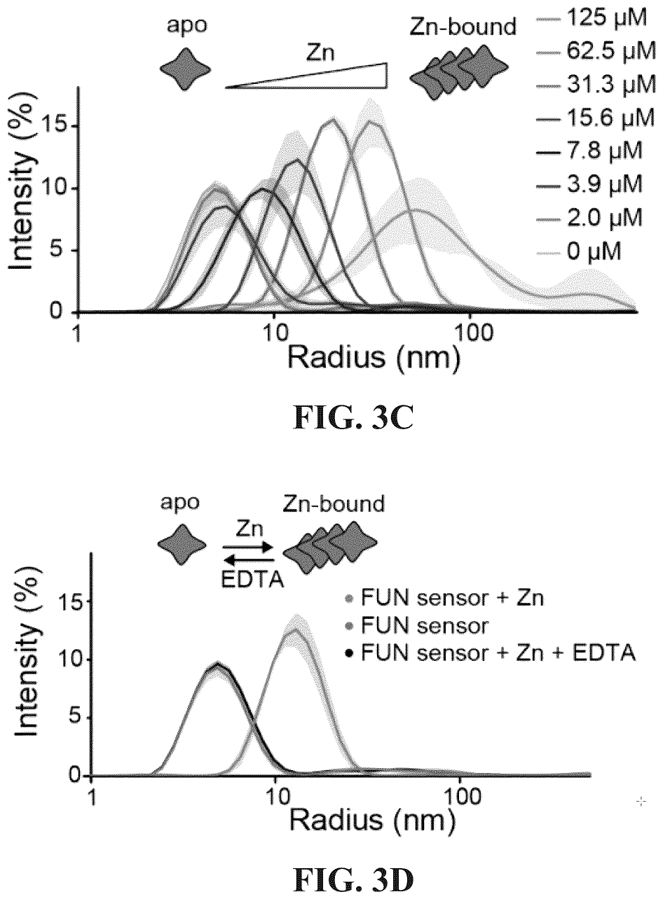

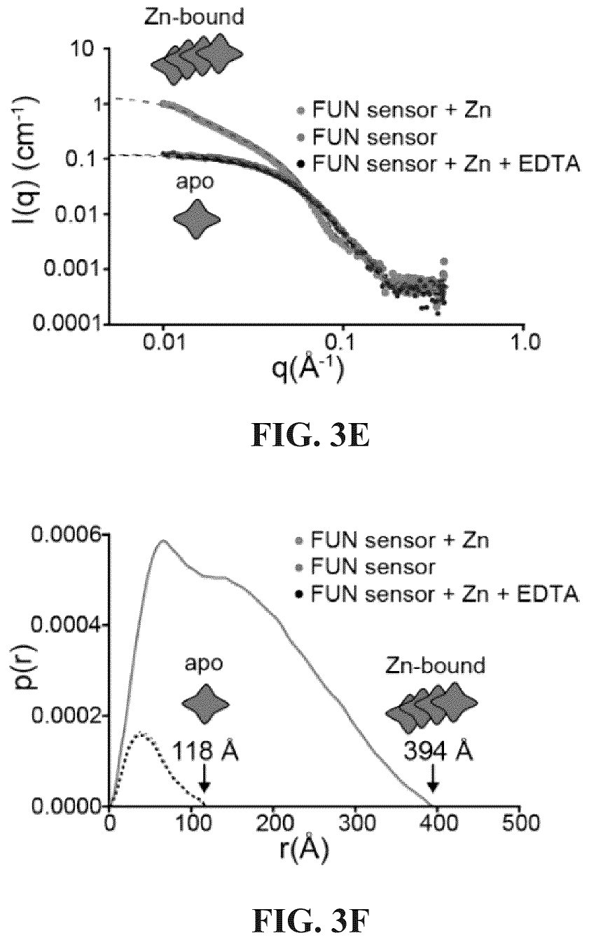

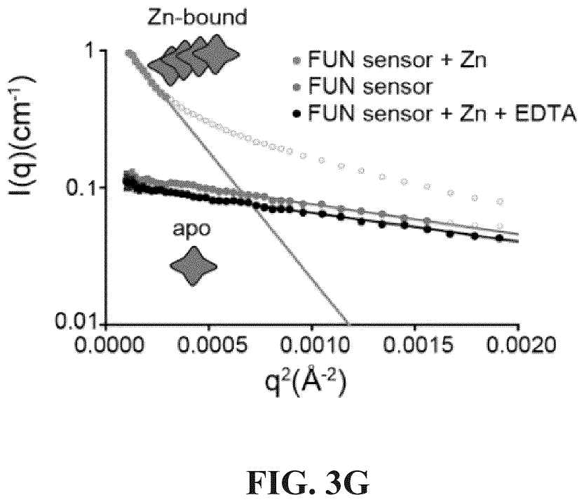

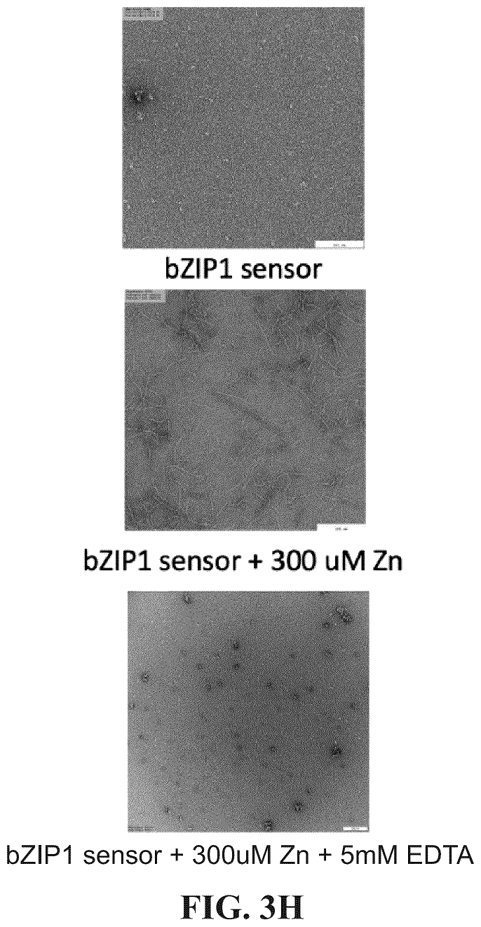

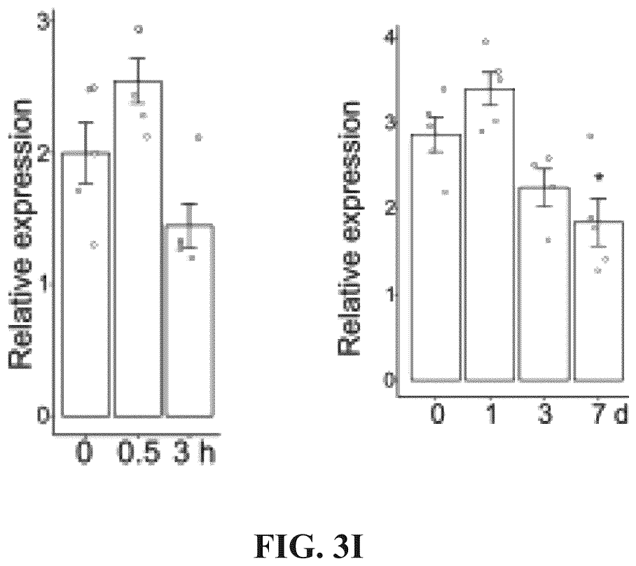

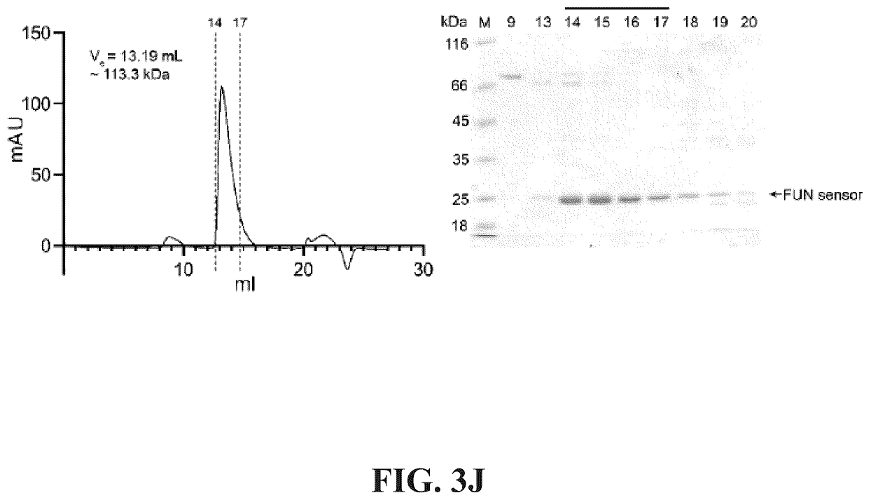

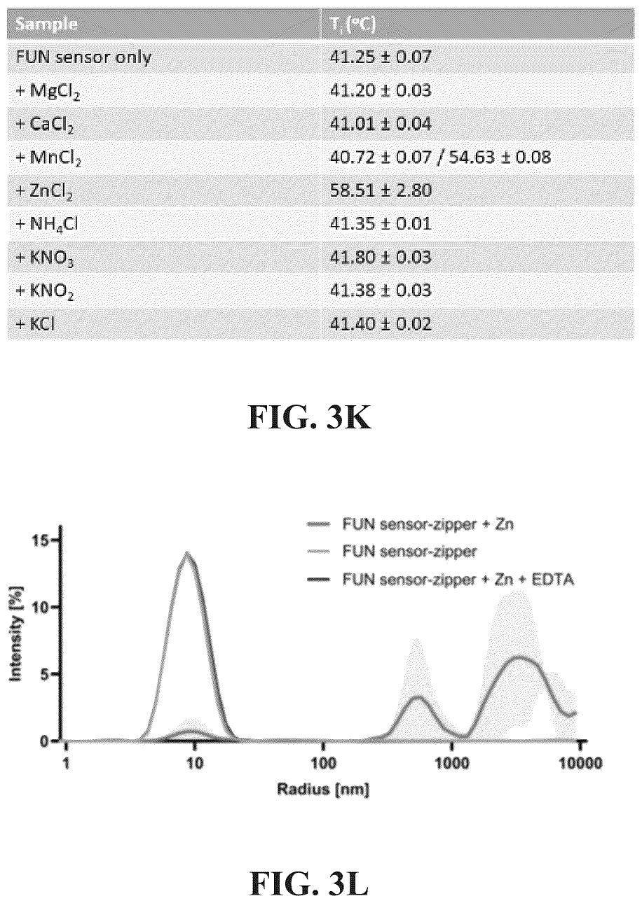

The FUN sensor domain had distant homology to metal binding proteins (Trepreau, J. et al. Structural basis for metal sensing by CnrX. J. Mol. Biol. 408, 766-779 (2011)) and since no transcriptional regulation of FUN was observed in nodules (FIG. 3I), it was hypothesized that the activity could be regulated at the protein level. To understand the mechanism, the FUN sensor domain was expressed and purified (FIG. 3J) and screened with common cellular metal ions and nitrogen compounds to see if these influenced the FUN sensor. It was found that both thermostability (nanoDSF; FIG. 3K) and molecular size by dynamic light scattering (DLS) (FIGS. 3A-3C) of FUN increased in the presence of zinc and manganese, whereas there were no changes in response to the other compounds tested. Dose-response experiments revealed that zinc increased the molecular size of the FUN sensor at low, physiologically relevant concentrations (3.9-7.8 μM) whereas only unnaturally high levels of manganese (2-4 mM) increased its size, showing that zinc was the relevant ligand (FIGS. 3A and 3B).

The changes induced by zinc were reversible when zinc was chelated using EDTA (FIG. 3D). Similar zinc sensitivity and reversibility with a protein containing both the DNA binding domain and sensor domain was also confirmed (FIG. 3L). Further investigation by small angle X-ray scattering experiments (SAXS) provided scattering data and pair distance-distribution functions (histograms of distances between pair of points within the structure) confirming that the FUN sensor shifted from a smaller molecular size into a larger oligomer form when zinc was present, and that this effect was reversible when removing zinc with EDTA (FIGS. 3E-3G).

The structure of the oligomeric form of the FUN sensor was investigated using electron microscopy. Negatively stained samples revealed that large filament structures formed when the FUN sensor was zinc-bound and that these filaments disassembled when zinc was removed using EDTA (FIG. 3H).

Together, these results showed that FUN bound low physiological concentrations of zinc, changing its oligomeric form into large filaments and that this process was dynamic and reversible, which could be a mechanism of regulating activity.

### Example 4: Zinc Regulation of FUN is Relevant In Vivo

The following example describes in vivo testing of zinc regulation of FUN.

### Materials and Methods

### Subcellular Localization Assay

The subcellular localization of the FUN-GFP construct described in Example 1 was examined in N. benthamiana (tobacco) leaves. Tobacco leaves that expressed pro35S:FUN-GFP protein were infiltrated with 500 μM mock, Mn, and Zn. Fluorescence images were taken by confocal microscope (Zeiss SP5) and nuclei were counted based on dots or homogenous distribution.

### Plant Lines and Growth Conditions

Plant lines and growth conditions were as described in Example 1.

### Bacterial Strains and Culture Conditions

The bacterial strains and culture conditions were as described in Example 3.

### Hairy Root Transformation

Hairy root transformation was as described in Example 1.

### Nodulation Assay

Nodulation assays were conducted as described in Example 1.

### Nitrogen Fixation Assay

Nitrogen fixation activity was quantified as described in Example 1.

### Leghemoglobin Content Assay

Leghemoglobin content was assayed as described in Example 1

### Transient Activation

The transient activation assay was as described in Example 2.

### Zinc Biosensors

The FRET-based Zn biosensors eCALWY (Lanquar, V., Grossmann, G., Vinkenborg, J. L., Merkx, M., Thomine, S., and Frommer, W. B. (2014). Dynamic imaging of cytosolic zinc in Arabidopsis roots combining FRET sensors and RootChip technology. New Phytol. 202: 198-208) and eCALWYnls were used to assay the response of nodule cells to Zn treatment. The biosensor eCALWYnls included a nuclear localization signal (nls).

### Expression Analysis

RNAseq was used to compare the expression of the putative zinc transporter genes Zip1 and Zip2 under normal and nitrate stress growth conditions. The original data was from the RNAseq analysis described in Example 3.

### Results

Zinc is a Second Messenger that Regulates FUN Activity

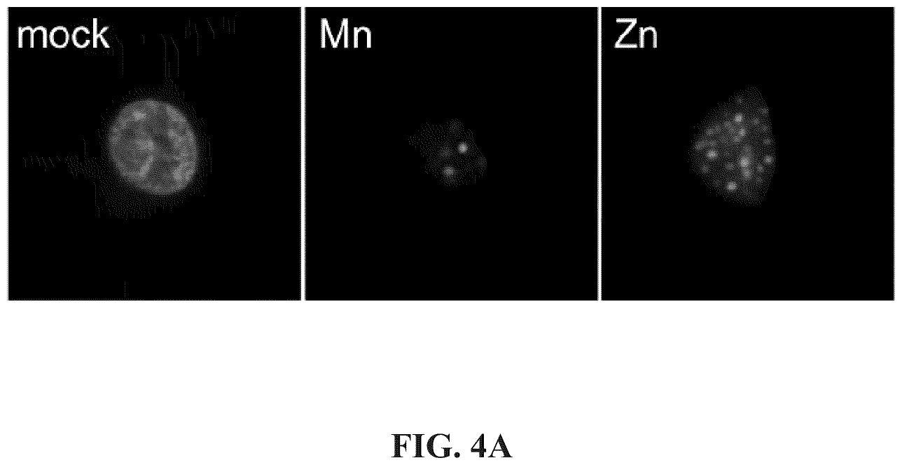

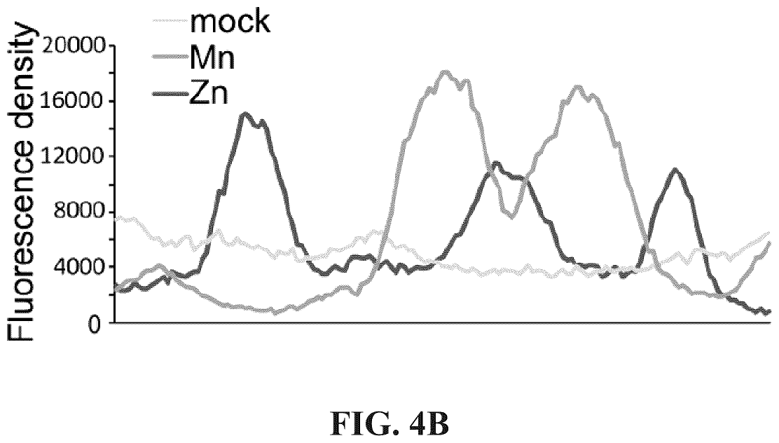

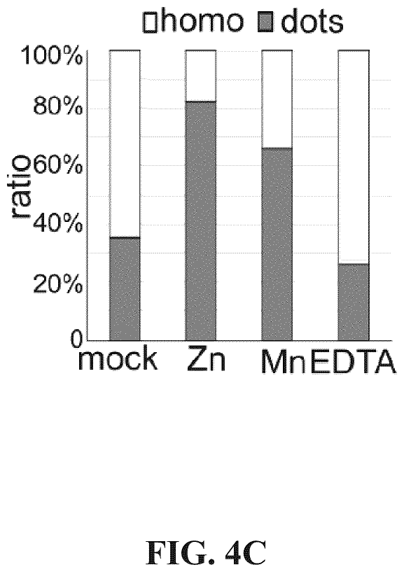

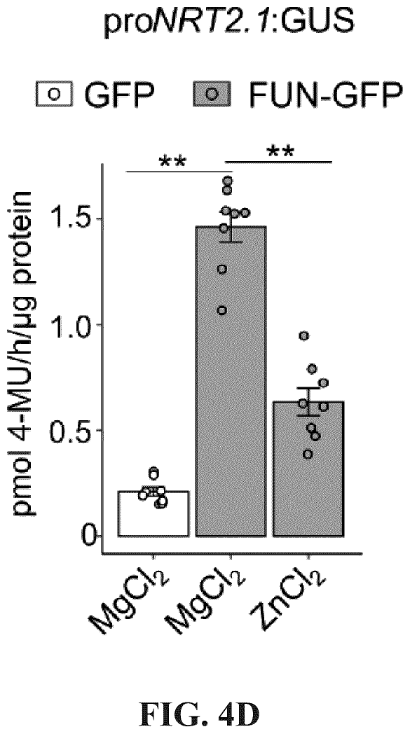

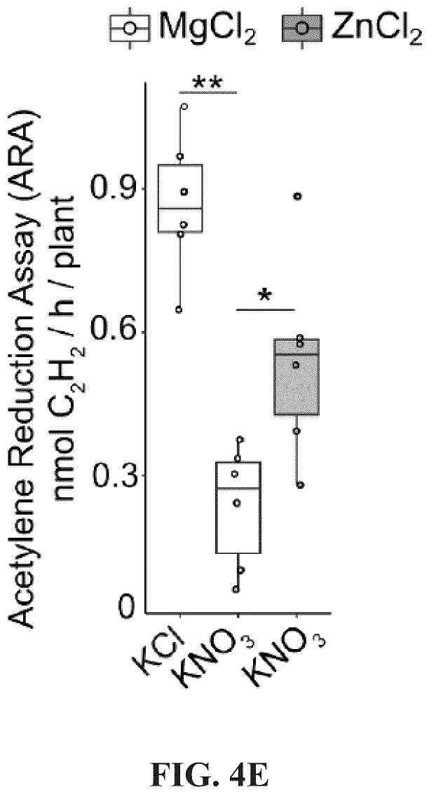

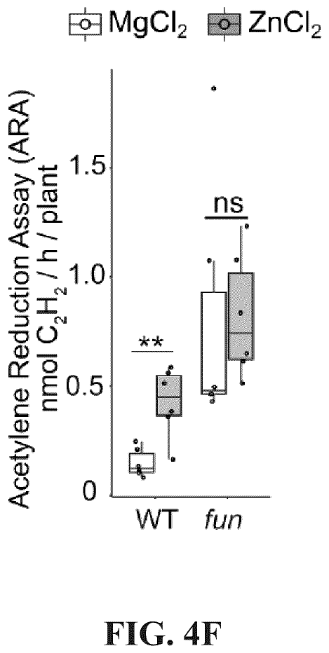

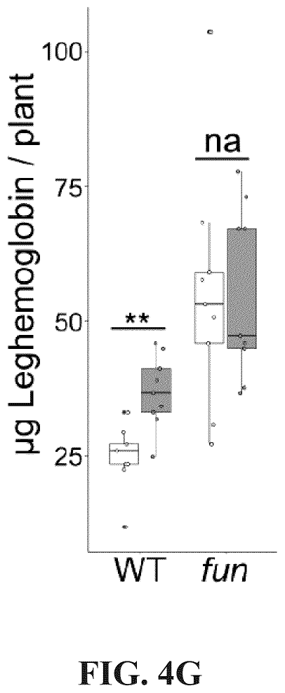

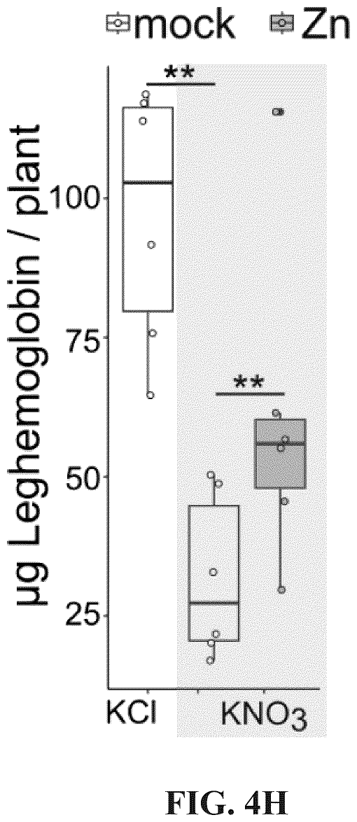

The identification of zinc-induced FUN filaments raised the possibility that this may play a role in modulating the activity of the protein. In particular, zinc infiltration triggered the alteration of nuclear fluorescence of FUN-GFP in N. benthamiana leaves. The subcellular localization assay results displaying altered fluorescence are shown in FIGS. 4A-4C, where the application of zinc facilitated the aggregation of FUN.

Using the NRT2.1 promoter as a readout for FUN activity, co-infiltration with zinc significantly reduced FUN activity relative to mock (MgCl₂) in N. benthamiana leaves (FIG. 4D). This indicated that the zinc-bound filamentous state of FUN is the inactive form of the protein. Given the fun mutant phenotypes, this indicated that zinc may act as a messenger linking nitrate with FUN activity and nodule regulation.

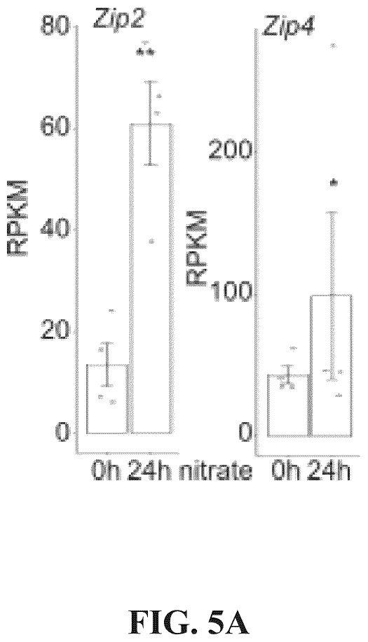

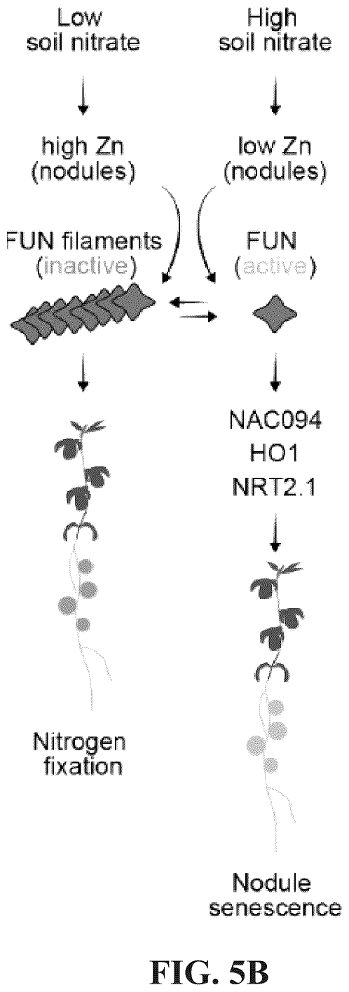

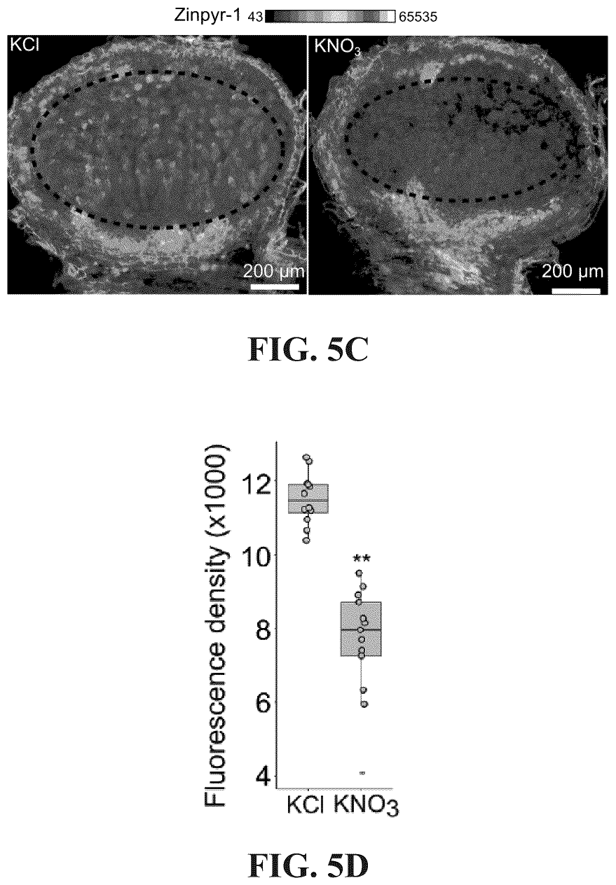

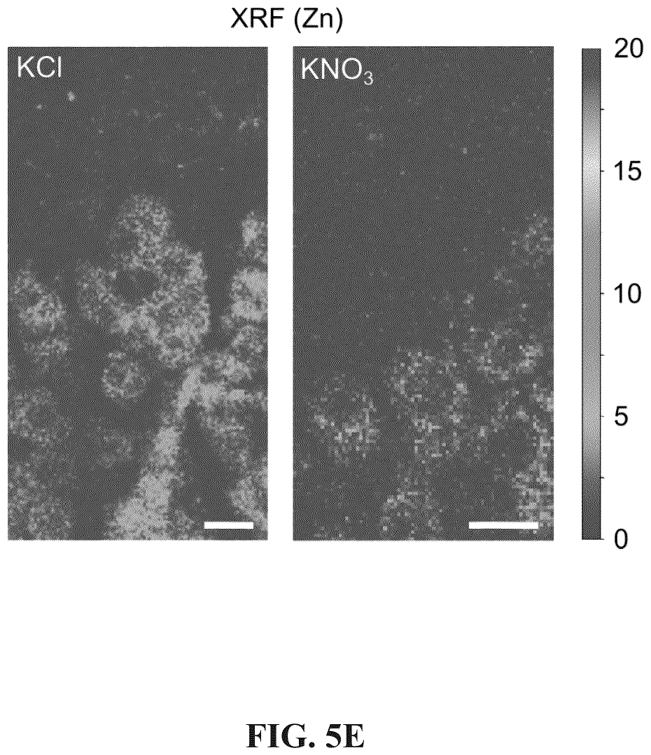

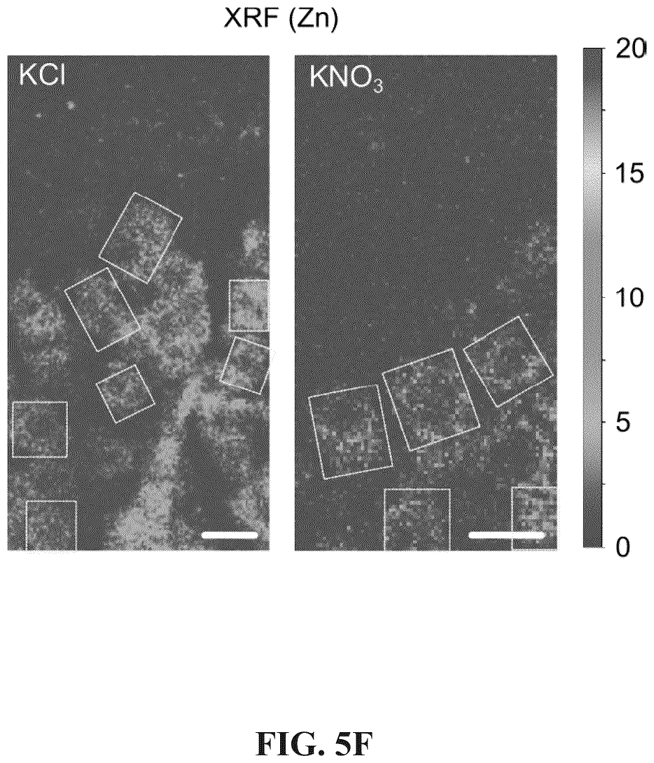

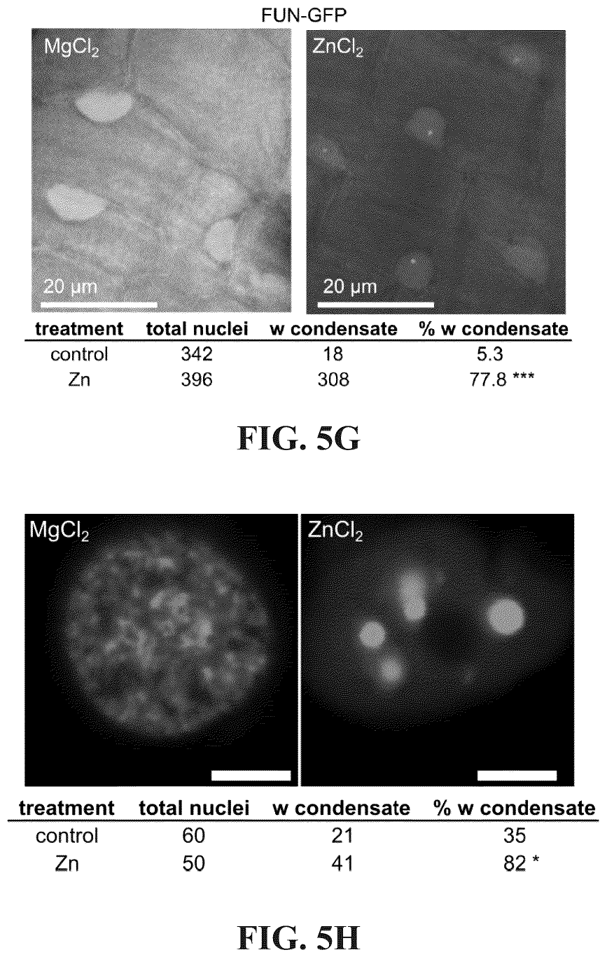

To test whether nitrate influenced cellular zinc levels, experiments were performed using the zinc-sensitive zinpyr-1 dye (Sinclair, S. A. et al. The use of the zinc-fluorophore, Zinpyr-1, in the study of zinc homeostasis in Arabidopsis roots. New Phytol. 174, 39-45 (2007)) to evaluate Lotus nodule sections from plants grown in nitrate-free conditions as well as nodules exposed to 10 mM KNO₃ for 24 hours (FIGS. 5C-5D). This revealed a marked reduction in zinc levels, particularly within the nitrogen fixation zone of nitrate treated nodules. Independent confirmation of this concentration reduction was obtained via micro-X-ray fluorescence microscopy conducted on sections of nodules treated with 10 mM KNO₃ for 24 hours, which showed ring-like distribution of zinc in infected cells associated with the symbiosome radial distribution and dense packaging (FIG. 5E-5F). Density measurements of 10 cells from each condition confirmed that zinc was reduced by half relative to untreated nodules (0.54±0.06; FIG. 5F). To confirm the in vivo relevance of zinc-dependent filamentation of FUN, a FUN-GFP construct was expressed in Lotus roots. FUN-GFP showed a disperse localization in the nucleus in the control condition (500 μM MgCl₂), whereas addition of zinc (500 μM ZnCl₂) triggered relocalization to distinct sub-nuclear condensates (FIG. 5G). Consistent with the impact of zinc on protein activity in N. benthamiana, a zinc-dependent increase in condensate frequency was also detected in leaves infiltrated with zinc alongside the FUN-GFP construct (FIG. 5H). Further confirming the link between nitrate, zinc and FUN activity, addition of 500 μM zinc significantly increased nodule function in nitrate-exposed WT plants, reproducing the phenotypes of fun knock-out mutants as determined by acetylene reduction (FIG. 4E) and Leghemoglobin content (FIG. 4H). This increase was dependent on the presence of FUN as no further increase in nodule function was observed in the fun mutant (FIGS. 4F and 4G). Finally, FIG. 5A shows the results of expression analysis of two putative zinc transporters, Zip2 and Zip4. Both Zip2 and Zip4 were induced in nodules after nitrate treatment.

Together, these results showed that alterations in zinc concentrations in response to soil nitrate were sufficient to alter FUN activity and thus the nitrogen fixation phenotype of the nodule.

### DISCUSSION

The genetic screen described in Example 1 identified a basic leucine zipper transcription factor, FUN, as a novel master regulator of nitrogen fixation in legumes. A sensor domain within FUN was identified as crucial for its activity and demonstrated that intracellular zinc levels determine protein activity via ligand-dependent protein filamentation. In Examples 2-4, it was shown that FUN formed inactive filaments under high zinc concentrations that act as a molecular reservoir from which active proteins can be released when zinc levels were lowered (FIG. 5J). Cellular zinc levels displayed an inverse relationship with nitrate, and it was shown that zinc acted as a second messenger to signal nitrate availability and control the transition between filamentous (inactive) and active states of the FUN protein.

In plants, it was demonstrated that altered zinc concentrations impacted the activity of the FUN protein and nodule function, acting to link soil nitrate supply to transcriptional modulation of nodule metabolism. It is thought that this post-translational regulation of FUN activity allows the plant to respond to a nitrate concentration gradient via a gradual decrease in zinc levels liberating greater quantities of active FUN to tune nodule function to the environment. The precise mechanism by which intracellular zinc concentrations are impacted by nitrate, e.g., via transporter regulation, organellar sequestration or cellular export, is still unknown. FUN is a transcription factor in the TGA family, whose members regulate a diverse array of important plant traits including nitrate uptake (Alvarez, J. M. et al. Systems approach identifies TGA1 and TGA4 transcription factors as important regulatory components of the nitrate response of Arabidopsis thaliana roots. Plant J. 80, 1-13 (2014), Ruffel, S. et al. Genome-wide analysis in response to nitrogen and carbon identifies regulators for root ArNRT2 transporters. Plant Physiol. 186, 696-714 (2021)), pathogen response (Kumar, S. et al. Structural basis of NPR1 in activating plant immunity. Nature 1-6 (2022)), and flower development (Maier, A. T., Stehling-Sun, S., Offenburger, S.-L. & Lohmann, J. U. The bZIP Transcription Factor PERIANTHIA: A Multifunctional Hub for Meristem Control. Front. Plant Sci. 2, 79 (2011)). Given the presence of the identified sensor domain within homologues of the TGA family, it is plausible that zinc, or other metal ions and metabolites, could provide similar graded responses to environmental stimuli, enabling a connection between the environment and plant development through metal ion signaling. Manipulation of metal ion accumulation or the responsiveness of protein filamentation to these metal ions may provide novel methods of optimizing these important plant traits.

Nitrogen fixation is an energy-demanding process requiring the provision of fixed carbon to symbiotic rhizobia. A regulated senescence program allows restriction of carbon supply to nodules and reprovisioning of nutrients to support plant growth and reproduction (Puppo, A. et al. Legume nodule senescence: roles for redox and hormone signaling in the orchestration of the natural aging process. New Phytol. 165, 683-701 (2005)). Recently, several NAC transcription factors have been shown to regulate pathways required for nodule senescence (Yu, H. et al. GmNAC039 and GmNAC018 activate the expression of cysteine protease genes to promote soybean nodule senescence. Plant Cell (2023) doi: 10.1093/plcell/koad129; Wang, L. et al. A transcription factor of the NAC family regulates nitrate-induced legume nodule senescence. New Phytol. (2023) doi: 10.1111/nph.18896). The identification of FUN as a new regulator of senescence-related processes through multiple pathways, including via NAC094, opens new avenues for fine-tuning these pathways to enhance tolerance of legumes to soil nitrate, and provides an opportunity to increase delivery of fixed nitrogen to agriculturally important crops. Importantly, the specificity of the identified pathway to nodule functional regulation ensures mutants do not show adverse impacts associated with other genetic pathways, such as nodule number regulation (Krusell, L., Madsen, L. H., Sato, S. & Aubert, G. Shoot control of root development and nodulation is mediated by a receptor-like kinase. Nature 420, 422-426 (2002), Nishimura, R. et al. HAR1 mediates systemic regulation of symbiotic organ development. Nature 420, 426-429 (2002), Huault, E. et al. Local and systemic regulation of plant root system architecture and symbiotic nodulation by a receptor-like kinase. PLoS Genet. 10, e1004891 (2014)) or nitrate acquisition and signaling (Lin, J.-S. et al. NIN interacts with NLPs to mediate nitrate inhibition of nodulation in Medicago truncatula. Nat Plants 4, 942-952 (2018), Misawa, F. et al. Nitrate transport via NRT2.1 mediates NIN-LIKE PROTEIN-dependent suppression of root nodulation in Lotus japonicus. Plant Cell 34, 1844-1862 (2022), Jiang, S. et al. NIN-like protein transcription factors regulate leghemoglobin genes in legume nodules. Science 374, 625-628 (2021)).

### Example 5: Identification of FUN and NAC094 Orthologues and Construction of Phylogenetic Trees

The following example describes the identification of FUN and NAC094 orthologues in other plant species, and the construction of phylogenetic trees using these sequences.

### Materials and Methods

### Identification of FUN and NAC094 Orthologues and Construction of Phylogenetic Trees

The FUN protein sequence was used as a BLAST query against target species. Similarly, the NAC094 protein sequence was used as a BLAST query against target species. Candidate BLAST hits were aligned to phylogenetic trees using Shoot.bio (Emms, D. M., Kelly, S. SHOOT: phylogenetic gene search and ortholog inference. Genome Biol 23, 85 (2022)).

The FUN orthologous protein sequences were SEQ ID NO: 1, SEQ ID NO: 2, SEQ ID NO: 3, SEQ ID NO: 4, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30.

The NAC094 orthologous protein sequences were SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, and SEQ ID NO: 73.

Protein sequences were aligned with MAFFT 7.490 and a tree was constructed using FastTree 2.1.11. The tree was visualized using iTOL 6.7.3 (Letunic, I. & Bork, P. Interactive Tree Of Life (iTOL) v5: an online tool for phylogenetic tree display and annotation. Nucleic Acids Res. 49, W293-W296 (2021)).

### Results

Phylogenetic analysis indicated that FUN is highly conserved in legumes, with legumes carrying both a FUN and FUN-like paralog in the PAN orthogroup (FIG. 6A). A legume duplication of FUN resulted in two copies of the gene in L. japonicus: FUN, which is expressed in the nodule and described in Example 1, and FUN-like, which is not expressed in the nodule and has non-legume orthologs. There was a second duplication in Glycine max with two orthologs for FUN and two orthologs for FUN-like.

The FUN orthologues were those within a phylogenetic clade with Lotus FUN (LotjaGi2glv0279100; SEQ ID NO: 1) and Glycine max (soybean) FUNa (Glyma.02G097900; SEQ ID NO: 8) and FUNb (Glyma.01G084200; SEQ ID NO: 9). The closest non-legume orthogroup member was Arabidopsis PAN (AT1G68640.1; SEQ ID NO: 4). Orthology was further confirmed by gene expression within nodules as determined by RNAseq (data not shown).

To assess if the Glycine max FUN orthologs also exhibited nodule specific expression, expression levels of the two orthologs, FUNa and FUNb, were measured in various soybean tissues (FIG. 6B). Indeed, both orthologs were predominantly expressed in symbiotic nodules. These observations suggested that the function of FUN may be conserved in soybean and other legumes.

FUN paralogues that did not function in nodule regulation were those within a phylogenetic clade with Lotus FUN-like (LotjaGi5glv0341400; SEQ ID NO: 83) and soybean FUN-like (Glyma.20G113600 (SEQ ID NO: 7) and Glyma.10G276100 (SEQ ID NO: 6)).

A phylogenetic tree for the NAC-domain containing protein Nac094 was also constructed to identify legume and non-legume orthologues and paralogues (FIG. 7). Nac094 (LotjaGi2glv0259200; SEQ ID NO: 31) was orthologous to soybean Glyma.19G021900.1 (SEQ ID NO: 42) and Glyma.13G063300.1 (SEQ ID NO: 41).

### Example 6:Fun Mutants in Soybean and Cowpea Exhibit Enhanced Nitrogen Fixation

The following example describes the generation and characterization of fun mutants in soybean and cowpea. Specifically, experiments assessing nitrogen fixation activity under varied stress conditions and yield performance of fun mutants in soybean and cowpea are described.

### Materials and Methods

### Plant Materials and Growth Conditions

Glycine max (soybean) and Vigna unguiculata (cowpea) lines are used for Agrobacterium transformation and regeneration of CRISPR fun knockout mutants.

### Nitrogen Fixation Activity

Nitrogen fixation is assessed using the methods of Example 1.

### Generation of Plant Expression Vectors

Expression constructs are generated to express CRISPR/Cas and multiple guide RNAs targeting the Fun gene coding sequences in G. max (soybean) and V. unguiculata (cowpea).

### Plant Transformation and Regeneration

Plant transformation and regeneration is done using standard methods for soybean and cowpea.

### Yield Performance

Yield performance is assessed using standard methods for soybean and cowpea in diverse conditions from low to high nitrogen application. Yield performance is also assessed in scenarios with companion crops.

### Results

### Improved Nitrogen Fixation in Soybean and Cowpea Increases Yield

To determine if the function of the Lotus Fun gene is conserved in legumes, CRISPR knockouts of the orthologous FUN genes in soybean (FIG. 6A) and cowpea will be created. Multiple knockout lines will be selected and bred to generate homozygous fun mutants. Once homozygous lines are generated, fun mutants in soybean and cowpea will be evaluated for nitrogen fixation activity in restrictive nitrate conditions. Nodule phenotypes and nitrogen fixation activity will be assayed similarly to the Lotus fun mutant. If the Lotus Fun gene function is conserved in soybean and cowpea, fun mutants in both species will exhibit a pink (active) nodule phenotype and increased nitrogen fixation activity in restrictive nitrate conditions. Further, to determine if the absence of a functional Fun gene enhances nitrogen fixation activity in other stress environments, rates of nitrogen fixation for fun mutants in Lotus, soybean, and cowpea will be evaluated under drought, heat stress, and waterlogging conditions.

Soybean and cowpea varieties with enhanced nitrogen fixation activity under stress conditions could sustainably enhance yields of both crops. Promising fun mutant lines in soybean and cowpea identified from the stress assays described above will be evaluated for field performance. Yield characteristics of fun mutants in soybean and cowpea will be assessed in the field to determine if enhanced nitrogen fixation translates to increased yield.

### Example 7: Engineered FUN Variants Improve Nitrogen Fixation Characteristics in Legumes

The following example describes the structure-function characterization of FUN for engineering and characterization of FUN variants in Lotus and target crops.

### Materials and Methods

### Plant Materials and Growth Conditions

The Lotus fun mutant described in Example 1 and the fun mutants identified in soybean and cowpea in Example 6 will be used for Agrobacterium transformation and regeneration of engineered FUN variants. Growth conditions are as described in Examples 1 and 6.

### Experimental Techniques

The experimental techniques are as described in any of Examples 1-6.

### Results

### Structure-Based Engineering of FUN Variants

Structural characterization of the fun mutant protein and its alleles will identify regions of the protein critical for function. Coupled with computational modeling, this structural analysis will pinpoint key residues in the FUN protein for engineering novel variants that further enhance nitrogen fixation characteristics. Engineered FUN protein variants will be introduced into Lotus and evaluated for nitrogen fixation characteristics.

### Function of Engineered FUN Variants is Conserved in Soybean and Cowpea

To assess if structure-based engineering of FUN translates to improvements in crop yield, mutations corresponding to the engineered FUN Lotus variants will be introduced into soybean and cowpea. Performance of engineered FUN soybean and cowpea lines will be evaluated under various stress environments and in the field.

### Example 8: FUN Sensor Domain Mechanism is Conserved in TGA Transcription Factors

The following example describes the structural characterization of TGA type transcription factors, which share a conserved domain structure with FUN. Further, experiments assessing the modulation of TGA sensor domain conformation by metal ion ligands and the identification of nitrate-responsive metal ion transporters are described.

### Materials and Methods

The experimental techniques are as described in any of Examples 1-7.

### Results

### Sensor Domain Filamentation is a Conserved Feature of TGA Transcription Factors

Lotus FUN is a member of the TGA family of transcriptional regulators, which play broad roles in plant development, immunity, and nitrate signaling. TGA family members all share a conserved domain structure with a DNA binding and sensor domain. To determine if sensor domain filamentation is conserved across the TGA family of transcription factors, sensor domain structures of representative TGA family members will be determined. The ability of metal ion ligands to induce sensor domain multimerization will also be investigated. If metal ion ligands can regulate sensor domain complex formation and activity similar to FUN, then modulation of the entire family could be achieved through metal ion treatments to target a variety of pathways relevant to crop improvement.

### Identification of Nitrate Responsive Metal Ion Transporters to Modulate Nitrogen Fixation

To identify metal ion transporter genes modulated by nitrate, differential gene expression in Lotus will be assayed for three sets of conditions: nitrate treatment vs mock, zinc treatment vs mock, and co-treatment with nitrate and zinc vs mock.

Differentially expressed candidate genes will be further evaluated for expression, activity and zinc concentration in nodules in response to nitrate conditions. Lotus knock-out lines for identified metal ion transporter genes will be acquired or created and evaluated for improved nitrogen fixation characteristics. Identification of these genes will provide mechanistic insight into zinc modulation by nitrate and additional means for modulating nitrogen fixation.

### Example 9: FUN Effects on Heat and Drought Tolerance

The following example describes the generation and characterization of fun mutants in Medicago and Lotus, as well as experiments assessing nitrogen fixation activity under heat and drought conditions and yield performance of fun mutants in Medicago and Lotus are described

### Materials and Methods

The experimental techniques are as described in any of Examples 1-8.

### Results

### Manipulation of FUN Improves Drought Tolerance

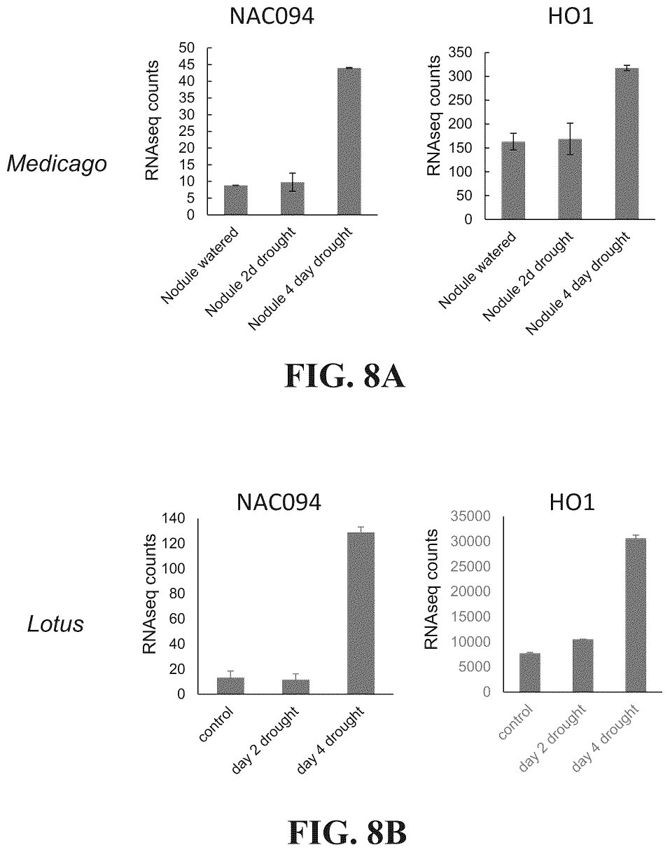

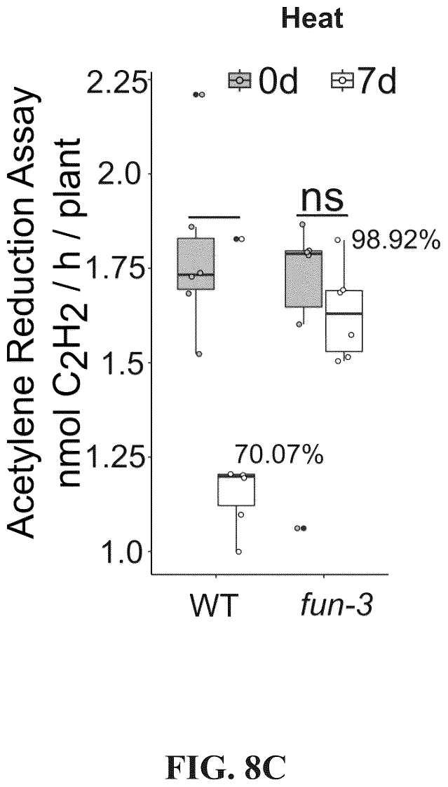

RNAseq revealed that NAC094 and HO1 are strongly upregulated after 4 days of drought in both Medicago and Lotus (FIG. 8A). To determine if FUN regulation of NAC094 and/or HO1 plays a role in drought tolerance, fun mutants are generated in Medicago and Lotus, and tested under drought conditions. Drought tolerance of the plants is assessed.

### Manipulation of FUN Improves Heat Tolerance

fun mutant plants show elevated nitrogen fixation activity after seven days of heat stress, as quantified using an acetylene reduction assay (ARA)(FIG. 8C). To determine if manipulation of FUN can improve heat tolerance, fun mutants are tested under heat conditions. Heat tolerance of the plants is assessed.

What is claimed is:

1. A genetically modified plant or part thereof comprising one or more genetic alterations that result in decreased activity or expression of a FUN protein as compared to the activity or expression of a FUN protein in a control plant grown under the same conditions, wherein the FUN protein comprises a polypeptide with at least 70% identity, at least 80% identity, at least 90% identity, at least 95% identity, or at least 99% identity to a protein selected from the group of SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, and SEQ ID NO: 82.

2. The genetically modified plant or part thereof of claim 1, wherein the FUN protein comprises SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, or SEQ ID NO: 82.

3. A genetically modified plant or part thereof comprising one or more genetic alterations that result in decreased activity or expression of one or more of a NRT3.1 protein, a bZIP28 protein, a NAC-domain containing protein, a HO1 protein, a NRT2.1 protein, or an AS1 protein as compared to the activity of a NRT3.1 protein, a bZIP28 protein, a NAC-domain containing protein, a HO1 protein, a NRT2.1 protein, or an AS1 protein in a control plant grown under the same conditions, wherein the protein is the NRT3.1 protein, and wherein the NRT3.1 protein comprises a polypeptide with at least 70% identity, at least 80% identity, at least 90% identity, at least 95% identity, or at least 99% identity to SEQ ID NO: 74; wherein the protein is the bZIP28 protein, and wherein the bZIP28 protein comprises a polypeptide with at least 70% identity, at least 80% identity, at least 90% identity, at least 95% identity, or at least 99% identity to SEQ ID NO: 75; wherein the protein is the NAC-domain containing protein, and wherein the NAC-domain containing protein comprises a polypeptide with at least 70% identity, at least 80% identity, at least 90% identity, at least 95% identity, or at least 99% identity to a protein selected from the group of SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, and SEQ ID NO: 73; wherein the protein is the HO1 protein, and wherein the HO1 protein comprises a polypeptide with at least 70% identity, at least 80% identity, at least 90% identity, at least 95% identity, or at least 99% identity to SEQ ID NO: 77; wherein the protein is the NRT2.1 protein, and wherein the NRT2.1 protein comprises a polypeptide with at least 70% identity, at least 80% identity, at least 90% identity, at least 95% identity, or at least 99% identity to SEQ ID NO: 78; or wherein the protein is the AS1 protein, and wherein the AS1 protein comprises a polypeptide with at least 70% identity, at least 80% identity, at least 90% identity, at least 95% identity, or at least 99% identity to SEQ ID NO: 79.

4. The genetically modified plant or part thereof of claim 3, wherein the NRT3.1 protein comprises SEQ ID NO: 74; wherein the bZIP28 protein comprises SEQ ID NO: 75; wherein the NAC-domain containing protein comprises SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, or SEQ ID NO: 73; wherein the HO1 protein comprises SEQ ID NO: 77; wherein the NRT2.1 protein comprises SEQ ID NO: 78, or wherein the AS1 protein comprises SEQ ID NO: 79.

5. A genetically modified plant or part thereof comprising one or more genetic alterations that result in decreased activity or expression of one or more of a FUN protein, a FUN-like protein, a NRT3.1 protein, a bZIP28 protein, a NAC-domain containing protein, a HO1 protein, a NRT2.1 protein, or an AS1 protein as compared to the activity or expression of a FUN protein, a FUN-like protein, a NRT3.1 protein, a bZIP28 protein, a NAC-domain containing protein, a HO1 protein, a NRT2.1 protein, or an AS1 protein in a control plant grown under the same conditions, wherein the FUN protein, the FUN-like protein, the NRT3.1 protein, the bZIP28 protein, the NAC-domain containing protein, the HO1 protein, the NRT2.1 protein, or the AS1 protein is selected from the group of a polypeptide with at least 70% identity, at least 80% identity, at least 90% identity, at least 95% identity, or at least 99% identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, or SEQ ID NO: 84, and wherein the FUN protein, the FUN-like protein, the NRT3.1 protein, the bZIP28 protein, the NAC-domain containing protein, the HO1 protein, the NRT2.1 protein, or the AS1 protein has enhanced expression in a root nodule absent the one or more genetic alterations.

6. The genetically modified plant or part thereof of any one of claims 1-5, wherein the decrease is at least 10%, at least 20%, at least 30%, at least 40%, at least 50%, at least 60%, at least 70%, at least 80%, at least 90%, or at least 100%, optionally wherein the decrease is due to knock-out of a gene for the protein, introduction of a premature stop codon in the coding sequence of the gene for the protein, RNAi silencing, knock-out of a domain of the protein, introduction of a transcriptional repressor protein binding site, or knock-out of a binding site in the promoter region of the gene and/or the genetic alterations comprise knock-out of a gene for the protein, introduction of a premature stop codon in the coding sequence of the gene for the protein, RNAi silencing, knock-out of a domain of the protein, introduction of a transcriptional repressor protein binding site, or knock-out of a binding site in the promoter region of the gene, preferably the binding site is a transcriptional activator protein binding site or a TATA-box.

7. The genetically modified plant or part thereof of any one of claims 1-6, wherein the growth conditions comprise a moderate nitrate level, a high nitrate level, or a nitrate level around the plant that reduces or suppresses nitrogen fixation, optionally wherein the nitrate level is between about 10 mM and about 250 mM nitrate or comprises at least about 10 mM nitrate, at least about 20 mM nitrate, at least about 30 mM nitrate, at least about 40 mM nitrate, at least about 50 mM nitrate, at least about 100 mM nitrate, at least about 150 mM nitrate, at least about 200 mM nitrate, or at least about 250 mM nitrate.

8. The genetically modified plant or part thereof of claim 7, wherein the genetically modified plant has increased nitrogen fixation as compared to the control plant when grown under the same growth conditions, optionally wherein the nitrogen fixation is increased at least 10%, at least 20%, at least 30%, at least 40%, at least 50%, at least 60%, at least 70%, at least 80%, at least 90%, at least 100%, at least 150%, at least 200%, at least 300%, at least 400%, or at least 500%.

9. The genetically modified plant or part thereof of any one of claims 1-8, wherein the plant forms nodules, and wherein the number of nodules is increased, hemoglobin content is increased, or the acetylene reduction assay (ARA) activity is increased compared to the control plant when grown under the same conditions.

10. A method of cultivating a genetically altered plant with increased nitrogen fixation under conditions comprising a nitrate level around the plant roots that suppresses nitrogen fixation, comprising:
a) providing the genetically altered plant, wherein the plant or a part thereof comprises one or more genetic alterations that result in decreased activity or expression of a FUN protein, a NRT3.1 protein, a bZIP28 protein, a NAC-domain containing protein, a HO1 protein, a NRT2.1 protein, or an AS1 protein, or any combination thereof as compared to an activity or expression of a FUN protein, a NRT3.1 protein, a bZIP28 protein, a NAC-domain containing protein, a HO1 protein, a NRT2.1 protein, or an AS1 protein in a control plant grown under the same conditions, and wherein the one or more genetic alterations reduce the nitrate level suppression of nitrogen fixation; and
b) cultivating the genetically altered plant under the nitrate level around the plant roots, wherein the genetically modified plant has increased nitrogen fixation as compared to the control plant grown under the same conditions.

11. The genetically modified plant or part thereof produced by the method of claim 10, wherein the number of nodules is increased or the hemoglobin content is increased compared to the control plant when grown under the same growth conditions, and wherein increased nitrogen fixation is measured using a method selected from the group of measuring the number of pink nodules per plant as compared to a control plant, measuring the amount of acetylene (C₂H₂) reduced to ethylene (C₂H₄) per hour (acetylene reduction assay (ARA)) as compared to a control plant, and measuring the micrograms of hemoglobin per plant as compared to a control plant.

12. A method of cultivating a genetically altered plant able to fix nitrogen when grown in nitrogen-fertilized conditions, comprising:
a) providing the genetically altered plant, wherein the plant or a part thereof comprises one or more genetic alterations that result in decreased activity or expression of a FUN protein, a NRT3.1 protein, a bZIP28 protein, a NAC-domain containing protein, a HO1 protein, a NRT2.1 protein, or an AS1 protein, or any combination thereof as compared to an activity or expression of a FUN protein, a NRT3.1 protein, a bZIP28 protein, a NAC-domain containing protein, a HO1 protein, a NRT2.1 protein, or an AS1 protein in a control plant grown under the same conditions, and wherein the one or more genetic alterations reduce the nitrate level suppression of nitrogen fixation;
b) cultivating the plant under conditions comprising a standard nitrate level around the plant roots; and
c) applying nitrogen fertilizer, thereby generating conditions comprising a nitrate level around the plant roots that suppresses nitrogen fixation, wherein the genetically modified plant has increased nitrogen fixation as compared to the control plant grown under the same conditions.

13. The method of claim 12, wherein the genetically altered plant is grown in an intercropping system with a plant that does not fix nitrogen or in a sequential system after a plant that does not fix nitrogen.

14. A method of delaying nodule senescence, comprising:
a) providing a genetically altered plant, wherein the plant or a part thereof comprises one or more genetic alterations that result in decreased activity or expression of a FUN protein, a NRT3.1 protein, a bZIP28 protein, a NAC-domain containing protein, a HO1 protein, a NRT2.1 protein, or an AS1 protein, or any combination thereof as compared to an activity or expression of a FUN protein, a NRT3.1 protein, a bZIP28 protein, a NAC-domain containing protein, a HO1 protein, a NRT2.1 protein, or an AS1 protein in a control plant grown under the same conditions, and wherein the one or more genetic alterations delay nodule senescence; and
b) cultivating the genetically altered plant under stress conditions, wherein the genetically altered plant has delayed nodule senescence as compared to the control plant grown under the same conditions.

15. The method of claim 14, wherein the stress conditions are selected from the group of a moderate nitrate level, a high nitrate level, a nitrate level around the plant that promotes nodule senescence, a moderate heat level, a high heat level, a heat level around the plant that promotes nodule senescence, a moderate water deficit level, a high water deficit level, a water deficit level around the plant that promotes nodule senescence, a moderate waterlogging level, a high waterlogging level, or a waterlogging level around the plant that promotes nodule senescence.

16. A method of inducing filamentation of a FUN protein, comprising:
a) providing the FUN protein; and
b) increasing an amount of zinc or manganese in an environment of the FUN protein, wherein the increased amount of zinc or manganese induces filamentation as compared to the control FUN protein in an environment without the increased amount of zinc or manganese, optionally wherein the filamentation is induced under high nitrate conditions, and/or the method is performed in vitro.

17. A method of inducing filamentation, comprising:
a) providing a plant comprising a FUN protein; and
b) cultivating the plant under increased zinc or manganese conditions, wherein filamentation of the FUN protein in the plant is induced as compared to a FUN protein in a control plant grown under conditions without increased zinc or manganese, optionally wherein the plant comprises a genetic alteration, and/or wherein the filamentation is induced under high nitrate conditions.

18. The method of claim 16 or claim 17, wherein the genetic alteration decreases the activity of the FUN protein without eliminating the activity of the FUN protein, and/or wherein the induction of filamentation results in increased nitrogen fixation in the genetically altered plant as compared to the control plant grown under the same conditions or reduces the activity or inactivates the FUN protein.

19. A method of tuning nodule function to the amount of available nitrogen in the soil, comprising:
a) providing a genetically altered plant comprising a FUN protein with altered activation by nitrate; and
b) cultivating the genetically altered plant under nitrate concentration conditions, wherein the genetically altered plant has reduced activity or expression of FUN and/or reduced active form of FUN as compared to a WT plant grown under the same nitrate conditions, optionally wherein
(i) altering FUN protein activation by nitrate comprises downregulating FUN, reducing FUN activity, knocking out FUN by mutation, knocking down FUN expression, knocking out or disrupting promoter elements of FUN, or a combination thereof;
(ii) altering FUN protein activation by nitrate comprises manipulating a level of environmental or cellular zinc or manganese, wherein this manipulation results in the FUN protein being maintained in inactive filament form; or
(iii) altering FUN protein activation by nitrate comprises genetically modifying the FUN protein sequence to alter sensitivity to zinc or manganese.

20. A method of making a genetically altered plant with increased nitrogen fixation under conditions comprising a nitrate level around the plant roots that suppresses nitrogen fixation, comprising introducing into the plant or a part thereof one or more genetic alterations that decrease activity or expression of a FUN protein as compared to the activity or expression of a FUN protein in a control plant grown under the same conditions.

21. A method of making a genetically altered plant with increased nitrogen fixation under conditions comprising a nitrate level around the plant roots that suppresses nitrogen fixation, comprising introducing into the plant or a part thereof one or more genetic alterations that decrease activity or expression of one or more of a NRT3.1 protein, a bZIP28 protein, a NAC-domain containing protein, a HO1 protein, a NRT2.1 protein, or an AS1 protein as compared to the activity or expression of a NRT3.1 protein, a bZIP28 protein, a NAC-domain containing protein, a HO1 protein, a NRT2.1 protein, or an AS1 protein in a control plant grown under the same conditions.

22. A method of making a genetically altered plant with increased nitrogen fixation under conditions comprising a nitrate level around the plant roots that suppresses nitrogen fixation, comprising introducing into the plant or a part thereof one or more genetic alterations that decrease activity or expression of one or more of a FUN protein, a FUN-like protein, a NRT3.1 protein, a bZIP28 protein, a NAC-domain containing protein, a HO1 protein, a NRT2.1 protein, or an AS1 protein as compared to the activity or expression of a FUN protein, a FUN-like protein, a NRT3.1 protein, a bZIP28 protein, a NAC-domain containing protein, a HO1 protein, a NRT2.1 protein, or an AS1 protein in a control plant grown under the same conditions.

23. A method of making the genetically modified plant or part thereof of any one of claims 1-9, comprising: introducing a genetic alteration to the plant cell that reduces or knocks out activity or expression of a FUN protein, a FUN-like protein, a NRT3.1 protein, a bZIP28 protein, a NAC-domain containing protein, a HO1 protein, a NRT2.1 protein, or an AS1 protein, wherein the genetic alteration comprises a first nucleic acid sequence able to reduce or knock out a second nucleic acid sequence encoding a FUN protein, a FUN-like protein, a NRT3.1 protein, a bZIP28 protein, a NAC-domain containing protein, a HO1 protein, a NRT2.1 protein, or an AS1 protein operably linked to a promoter, and wherein the genetically altered plant is selected from one or more of the group consisting of alfalfa, Bambara groundnut, bean (e.g., kidney beans, black beans, etc.), black currant, chickpea, clover, cowpea, forage legumes, legume trees, lentil, lotus, lupin, Medicago spp., pea, peanut, pigeon pea, soybean, Parasponia, alder trees, and elm trees.

24. A method of making the genetically modified plant or part thereof of any one of claims 1-17, comprising genetically modifying the plant cell by transforming the plant cell with one or more gene editing components that target an endogenous nuclear genome sequence encoding a FUN protein, a FUN-like protein, a NRT3.1 protein, a bZIP28 protein, a NAC-domain containing protein, a HO1 protein, a NRT2.1 protein, or an AS1 protein, wherein the endogenous nuclear genome sequence or a part thereof is knocked out, wherein the one or more gene editing components comprise a ribonucleoprotein complex that targets the nuclear genome sequence; a vector comprising a TALEN protein encoding sequence, wherein the TALEN protein targets the nuclear genome sequence; a vector comprising a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence.

25. An expression vector or isolated DNA molecule comprising (i) one or more nucleotide sequences encoding a FUN protein, a FUN-like protein, a HO1 protein, a NAC-domain containing protein, a bZIP28 protein, a NRT2.1 protein, a NRT3.1 protein, an AS1 protein, or a combination thereof, wherein the one or more nucleotide sequences are operably linked to at least one expression control sequence, (ii) one or more nucleotide sequences able to reduce or knock out a nucleic acid sequence encoding a FUN protein, a FUN-like protein, a HO1 protein, a NAC-domain containing protein, a bZIP28 protein, a NRT2.1 protein, a NRT3.1 protein, an AS1 protein, or a combination thereof, wherein the one or more nucleotide sequences are operably linked to at least one expression control sequence, or (iii) one or more nucleotide sequences comprising a mutation in a gene for a FUN protein, a FUN-like protein, a HO1 protein, a NAC-domain containing protein, a bZIP28 protein, a NRT2.1 protein, a NRT3.1 protein, an AS1 protein, or a combination thereof, wherein the mutation reduces or knocks out the activity or expression of the protein and the one or more nucleotide sequences are operably linked to at least one homologous nucleic acid sequence that hybridizes adjacent to the mutation site in the gene.

26. The expression vector or isolated DNA molecule of claim 25, wherein the protein is a FUN protein, and wherein the FUN protein comprises a polypeptide with at least 70% identity, at least 80% identity, at least 90% identity, at least 95% identity, or at least 99% identity to a protein selected from the group of SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, and SEQ ID NO: 82; wherein the FUN protein comprises SEQ ID NO: 1, SEQ ID NO: 2, SEQ ID NO: 3, SEQ ID NO: 5, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 80, SEQ ID NO: 81, and SEQ ID NO: 82; or wherein the FUN protein comprises SEQ ID NO: 1, SEQ ID NO: 8, or SEQ ID NO: 9;
wherein the protein is a FUN-like protein, and wherein the FUN-like protein comprises a polypeptide with at least 70% identity, at least 80% identity, at least 90% identity, at least 95% identity, or at least 99% identity to a protein selected from the group of SEQ ID NO: 4, SEQ ID NO: 6, SEQ ID NO: 7, SEQ ID NO: 83, and SEQ ID NO: 84; wherein the FUN-like protein comprises SEQ ID NO: 4, SEQ ID NO: 6, SEQ ID NO: 7, SEQ ID NO: 83, or SEQ ID NO: 84; and/or
wherein the protein is the NRT3.1 protein, and wherein the NRT3.1 protein comprises a polypeptide with at least 70% identity, at least 80% identity, at least 90% identity, at least 95% identity, or at least 99% identity to SEQ ID NO: 74; wherein the protein is the bZIP28 protein, and wherein the bZIP28 protein comprises a polypeptide with at least 70% identity, at least 80% identity, at least 90% identity, at least 95% identity, or at least 99% identity to SEQ ID NO: 75; wherein the protein is the NAC-domain containing protein, and wherein the NAC-domain containing protein comprises a polypeptide with at least 70% identity, at least 80% identity, at least 90% identity, at least 95% identity, or at least 99% identity to a protein selected from the group of SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, and SEQ ID NO: 73; wherein the protein is the HO1 protein, and wherein the HO1 protein comprises a polypeptide with at least 70% identity, at least 80% identity, at least 90% identity, at least 95% identity, or at least 99% identity to SEQ ID NO: 77; wherein the protein is the NRT2.1 protein, and wherein the NRT2.1 protein comprises a polypeptide with at least 70% identity, at least 80% identity, at least 90% identity, at least 95% identity, or at least 99% identity to SEQ ID NO: 78; or wherein the protein is the AS1 protein, and wherein the AS1 protein comprises a polypeptide with at least 70% identity, at least 80% identity, at least 90% identity, at least 95% identity, or at least 99% identity to SEQ ID NO: 79; wherein the NRT3.1 protein comprises SEQ ID NO: 74; wherein the bZIP28 protein comprises SEQ ID NO: 75; wherein the NAC-domain containing protein comprises SEQ ID NO: 76, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, or SEQ ID NO: 73; wherein the HO1 protein comprises SEQ ID NO: 77; wherein the NRT2.1 protein comprises SEQ ID NO: 78, or wherein the AS1 protein comprises SEQ ID NO: 79; or wherein the NAC-domain containing protein comprises SEQ ID NO: 31, SEQ ID NO: 41, or SEQ ID NO: 42.

27. A bacterial cell or an Agrobacterium cell comprising the expression vector or isolated DNA molecule of claim 25 or claim 26.

28. A genetically modified plant, plant part, plant cell, or seed comprising the expression vector or isolated DNA molecule of claim 25 or claim 26.

29. A kit comprising the expression vector or isolated DNA molecule of claim 25 or claim 26 or the bacterial cell or the Agrobacterium cell of claim 27.

30. A method of increasing nitrogen fixation, delaying nodule senescence, or inducing FUN filamentation in a plant, comprising: (a) introducing a genetic alteration via an expression vector or isolated DNA molecule of claim 25 or claim 26; and optionally (b) treating the plant with zinc or manganese or growing the plant under high zinc, high manganese, or high nitrate conditions.

31. A genetically altered plant genome comprising (i) the one or more genetic alterations in the genetically modified plant or part thereof of any one of claims 1-9, or (ii) the one or more genetic alterations in the genetically modified plant or part produced by the method of any one of claims 20-24.

32. A non-regenerable part or cell of the genetically modified plant or part thereof of any one of claims 1-9.
